(19) United States
(12) Patent Application Publication
(10) Pub. No.: US 2026/0193320 A1
(43) Pub. Date: Jul. 9, 2026

(54) CHIMERIC FACTOR VIII POLYPEPTIDES AND USES THEREOF

(71) Applicant: BIOVERATIV THERAPEUTICS INC., Waltham, MA (US)

(72) Inventors: Ekta Seth CHHABRA, Waltham, MA (US); Tongyao LIU, Waltham, MA (US); Robert T. PETERS, Waltham, MA (US); Haiyan JIANG, Waltham, MA (US)

(21) Appl. No.: 19/397,119

(22) Filed: Nov. 21, 2025

Related U.S. Application Data
(62) Division of application No. 17/826,932, filed on May 27, 2022.
Application No. 17/826,932 is a division of application No. 14/371,948, filed on Jan. 12, 2013, now Pat. No. 11,370,827.
(60) Provisional application No. 61/586,099, filed on Jan. 12, 2012.
(60) Provisional application No. 61/586,654, filed on Jan. 13, 2012.
(60) Provisional application No. 61/667,901, filed on Jul. 3, 2012.
(60) Provisional application No. 61/734,954, filed on Dec. 7, 2012.

(51) Int. Cl. C07K 14/755; A61K 38/37; A61K 47/62; C07K 16/00

(52) U.S. Cl. CPC C07K 14/755; A61K 47/62; C07K 16/00; A61K 38/37; C07K 2317/94; C07K 2319/00; C07K 2319/31

(57) ABSTRACT

The present invention provides a VWF fragment comprising the D′ domain and D3 domain of VWF, a chimeric protein comprising the VWF fragment and a heterologous moiety, or a chimeric protein comprising the VWF fragment and a FVIII protein and methods of using the same. A polypeptide chain comprising a VWF fragment of the invention binds to or is associated with a polypeptide chain comprising a FVIII protein and the polypeptide chain comprising the VWF fragment can prevent or inhibit binding of endogenous VWF to the FVIII protein. By preventing or inhibiting binding of endogenous VWF to the FVIII, which is a half-life limiting factor for FVIII, the VWF fragment can induce extension of half-life of the FVIII protein. The invention also includes nucleotides, vectors, host cells, methods of using the VWF fragment, or the chimeric proteins.

### RELATED APPLICATIONS

This application is a divisional of U.S. patent application Ser. No. 17/826,932, filed May 27, 2022, which is a divisional of U.S. patent application Ser. No. 14/371,948, filed Jul. 11, 2014, now U.S. Pat. No. 11,370,827, which is a 35 U.S.C. § 371 filing of International Patent Application No. PCT/US2013/021330, filed Jan. 12, 2013, which claims priority to U.S. Provisional Patent Application Ser. Nos. 61/734,954, filed Dec. 7, 2012; 61/667,901, filed Jul. 3, 2012; 61/586,654, filed Jan. 13, 2012; and 61/586,099, filed Jan. 12, 2012, the entire disclosures of which are hereby incorporated herein by reference.

### BACKGROUND OF THE INVENTION

### Sequence Listing

The instant application contains a Sequence Listing which has been submitted electronically in XML format and is hereby incorporated by reference in its entirety. Said XML file, created on Mar. 23, 2026, is named 772091_SA9-417USDIV2_ST26.xml and is 236,621 bytes in size.

Coagulation is a complex process by which blood forms clots. It is an important part of hemostasis, the cessation of blood loss from a damaged vessel, wherein a damaged blood vessel wall is covered by a platelet and fibrin-containing clot to stop bleeding and begin repair of the damaged vessel. Disorders of coagulation can lead to an increased risk of bleeding (hemorrhage) or obstructive clotting (thrombosis).

Coagulation begins almost instantly after an injury to the blood vessel has damaged the endothelium lining of the vessel. Exposure of the blood to proteins such as tissue factor initiates changes to blood platelets and the plasma protein fibrinogen, a clotting factor. Platelets immediately form a plug at the site of injury; this is called primary hemostasis. Secondary hemostasis occurs simultaneously: Proteins in the blood plasma, called coagulation factors or clotting factors, respond in a complex cascade to form fibrin strands, which strengthen the platelet plug. Non-limiting coagulation factors include, but are not limited to, factor I (fibrinogen), factor II (prothrombin), Tissue factor, factor V (proaccelerin, labile factor), factor VII (stable factor, proconvertin), factor VIII (Antihemophilic factor A), factor IX (Antihemophilic factor B or Christmas factor), factor X (Stuart-Prower factor), factor XI (plasma thromboplastin antecedent), factor XII (Hageman factor), factor XIII (fibrin-stabilizing factor), VWF, prekallikrein (Fletcher factor), high-molecular-weight kininogen (HMWK) (Fitzgerald factor), fibronectin, antithrombin III, heparin cofactor II, protein C, protein S, protein Z, plasminogen, alpha 2-antiplasmin, tissue plasminogen activator (tPA), urokinase, plasminogen activator inhibitor-1 (PAI1), and plasminogen activator inhibitor-2 (PAI2).

Haemophilia A is a bleeding disorder caused by defects in the gene encoding coagulation factor VIII (FVIII) and affects 1-2 in 10,000 male births. Graw et al., Nat. Rev. Genet. 6(6): 488-501 (2005). Patients affected with hemophilia A can be treated with infusion of purified or recombinantly produced FVIII. All commercially available FVIII products, however, are known to have a half-life of about 8-12 hours, requiring frequent intravenous administration to the patients. See Weiner M. A. and Cairo, M. S., Pediatric Hematology Secrets, Lee, M. T., 12. Disorders of Coagulation, Elsevier Health Sciences, 2001; Lillicrap, D. Thromb. Res. 122 Suppl 4: S2-8 (2008). In addition, a number of approaches have been tried in order to extend the FVIII half-life. For example, the approaches in development to extend the half-life of clotting factors include pegylation, glycopegylation, and conjugation with albumin. See Dumont et al., Blood. 119(13): 3024-3030 (Published online Jan. 13, 2012). Regardless of the protein engineering used, however, the long acting FVIII products currently under development have improved half-lives, but the half-lives are reported to be limited-only to about 1.5 to 2 fold improvement in preclinical animal models. See Id. Consistent results have been demonstrated in humans, for example, rFVIIIFc was reported to improve half-life up to ~1.7 fold compared with ADVATE® in hemophilia A patients. See Id. Therefore, the half-life increases, despite minor improvements, may indicate the presence of other T1/2 limiting factors. See Liu, T. et al., 2007 ISTH meeting, abstract #P-M-035; Henrik, A. et al., 2011 ISTH meeting, abstract #P=MO-181; Liu, T. et al., 2011 ISTH meeting abstract #P-WE-131.

Plasma von Willebrand Factor (VWF) has a half-life of approximately 12 hours (ranging from 9 to 15 hours). http://www.nhlbi.nih.gov/guidelines/vwd/2_scientificoverview.htm (last visited Oct. 22, 2011). The VWF half-life may be affected by a number of factors: glycosylation pattern, ADAMTS-13 (a disintegrin and metalloprotease with thrombospondin motif-13), and various mutations in VWF.

In plasma, 95-98% of FVIII circulates in a tight non-covalent complex with full-length VWF. The formation of this complex is important for the maintenance of appropriate plasma levels of FVIII in vivo. Lenting et al., Blood. 92(11): 3983-96 (1998); Lenting et al., J. Thromb. Haemost. 5(7): 1353-60 (2007). The full-length wild-type FVIII is mostly present as a heterodimer having a heavy chain (MW 200 kd) and a light chain (MW 73 kd). When FVIII is activated due to proteolysis at positions 372 and 740 in the heavy chain and at position 1689 in the light chain, the VWF bound to FVIII is removed from the activated FVIII. The activated FVIII, together with activated factor IX, calcium, and phospholipid (“tenase complex”), involves in the activation of factor X, generating large amounts of thrombin. Thrombin, in turn, then cleaves fibrinogen to form soluble fibrin monomers, which then spontaneously polymerize to form the soluble fibrin polymer. Thrombin also activates factor XIII, which, together with calcium, serves to crosslink and stabilize the soluble fibrin polymer, forming cross-linked (insoluble) fibrin. The activated FVIII is cleared fast from the circulation by proteolysis.

Due to the frequent dosing and inconvenience caused by the dosing schedule, there is still a need to develop FVIII products requiring less frequent administration, i.e., a FVIII product that has a half-life longer than the 1.5 to 2 fold half-life limitation.

### BRIEF SUMMARY OF THE INVENTION

The present invention is drawn to a chimeric protein comprising a Factor VIII (“FVIII”) protein and an adjunct moiety (“AM”), wherein the adjunct moiety inhibits or prevents endogenous VWF from binding to the FVIII protein. The FVIII protein and the adjunct moiety are linked to each other by a covalent bond in order to prevent dissociation of the adjunct moiety in the presence of endogenous VWF. In one embodiment, the covalent bond is a peptide bond, a disulfide bond, or a linker, which is strong enough to prevent dissociation of the adjunct moiety from the FVIII protein in the presence of endogenous VWF. In another embodiment, the adjunct moiety prevents the FVIII protein from being cleared through a VWF clearance pathway. In other embodiments, the adjunct moiety inhibits or prevents endogenous VWF from binding to the FVIII protein by shielding or blocking a VWF binding site on the FVIII protein. For example, VWF binding site is located in the A3 domain or the C2 domain of the FVIII protein or both the A3 domain and the C2 domain.

In some embodiments, the chimeric protein includes a construct comprising a FVIII protein and an adjunct moiety linked to each other by a covalent bond, wherein the chimeric protein does not comprise a FVIII half-life limiting factor, which induces a half-life limitation of the FVIII protein, e.g., a full-length VWF protein or a mature VWF protein. Therefore, in some embodiments, the half-life of the FVIII protein of the chimeric protein is extendable beyond the half-life limitation of the FVIII protein in the presence of endogenous VWF.

In certain embodiments, the adjunct moiety has at least one VWF-like FVIII protecting property. Examples of the VWF-like FVIII protecting property include, but are not limited to, protecting the FVIII protein from one or more protease cleavages, protecting the FVIII protein from activation, stabilizing the heavy chain and/or the light chain of the FVIII protein, or preventing clearance of the FVIII protein by one or more scavenger receptors. In one embodiment, the adjunct moiety comprises a polypeptide, a non-polypeptide moiety, or both. In another embodiment, the adjunct moiety can be a polypeptide comprising an amino acid sequence of at least about 40, at least about 50, at least about 60, at least about 70, at least about 80, at least about 90, at least about 100, at least about 110, at least about 120, at least about 130, at least about 140, at least about 150, at least about 200, at least about 250, at least about 300, at least about 350, at least about 400, at least about 450, at least about 500, at least about 550, at least about 600, at least about 650, at least about 700, at least about 750, at least about 800, at least about 850, at least about 900, at least about 950, or at least about 1000 amino acids in length. In certain embodiments, the adjunct moiety comprises a VWF fragment, an immunoglobulin constant region or a portion thereof, albumin or a fragment thereof, an albumin binding moiety, a PAS sequence, a HAP sequence, transferrin or a fragment thereof, or any combinations thereof. In other embodiments, the adjunct moiety is a non-polypeptide moiety comprising polyethylene glycol (PEG), polysialic acid, hydroxyethyl starch (HES), a derivative thereof, or any combinations thereof.

In certain embodiments, the adjunct moiety comprises a VWF fragment comprising a D′ domain and a D3 domain of VWF, wherein the VWF fragment is associated with the FVIII protein by a non-covalent bond in addition to the covalent bond between the FVIII protein and the adjunct moiety (VWF fragment). In one example, the VWF fragment is a monomer. In another example, the VWF fragment comprises two, three, four, five, or six VWF fragments linked to one or more of each other.

In one aspect, the chimeric protein comprises an adjunct moiety, e.g., a VWF fragment, and at least one heterologous moiety (H1) and an optional linker between the adjunct moiety, e.g., VWF fragment, and the heterologous moiety (H1). In one embodiment, the heterologous moiety (H1) can comprise a moiety that extends the half-life of the FVIII protein, e.g., a polypeptide selected from the group consisting of an immunoglobulin constant region or a portion thereof, albumin or a fragment thereof, an albumin binding moiety, a PAS sequence, a HAP sequence, transferrin or a fragment thereof, and any combinations thereof or a non-polypeptide moiety selected from the group consisting of polyethylene glycol (PEG), polysialic acid, hydroxyethyl starch (HES), a derivative thereof, and any combinations thereof. In one embodiment, the heterologous moiety (H1) comprises a first Fc region. In another embodiment, the heterologous moiety (H1) comprises an amino acid sequence comprising at least about 50 amino acids, at least about 100 amino acids, at least about 150 amino acids, at least about 200 amino acids, at least about 250 amino acids, at least about 300 amino acids, at least about 350 amino acids, at least about 400 amino acids, at least about 450 amino acids, at least about 500 amino acids, at least about 550 amino acids, at least about 600 amino acids, at least about 650 amino acids, at least about 700 amino acids, at least about 750 amino acids, at least about 800 amino acids, at least about 850 amino acids, at least about 900 amino acids, at least about 950 amino acids, or at least about 1000 amino acids. In other embodiments, the chimeric protein comprises a linker between the adjunct moiety, e.g., a VWF fragment, and the heterologous moiety (H1), which is a cleavable linker.

In another aspect, the FVIII protein in the chimeric protein comprises FVIII and at least one heterologous moiety (H2). In one embodiment, the heterologous moiety (H2) is capable of extending the half-life of the FVIII protein, e.g., a polypeptide selected from the group consisting of an immunoglobulin constant region or a portion thereof, albumin or a fragment thereof, an albumin binding moiety, a PAS sequence, a HAP sequence, transferrin or a fragment thereof, and any combinations thereof or a non-polypeptide moiety comprising polyethylene glycol (PEG), polysialic acid, hydroxyethyl starch (HES), a derivative thereof, and any combinations thereof. In a particular embodiment, the heterologous moiety (H2) comprises a second Fc region.

In some embodiments, the chimeric protein comprises a first polypeptide chain comprising the VWF fragment, a first heterologous moiety, and a linker and a second polypeptide chain comprising the FVIII protein and a second heterologous moiety, wherein the first polypeptide chain and the second polypeptide chain are linked to each other by a covalent bond. In one example, the first heterologous moiety and the second heterologous moiety are linked to each other by the covalent bond, e.g., a disulfide bond, a peptide bond, or a linker, wherein the covalent bond prevents replacement of the VWF fragment in the first polypeptide chain with endogenous VWF in vivo. In some embodiments, the linker between the FVIII protein and the second heterologous moiety is a cleavable linker.

In certain embodiments, the first heterologous moiety (H1) linked to the VWF fragment and the second heterologous moiety (H2) linked to the FVIII protein are linked by a linker, e.g., a scFc linker, which is a processable linker.

In yet other embodiments, the FVIII protein in the chimeric protein further comprises a third heterologous moiety (H3), a fourth heterologous moiety (H4), a fifth heterologous moiety (H5), a sixth heterologous moiety (H6), or any combinations thereof. In one embodiment, one or more of the third heterologous moiety (H3), the fourth heterologous moiety (H4), the fifth heterologous moiety (H5), the sixth heterologous moiety (H6) are capable of extending the half-life of the FVIII protein. In another embodiments, the third heterologous moiety (H3), the fourth heterologous moiety (H4), the fifth heterologous moiety (H5), and the sixth heterologous moiety (H6) are linked to the C terminus or N terminus of FVIII or inserted between two amino acids of FVIII. In other embodiments, one or more of the third heterologous moiety (H3), the fourth heterologous moiety (H4), the fifth heterologous moiety (H5), or the sixth heterologous moiety (H6) comprises an amino acid sequence comprising at least about 50 amino acids, at least about 100 amino acids, at least about 150 amino acids, at least about 200 amino acids, at least about 250 amino acids, at least about 300 amino acids, at least about 350 amino acids, at least about 400 amino acids, at least about 450 amino acids, at least about 500 amino acids, at least about 550 amino acids, at least about 600 amino acids, at least about 650 amino acids, at least about 700 amino acids, at least about 750 amino acids, at least about 800 amino acids, at least about 850 amino acids, at least about 900 amino acids, at least about 950 amino acids, or at least about 1000 amino acids.

In some embodiments, the linker between the FVIII protein and the second heterologous moiety or the linker between the VWF fragment and the first heterologous moiety further comprises a first cleavage site (P1) at the N-terminal region of the linker, a second cleavage site (P2) at the C-terminal region of the linker, or both. In other embodiments, one or more of the linker between the FVIII protein and the adjunct moiety, the linker between the FVIII protein and the second heterologous moiety, and the linker between the VWF fragment and the first heterologous moiety have a length of about 1 to about 2000 amino acids.

In other embodiments, the chimeric protein comprises a FVIII protein and an adjunct moiety, which are linked by a linker between the FVIII protein and the adjunct moiety, wherein the linker further comprises a sortase recognition motif, e.g., the sequence of LPXTG (SEQ ID NO: 106).

The present invention is directed to a von Willebrand Factor (VWF) fragment comprising the D′ domain and the D3 domain of VWF, wherein the VWF fragment binds to Factor VIII (FVIII) and inhibits binding of endogenous VWF to a FVIII protein. In one embodiment, the VWF fragment of the invention is not amino acids 764 to 1274 of SEQ ID NO: 2. In one embodiment, the FVIII protein, without the VWF fragment, has a half-life comparable to wild-type FVIII. In another embodiment, the FVIII protein is a fusion protein comprising FVIII and a heterologous moiety that is capable of extending half-life of FVIII. The heterologous moiety can be a polypeptide, a non-polypeptide moiety, or both. The heterologous polypeptide moiety can be selected from the group consisting of an immunoglobulin constant region or a portion thereof, albumin or a fragment thereof, an albumin binding moiety, a PAS sequence, a HAP sequence, transferrin or a fragment thereof, and any combination thereof. In other embodiments, the heterologous moiety is an immunoglobulin constant region or a portion thereof, e.g., an Fc region. In still other embodiments, the non-polypeptide moiety is selected from the group consisting of polyethylene glycol (PEG), polysialic acid, hydroxyethyl starch (HES), a derivative thereof, and any combinations thereof. In certain embodiments, The FVIII protein comprises a first polypeptide chain and a second polypeptide chain, wherein the first polypeptide chain comprises FVIII and a first Fc region and the second polypeptide chain comprises a second Fc region without FVIII.

In another embodiment, the VWF fragment extends a half-life of FVIII. The amino acid sequence of the D′ domain can be at least 90%, 95%, 96%, 97%, 98%, 99%, or 100% identical to amino acids 764 to 866 of SEQ ID NO: 2. Also, the amino acid sequence of the D3 domain can be at least 90%, 95%, 96%, 97%, 98%, 99%, or 100% identical to amino acids 867 to 1240 of SEQ ID NO: 2. In certain embodiments, the VWF fragment contains at least one amino acid substitution at a residue corresponding to residue 1099, residue 1142, or both of SEQ ID NO: 2. In a particular embodiment, a VWF fragment comprises, consisting essentially of, or consists of amino acids 764 to 1240 of SEQ ID NO: 2. The VWF fragment can further comprise the D1 domain, the D2 domain, or the D1 and D2 domains of VWF. In some embodiments, the VWF fragment further comprises a VWF domain selected from the group consisting of the A1 domain, the A2 domain, the A3 domain, the D4 domain, the B1 domain, the B2 domain, the B3 domain, the C1 domain, the C2 domain, the CK domain, one or more fragments thereof, and any combinations thereof. In other embodiments, the VWF fragment is pegylated, glycosylated, hesylated, or polysialylated.

The present invention is also directed to a chimeric protein comprising a VWF fragment described herein, a heterologous moiety, and an optional linker between the VWF fragment and the heterologous moiety. The heterologous moiety can be a polypeptide, a non-polypeptide moiety, or both. In one embodiment, the heterologous polypeptide moiety is selected from the group consisting of an immunoglobulin constant region or a portion thereof, albumin or a fragment thereof, an albumin binding moiety, a PAS sequence, a HAP sequence, transferrin or a fragment thereof, and any combination thereof. In another embodiment, the heterologous non-polypeptide moiety is selected from group consisting of polyethylene glycol (PEG), polysialic acid, hydroxyethyl starch (HES), a derivative thereof, and any combinations thereof. In a particular embodiment, the heterologous moiety is a first Fc region. The chimeric protein can further comprise a second Fc region, wherein the second Fc region is linked to or associated with the first Fc region or linked to or associated with the VWF fragment.

In one aspect, a chimeric protein of the invention comprises a formula selected from the group consisting of:

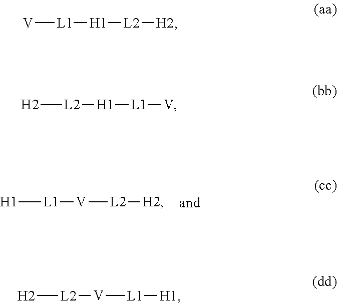

wherein the V is one or more of the VWF fragments described herein,
each of L1 and L2 is an optional linker;
H1 is a first heterologous moiety;
(-) is a peptide bond or one or more amino acids; and
H2 is an optional second heterologous moiety.

In one embodiment, H1 is a first heterologous moiety, e.g., a half-life extending molecule which is known in the art. In one embodiment, the first heterologous moiety is a polypeptide. The first heterologous polypeptide moiety is selected from the group consisting of an immunoglobulin constant region or a portion thereof, albumin or a fragment thereof, an albumin binding moiety, a PAS sequence, a HAP sequence, transferrin or a fragment thereof, and any combinations thereof. In another embodiment, H1 is a non-polypeptide moiety selected from the group consisting of polyethylene glycol (PEG), polysialic acid, hydroxyethyl starch (HES), a derivative thereof, and any combinations thereof. H2 is an optional second heterologous moiety, e.g., a half-life extending molecule which is known in the art. In one embodiment, the second heterologous moiety can be selected from the group consisting of an immunoglobulin constant region or a portion thereof, albumin or a fragment thereof, an albumin binding moiety, a PAS sequence, a HAP sequence, transferrin or a fragment thereof, and any combination thereof. In another embodiment, H2 is a non-polypeptide moiety, which is selected from the group consisting of polyethylene glycol (PEG), polysialic acid, hydroxyethyl starch (HES), a derivative thereof, and any combinations thereof. In certain embodiments, H1 is a first Fc region and H2 is a second Fc region. The first Fc region and the second Fc region can be the same or different and can be linked to each other by a linker or a covalent bond, e.g., a disulfide bond. In another embodiment, the second Fc region is linked to or associated with a Factor VIII protein. Optionally, there could be a third heterologous moiety, H3, which is a half-life extender, which is linked to the VWF fragment, the first heterologous moiety, or the second heterologous moiety. Non-limiting examples of the third heterologous moiety can include a polypeptide or a non-polypeptide moiety or both. In one embodiment, the third heterologous polypeptide moiety can be selected from the group consisting of an immunoglobulin constant region or a portion thereof, albumin or a fragment thereof, an albumin binding moiety, a PAS sequence, a HAP sequence, transferrin or a fragment thereof, or any combinations thereof. In another embodiment, H2 is a non-polypeptide moiety, which is be selected from the group consisting of polyethylene glycol (PEG), polysialic acid, hydroxyethyl starch (HES), a derivative thereof, and any combinations thereof. In some embodiments, H3 is linked to the VWF fragment or the first or the second heterologous moiety by a cleavable linker, e.g., a thrombin cleavable linker. Non-limiting examples of the linkers are disclosed elsewhere herein.

In another aspect, the invention provides a chimeric protein comprising a VWF fragment described herein, a FVIII protein, and an optional linker between the VWF fragment and the FVIII protein. The VWF fragment can be bound to the FVIII protein. In one embodiment, a chimeric protein comprises a VWF fragment described herein, which is linked to a heterologous moiety. The heterologous moiety can be a moiety that extends the half-life of the protein, which comprises a polypeptide, a non-polypeptide moiety, or both. Examples of such a heterologous polypeptide moiety include, e.g., an immunoglobulin constant region or a portion thereof, albumin or a fragment thereof, an albumin binding moiety, a PAS sequence, a HAP sequence, any derivatives or variants thereof, or any combinations thereof. Examples of a non-polypeptide moiety include, e.g., polyethylene glycol (PEG), polysialic acid, hydroxyethyl starch (HES), a derivative thereof, or any combinations thereof. In another embodiment, the heterologous moiety is a first Fc region linked to the VWF fragment. In other embodiments, the chimeric protein further comprises a second Fc region linked to the FVIII protein. The VWF fragment or the FVIII protein can be linked to the first Fc region or the second Fc region, respectively, by a linker. In still other embodiments, a chimeric protein comprises a VWF fragment described herein linked to a first heterologous moiety, e.g., first Fc region, and a FVIII protein linked to a second heterologous moiety, e.g., second Fc region, wherein the VWF fragment is further linked to the second heterologous moiety (e.g., second Fc region) or the FVIII protein by a linker or by covalent bond or the first heterologous moiety (e.g., Fc region) is further linked to the FVIII protein or the second heterologous moiety (e.g., second Fc region) by a linker or a covalent bond. In some embodiments, the FVIII of the chimeric protein has a partial B-domain. In some embodiments, the FVIII protein with a partial B-domain is FVIII198 (SEQ ID NO: 105). In other embodiments, the chimeric protein further comprises a sortase recognition motif.

In some embodiments, as a result of the invention the half-life of the FVIII protein is extended compared to a FVIII protein without the VWF fragment or wildtype FVIII. The half-life of the FVIII protein is at least about 1.5 times, at least about 2 times, at least about 2.5 times, at least about 3 times, at least about 4 times, at least about 5 times, at least about 6 times, at least about 7 times, at least about 8 times, at least about 9 times, at least about 10 times, at least about 11 times, or at least about 12 times longer than the half-life of a FVIII protein without the VWF fragment. In one embodiment, the half-life of FVIII is about 1.5-fold to about 20-fold, about 1.5 fold to about 15 fold, or about 1.5 fold to about 10 fold longer than the half-life of wild-type FVIII. In another embodiment, the half-life of the FVIII is extended about 2-fold to about 10-fold, about 2-fold to about 9-fold, about 2-fold to about 8-fold, about 2-fold to about 7-fold, about 2-fold to about 6-fold, about 2-fold to about 5-fold, about 2-fold to about 4-fold, about 2-fold to about 3-fold, about 2.5-fold to about 10-fold, about 2.5-fold to about 9-fold, about 2.5-fold to about 8-fold, about 2.5-fold to about 7-fold, about 2.5-fold to about 6-fold, about 2.5-fold to about 5-fold, about 2.5-fold to about 4-fold, about 2.5-fold to about 3-fold, about 3-fold to about 10-fold, about 3-fold to about 9-fold, about 3-fold to about 8-fold, about 3-fold to about 7-fold, about 3-fold to about 6-fold, about 3-fold to about 5-fold, about 3-fold to about 4-fold, about 4-fold to about 6 fold, about 5-fold to about 7-fold, or about 6-fold to about 8 fold as compared to wild-type FVIII or a FVIII protein without the VWF fragment. In other embodiments, the half-life of FVIII is at least about 17 hours, at least about 18 hours, at least about 19 hours, at least about 20 hours, at least about 21 hours, at least about 22 hours, at least about 23 hours, at least about 24 hours, at least about 25 hours, at least about 26 hours, at least about 27 hours, at least about 28 hours, at least about 29 hours, at least about 30 hours, at least about 31 hours, at least about 32 hours, at least about 33 hours, at least about 34 hours, at least about 35 hours, at least about 36 hours, at least about 48 hours, at least about 60 hours, at least about 72 hours, at least about 84 hours, at least about 96 hours, or at least about 108 hours. In still other embodiments, the half-life of FVIII is about 15 hours to about two weeks, about 16 hours to about one week, about 17 hours to about one week, about 18 hours to about one week, about 19 hours to about one week, about 20 hours to about one week, about 21 hours to about one week, about 22 hours to about one week, about 23 hours to about one week, about 24 hours to about one week, about 36 hours to about one week, about 48 hours to about one week, about 60 hours to about one week, about 24 hours to about six days, about 24 hours to about five days, about 24 hours to about four days, about 24 hours to about three days, or about 24 hours to about two days.

In some embodiments, the average half-life of the FVIII protein per subject is about 15 hours, about 16 hours, about 17 hours, about 18 hours, about 19 hours, about 20 hours, about 21 hours, about 22 hours, about 23 hours, about 24 hours (1 day), about 25 hours, about 26 hours, about 27 hours, about 28 hours, about 29 hours, about 30 hours, about 31 hours, about 32 hours, about 33 hours, about 34 hours, about 35 hours, about 36 hours, about 40 hours, about 44 hours, about 48 hours (2 days), about 54 hours, about 60 hours, about 72 hours (3 days), about 84 hours, about 96 hours (4 days), about 108 hours, about 120 hours (5 days), about six days, about seven days (one week), about eight days, about nine days, about 10 days, about 11 days, about 12 days, about 13 days, or about 14 days.

In another aspect, a chimeric protein of the invention comprises a formula selected from the group consisting of:

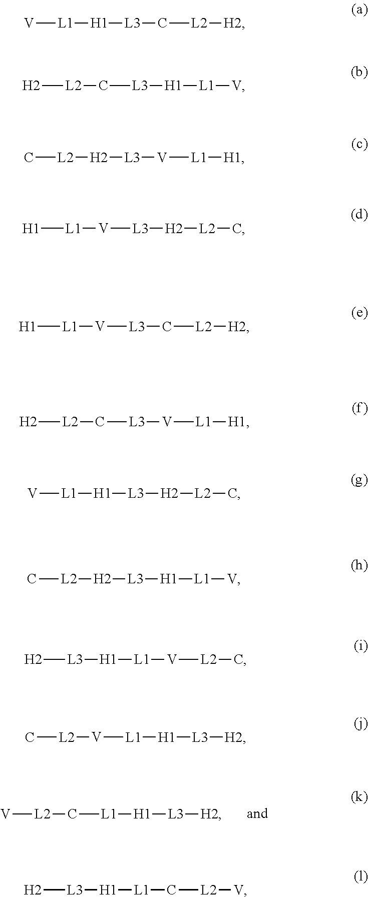

wherein V is a VWF fragment described herein;
each of L1 or L2, is an optional linker, e.g., a thrombin cleavable linker;
L3 is an optional linker, e.g., scFc linker, e.g., a processable linker;
each of H1 or H2 is an optional heterologous moiety; and
C is a FVIII protein; and
(-) is a peptide bond or one or more amino acids.

In other aspects, a chimeric protein of the invention comprises a formula selected from the group consisting of:

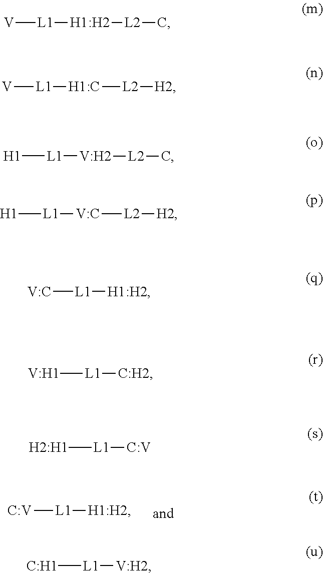

wherein V is a VWF fragment described herein;
each of L1 or L2, is an optional linker, e.g., a thrombin cleavable linker;
each of H1 or H2 is an optional heterologous moiety; and
C is a FVIII protein;
(-) is a peptide bond or one or more amino acids; and
(:) is a chemical or physical association between H1 and H2, between V and C, and between V and H1 and C and H2. (:) represents a chemical association, e.g., at least one non-peptide bond. In certain embodiments, the chemical association, i.e., (:) is a covalent bond. In some embodiments, the association between H1 and H2 is a covalent bond, e.g., a disulfide bond. In other embodiments, the chemical association, i.e., (:) is a non-covalent interaction, e.g., an ionic interaction, a hydrophobic interaction, a hydrophilic interaction, a Van der Waals interaction, a hydrogen bond. In certain embodiments, the association between the FVIII protein and the VWF fragment is a non-covalent bond. In other embodiments, (:) is a non-peptide covalent bond. In still other embodiments, (:) is a peptide bond. In one embodiment, H1 is a first heterologous moiety. In one embodiment, the first heterologous moiety is capable of extending half-life of the FVIII activity. In another embodiment, the first heterologous moiety is a polypeptide, a non-polypeptide moiety, or both. In one embodiment, the first heterologous polypeptide moiety can be selected from the group consisting of an immunoglobulin constant region or a portion thereof, albumin or fragment thereof, an albumin binding moiety, a PAS sequence, a HAP sequence, transferrin or a fragment thereof, and any combinations thereof. In another embodiment, the non-polypeptide moiety is selected from the group consisting of polyethylene glycol (PEG), polysialic acid, hydroxyethyl starch (HES), a derivative thereof, and any combinations thereof. In some embodiments, H2 is a second heterologous moiety. The second heterologous moiety can also be a half-life extender known in the art and can be a polypeptide, a non-polypeptide moiety, or a combination of both. In one embodiment, the second heterologous moiety is selected from the group consisting of an immunoglobulin constant region or a portion thereof, albumin or fragment thereof, an albumin binding moiety, a PAS sequence, a HAP sequence, transferrin or a fragment thereof, and any combinations thereof. In certain embodiments, the non-polypeptide moiety is selected from the group consisting of polyethylene glycol (PEG), polysialic acid, hydroxyethyl starch (HES), a derivative thereof, and any combinations thereof. In a particular embodiment, H1 is a first Fc region. In some embodiments, H2 is a second Fc region. Optionally, there could be a third heterologous moiety, H3, which is a half-life extender. H3 can be linked to one or more of V, C, H1, or H2 by an optional linker, e.g., a cleavable linker, e.g., a thrombin cleavable linker. Non-limiting examples of the third heterologous moiety can include an immunoglobulin constant region or a portion thereof, albumin or a fragment thereof, polyethylene glycol (PEG), a PAS sequence, and hydroxyethyl starch (HES) or a derivative thereof.

In certain embodiments, one or more of the linkers used to connect the VWF fragment, the FVIII protein, the first heterologous moiety, and/or the second heterologous moiety of formulas (a) to (u) to each other is a cleavable linker. One or more of the cleavage sites used in the chimeric protein can be cleaved by a protease selected from the group consisting of factor XIa, factor XIIa, kallikrein, factor VIIa, factor IXa, factor Xa, factor IIa (thrombin), Elastase-2, Granzyme-B, TEV, Enterokinase, Protease 3C, Sortase A, MMP-12, MMP-13, MMP-17, and MMP-20. In other embodiments, one or more linkers used in formulas (a) to (1) (e.g., L3) comprise a processable linker. The processable linkers can be cleaved by an intracellular enzyme upon secretion. The processable linker can comprise a first cleavage site (P1) at the N-terminal region of the linker, a second cleavage site (P2) at the C-terminal region of the linker, or both.

In some embodiments, one or more of the linkers used in the invention have a length of at least about 1 to 2000 amino acids. In a specific embodiment, one or more of the linkers used in the invention have a length of at least about 20, 35, 42, 48, 73, 98, 144, 288, 324, 576, or 864 amino acids. In a particular embodiment, one or more of the linkers comprise a gly/ser peptide. The gly/ser peptide can be (Gly4 Ser)₃ or (Gly4 Ser)₄.

In other aspects, a FVIII protein in a chimeric protein is a functional Factor VIII protein. The FVIII protein can comprise one or more domains of FVIII selected from the group consisting of the A1 domain, the A2 domain, the B domain, the A3 domain, the C1 domain, the C2 domain, one or more fragment thereof, and any combinations thereof. In one embodiment, the FVIII protein comprises the B domain or a portion thereof. In another embodiment, the FVIII protein is SQ B domain deleted FVIII. In other embodiments, the FVIII protein comprises single chain FVIII. In still other embodiments, the FVIII protein comprises a heavy chain of FVIII and a light chain of Factor VIII, wherein the heavy chain and the light chain are associated with each other by a metal bond. In certain embodiments, the FVIII protein has a low affinity to or does not bind to a low-density lipoprotein receptor-related protein (LRP). For example, a FVIII protein useful for the invention can contain at least one amino acid substitution that lowers the affinity to or eliminates the binding to the LRP. Non-limiting examples of the at least one amino acid substitution is at a residue corresponding to residue 471, residue 484, residue 487, residue 490, residue 497, residue 2092, residue 2093 or two or more combinations thereof of full-length mature FVIII. In some embodiments, the FVIII protein in a chimeric protein of this invention contains at least one amino acid substitution, which induces the FVIII protein to be more stable than a FVIII protein without the substitution. In other embodiments, the FVIII protein contains at least one amino acid substitution in the A2 domain and at least one amino acid substitution in the A3 domain, wherein the A2 domain and the A3 domain are associated to each other by a covalent bond. Non-limiting examples of the amino acid substitution in the A2 domain is at a residue corresponding residue 662 or 664 of full-length mature FVIII. In addition, non-limiting examples of the amino acid substitution in the A3 domain is at a residue corresponding to residue 1826 or 1828 of full-length mature FVIII is polysialylated.

In further aspects, the invention provides a polynucleotide encoding a VWF fragment described herein or a chimeric protein described herein, or a set of polynucleotides comprising a first nucleotide chain and a second nucleotide chain, wherein the first nucleotide chain encodes the VWF fragment and the second nucleotide chain encodes the second Fc region or the clotting factor or fragment thereof of the chimeric protein. In one embodiment, the set of polynucleotides further comprises a third polynucleotide chain, which encodes a proprotein convertase belongs to the subtilisin-like proprotein convertase family. Non-limiting examples of the proprotein convertase include proprotein convertase subtilisin/kexin type 3 (PACE or PCSK3), proprotein convertase subtilisin/kexin type 5 (PCSK5 or PC5), proprotein convertase subtilisin/kexin type 7 (PCSK7 or PC7), or a yeast Kex 2. In still other aspects, the invention includes a vector comprising the polynucleotide or the set of polynucleotides and one or more promoters operably linked to the polynucleotide or the set of polynucleotides or a set of vectors comprising a first vector and a second vector, wherein the first vector encodes the first polynucleotide chain of the set of polynucleotides and the second vector encodes the second polynucleotide chain of the set of polynucleotides. The set of vectors can further comprise a third vector, which comprises a third polynucleotide chain encoding PC5 or PC7. In some embodiments, the vector further comprises PACE. In some embodiments, PACE cleaves the DID2 domains of the VWF fragment.

In some aspects, the invention is directed to a pharmaceutical composition comprising the VWF fragment, the chimeric protein, the polynucleotide, the set of polynucleotides, the vector, or the set of vectors, and a pharmaceutically acceptable carrier. The composition of this invention can extend the half-life of Factor VIII. In other aspects, the invention includes a host cell comprising the polynucleotide, the set of polynucleotides, the vector, or the sets of vectors.

In other aspects, the present invention is drawn to a chimeric protein comprising a FVIII protein, an adjunct moiety and an optional linker, wherein the adjunct moiety inhibits or prevents endogenous VWF from binding to the FVIII protein and has at least one VWF-like FVIII protecting property. The VWF-like FVIII protecting property comprises protecting the FVIII protein from one or more protease cleavages, protecting the FVIII protein from activation, stabilizing the heavy chain and/or the light chain of the FVIII protein, or preventing clearance of the FVIII protein by one or more scavenger receptors.

The adjunct moiety in the chimeric protein can inhibit or prevent endogenous VWF from binding to the FVIII protein by shielding or blocking a VWF binding site on the FVIII protein. In some embodiments, the VWF binding site is located in the A3 domain or the C2 domain of the FVIII protein or both A3 domain and C2 domain of the FVIII protein. In another embodiment, the VWF binding site is the amino acid sequence corresponding to amino acids 1669 to 1689 and 2303 to 2332 of SEQ ID NO: 16. In some embodiments, the adjunct moiety is a polypeptide, a non-polypeptide moiety, or both. The polypeptide useful as the adjunct moiety can comprise an amino acid sequence of at least 40, 50, 60, 70, 80, 90, 100, 110, 120, 130, 140, 150, 200, 250, 300, 350, 400, 450, 500, 550, 600, 650, 700, 750, 800, 850, 900, 950, or 1000 amino acids in length. For example, the polypeptide useful as an adjunct moiety can be selected from the group consisting of a VWF fragment, an immunoglobulin constant region or a portion thereof, albumin or a fragment thereof, an albumin binding moiety, a PAS sequence, a HAP sequence, other half-life extending technologies, and any combinations thereof. The non-polypeptide moiety useful as an adjunct moiety can be selected from the group consisting of polyethylene glycol (PEG), polysialic acid, hydroxyethyl starch (HES) or a derivative thereof, and any combinations thereof. In one embodiment, the adjunct moiety is the VWF fragment described herein. The adjunct moiety and the FVIII protein can be linked, e.g., by a linker, or associated with each other. The linker can comprise a cleavable linker, e.g., a thrombin cleavable linker.

In one aspect, the invention provides a method of preventing or inhibiting binding of a FVIII protein with endogenous VWF comprising adding an effective amount of the VWF fragment, the chimeric protein, the polynucleotide, or the set of polynucleotides to a cell comprising a FVIII protein or a polynucleotide encoding the FVIII protein, wherein the VWF fragment binds to the FVIII protein. In another aspect, the invention includes a method of preventing or inhibiting binding of the FVIII protein with endogenous VWF comprising adding an effective amount of the chimeric protein, the polynucleotide, or the set of polynucleotides to a subject in need thereof, wherein the VWF fragment binds to the FVIII protein and thus prevents or inhibits binding of the FVIII protein. In some aspects, the invention includes a method of extending or increasing half-life of a FVIII protein, wherein the method comprises adding an effective amount of the VWF fragment, the chimeric protein, the polynucleotide, or the set of polynucleotides to a cell comprising a FVIII protein or a polynucleotide encoding the FVIII protein or to a subject in need thereof, wherein the VWF fragment binds to the FVIII protein. In other aspects, the invention is drawn to a method of preventing or inhibiting clearance of a FVIII protein from a cell, wherein the method comprises adding an effective amount of the VWF fragment, the chimeric protein, the polynucleotide, or the set of polynucleotides to a cell comprising a FVIII protein or a polynucleotide encoding the FVIII protein or to a subject in need thereof, wherein the VWF fragment binds to the FVIII protein.

In another aspect, the invention is directed to a method of treating a bleeding disease or disorder in a subject in need thereof comprising administering an effective amount of the VWF fragment, the chimeric protein, the polynucleotide, or the set of polynucleotides, wherein the bleeding disease or disorder is selected from the group consisting of a bleeding coagulation disorder, hemarthrosis, muscle bleed, oral bleed, hemorrhage, hemorrhage into muscles, oral hemorrhage, trauma, trauma capitis, gastrointestinal bleeding, intracranial hemorrhage, intra-abdominal hemorrhage, intrathoracic hemorrhage, bone fracture, central nervous system bleeding, bleeding in the retropharyngeal space, bleeding in the retroperitoneal space, and bleeding in the illiopsoas sheath. In other embodiments, the treatment is prophylactic or on-demand. In still other embodiments, the invention is a method of treating a disease or disorder associated with Type 2N von Willebrand's disease to a subject in need thereof, comprising administering an effective amount of the VWF fragment, the chimeric protein, the polynucleotide, or the set of polynucleotides, wherein the disease or disorder is treated.

### BRIEF DESCRIPTION OF THE DRAWINGS/FIGURES

FIG. 1A-F. Schematic diagrams of VWF proteins. FIG. 1A shows two VWF fragments containing amino acids 1 to 276 of SEQ ID NO: 73 (amino acids 764 to 1039 of SEQ ID NO: 2). VWF-001 is synthesized without the pre/propeptide sequences of VWF, while VWF-009 is synthesized with the pre/propeptide sequences (D1 and D2 domains). The prepeptide of VWF-009 is cleaved during synthesis, and VWF-009 contains the propeptide with the D′ and D3 domain sequences. FIG. 1B shows three VWF fragments containing amino acids 1 to 477 of SEQ ID NO: 73 (amino acids 764 to 1240 of SEQ ID NO: 2). VWF-002 is synthesized without the pre/propeptide sequences. VWF-010 contains the DID2 domains in addition to the D′D3 domains. VWF-013 contains the DID2D′D3 domains in addition to alanine residues substituting cysteines at residues 336 and 379 of SEQ ID NO: 72. FIG. 1C shows two VWF fragments containing the D′D3 domains and a portion of the A1 domain. VWF-003 has amino acids 764 to 1274 of SEQ ID NO: 2). VWF-011 contains the DID2 domains in addition to the D′D3 domains. FIG. 1D shows two constructs, VWF-004 and VWF-012. VWF-004 contains the D′D3 domains and the complete sequence of A1 domain. VWF-012 contains the DID2D′D3 domains and the complete sequence of A1 domain. FIG. 1E shows three constructs. VWF-006 contains the DID2D′D3 domains and the CK domain of VWF (cysteine knot domain). VWF-008 is the full-length VWF. VWF-031 (VWF-Fc) shows a construct containing the DID2D′D3 domains linked to a single Fc region by a cleavable linker. VWF-053 is the DID2 domains. FIG. 1F shows full-length VWF protein comprising propeptide (the D1 and D2 domains) and mature subunits (the D′, D3, A1, A2, A3, D4, B1-3, C1-2 domains). The VWF protein is about 250 kDa protein and forms multimers (>20 MDa) by disulfide bonding. The VWF protein associates with FVIII (95-98%) in non-covalent complex and then extends half-life of FVIII by protecting FVIII from protease cleavage/activation, stabilizing heavy & light chain, and preventing clearance of FVIII by scavenger receptors. The VWF protein also can limit half-life of FVIII by clearance of FVIII-VWF complex through VWF receptors and preventing pinocytosis and recycling of rFVIIIFc.

FIGS. 2A-2B. Schematic diagrams of examples of VWF:FVIII heterodimer constructs. The left construct shows a VWF fragment having the D′D3 domains of full-length VWF (amino acids 1-477 of SEQ ID NO: 73) and containing alanine substitutions at residues 336 and 379 of SEQ ID NO: 72. The chimeric protein construct (FVIII 064/065) comprises the C-terminus of a VWF fragment linked to a first Fc region by a linker and FVIII is linked to a second Fc region, wherein the second Fc region is further linked to the N-terminus of a VWF fragment by a linker (e.g., formula C-H1-L1-V-L2-H2, wherein V is a VWF fragment, C is FVIII, H1 and H2 are Fc regions, and L1 and L2 are cleavable linkers). The construct in FIG. 2b is an intracellularly processed VWF:FVIII heterodimer construct where the linker between the second Fc and the N-terminus of the VWF fragment has been cleaved. FVIII-064 contains the D′D3 domains of VWF (amino acids 1 to 477 of SEQ ID NO: 73 with C336A and C379 substitutions). FVIII-065 contains the D′D3 domains of VWF (amino acids 1 to 276 of SEQ ID NO: 73). FVIII-136 contains FVIIIFc linked to the D′D3 fragment-Fc by a linker that can be processed by an intracellular protease enzyme. When FVIII-136 is expressed, the enzyme cleaves the linker between the second Fc (fused to FVIII-LC) and the VWF D′D3 fragment (fused to the first Fc), while the Fc region fused to (or linked to) FVIII-LC forms a covalent bond (e.g., a disulfide bond) with the first Fc fused to (or linked to) the VWF fragment. FVIII-148 is single chain FVIIIFc with the D′D3 fragment (a single chain FVIII by introducing R1645A/R1648A mutation into FVIII gene).

FIG. 3. Schematic diagrams of examples of VWF:FVIII heterodimer constructs containing examples of variable linkers between VWF and Fc. The constructs (FVIII-064, FVIII-159, FVIII-160, FVIII-178, and FVIII-179) have the common structure represented as formula C-H1-L1-V-L2-H2, but contain examples of different linkers or amino acid substitutions. The constructs shown contain the same VWF fragment, which is the D′ and D3 domains of VWF (i.e., amino acids 1 to 477 of SEQ ID NO: 73 with amino acid substitutions C336A and C379A). Construct FVIII 64 has a thrombin cleavable linker (i.e., L2) between the VWF fragment and the Fc (i.e., H2), which has 20 amino acids. Construct FVIII 159 has a thrombin cleavable linker (i.e., L2) between the VWF fragment and the Fc (i.e., H2), which has 35 amino acids. Construct FVIII 160 has a thrombin cleavable linker (i.e., L2) between the VWF fragment and the Fc (i.e., H2), which has 48 amino acids. Constructs FVIII-180, FVIII-181, and FVIII-182 are derivatives of FVIII-160 containing K2092A mutation in FVIII C1 domain, K2093A mutation in FVIII C1 domain, and K2092A/K2093A mutations in FVIII C1 domain, respectively. Construct FVIII-178 has a thrombin cleavable linker (i.e., L2) between the VWF fragment and the Fc (i.e., H2), which has 73 amino acids. Construct FVIII-179 has a thrombin cleavable linker (i.e., L2) between the VWF fragment and the Fc (i.e., H2), which has 98 amino acids.

FIGS. 4A-4H: Schematic diagrams of examples of FVIII-VWF constructs, in which VWF is DID2D′D3 fragment of VWF, the Linker is a variable length linker containing a cleavage site, e.g., a thrombin cleavage site, SC FVIII is a single chain FVIII, which contains the R1645A/R1648A substitutions, H is a heterologous moiety, e.g., an immunoglobulin constant region or a portion thereof, a moiety for conjugating polyethylene glycol (PEG) and/or PEG, an albumin or albumin fragment, an albumin binding moiety, a HAP sequence, a moiety for polysialylation and/or polysialic acid, a moiety for hydroxyethyl starch (HES) and/or HES, or a PAS sequence, etc., HC FVIII is a heavy chain of FVIII, LC FVIII is a light chain of FVIII, and Fc is an Fc region of an immunoglobulin constant region. FIG. 4A has a formula of VWF-Linker-SC FVIII. FIG. 4B has a formula of VWF-Linker-H-Linker-SC FVIII. The linkers (the first linker between VWF and H and the second linker between H and SC FVIII) can be identical or different. FIG. 4C has a formula of VWF-Linker-SC FVIII-Linker-H. The linkers (the first linker between VWF and SC FVIII and the second linker between SC FVIII and H) can be identical or different. FIG. 4D has a formula of VWF-Linker-HC FVIII-H-Linker-LC FVIII. The linkers (the first linker between VWF and HC FVIII and the second linker between H and LC FVIII) can be identical or different. FIG. 4E has a formula of HC FVIII-H-LC FVIII-Linker-first Fc-Linker-VWF-Linker-second Fc. The linkers (the first linker between LC FVIII and first Fc, the second linker between first Fc and VWF, and the third linker between VWF and second Fc) can be identical or different. The linkers can be a cleavable linker. For example, the linker between first Fc and VWF can be a cleavable linker comprising a cleavage site at the N-terminus and/or the C-terminus of the linker. The first Fc and the second Fc can be identical or different. FIG. 4F has a formula of HC FVIII-H-LC FVIII-Linker-first Fc-Linker-VWF-Linker-second Fc. The linkers (the first linker between LC FVIII and first Fc, the second linker between first Fc and VWF, and the third linker between VWF and second Fc) can be identical or different. One or more linkers can be a cleavable linker. For example, the linker between the first Fc and VWF can be a cleavable linker comprising a cleavage site at the N-terminus and/or the C-terminus of the linker. The first Fc and the second Fc can be identical or different. FIG. 4G has a formula of SC FVIII-Linker-Fc-Linker-VWF-H-Linker-Fc. FIG. 4H has a formula of Pegylated or Hesylated SC FVIII-Linker-Fc-Linker-VWF-H-Linker-Fc. The linkers (the first linker between SC FVIII and first Fc, the second linker between first Fc and VWF, and the third linker between H and second Fc) can be identical or different. One or more linkers can be a cleavable linker. For example, the linker between the first Fc and VWF can be a cleavable linker comprising a cleavage site at the N-terminus and/or the C-terminus of the linker. The first Fc and the second Fc can be identical or different.

FIG. 5. Schematic diagrams of FVIII-VWF heterodimer co-transfection system. Construct FVIII-155 contains the full-length FVIII sequence (with an alanine residue substituting the arginine residues at 1645 and 1648) linked to an Fc region. VWF-031 contains the DID2D′D3 fragment (with an alanine residue substituting the Cysteine residues at 336 and 379) which is linked to another Fc region with a 48 thrombin cleavable linker. After intracellular processing, construct FVIII-155 produces a full length single chain FVIII (SCFVIII) fused to one Fc fragment, construct VWF-031 produces a 477 amino acids D′D3 fragment linked to another Fc fragment. Two covalent bonds can be formed between the Fc fragments that are linked to the SC FVIII or the D′D3 fragment, this in turn allows a covalent association of FVIII and D′D3, which is the main character of the desired final product.

FIG. 6 is the non-reducing and reducing SDS PAGE of VWF-009 (D1D2D′D3 1-276 aa×6 HIS), which shows VWF-009 exists as a monomer. Unprocessed means VVF-009 with the propeptide (the DID2 domains).

FIG. 7 is the non-reducing and reducing SDS PAGE of VWF-002 (D′D3 1-477 aa×6 his) or VWF-010 (D1D2D′D3 1-477 aa×6 his), which shows VWF-002 exists as a monomer and VWF-010 exists as a dimer.

FIG. 8 shows thrombin digestion of FVIII-VWF heterodimer shown in FIG. 2 (b). Lane 1 shows marker. Lane 2 is rFVIII-Fc without thrombin. Lane 3 is rFVIII-Fc with thrombin. Lane 5 is FVIIIFc-VWF. Lane 6 shows FVIIIFc-VWF and thrombin. A1 indicates A1 domain of FVIII, A2 indicates A2 domain of FVIII, and Δa3 LC indicates the light chain of FVIII.

FIG. 9A-B shows the FVIII activity measured by a FVIII chromogenic assay. FIG. 9A shows pharmacokinetic profile of rFVIII and rFVIIIFc in HemA mouse. FIG. 9B shows PK profile of rFVIII and rFVIIIFc in FVIII/VWF Double knockout (DKO) mouse. The Y axis shows FVIII activity in mIU/mL, and the X axis shows time.

FIG. 10A-B shows FVIII protection by the D′D3 fragments as shown by mFVIII plasma level (mIU/mL) and VWF expression level (nM/mL) 48 hours post plasmid injection. The VWF fragments used to show FVIII protection are VWF-001 (276aa, monomer), VWF-009 (276aa, monomer), VWF-002 (477aa, monomer), VWF-010 (477aa,dimer), VWF-003 (511aa, monomer), VWF-011 (511aa, dimer), VWF-004 (716aa, monomer), VWF-012 (716aa, dimer), VWF-006, and VWF-008.

FIGS. 11A-11B show the pharmacokinetic profile of rBDD-FVIII in FVIII-VWF DKO mice when co-administered with D′D3 fragments. FIG. 11A shows FVIII activity (mIU/mL) measured by a FVIII chromogenic assay after co-administration of rBDD-FVIII and VWF-002 or rBDD-FVIII and VWF-010 or rBDD-FVIII alone in FVIII/VWF DKO mice. FIG. 11B shows VWF-002 and VWF-010 plasma level (ng/mL) after administration. The X axis represents time in hours.

FIGS. 12A-12C show pharmacokinetic profile of rFVIIIFc in VWF D′D3 expressing mice. FIG. 12A shows the timeline of hydrodynamic injection (HDI) of the D′D3 domain encoding plasmid DNA (day-5), intravenous dosing of rFVIIIFc (day 0), and PK sample collection (day0-day3). FIG. 12B shows post rFVIIIFc infusion plasma FVIII activity (mIU/mL) measured by a FVIII chromogenic assay in FVIII/VWF DKO mice with HDI of the DID2D′D3 domains (477aa) (circle) and the DID2D′D3 domains (477aa) with cysteine substitutions (rectangle) in FVIII/VWF DKO mice. The FVIII activity in control mice without HDI of the D′D3 domains is shown as triangle. FIG. 12C show the D′D3 plasma level (ng/ml) after HDI administration of the D1D2D′D3 dimer or the DID2D′D3 monomer DNA construct. The X axis represents time in hours.

FIG. 13 shows D′D3-Fc linker selection by HDI in FVIII/VWF DKO mice. Different lengths of the linkers (20aa (FVIII-064), 35aa (FVIII-159), or 48aa (FVIII-160)) were inserted between the D′D3 domains and the Fc region. The FVIII activity (mIU/ml) was measured by a FVIII chromogenic assay after HDI in FVIII/VWF DKO mice.

FIG. 14 shows HDI of Single Chain FVIIIFc/D′D3 heterodimer in FVIII/VWF DKO mice. The FVIII activities of processed (dual chain) rFVIIIFc-D′D3 (pSYN-FVIII-136) and Single Chain rFVIIIFc-D′D3 (pSYN-FVIII-148) were measured 24 hours and 48 hours after HDI.

FIG. 15 shows binding affinity of FVIII-155/VWF-031 heterodimer to immobilized hVWF by Octet assay. FVIIIFc, FVIII, and IgG were also used as controls. The x-axis shows time in seconds, and the y-axis shows the binding in nanometer (nm).

FIG. 16 shows FVIII-155/VWF-031 pharmacokinetics in FVIII/VWF deficient (FVIII/VWF DKO) mice. The x-axis indicates time in hours, and the y-axis indicates FVIII recovery v. input in percent.

FIGS. 17A-17C: Schematic diagrams of examples of VWF fragment constructs, in which VWF is DID2D′D3 fragment of VWF; the Linker is a variable length linker containing a cleavage site, e.g., a thrombin cleavage site; H is a heterologous moiety, e.g., an immunoglobulin constant region or a portion thereof, a moiety for conjugating polyethylene glycol (PEG) and/or PEG, an albumin or albumin fragment, an albumin binding moiety, a HAP sequence, a moiety for polysialylation and/or polysialic acid, a moiety for hydroxyethyl starch (HES) and/or HES, or a PAS sequence, etc.; and Fc is an Fc region of an immunoglobulin. FIG. 17A has a formula of DID2-D′partial D3-H-Partial D3-Linker-Fc. FIG. 17B has a formula of DID2-Partial D′-H-partial D′D3-Linker-Fc. FIG. 17C has a formula of DID2-Pegylated or Hesylated D′D3-Linker-Fc. The linker can be optionally cleaved.

FIGS. 18A-18B: FIG. 18A shows FVIIIFc loses FVIII activity in both HemA (diamond) and DKO (square) plasma over time. FVIII activity is measured by chromogenic assay. X-axis shows time in hours, and γ-axis shows relative activity. FIG. 18B shows that the loss in FVIII activity is due to the dissociation or degradation of the heavy chain (HC). The left panel shows an immuno-precipitation assay using sheep anti-FVIII polyclonal antibody in Bio-rad 4-15% gel. The gel was reduced and imaged by Bio-rad system. Lane 1 shows Bio-rad unstain marker; lane 2 shows FVIIIFc and PBS; lane 3 shows FVIIIFc and DKO plasma; and lane 5 shows sheep anti-FVIII polyclonal antibody alone. The right panel shows Western analysis of the gel using FVIII anti-heavy chain antibody (GMA012). Lane 1 shows Bio-rad unstain marker; lane 2 shows FVIIIFc and PBS; lane 3 shows FVIIIFc and DKO plasma; and lane 4 shows sheep anti-FVIII polyclonal antibody alone.

FIG. 19: shows FVIII activity of wild type FVIIIFc (circle), scFVIIIFc (single chain FVIII) (filled triangle), or FVIII:VWF heterodimer (e.g., FVIII155/VWF31) (empty triangle) by chromogenic assay in DKO mouse plasma (left panel) and HemA mouse plasma (right panel) as a function of time. Y axis shows relative FVIII activity. Wild type FVIIIFc contains dual chain of FVIII (i.e., FVIII heavy chain and FVIII light chain held together non-covalently) and thus has three chains, a FVIII heavy chain, a FVIII light chain fused to an Fc, and an Fc alone. ScFVIIIFc contains a FVIII single chain and thus has two chains, one with a single chain FVIII fused to an Fc and another with an Fc alone. The FVIII:VWF heterodimer (e.g., FVIII155/VWF031) contains single chain FVIII fused to an Fc and a VWF fragment (D′D3) fused to an Fc.

FIG. 20 shows processing of DID2 domain from VWF fragment (e.g., VWF-031 (DID2D′D3Fc)) by PC5 or PACE (Furin) at different concentrations. The DID2 processing is shown on a Bio-rad 4-15% gel at a reduced condition by Bio-rad imager. Lane 1 shows VWF031 alone; lane 2 shows PC5 alone; lane 3 shows PACE alone; lane 4 shows VWF031 and PC5 at 2.5%; lane 5 shows VWF031 and PC5 at 5%; lane 6 shows VWF031 and PC5 at 7.5%; lane 7 shows VWF031 and PC5 at 10%; lane 8 shows VWF031 and PACE at 2.5%; lane 9 shows VWF031 and PACE at 5%; lane 10 shows VWF031 at 7.5%; and lane 11 shows VWF031 and PACE at 10%.

FIGS. 21A-21B: FIG. 21A shows that a binding assay of a FVIII:VWF heterodimer (e.g., FVIII-155/VWF-031) by ForteBio octet instrument. For the assay, full length VWF was captured by using APS sensor. The binding of FVIIIFc and FVIII to the full-length VWF is shown at the lower left panel. The lack of binding of FVIIIY1680 (a mutant having no affinity for VWF) and FVIII:VWF heterodimer (FVIII155/VWF031) is shown at the lower right panel. FIG. 21B shows another binding assay of a FVIII:VWF heterodimer (e.g., FVIII-155/VWF-031). In this assay, the constructs (VWF031 construct, FVIII-155/VWF031, or FVIII) were immobilized on protein G sensor. The binding of the constructs to FVIII was measured.

FIG. 22 shows binding affinity of VWF D′D3 domains with FVIII molecule measured by a surface plasma resonance experiment. The VWF031 construct (100RU) was captured by 1000RU anti-human IgG. B-domain deleted FVIII was applied in single cycle kinetics mode in 1:1 fit. The total number was 4.

FIG. 23 shows effects of different linker length in the FVIIIFc/VWF heterodimer constructs on pharmacokinetics when administered in FVIII/VWF DKO mice. Three different linkers (48 aa, 73aa, or 98aa) were inserted between the D′D3 and the Fc, i.e., VWF031, VWF035, and VWF036. The FVIII activity normalized to 5 min value (%) is shown in Y-axis.

FIGS. 24A-24E show examples of sortase ligation of a VWF fragment with FVIII. FIG. 24A shows two ligation constructs, (1) a VWF fragment fused to a sortase recognition motif (e.g., LPXTG) at the C-terminus and (2) FVIII having glycine (n) at the N-terminus. After reaction with sortase, the VWF fragment and the sortase recognition motif are ligated to the N-terminus of FVIII. FIG. 24B shows two ligation constructs, (1) FVIII fused to a sortase recognition motif at its C-terminus and (2) a VWF fragment having glycine (n) at its N-terminus. After reaction with sortase, FVIII and the sortase recognition motif are fused to the VWF fragment at the N-terminus of the VWF fragment. FIG. 24C shows two ligation constructs, (1) a VWF fragment fused to a sortase recognition motif by a variable length linker and (2) FVIII fused to glycine (n) at its N-terminus. After reaction with sortase, the VWF fused by a linker to the sortase recognition motif is ligated to the N-terminus of FVIII. FIG. 24D shows two ligation constructs, (1) FVIII fused by a variable length linker to a sortase recognition motif and (2) a VWF fragment fused to glycine (n) at its N-terminus. After reaction with sortase, FVIII fused by a linker to the sortase recognition motif is ligated to the N terminus of VWF fragment. FIG. 24E shows a ligation construct containing a VWF fragment fused by a variable length linker to a sortase recognition motif, which is also fused to a protease cleavage site (e.g., Thrombin cleavage site) fused by a variable length linker to an Fc.

FIG. 25 shows a schematic comparison of FVIII155 and FVIII198. FVIII155 encodes a single chain FVIIIFc protein. FVIII198 is a partial B-domain containing single chain FVIIIFc molecule-226N6. 226 represents the N-terminus 226 amino acid of the FVIII B-domain, and N6 represents six N-glycosylation sites in the B-domain.

FIG. 26A shows a stability assay measuring the relativity activity of FVIII155 and FVIII198 in DKO plasma as a function of time. As can be seen in the figure, the presence of the partial B-domain in FVIII198 increased the stability of single chain FVIIIFc in comparison to FVIII155; FIG. 26B shows a comparison of the half-lives of FVIII198, FVIII155, and dual chain (dcFVIIIFc) in DKO mice. As can be seen in the figure, single chain FVIII (FVIII155) has a 1.5 fold increase in half life in comparison to dual chain FVIII. Single chain FVIII with the 266N6 B-domain (FVIII198) had a further 1.5 fold increase in half life. The graph shows the FVIII recovery v. the 5 minute value (%) as a function of time.

### DETAILED DESCRIPTION OF THE INVENTION

### Definitions

It is to be noted that the term “a” or “an” entity refers to one or more of that entity; for example, “a nucleotide sequence,” is understood to represent one or more nucleotide sequences. As such, the terms “a” (or “an”), “one or more,” and “at least one” can be used interchangeably herein.

The term “polynucleotide” or “nucleotide” is intended to encompass a singular nucleic acid as well as plural nucleic acids, and refers to an isolated nucleic acid molecule or construct, e.g., messenger RNA (mRNA) or plasmid DNA (pDNA). In certain embodiments, a polynucleotide comprises a conventional phosphodiester bond or a non-conventional bond (e.g., an amide bond, such as found in peptide nucleic acids (PNA)). The term “nucleic acid” refers to any one or more nucleic acid segments, e.g., DNA or RNA fragments, present in a polynucleotide. By “isolated” nucleic acid or polynucleotide is intended a nucleic acid molecule, DNA or RNA, which has been removed from its native environment. For example, a recombinant polynucleotide encoding a Factor VIII polypeptide contained in a vector is considered isolated for the purposes of the present invention. Further examples of an isolated polynucleotide include recombinant polynucleotides maintained in heterologous host cells or purified (partially or substantially) from other polynucleotides in a solution. Isolated RNA molecules include in vivo or in vitro RNA transcripts of polynucleotides of the present invention. Isolated polynucleotides or nucleic acids according to the present invention further include such molecules produced synthetically. In addition, a polynucleotide or a nucleic acid can include regulatory elements such as promoters, enhancers, ribosome binding sites, or transcription termination signals.

As used herein, a “coding region” or “coding sequence” is a portion of polynucleotide which consists of codons translatable into amino acids. Although a “stop codon” (TAG, TGA, or TAA) is typically not translated into an amino acid, it may be considered to be part of a coding region, but any flanking sequences, for example promoters, ribosome binding sites, transcriptional terminators, introns, and the like, are not part of a coding region. The boundaries of a coding region are typically determined by a start codon at the 5′ terminus, encoding the amino terminus of the resultant polypeptide, and a translation stop codon at the 3′terminus, encoding the carboxyl terminus of the resulting polypeptide. Two or more coding regions of the present invention can be present in a single polynucleotide construct, e.g., on a single vector, or in separate polynucleotide constructs, e.g., on separate (different) vectors. It follows, then, that a single vector can contain just a single coding region, or comprise two or more coding regions, e.g., a single vector can separately encode a binding domain-A and a binding domain-B as described below. In addition, a vector, polynucleotide, or nucleic acid of the invention can encode heterologous coding regions, either fused or unfused to a nucleic acid encoding a binding domain of the invention. Heterologous coding regions include without limitation specialized elements or motifs, such as a secretory signal peptide or a heterologous functional domain.

Certain proteins secreted by mammalian cells are associated with a secretory signal peptide which is cleaved from the mature protein once export of the growing protein chain across the rough endoplasmic reticulum has been initiated. Those of ordinary skill in the art are aware that signal peptides are generally fused to the N-terminus of the polypeptide, and are cleaved from the complete or “full-length” polypeptide to produce a secreted or “mature” form of the polypeptide. In certain embodiments, a native signal peptide, e.g., an immunoglobulin heavy chain or light chain signal peptide is used, or a functional derivative of that sequence that retains the ability to direct the secretion of the polypeptide that is operably associated with it. Alternatively, a heterologous mammalian signal peptide, e.g., a human tissue plasminogen activator (TPA) or mouse β-glucuronidase signal peptide, or a functional derivative thereof, can be used.

The term “downstream” refers to a nucleotide sequence that is located 3′ to a reference nucleotide sequence. In certain embodiments, downstream nucleotide sequences relate to sequences that follow the starting point of transcription. For example, the translation initiation codon of a gene is located downstream of the start site of transcription.

The term “upstream” refers to a nucleotide sequence that is located 5′ to a reference nucleotide sequence. In certain embodiments, upstream nucleotide sequences relate to sequences that are located on the 5′ side of a coding region or starting point of transcription. For example, most promoters are located upstream of the start site of transcription.

As used herein, the term “regulatory region” refers to nucleotide sequences located upstream (5′ non-coding sequences), within, or downstream (3′ non-coding sequences) of a coding region, and which influence the transcription, RNA processing, stability, or translation of the associated coding region. Regulatory regions may include promoters, translation leader sequences, introns, polyadenylation recognition sequences, RNA processing sites, effector binding sites and stem-loop structures. If a coding region is intended for expression in a eukaryotic cell, a polyadenylation signal and transcription termination sequence will usually be located 3′ to the coding sequence.

A polynucleotide which encodes a gene product, e.g., a polypeptide, can include a promoter and/or other transcription or translation control elements operably associated with one or more coding regions. In an operable association a coding region for a gene product, e.g., a polypeptide, is associated with one or more regulatory regions in such a way as to place expression of the gene product under the influence or control of the regulatory region(s). For example, a coding region and a promoter are “operably associated” if induction of promoter function results in the transcription of mRNA encoding the gene product encoded by the coding region, and if the nature of the linkage between the promoter and the coding region does not interfere with the ability of the promoter to direct the expression of the gene product or interfere with the ability of the DNA template to be transcribed. Other transcription control elements, besides a promoter, for example enhancers, operators, repressors, and transcription termination signals, can also be operably associated with a coding region to direct gene product expression.

A variety of transcription control regions are known to those skilled in the art. These include, without limitation, transcription control regions which function in vertebrate cells, such as, but not limited to, promoter and enhancer segments from cytomegaloviruses (the immediate early promoter, in conjunction with intron-A), simian virus 40 (the early promoter), and retroviruses (such as Rous sarcoma virus). Other transcription control regions include those derived from vertebrate genes such as actin, heat shock protein, bovine growth hormone and rabbit β-globin, as well as other sequences capable of controlling gene expression in eukaryotic cells. Additional suitable transcription control regions include tissue-specific promoters and enhancers as well as lymphokine-inducible promoters (e.g., promoters inducible by interferons or interleukins).

Similarly, a variety of translation control elements are known to those of ordinary skill in the art. These include, but are not limited to ribosome binding sites, translation initiation and termination codons, and elements derived from picornaviruses (particularly an internal ribosome entry site, or IRES, also referred to as a CITE sequence).

The term “expression” as used herein refers to a process by which a polynucleotide produces a gene product, for example, an RNA or a polypeptide. It includes without limitation transcription of the polynucleotide into messenger RNA (mRNA), transfer RNA (tRNA), small hairpin RNA (shRNA), small interfering RNA (siRNA) or any other RNA product, and the translation of an mRNA into a polypeptide. Expression produces a “gene product.” As used herein, a gene product can be either a nucleic acid, e.g., a messenger RNA produced by transcription of a gene, or a polypeptide which is translated from a transcript. Gene products described herein further include nucleic acids with post transcriptional modifications, e.g., polyadenylation or splicing, or polypeptides with post translational modifications, e.g., methylation, glycosylation, the addition of lipids, association with other protein subunits, or proteolytic cleavage.

A “vector” refers to any vehicle for the cloning of and/or transfer of a nucleic acid into a host cell. A vector may be a replicon to which another nucleic acid segment may be attached so as to bring about the replication of the attached segment. A “replicon” refers to any genetic element (e.g., plasmid, phage, cosmid, chromosome, virus) that functions as an autonomous unit of replication in vivo, i.e., capable of replication under its own control. The term “vector” includes both viral and nonviral vehicles for introducing the nucleic acid into a cell in vitro, ex vivo or in vivo. A large number of vectors are known and used in the art including, for example, plasmids, modified eukaryotic viruses, or modified bacterial viruses. Insertion of a polynucleotide into a suitable vector can be accomplished by ligating the appropriate polynucleotide fragments into a chosen vector that has complementary cohesive termini.

Vectors may be engineered to encode selectable markers or reporters that provide for the selection or identification of cells that have incorporated the vector. Expression of selectable markers or reporters allows identification and/or selection of host cells that incorporate and express other coding regions contained on the vector. Examples of selectable marker genes known and used in the art include: genes providing resistance to ampicillin, streptomycin, gentamycin, kanamycin, hygromycin, bialaphos herbicide, sulfonamide, and the like; and genes that are used as phenotypic markers, i.e., anthocyanin regulatory genes, isopentanyl transferase gene, and the like. Examples of reporters known and used in the art include: luciferase (Luc), green fluorescent protein (GFP), chloramphenicol acetyltransferase (CAT), -galactosidase (LacZ), -glucuronidase (Gus), and the like. Selectable markers may also be considered to be reporters.

The term “plasmid” refers to an extra-chromosomal element often carrying a gene that is not part of the central metabolism of the cell, and usually in the form of circular double-stranded DNA molecules. Such elements may be autonomously replicating sequences, genome integrating sequences, phage or nucleotide sequences, linear, circular, or supercoiled, of a single- or double-stranded DNA or RNA, derived from any source, in which a number of nucleotide sequences have been joined or recombined into a unique construction which is capable of introducing a promoter fragment and DNA sequence for a selected gene product along with appropriate 3′ untranslated sequence into a cell.

Eukaryotic viral vectors that can be used include, but are not limited to, adenovirus vectors, retrovirus vectors, adeno-associated virus vectors, poxvirus, e.g., vaccinia virus vectors, baculovirus vectors, or herpesvirus vectors. Non-viral vectors include plasmids, liposomes, electrically charged lipids (cytofectins), DNA-protein complexes, and biopolymers.

A “cloning vector” refers to a “replicon,” which is a unit length of a nucleic acid that replicates sequentially and which comprises an origin of replication, such as a plasmid, phage or cosmid, to which another nucleic acid segment may be attached so as to bring about the replication of the attached segment. Certain cloning vectors are capable of replication in one cell type, e.g., bacteria and expression in another, e.g., eukaryotic cells. Cloning vectors typically comprise one or more sequences that can be used for selection of cells comprising the vector and/or one or more multiple cloning sites for insertion of nucleic acid sequences of interest.

The term “expression vector” refers to a vehicle designed to enable the expression of an inserted nucleic acid sequence following insertion into a host cell. The inserted nucleic acid sequence is placed in operable association with regulatory regions as described above.

Vectors are introduced into host cells by methods well known in the art, e.g., transfection, electroporation, microinjection, transduction, cell fusion, DEAE dextran, calcium phosphate precipitation, lipofection (lysosome fusion), use of a gene gun, or a DNA vector transporter.

“Culture,” “to culture” and “culturing,” as used herein, means to incubate cells under in vitro conditions that allow for cell growth or division or to maintain cells in a living state. “Cultured cells,” as used herein, means cells that are propagated in vitro.

As used herein, the term “polypeptide” is intended to encompass a singular “polypeptide” as well as plural “polypeptides,” and refers to a molecule composed of monomers (amino acids) linearly linked by amide bonds (also known as peptide bonds). The term “polypeptide” refers to any chain or chains of two or more amino acids, and does not refer to a specific length of the product. Thus, peptides, dipeptides, tripeptides, oligopeptides, “protein,” “amino acid chain,” or any other term used to refer to a chain or chains of two or more amino acids, are included within the definition of “polypeptide,” and the term “polypeptide” can be used instead of, or interchangeably with any of these terms. The term “polypeptide” is also intended to refer to the products of post-expression modifications of the polypeptide, including without limitation glycosylation, acetylation, phosphorylation, amidation, derivatization by known protecting/blocking groups, proteolytic cleavage, or modification by non-naturally occurring amino acids. A polypeptide can be derived from a natural biological source or produced recombinant technology, but is not necessarily translated from a designated nucleic acid sequence. It can be generated in any manner, including by chemical synthesis.

An “isolated” polypeptide or a fragment, variant, or derivative thereof refers to a polypeptide that is not in its natural milieu. No particular level of purification is required. For example, an isolated polypeptide can simply be removed from its native or natural environment. Recombinantly produced polypeptides and proteins expressed in host cells are considered isolated for the purpose of the invention, as are native or recombinant polypeptides which have been separated, fractionated, or partially or substantially purified by any suitable technique.

Also included in the present invention are fragments or variants of polypeptides, and any combination thereof. The term “fragment” or “variant” when referring to polypeptide binding domains or binding molecules of the present invention include any polypeptides which retain at least some of the properties (e.g., FcRn binding affinity for an FcRn binding domain or Fc variant, coagulation activity for an FVIII variant, or FVIII binding activity for the VWF fragment) of the reference polypeptide. Fragments of polypeptides include proteolytic fragments, as well as deletion fragments, in addition to specific antibody fragments discussed elsewhere herein, but do not include the naturally occurring full-length polypeptide (or mature polypeptide). Variants of polypeptide binding domains or binding molecules of the present invention include fragments as described above, and also polypeptides with altered amino acid sequences due to amino acid substitutions, deletions, or insertions. Variants can be naturally or non-naturally occurring. Non-naturally occurring variants can be produced using art-known mutagenesis techniques. Variant polypeptides can comprise conservative or non-conservative amino acid substitutions, deletions or additions.

The term “VWF fragment” or “VWF fragments” used herein means any VWF fragments that interact with FVIII and retain at least one or more properties that are normally provided to FVIII by full-length VWF, e.g., preventing premature activation to FVIIIa, preventing premature proteolysis, preventing association with phospholipid membranes that could lead to premature clearance, preventing binding to FVIII clearance receptors that can bind naked FVIII but not VWF-bound FVIII, and/or stabilizing the FVIII heavy chain and light chain interactions. The term “VWF fragment” as used herein does not include full length- or mature VWF protein. In a particular embodiment, the “VWF fragment” as used herein comprises a D′ domain and a D3 domain of the VWF protein, but does not include the A1 domain, the A2 domain, the A3 domain, the D4 domain, the B1 domain, the B2 domain, the B3 domain, the C1 domain, the C2 domain, and the CK domain of the VWF protein.

The term “half-life limiting factor” or “FVIII half-life limiting factor” as used herein indicates a factor that prevents the half-life of a FVIII protein from being longer than 1.5 fold or 2 fold compared to wild-type FVIII (e.g., ADVATE® or REFACTO®). For example, full length or mature VWF can act as a FVIII half-life limiting factor by inducing the FVIII and VWF complex to be cleared from system by one or more VWF clearance pathways. In one example, endogenous VWF is a FVIII half-life limiting factor. In another example, a full-length recombinant VWF molecule non-covalently bound to a FVIII protein is a FVIII-half-life limiting factor.

The term “endogenous VWF” as used herein indicates VWF molecules naturally present in plasma. The endogenous VWF molecule can be multimer, but can be a monomer or a dimer. Endogenous VWF in plasma binds to FVIII and forms a non-covalent complex with FVIII.

A “conservative amino acid substitution” is one in which the amino acid residue is replaced with an amino acid residue having a similar side chain. Families of amino acid residues having similar side chains have been defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, if an amino acid in a polypeptide is replaced with another amino acid from the same side chain family, the substitution is considered to be conservative. In another embodiment, a string of amino acids can be conservatively replaced with a structurally similar string that differs in order and/or composition of side chain family members.

As known in the art, “sequence identity” between two polypeptides is determined by comparing the amino acid sequence of one polypeptide to the sequence of a second polypeptide. When discussed herein, whether any particular polypeptide is at least about 50%, 60%, 70%, 75%, 80%, 85%, 90%, 95%, 99%, or 100% identical to another polypeptide can be determined using methods and computer programs/software known in the art such as, but not limited to, the BESTFIT program (Wisconsin Sequence Analysis Package, Version 8 for Unix, Genetics Computer Group, University Research Park, 575 Science Drive, Madison, WI 53711). BESTFIT uses the local homology algorithm of Smith and Waterman, Advances in Applied Mathematics 2:482-489 (1981), to find the best segment of homology between two sequences. When using BESTFIT or any other sequence alignment program to determine whether a particular sequence is, for example, 95% identical to a reference sequence according to the present invention, the parameters are set, of course, such that the percentage of identity is calculated over the full-length of the reference polypeptide sequence and that gaps in homology of up to 5% of the total number of amino acids in the reference sequence are allowed.

As used herein, an “amino acid corresponding to” or an “equivalent amino acid” in a VWF sequence or a FVIII protein sequence is identified by alignment to maximize the identity or similarity between a first VWF or FVIII sequence and a second VWF or FVIII sequence. The number used to identify an equivalent amino acid in a second VWF or FVIII sequence is based on the number used to identify the corresponding amino acid in the first VWF or FVIII sequence.

A “fusion” or “chimeric” protein comprises a first amino acid sequence linked to a second amino acid sequence with which it is not naturally linked in nature. The amino acid sequences which normally exist in separate proteins can be brought together in the fusion polypeptide, or the amino acid sequences which normally exist in the same protein can be placed in a new arrangement in the fusion polypeptide, e.g., fusion of a Factor VIII domain of the invention with an immunoglobulin Fc domain. A fusion protein is created, for example, by chemical synthesis, or by creating and translating a polynucleotide in which the peptide regions are encoded in the desired relationship. A chimeric protein can further comprises a second amino acid sequence associated with the first amino acid sequence by a covalent, non-peptide bond or a non-covalent bond.

As used herein, the term “half-life” refers to a biological half-life of a particular polypeptide in vivo. Half-life may be represented by the time required for half the quantity administered to a subject to be cleared from the circulation and/or other tissues in the animal. When a clearance curve of a given polypeptide is constructed as a function of time, the curve is usually biphasic with a rapid α-phase and longer β-phase. The α-phase typically represents an equilibration of the administered Fc polypeptide between the intra- and extra-vascular space and is, in part, determined by the size of the polypeptide. The β-phase typically represents the catabolism of the polypeptide in the intravascular space. In some embodiments, FVIII and chimeric proteins comprising FVIII are monophasic, and thus do not have an alpha phase, but just the single beta phase. Therefore, in certain embodiments, the term half-life as used herein refers to the half-life of the polypeptide in the β-phase. The typical β phase half-life of a human antibody in humans is 21 days.

The term “heterologous” as applied to a polynucleotide or a polypeptide, means that the polynucleotide or polypeptide is derived from a distinct entity from that of the entity to which it is being compared. Therefore, a heterologous polypeptide linked to a VWF fragment means a polypeptide chain that is linked to a VWF fragment and is not a naturally occurring part of the VWF fragment. For instance, a heterologous polynucleotide or antigen can be derived from a different species, different cell type of an individual, or the same or different type of cell of distinct individuals.

The term “linked” as used herein refers to a first amino acid sequence or nucleotide sequence covalently or non-covalently joined to a second amino acid sequence or nucleotide sequence, respectively. The term “covalently linked” or “covalent linkage” refers to a covalent bond, e.g., a disulfide bond, a peptide bond, or one or more amino acids, e.g., a linker, between the two moieties that are linked together. The first amino acid or nucleotide sequence can be directly joined or juxtaposed to the second amino acid or nucleotide sequence or alternatively an intervening sequence can covalently join the first sequence to the second sequence. The term “linked” means not only a fusion of a first amino acid sequence to a second amino acid sequence at the C-terminus or the N-terminus, but also includes insertion of the whole first amino acid sequence (or the second amino acid sequence) into any two amino acids in the second amino acid sequence (or the first amino acid sequence, respectively). In one embodiment, the first amino acid sequence can be joined to a second amino acid sequence by a peptide bond or a linker. The first nucleotide sequence can be joined to a second nucleotide sequence by a phosphodiester bond or a linker. The linker can be a peptide or a polypeptide (for polypeptide chains) or a nucleotide or a nucleotide chain (for nucleotide chains) or any chemical moiety (for both polypeptide and polynucleotide chains). The covalent linkage is sometimes indicated as (-) or hyphen.

As used herein the term “associated with” refers to a covalent or non-covalent bond formed between a first amino acid chain and a second amino acid chain. In one embodiment, the term “associated with” means a covalent, non-peptide bond or a non-covalent bond. In some embodiments this association is indicated by a colon, i.e., (:). In another embodiment, it means a covalent bond except a peptide bond. In other embodiments, the term “covalently associated” as used herein means an association between two moieties by a covalent bond, e.g., a disulfide bond, a peptide bond, or one or more amino acids (e.g., a linker). For example, the amino acid cysteine comprises a thiol group that can form a disulfide bond or bridge with a thiol group on a second cysteine residue. In most naturally occurring IgG molecules, the CH1 and CL regions are associated by a disulfide bond and the two heavy chains are associated by two disulfide bonds at positions corresponding to 239 and 242 using the Kabat numbering system (position 226 or 229, EU numbering system). Examples of covalent bonds include, but are not limited to, a peptide bond, a metal bond, a hydrogen bond, a disulfide bond, a sigma bond, a pi bond, a delta bond, a glycosidic bond, an agnostic bond, a bent bond, a dipolar bond, a Pi backbond, a double bond, a triple bond, a quadruple bond, a quintuple bond, a sextuple bond, conjugation, hyperconjugation, aromaticity, hapticity, or antibonding. Non-limiting examples of non-covalent bond include an ionic bond (e.g., cation-pi bond or salt bond), a metal bond, an hydrogen bond (e.g., dihydrogen bond, dihydrogen complex, low-barrier hydrogen bond, or symmetric hydrogen bond), van der Walls force, London dispersion force, a mechanical bond, a halogen bond, aurophilicity, intercalation, stacking, entropic force, or chemical polarity.

The term “monomer-dimer hybrid” used herein refers to a chimeric protein comprising a first polypeptide chain and a second polypeptide chain, which are associated with each other by a disulfide bond, wherein the first chain comprises a clotting factor, e.g., Factor VIII, and an Fc region and the second chain comprises, consists essentially of, or consists of an Fc region without the clotting factor. The monomer-dimer hybrid construct thus is a hybrid comprising a monomer aspect having only one clotting factor and a dimer aspect having two Fc regions.

As used herein, the term “cleavage site” or “enzymatic cleavage site” refers to a site recognized by an enzyme. Certain enzymatic cleavage sites comprise an intracellular processing site. In one embodiment, a polypeptide has an enzymatic cleavage site cleaved by an enzyme that is activated during the clotting cascade, such that cleavage of such sites occurs at the site of clot formation. Exemplary such sites include e.g., those recognized by thrombin, Factor XIa or Factor Xa. Exemplary FXIa cleavage sites include, e.g, TQSFNDFTR (SEQ ID NO: 47) and SVSQTSKLTR (SEQ ID NO: 48). Exemplary thrombin cleavage sites include, e.g, DFLAEGGGVR (SEQ ID NO: 49), TTKIKPR (SEQ ID NO: 50), LVPRG (SEQ ID NO: 55) and ALRPR (amino acids 1 to 5 of SEQ ID NO: 51). Other enzymatic cleavage sites are known in the art.

As used herein, the term “processing site” or “intracellular processing site” refers to a type of enzymatic cleavage site in a polypeptide which is the target for enzymes that function after translation of the polypeptide. In one embodiment, such enzymes function during transport from the Golgi lumen to the trans-Golgi compartment. Intracellular processing enzymes cleave polypeptides prior to secretion of the protein from the cell. Examples of such processing sites include, e.g., those targeted by the PACE/furin (where PACE is an acronym for Paired basic Amino acid Cleaving Enzyme) family of endopeptidases. These enzymes are localized to the Golgi membrane and cleave proteins on the carboxy terminal side of the sequence motif Arg-[any residue]-(Lys or Arg)-Arg. As used herein the “furin” family of enzymes includes, e.g., PCSK1 (also known as PC1/Pc3), PCSK2 (also known as PC2), PCSK3 (also known as furin or PACE), PCSK4 (also known as PC4), PCSK5 (also known as PC5 or PC6), PCSK6 (also known as PACE4), or PCSK7 (also known as PC7/LPC, PC8, or SPC7). Other processing sites are known in the art.

The term “Furin” refers to the enzymes corresponding to EC No. 3.4.21.75. Furin is subtilisin-like proprotein convertase, which is also known as PACE (Paired basic Amino acid Cleaving Enzyme). Furin deletes sections of inactive precursor proteins to convert them into biologically active proteins. During its intracellular transport, pro-peptide is cleaved from mature VWF molecule by a Furin enzyme in the Golgi.

In constructs that include more than one processing or cleavage site, it will be understood that such sites may be the same or different.

Hemostatic disorder, as used herein, means a genetically inherited or acquired condition characterized by a tendency to hemorrhage, either spontaneously or as a result of trauma, due to an impaired ability or inability to form a fibrin clot. Examples of such disorders include the hemophilias. The three main forms are hemophilia A (factor VIII deficiency), hemophilia B (factor IX deficiency or “Christmas disease”) and hemophilia C (factor XI deficiency, mild bleeding tendency). Other hemostatic disorders include, e.g., Von Willebrand disease, Factor XI deficiency (PTA deficiency), Factor XII deficiency, deficiencies or structural abnormalities in fibrinogen, prothrombin, Factor V, Factor VII, Factor X or factor XIII, Bernard-Soulier syndrome, which is a defect or deficiency in GPIb. GPIb, the receptor for VWF, can be defective and lead to lack of primary clot formation (primary hemostasis) and increased bleeding tendency), and thrombasthenia of Glanzman and Naegeli (Glanzmann thrombasthenia). In liver failure (acute and chronic forms), there is insufficient production of coagulation factors by the liver; this may increase bleeding risk.

The chimeric molecules of the invention can be used prophylactically. As used herein the term “prophylactic treatment” refers to the administration of a molecule prior to a bleeding episode. In one embodiment, the subject in need of a general hemostatic agent is undergoing, or is about to undergo, surgery. The chimeric protein of the invention can be administered prior to or after surgery as a prophylactic. The chimeric protein of the invention can be administered during or after surgery to control an acute bleeding episode. The surgery can include, but is not limited to, liver transplantation, liver resection, dental procedures, or stem cell transplantation.

The chimeric protein of the invention is also used for on-demand (also referred to as “episodic”) treatment. The term “on-demand treatment” or “episodic treatment” refers to the administration of a chimeric molecule in response to symptoms of a bleeding episode or before an activity that may cause bleeding. In one aspect, the on-demand (episodic) treatment can be given to a subject when bleeding starts, such as after an injury, or when bleeding is expected, such as before surgery. In another aspect, the on-demand treatment can be given prior to activities that increase the risk of bleeding, such as contact sports.

As used herein the term “acute bleeding” refers to a bleeding episode regardless of the underlying cause. For example, a subject may have trauma, uremia, a hereditary bleeding disorder (e.g., factor VII deficiency) a platelet disorder, or resistance owing to the development of antibodies to clotting factors.

Treat, treatment, treating, as used herein refers to, e.g., the reduction in severity of a disease or condition; the reduction in the duration of a disease course; the amelioration of one or more symptoms associated with a disease or condition; the provision of beneficial effects to a subject with a disease or condition, without necessarily curing the disease or condition, or the prophylaxis of one or more symptoms associated with a disease or condition. In one embodiment, the term “treating” or “treatment” means maintaining a FVIII trough level at least about 1 IU/dL, 2 IU/dL, 3 IU/dL, 4 IU/dL, 5 IU/dL, 6 IU/dL, 7 IU/dL, 8 IU/dL, 9 IU/dL, 10 IU/dL, 11 IU/dL, 12 IU/dL, 13 IU/dL, 14 IU/dL, 15 IU/dL, 16 IU/dL, 17 IU/dL, 18 IU/dL, 19 IU/dL, or 20 IU/dL in a subject by administering a chimeric protein or a VWF fragment of the invention. In another embodiment, treating or treatment means maintaining a FVIII trough level between about 1 and about 20 IU/dL, about 2 and about 20 IU/dL, about 3 and about 20 IU/dL, about 4 and about 20 IU/dL, about 5 and about 20 IU/dL, about 6 and about 20 IU/dL, about 7 and about 20 IU/dL, about 8 and about 20 IU/dL, about 9 and about 20 IU/dL, or about 10 and about 20 IU/dL. Treatment or treating of a disease or condition can also include maintaining FVIII activity in a subject at a level comparable to at least about 1%, 2%, 3%, 4%, 5%, 6%, 7%, 8%, 9%, 10%, 11%, 12%, 13%, 14%, 15%, 16%, 17%, 18%, 19%, or 20% of the FVIII activity in a non-hemophiliac subject. The minimum trough level required for treatment can be measured by one or more known methods and can be adjusted (increased or decreased) for each person.

### Chimeric Proteins

The present invention is directed to extending the half-life of a Factor VIII protein by preventing or inhibiting a FVIII half-life limiting factor (e.g. endogenous VWF) in vivo from associating with the FVIII protein. Endogenous VWF associates with about 95% to about 98% of FVIII in non-covalent complexes. The endogenous VWFs bound to a FVIII protein are known to protect FVIII in various ways. For example, full length VWF (as a multimer having about 250 kDa) can protect FVIII from protease cleavage and FVIII activation, stabilize the FVIII heavy chain and/or light chain, and prevent clearance of FVIII by scavenger receptors. However, at the same time, endogenous VWF limits the FVIII half-life by preventing pinocytosis and by clearing FVIII-VWF complex from the system through the VWF clearance pathway. It is believed, as shown in the examples, that endogenous VWF is the half-life limiting factor that prevents the half-life of a FVIII protein fused to a half-life extender from being longer than about two-fold of wild-type FVIII. Therefore, the present invention prevents or inhibits interaction between endogenous VWF and a FVIII protein using an adjunct moiety, thereby preventing the FVIII protein from being cleared through the VWF clearance pathway and/or inducing pinocytosis. In one embodiment, the adjunct moiety is capable of preventing or inhibiting binding of the FVIII protein with endogenous VWF and has at least one VWF-like FVIII protecting property. In addition, the adjunct moiety reduces clearance of FVIII from the system by preventing or inhibiting interaction with endogenous VWF. The adjunct moieties of the present invention bind to or are associated with (e.g., via non-covalent bonding) a FVIII protein and/or physically or chemically block the VWF binding site on the FVIII protein. The FVIII protein associated with the adjunct moiety is thus cleared from the circulation more slowly by one or more VWF clearance receptors, as compared to wild type FVIII or FVIII not associated with an adjunct moiety.

Examples of the adjunct moieties of the present invention include, e.g., polypeptides or chemical or physical modifications, additions, deletions, or variations of the FVIII protein. The adjunct moiety useful in the present invention can comprise a polypeptide, a non-polypeptide moiety, or both. Non-limiting examples of the polypeptide useful as an adjunct moiety include, e.g., a VWF fragment described herein, an immunoglobulin constant region or a portion thereof, transferrin or a fragment thereof, albumin or a fragment thereof, an albumin binding moiety, a HAP sequence, a PAS sequence, or any combinations thereof. Non-limiting examples of the non-polypeptide moiety includes polyethylene glycol (PEG), polysialic acid, hydroxyethyl starch (HES), a derivative thereof, or any combination thereof. Other such moieties useful in present invention are known in the art.

In one embodiment, the adjunct moiety is associated (or linked) with the FVIII protein by a covalent or a non-covalent bond. In some instances, however, the physical blockage or chemical association (e.g., non-covalent bonding) between the adjunct moiety and the FVIII protein may not be strong enough to provide a stable complex comprising the FVIII protein and the adjunct moiety in the presence of endogenous VWF. For example, a VWF fragment forming a non-covalent bond with a FVIII protein without any other connections may readily be dissociated in vivo from the FVIII protein in the presence of endogenous VWF, replacing the VWF fragment (e.g., recombinant VWF, i.e., rVWF) with endogenous VWF. Therefore, the FVIII protein non-covalently bound to endogenous VWF would undergo the VWF clearance pathway and be cleared from the system. In order to prevent the dissociation of the adjunct moiety with the FVIII protein, in some embodiments, the linkage between the FVIII protein and the adjunct moiety is a covalent bond, e.g., a peptide bond, one or more amino acids, or a disulfide bond. In certain embodiments, the association (i.e., linkage) between the adjunct moiety and the FVIII protein is a peptide bond or a linker between the FVIII protein and the adjunct moiety (“FVIII/AM linker”). Non-limiting examples of the linker is described elsewhere herein. In some embodiments, the adjunct moiety is a polypeptide comprising, consisting essentially of, or consisting of at least about 10, 100, 200, 300, 400, 500, 600, 700, 800, 900, 1000, 1100, 1200, 1300, 1400, 1500, 1600, 1700, 1800, 1900, 2000, 2500, 3000, or 4000 amino acids. In other embodiments, the adjunct moiety is a polypeptide comprising, consisting essentially of, or consisting of about 100 to about 200 amino acids, about 200 to about 300 amino acids, about 300 to about 400 amino acids, about 400 to about 500 amino acids, about 500 to about 600 amino acids, about 600 to about 700 amino acids, about 700 to about 800 amino acids, about 800 to about 900 amino acids, or about 900 to about 1000 amino acids. In some embodiments, the adjunct moiety covalently associated with the FVIII protein is a VWF fragment described elsewhere herein.

In certain embodiments, the adjunct moiety chemically (e.g., non-covalently) binds to or physically blocks one or more VWF binding sites on a FVIII protein. The VWF binding site on a FVIII protein is located within the A3 domain or the C2 domain of the FVIII protein. In still other embodiments, the VWF binding site on a FVIII protein is located within the A3 domain and C2 domain. For example, the VWF binding site on a FVIII protein can correspond to amino acids 1669 to 1689 and/or 2303 to 2332 of SEQ ID NO: 16 [full-length mature FVIII].

In other embodiments, a chimeric protein of the invention comprises a FVIII protein linked to an adjunct moiety, wherein the adjunct moiety is a VWF molecule, e.g. a VWF fragment comprising a D′ domain and a D3 domain, but not containing the VWF clearance receptor binding site, and shields or protects the VWF binding site on the FVIII protein, thereby inhibiting or preventing interaction of the FVIII protein with endogenous VWF. In certain embodiments, the adjunct moiety is a VWF fragment. The VWF fragment useful for the present invention contains the D′ domain and the D3 domain, still providing one or more advantages of VWF-like property to the FVIII protein, but the VWF fragment does not undergo the VWF clearance pathway. The FVIII protein and the adjunct moiety can be covalently associated by a linker (e.g., FVIII/AM linker). In one embodiment, the linker can be a cleavable linker. Non-limiting examples of the linkers are disclosed elsewhere herein.

In still other embodiments, a chimeric protein of the invention comprises a FVIII protein and an immunoglobulin constant region or a portion thereof (i.e., an adjunct moiety), wherein the immunoglobulin constant region or a portion thereof shields or protects the VWF binding site on the FVIII protein, thereby inhibiting or preventing interaction of the FVIII protein with endogenous VWF. In yet other embodiments, the immunoglobulin constant region or a portion thereof is an Fc region.

In one aspect, the present invention is directed to a chimeric or fusion protein or hybrid comprising one or more of the VWF fragments disclosed herein and uses of the same. The chimeric or fusion protein can be fused or linked to one or more heterologous moiety (sometimes indicated herein as H or H1). In one embodiment, the heterologous moiety (H1) is a heterologous peptide or a heterologous polypeptide that would not naturally occur with and/or is linked to the VWF fragment. In another embodiment, the heterologous moiety (H1) is a non-polypeptide moiety, e.g., chemical modification or a combination of a peptide or polypeptide and a non-polypeptide moiety. In some embodiments, the VWF fragments are linked or connected to the heterologous moiety (H1) by a linker (also referred to herein as “VWF linker”). In one embodiment, the VWF linker is a cleavable linker. Non-limiting examples of the linker between the VWF fragment and the heterologous moiety (H1) are disclosed elsewhere herein.

In one embodiment, the heterologous moiety (H1) useful in the invention improves one or more pharmacokinetic properties of the VWF fragments without significantly affecting the VWF fragments' biological activity or function (e.g., its binding to or association with a FVIII protein). In another embodiment, the heterologous moiety (H1) linked to the VWF fragment can extend the half-life of the VWF fragments. Non-limiting examples of the heterologous polypeptide moiety comprises an immunoglobulin constant region or a portion thereof, albumin or a fragment thereof, an albumin binding moiety, a PAS sequence, a HAP sequence, transferrin or a fragment thereof, or two or more combinations thereof. Non-limiting examples of the heterologous non-polypeptide moiety include polyethylene glycol (PEG), polysialic acid, hydroxyethyl starch (HES), a derivative thereof, or any combinations thereof.

In some embodiments, a heterologous moiety (H1) can be used to connect the VWF fragment and the FVIII protein by a covalent bond. Examples of the heterologous moiety that can provide the covalently linkage include, but are not limited to, an immunoglobulin constant region or a portion thereof comprising a hinge region, e.g., an Fc region or an FcRn binding partner. In a specific example, the FVIII protein is linked to a first Fc region, and the VWF fragment is linked to a second Fc region, wherein the first Fc region and the second Fc region form one or more disulfide bond.

In some embodiments, the heterologous moiety (sometimes indicated herein by “H” or “H1”) is an immunoglobulin constant region or a portion thereof. Non-limiting examples of the immunoglobulin constant region or a portion thereof can be selected from the group consisting of a CH1 domain, a CH2 domain, a CH3 domain, a CH4 domain, a hinge domain, and two or more combinations thereof. In one embodiment, the immunoglobulin constant region or a portion thereof comprises at least one CH1 domain, at least one CH2 domain, at least one CH3 domain, at least one CH4 domain, or the functional fragments thereof. In another embodiment, the immunoglobulin constant region or a portion thereof comprises at least one hinge domain or a portion thereof and at least one CH2 domain or a portion thereof (e.g., in the hinge-CH2 orientation). In other embodiments, the immunoglobulin constant domain or a portion thereof comprises at least one CH2 domain or a portion thereof and at least one CH3 domain or a portion thereof (e.g., in the CH2-CH3 orientation.) Examples of the combination include, but are not limited to, a CH2 domain, a CH3 domain, and a hinge domain, which are also known as an Fc region (or Fc domain), e.g., a first Fc region. In other embodiments, the heterologous moiety (H1) is linked to the VWF fragment by a linker. In certain embodiments, the heterologous moiety (H1) is an FcRn binding partner as described elsewhere herein. In other embodiments, the heterologous moiety (H1) is a hinge region.

In certain embodiments, the chimeric protein further comprises a second (or additional) heterologous moiety (sometimes indicated herein by “H2”). It is noted that the first heterologous moiety (H1) and the second heterologous moiety (H2) can be used interchangeably and can be the same or different. The second heterologous moiety (H2) can be linked to the FVIII protein or elsewhere in the chimeric protein by a peptide bond, one or more amino acids, or by a linker (e.g., FVIII linker if linked to FVIII). Such constructs can sometimes be referred to as FVIII/VWF heterodimer. In one embodiment, the heterologous moiety (H2) comprises a heterologous polypeptide. In another embodiment, the heterologous moiety (H2) comprises a non-polypeptide moiety. In other embodiments, the heterologous moiety (H2) comprises a combination of a heterologous moiety and a non-polypeptide moiety. The second heterologous moiety (H2) can be a half-life extender. Non-limiting examples of the second heterologous polypeptide moiety (H2) include an immunoglobulin constant region or a portion thereof, albumin or a fragment thereof, an albumin binding moiety, a PAS sequence, a HAP sequence, transferrin or a fragment thereof, or two or more combinations thereof. Non-limiting examples of the heterologous non-polypeptide moiety include polyethylene glycol (PEG), polysialic acid, hydroxyethyl starch (HES), a derivative thereof, or any combinations thereof. In certain embodiments, the first heterologous moiety (H1) and the second heterologous moiety are the same or different. Either or both of the first heterologous moiety (H1) and the second heterologous moiety (H2) can confer half-life extension to the FVIII protein in a chimeric protein, provide a connection stronger than non-covalent association, i.e., by one or more covalent bonds between the FVIII protein and the VWF fragment in a chimeric protein, or both. Once the VWF fragment fused or linked to the first heterologous moiety (H1) removes the half-life ceiling by preventing or inhibiting interaction between the FVIII protein and the endogenous VWF protein, the FVIII protein fused to the heterologous moieties can reach to its full potential and can have a half-life of longer than two-fold compared to wild type FVIII.

In certain embodiments, the first heterologous moiety (e.g., a first Fc region) linked to the VWF fragment and the second heterologous moiety (e.g., a second Fc region) linked to the FVIII protein are associated with each other such that the association prevents replacement of the VWF fragment by endogenous VWF in vivo. In one embodiment, the second heterologous moiety is a second Fc region, wherein the second Fc region is linked to or associated with the first heterologous moiety, e.g., the first Fc region, by a covalent bond, e.g., disulfide bond, a peptide bond, or a linker (one or more amino acids). For example, the second heterologous moiety (e.g., the second Fc region) linked to the FVIII protein at one end can be further linked to the first heterologous moiety (e.g., the first Fc region) linked to the VWF fragment by a linker (e.g., scFc linker) or associated with the first heterologous moiety by a covalent or non-covalent bond. In another embodiment, the second heterologous moiety (e.g., the second Fc region) is linked to the VWF fragment that is already linked to first heterologous moiety. In some embodiments, the chimeric protein comprises a first polypeptide chain comprising a VWF fragment and a first heterologous moiety and a second polypeptide chain comprising a FVIII protein and a second heterologous moiety, wherein the first polypeptide chain and the second polypeptide chain are associated, wherein the association between the first polypeptide chain comprising the first heterologous moiety and the second polypeptide chain comprising the second heterologous moiety is a covalent bond, thus allowing the VWF fragment and the FVIII protein maintain its interaction with each other. At the same time, endogenous VWF, which can form a non-covalent bond with the FVIII protein cannot replace the covalently linked polypeptide chain comprising the VWF fragment.

The linker between the first heterologous moiety (H1) and the VWF fragment (e.g., VWF linker) can be a cleavable linker, e.g., a thrombin cleavable linker. The cleavable linkers can be cleaved by a protease selected from the group consisting of factor XIa, factor XIIa, kallikrein, factor VIIa, factor IXa, factor Xa, factor IIa (thrombin), Elastase-2, Granzyme-B, TEV, Enterokinase, Protease 3C, Sortase A, MMP-12, MMP-13, MMP-17, MMP-20, and any combinations thereof. These cleavable linkers allow the VWF fragment to be cleaved and dissociated from the FVIII protein upon activation of the clotting cascade, resulting in a FVIII protein with full activity potential.

In other embodiments, the chimeric protein is produced as a single polypeptide chain comprising a VWF fragment, a cleavable linker, a first heterologous moiety (H1), a processable linker, a FVIII protein, and a second heterologous moiety (H2) in any order. After synthesis, the processable linker can be cleaved by an intracellular protease enzyme before secretion, thus making two polypeptide chains as described above. In the single chain construct before secretion, the second heterologous moiety (e.g., the second Fc region) can be linked to the VWF fragment by a processable linker. In certain embodiments, one or more linkers can comprise one or more cleavage sites.

In some embodiments, the chimeric protein of the invention further comprises a third heterologous moiety (sometimes indicated herein by “H3”). The third heterologous moiety (H3) can be a half-life extender. The heterologous moiety (H3) can comprise a heterologous polypeptide, a non-polypeptide moiety, or a combination of both. Non-limiting examples of the third heterologous moiety (H3) include an immunoglobulin constant region or a portion thereof, albumin or a fragment thereof, an albumin binding moiety, a PAS sequence, a HAP sequence, transferrin or a fragment thereof, any derivatives or variants thereof, or two or more combinations thereof. Non-limiting examples of the non-polypeptide moiety include polyethylene glycol (PEG), polysialic acid, hydroxyethyl starch (HES), a derivative thereof, or any combinations thereof. The first heterologous moiety (H1) linked to the VWF fragment, the second heterologous moiety (H2) linked to the FVIII protein, and the third heterologous moiety (H3) can be the same or different. In one embodiment, the first heterologous moiety (H1) is identical to the second heterologous moiety (H2), but is different from the third heterologous moiety (H3). In another embodiment, the third heterologous moiety (H3) is fused or linked to a FVIII protein or a VWF fragment of the chimeric protein. In some embodiments, the third heterologous moiety is inserted within one or more domains of the FVIII protein or between two domains of the FVIII protein.

In one embodiment, a chimeric protein comprises a first polypeptide chain and a second polypeptide chain, wherein the first chain comprises a FVIII protein linked to a first heterologous moiety (H1), e.g., a first Fc region, by an optional linker (e.g., FVIII linker) and the second chain comprises a VWF fragment linked to a second heterologous moiety (H2), e.g., a second Fc region, by an optional linker (e.g., VWF linker). The FVIII protein can further comprise a third heterologous moiety (H3), e.g., any half-life extending moiety, e.g., albumin, or a PAS sequence, between FVIII heavy chain and FVIII light chain (i.e., amino acid residue 1648 of SEQ ID NO: 16), thus being a single chain FVIII protein. Alternatively, the FVIII protein can be a dual chain protein, i.e., the FVIII heavy chain and the FVIII light chain associated with each other by a covalent or non-covalent bond (e.g., a metal bond), wherein the heavy chain is further linked to a third heterologous moiety (H3), e.g., a non-structural half-life extending polypeptide, albumin or a fragment thereof or a PAS sequence. In another embodiment, a chimeric protein comprises a first polypeptide chain and a second polypeptide chain, wherein the first chain comprises a FVIII protein linked to a first heterologous moiety (H1), e.g., a first Fc region, by an optional linker (e.g, FVIII linker) and the second chain comprises a VWF fragment linked to a third heterologous moiety (H3), e.g., a non-structural half-life extending polypeptide, albumin or a PAS sequence, which is linked to a second heterologous moiety (H2), e.g., a second Fc region, by an optional linker. In some embodiments, the third heterologous moiety (H3) (e.g., a half-life extending polypeptide) can be linked to the C-terminus or N-terminus of the FVIII protein or inserted between two domains of the FVIII protein or between two amino acids in a domain of the FVIII protein.

In other embodiments, the chimeric protein of the invention further comprises a fourth heterologous moiety (sometimes indicated herein by “H4”) and/or a fifth heterologous moiety (sometimes indicated herein by “H5”). The fourth or fifth heterologous moiety can also be a half-life extender. The fourth heterologous moiety and/or the fifth heterologous moiety can be the same or different from the third heterologous moiety. The heterologous moiety can comprise a heterologous polypeptide, a non-polypeptide moiety, or a combination of both. Non-limiting examples of the fourth or fifth heterologous moiety include an immunoglobulin constant region or a portion thereof, albumin or a fragment thereof, an albumin binding moiety, a PAS sequence, a HAP sequence, transferrin or a fragment thereof, any derivatives or variants thereof, or two or more combinations thereof. Non-limiting examples of the non-polypeptide moiety include polyethylene glycol (PEG), polysialic acid, hydroxyethyl starch (HES), a derivative thereof, or any combinations thereof. The first heterologous moiety, the second heterologous moiety, the third heterologous moiety, the fourth heterologous moiety, and the fifth heterologous moiety can be the same or different. In some embodiments, the fourth heterologous moiety (e.g., a half-life extending polypeptide) can be linked to the C-terminus or N-terminus of the FVIII protein or inserted between two domains of the FVIII protein or between two amino acids in a domain of the FVIII protein. In other embodiments, the fifth heterologous moiety (e.g., a half-life extending polypeptide) can also be linked to the C-terminus or N-terminus of the FVIII protein or inserted between two domains of the FVIII protein or between two amino acids in a domain of the FVIII protein.

In certain embodiments, the chimeric protein comprises a FVIII protein, a VWF fragment, a first heterologous moiety, a second heterologous moiety, a third heterologous moiety, a fourth heterologous moiety, and a fifth heterologous moiety, wherein the first heterologous moiety and the second heterologous moiety forms a bond (e.g., a covalent bond) between the chain comprising the FVIII protein and the chain comprising the VWF fragment, and the third heterologous moiety, the fourth heterologous moiety, and the fifth heterologous moiety are half-life extenders, and wherein the bond between the chain comprising the FVIII protein and the chain comprising the VWF fragment is stronger than the non-covalent interaction between the FVIII and the VWF fragment, thereby preventing binding of endogenous VWF to the FVIII protein in vivo, in vitro, or ex vivo.

In other embodiments, the chimeric protein comprises a FVIII protein, a VWF fragment, a first heterologous moiety, a second heterologous moiety, a third heterologous moiety, a fourth heterologous moiety, a fifth heterologous moiety, and a sixth heterologous moiety (sometimes indicated herein as “H6”), wherein the first heterologous moiety and the second heterologous moiety forms a bond between the chain comprising the FVIII protein and the chain comprising the VWF fragment, and the third heterologous moiety, the fourth heterologous moiety, the fifth heterologous moiety, and the sixth heterologous moiety are half-life extenders, and wherein the bond between the chain comprising the FVIII protein and the chain comprising the VWF fragment is stronger than the interaction between the FVIII and the VWF fragment, thereby preventing binding of endogenous VWF to the FVIII protein in vivo, in vitro, or ex vivo.

In some embodiments, a chimeric protein comprises a formula selected from the group consisting of:

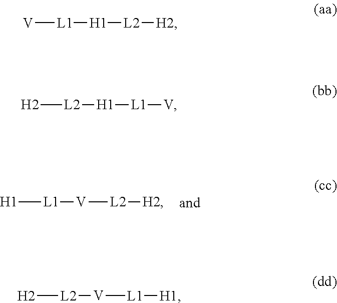

wherein V comprises a VWF fragment described herein;
Each of L1 and L2 comprises an optional linker; and
H1 comprises a first heterologous moiety; and
H2 comprises an optional second heterologous moiety. Either or both of the first heterologous moiety and the second heterologous moiety can be a half-life extending moiety. In one embodiment, H1 comprises a polypeptide, a non-polypeptide moiety, or both. The polypeptide useful as H1 can comprise an immunoglobulin constant region or a portion thereof, albumin or a fragment thereof, an albumin binding moiety, a PAS sequence, a HAP sequence, any derivatives or variants, or any combinations thereof. The non-polypeptide moiety can comprise polyethylene glycol (PEG), polysialic acid, and hydroxyethyl starch (HES), a derivative or variant thereof, or any combinations thereof. In another embodiment, H2 comprises a polypeptide, a non-polypeptide moiety, or both. The polypeptide useful as H2 can comprise an immunoglobulin constant region or a portion thereof, albumin or a fragment thereof, an albumin binding moiety, a PAS sequence, a HAP sequence, any derivatives or variants, or any combinations thereof. The non-polypeptide moiety can comprise polyethylene glycol (PEG), polysialic acid, hydroxyethyl starch (HES), a derivative or variant thereof, or any combinations thereof. In certain embodiments, the linker between H1 and H2 in formulas (aa) and (bb) is a processable linker. In other embodiments, the linker between the VWF fragment and H1 in formulas (aa) and (bb) is a cleavable linker, e.g., a thrombin cleavable linker that can be cleaved by thrombin.

The orientation of the polypeptide formulas herein is listed from N-terminus (left) to C-terminus (right). For example, formula H-L-V means formula NH2-H-L-V-COOH. In one embodiment, the formulas described herein can comprise additional sequences between the two moieties. For example, formula V-L1-H1-L2-H2 can further comprise sequences at the N-terminus of V, between V and L1, between L1 and H1, between H1 or L2, between L2 or H2, or at the C-terminus of H2 unless otherwise specified. In another embodiment, the hyphen (-) indicates a peptide bond or one or more amino acids.

In specific embodiments, a chimeric protein comprises, consists essentially of, or consists of one or more formulas selected from the group consisting of (a1) V-H, (a2) H-V, (a3) V-L-H, (a4) H-L-V, (a5) V-L1-H1-H2, (a6) H2-H1-L1-V, (a7) V-L1-H1:H2, (a8) H2:H1-L1-V, (a9) V-H1:H2, (b1) H2:H1-V, (b2) V-L1-H1-L2-H2, (b3) H2-L2-H1-L1-V, (b4) H1-V-H2, (b5) H1-L1-V-L2-H2, and (b6) H2-L2-V-L1-H1, wherein V comprises one or more of the VWF fragments described herein, L, L1, or L2 comprises a linker, H or H1 comprises a first heterologous moiety. In one embodiment, the first heterologous moiety (H1) can be a polypeptide, a non-polypeptide moiety, or both. The heterologous polypeptide moiety can comprises an immunoglobulin constant region or a portion thereof, albumin or a fragment thereof, an albumin binding moiety, a PAS sequence, a HAP sequence, or any combinations thereof. Non-limiting examples of the non-polypeptide moiety useful as H1 include polyethylene glycol (PEG), polysialic acid, hydroxyethyl starch (HES), a derivative thereof, or any combinations thereof. In another embodiment, H2 comprises a second heterologous moiety. The second heterologous moiety can be a polypeptide, a non-polypeptide moiety, or both. The heterologous polypeptide moiety can comprises an immunoglobulin constant region or a portion thereof, albumin or a fragment thereof, an albumin binding moiety, a PAS sequence, a HAP sequence, or any combinations thereof. Non-limiting examples of the non-polypeptide moiety useful as H1 include polyethylene glycol (PEG), polysialic acid, hydroxyethyl starch (HES), a derivative thereof, or any combinations thereof. In certain embodiments, the linker between the first heterologous moiety and the second heterologous moiety is a processable linker. In other embodiments, the linker between the VWF fragment and the first heterologous moiety or the second heterologous moiety is a cleavable linker, which comprises one or more cleavage sites, e.g., a thrombin cleavable linker.

The chimeric protein of the present invention comprises a formula selected from the group consisting of (aa), (bb), (cc), (dd), (a1), (a2), (a3), (a4), (a5), (a6), (a7), (a8), (a9), (b1), (b2), (b3), (b4), (b5), and (b6) and a FVIII protein, which is covalently linked to or covalently associated with the VWF fragment, the first heterologous moiety (e.g., a first Fc region), or the second heterologous moiety (e.g., a second Fc region) of the formula. In one embodiment, the FVIII protein is linked to or associated with the VWF fragment by a covalent or non-covalent bond or by a linker. In another embodiment, the FVIII protein can be linked to the first heterologous moiety or the second heterologous moiety by a covalent or non-covalent bond or by a linker.

In one embodiment, a chimeric protein of the present invention comprises a VWF fragment described herein covalently linked to or covalently associated with a FVIII protein. For example, the chimeric protein can comprise a VWF fragment and a FVIII protein, wherein the VWF fragment and the FVIII protein are bound by a covalent non-peptide bond, a peptide bond, a non-covalent bond, or by a linker, e.g., a cleavable linker. In a specific embodiment, the VWF fragment and the FVIII protein are bound to or interact with each other by one or more disulfide bonds. In another specific embodiment, the VWF fragment is bound to or interacts with the FVIII protein at the A3 domain of FVIII, the C2 domain of FVIII, or both the A3 domain and the C2 domain of FVIII by a non-covalent bond. In another embodiment, the VWF fragment bound to or interacting with the FVIII protein is linked or fused to a first heterologous moiety. In other embodiments, the FVIII protein bound to or interacting with the VWF fragment is further linked to a second heterologous moiety. In some embodiments, the VWF fragment bound to or interacting with the FVIII protein is further linked to a first heterologous moiety and the FVIII protein is further linked to a second heterologous moiety. In certain embodiments, the first polypeptide chain comprising the VWF fragment and the first heterologous moiety and the second polypeptide chain comprising the FVIII protein and the second heterologous moiety are associated with each other such that the association does not allow interaction of the FVIII protein with other moieties, e.g., endogenous VWF. In one embodiment, the association is a covalent bond, e.g., a disulfide bond.

Each of the VWF fragment or the FVIII protein can be joined or connected to the first and second heterologous moiety by a linker, e.g., a cleavable linker, e.g., a thrombin cleavable linker. The linker between the VWF fragment and the first heterologous moiety can be denoted herein as a VWF linker. The linker between the FVIII protein and the second heterologous moiety can be denoted herein as a FVIII linker. Or, both of the VWF fragment or the FVIII protein can be joined or connected to the first and second heterologous moiety by a linker, e.g., a cleavable linker, e.g., a thrombin cleavable linker. In certain embodiments, the first heterologous moiety linked to the VWF fragment comprises a polypeptide, a non-polypeptide moiety, or both. Non-limiting examples of the first heterologous polypeptide moiety includes an immunoglobulin constant region or a portion thereof, albumin or a fragment thereof, an albumin binding moiety, a PAS sequence, a HAP sequence, transferrin or a fragment thereof, or two or more combinations thereof. Non-limiting examples of the non-polypeptide moiety includes polyethylene glycol (PEG), polysialic acid, hydroxyethyl starch (HES or HAES), a derivative or variant thereof, or any combinations thereof. In other embodiments, the second heterologous moiety linked to the FVIII protein comprises a polypeptide, a non-polypeptide moiety, or both. Non-limiting examples of the second heterologous moiety includes an immunoglobulin constant region or a portion thereof, albumin or a fragment thereof, an albumin binding moiety, a PAS sequence, a HAP sequence, transferrin or a fragment thereof, or two or more combinations thereof. Non-limiting examples of the non-polypeptide moiety includes polyethylene glycol (PEG), polysialic acid, hydroxyethyl starch (HES or HAES), a derivative or variant thereof, or any combinations thereof. In some embodiments, the VWF fragment is attached to FVIII using sortase mediated in vitro protein ligation. In some embodiments, a sortase recognition motif is used.

In one embodiment, the first heterologous moiety is an immunoglobulin constant region or a portion thereof. In a particular embodiment, the first heterologous moiety is a first Fc region. In some embodiments, the second heterologous moiety is an immunoglobulin constant region or a portion thereof. In a specific embodiment, the second heterologous moiety is a second Fc region. In a particular embodiment, the chimeric protein comprises a VWF fragment described herein and a FVIII protein, wherein the VWF fragment is linked to an immunoglobulin constant region or a portion thereof, which is an Fc region. In another embodiment, the chimeric protein comprises a VWF fragment described herein and a FVIII protein, wherein the FVIII protein is linked to an immunoglobulin constant region or a portion thereof, which is an Fc region. In other embodiments, a chimeric protein comprises a VWF fragment described herein and a FVIII protein, wherein the VWF fragment is linked to a first immunoglobulin constant region, which is a first Fc region, and the FVIII protein is linked to a second immunoglobulin constant region, which is a second Fc region, and wherein the VWF fragment and the FVIII protein is bound to or interact with each other by a non-covalent bond or the first Fc region or the second Fc region are associated with each other by a covalent bond. In still other embodiments, the VWF fragment linked to the first heterologous moiety is further linked to the second heterologous moiety, e.g., a second Fc region, by a linker, e.g., a processable linker. In one aspect, the VWF fragment is linked to the first heterologous moiety by a linker, e.g., VWF linker, e.g., a cleavable linker. In another aspect, the FVIII protein is linked to the second heterologous moiety by a linker, e.g., FVIII linker, e.g., a cleavable linker. Non-limiting examples of the heterologous moieties are disclosed elsewhere herein, e.g., immunoglobulin constant region or a portion thereof at paragraphs [0165]-[0193], albumin, fragment or variant thereof at paragraphs [0194]-[0198], HAP sequences at paragraph [0293], transferrin, fragments, or variants thereof at paragraphs [0204]-[0205], polymer, e.g., polyethylene glycol, at paragraphs [0206]-[0213], HES at paragraphs [0214]-[0219], or PSA at paragraph [0220]- and PAS sequences at paragraphs [0199]-[0202].

In some embodiments, a chimeric protein of the present invention comprises, consists essentially of, or consists of a formula selected from the group consisting of:

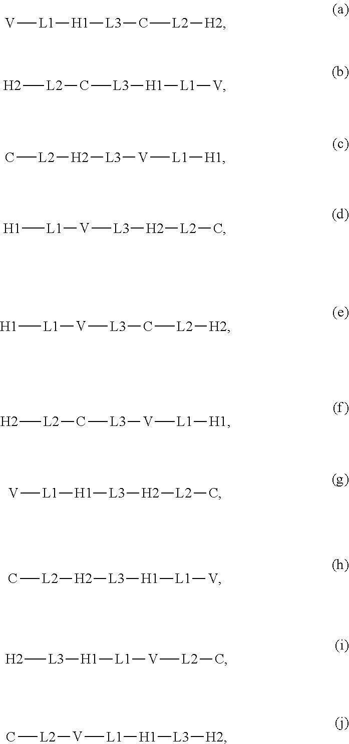

wherein V is a VWF fragment described herein;
each of L1 or L2 is an optional linker, e.g., a cleavable linker, e.g., a thrombin cleavable linker;
L3 is an optional linker, e.g., a processable linker
each of H1 and H2 is an optional heterologous moiety;
C is a FVIII protein; and
(-) is a peptide bond or one or more amino acids.

In other aspects, a chimeric protein of the invention comprises a formula selected from the group consisting of:

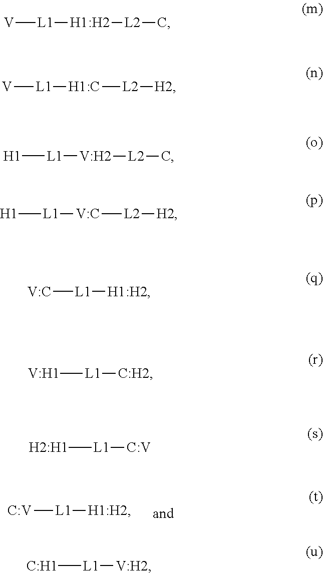

wherein V is a VWF fragment described herein;
each of L1 or L2, is an optional linker, e.g., a thrombin cleavable linker;
each of H1 or H2 is an optional heterologous moiety;
(-) is a peptide bond or one or more amino acids; and
C is a FVIII protein; and (:) is a chemical or physical association between H1 and H2.

In one embodiment, one or more of the heterologous moieties are a half-life extender. Half-life extenders are known in the art, and non-limiting examples of such half-life extenders include an immunoglobulin constant region or a portion thereof, albumin or fragment thereof, an albumin binding moiety, a PAS sequence, a HAP sequence, transferrin or a fragment thereof, a derivative or variant thereof, or two or more combinations thereof. The non-polypeptide moiety can comprise polyethylene glycol (PEG), polysialic acid, hydroxyethyl starch (HES), a derivative thereof, or any combinations thereof.

In one embodiment, (:) in formulas (m) to (u) represents a chemical association, e.g., at least one non-peptide bond. In certain embodiments, the chemical association, i.e., (:) is a covalent bond. In other embodiments, the chemical association, i.e., (:) is a non-covalent interaction, e.g., an ionic interaction, a hydrophobic interaction, a hydrophilic interaction, a Van der Waals interaction, a hydrogen bond. In other embodiments, (:) is a non-peptide covalent bond. In still other embodiments, (:) is a peptide bond. In yet other embodiments, (:) in formulas (m) to (u) represents a physical association between two sequences, wherein a portion of a first sequence is in close proximity to a second sequence such that the first sequence shields or blocks a portion of the second sequence from interacting with another moiety, and further that this physical association is maintained without allowing the second sequence to interact with other moieties.

Formulas (a)-(u) are included herein merely as non-limiting examples of constructs of the present invention. The orientation of the polypeptide formulas is shown from N-terminus (left) to C-terminus (right). For example, formula V-L1-H1-L3-C-L2-H2 means formula NH2-V-L1-H1-L3-C-L2-H2-COOH). In addition, (:) can be an association or interaction between two polypeptide chains by a covalent bond or a non-covalent bond between any part of the first chain and any part of the second chain unless otherwise noted. For example, formula V-H1:H2-C has two polypeptide chains, the first chain being V-H1 and the second chain being C-H2, wherein V in the first chain interacts or associates with C in the second chain and/or H1 in the first chain interacts or associates with H2 in the second chain. In some embodiments, (:) means a covalent, non-peptide bond or non-covalent bond.

In certain embodiments, a chimeric protein comprises, consists essentially of, or consists of a formula selected from the group consisting of:

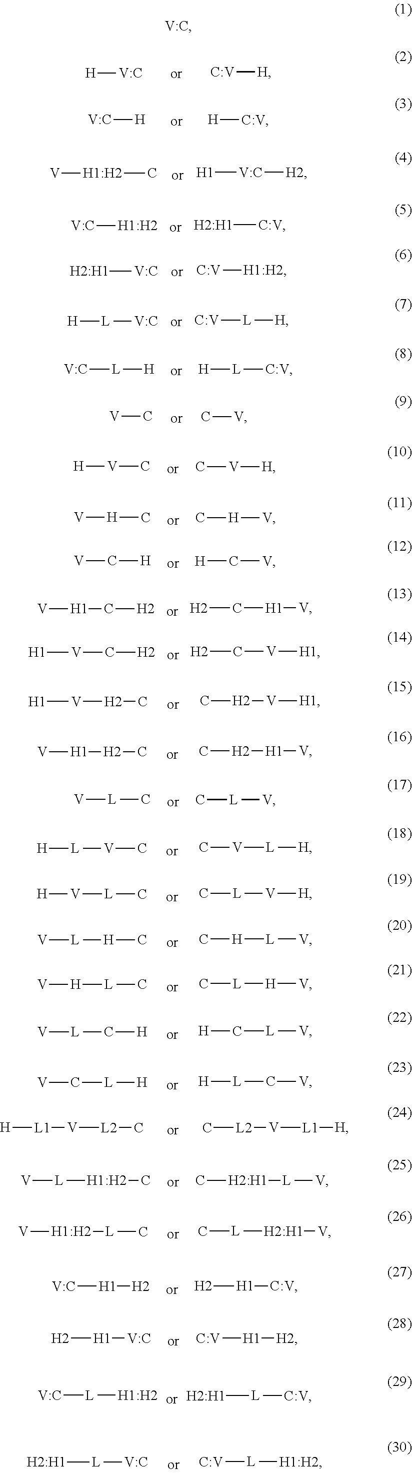

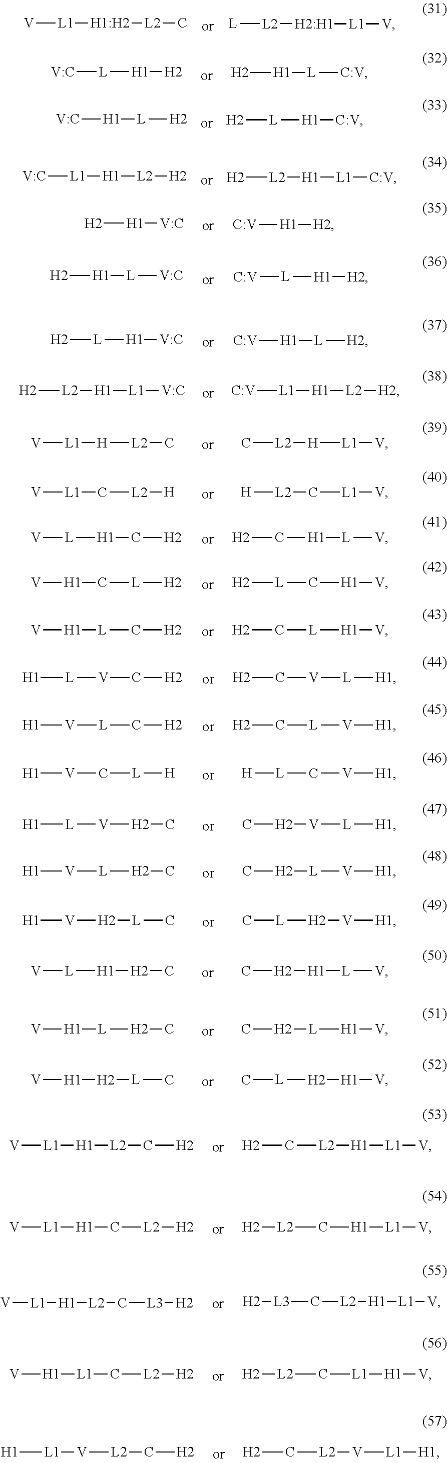

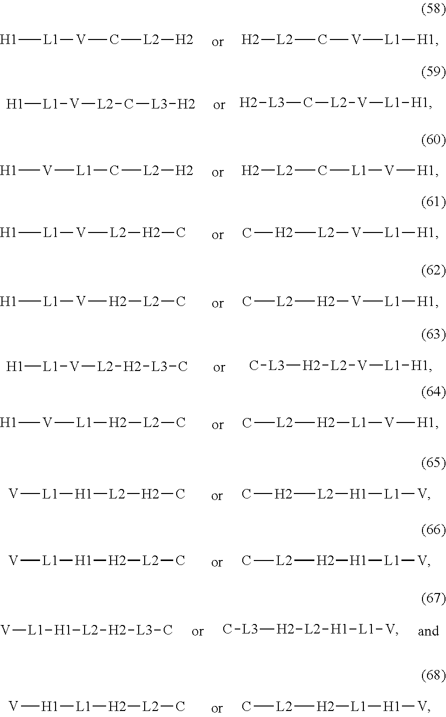

V is a VWF fragment described herein;
C is a FVIII protein;
H or H1 is a heterologous moiety or a first heterologous moiety;
H2 is a second heterologous moiety; the first and second heterologous moieties can be the same or different;
Each of L, L1 or L2 is an optional linker;
(-) is a peptide bond or one or more amino acids; and
   (:) is a chemical or physical association. The linkers can each be the same or different and each can be a cleavable linker, comprising one or more enzymatic cleavage site. The heterologous moieties can be a half-life extension technology that is known in the art, a polypeptide, a non-polypeptide moiety, or both. A polypeptide moiety can comprise an immunoglobulin constant region or a portion thereof, albumin or a fragment thereof, an albumin binding moiety, a PAS sequence, a HAP sequence, any derivatives or variants thereof, or any combinations thereof (e.g., an Fc region). A non-polypeptide moiety can comprise polyethylene glycol (PEG), polysialic acid, hydroxyethyl starch (HES), a derivative or variant thereof, or any combinations thereof. Each of the H, H1, or H2 can be individually selected based on the characteristics and can be all the same, or each one different. Non-limiting examples of the heterologous moieties are disclosed elsewhere herein, e.g., immunoglobulin constant region or a portion thereof at paragraphs [0126]-[0153], albumin or fragment or variant thereof at paragraphs [0154]-[0157], polymer, e.g., polyethylene glycol, at paragraphs [0166]-[0173], and PAS sequences at paragraphs [0159]-[0162]. Formulas (1)-(68) are included herein merely as non-limiting examples of constructs of the present invention.

In one embodiment, (:) represents a chemical association, e.g., at least one non-peptide bond. In certain embodiments, the chemical association, i.e., (:) is a covalent bond. In other embodiments, the chemical association, i.e., (:) is a non-covalent interaction, e.g., an ionic interaction, a hydrophobic interaction, a hydrophilic interaction, a Van der Waals interaction, a hydrogen bond. In other embodiments, (:) is a non-peptide covalent bond. In still other embodiments, (:) is a peptide bond. In yet other embodiments, (:) represents a physical association between two sequences, wherein a portion of a first sequence is in close proximity to a second sequence such that the first sequence shields or blocks a portion of the second sequence from interacting with another moiety, and further that this physical association is maintained without allowing the second sequence to interact with other moieties.

In one embodiment, the first heterologous moiety (H or H1) linked to the VWF fragment in the chimeric protein is a first Fc region. In another embodiment, the second heterologous moiety (or H2) linked to the FVIII protein in the chimeric protein is a second Fc region.

In certain embodiments, a chimeric protein of the invention comprises two polypeptide chains, a first chain comprising, consisting essentially of, or consisting of an amino acid sequence encoding FVIII (e.g., single chain FVIII) and a first heterologous moiety (e.g., a first Fc region) and a second chain comprising, consisting essentially of, or consisting of an amino acid sequence encoding a VWF fragment comprising D′ domain and D3 domain, a second heterologous moiety (e.g., a second Fc region), and a linker between the VWF fragment and the second Fc domain (e.g., VWF linker). The linker between the VWF fragment and the second Fc domain can be a thrombin cleavable linker. In some embodiments, the single chain FVIII protein comprises a third heterologous moiety, e.g., a half-life extender, which is linked to the N-terminus, C-terminus, or one or more sites within the FVIII sequence.

In other embodiments, a chimeric protein of the invention comprises three polypeptide chains, wherein a first chain comprises, consists essentially of, or consists of a heavy chain of FVIII, a second chain comprises, consists essentially of, or consists of a light chain of FVIII fused to a first heterologous moiety (e.g., a first Fc region), and a third polypeptide chain comprises, consists essentially of, or consists of a VWF fragment comprising the D′ domain and the D3 domain, a second heterologous moiety (e.g, a second Fc region), and a linker. The linker between the VWF fragment and the second heterologous moiety can be a thrombin cleavable linker. In some embodiments, the heavy chain FVIII is linked to a third heterologous moiety, e.g., a half-life extender, which can be linked to the N-terminus, C-terminus, or one or more sites within the FVIII sequence.

In yet other embodiments, a chimeric protein of the invention comprises two polypeptide chains, a first chain comprising, consisting essentially of, or consisting of a heavy chain of FVIII and a second chain comprising, consisting essentially of, or consisting of a light chain of FVIII, a first heterologous moiety (e.g., a first Fc region), a first linker (e.g., a protease cleavage site comprising one or more intracellular processing sites), a VWF fragment, a second linker (e.g., a thrombin cleavable linker), and a second heterologous moiety (e.g., a second Fc region), wherein the light chain of FVIII is linked to the first heterologous moiety (e.g., the first Fc region), which is further linked to the VWF fragment by the first linker (e.g. a processable linker having a protease cleavage site comprising one or more intracellular processing sites), and wherein the VWF fragment is linked to the second Fc region by the second linker (e.g., a thrombin cleavable linker). In certain embodiments, the first linker and the second linker are identical or different.

In certain embodiments, a chimeric protein of the invention comprises one polypeptide chain, which comprises a single chain FVIII protein, a first heterologous moiety (e.g., a first Fc region), a first linker (e.g., a thrombin cleavable linker), a VWF fragment, a second linker (e.g., a thrombin cleavable linker), and a second heterologous moiety (e.g., a second Fc region), wherein the single chain FVIII protein is linked to the first heterologous moiety, which is also linked to the VWF fragment by the first linker, and the VWF fragment is linked to the second Fc region by the second linker. In one embodiment, the first linker is a cleavable linker comprising a first cleavable site and a second cleavable site. In another embodiment, the second linker is a cleavable linker comprising one or two cleavable sites. In a specific embodiment, the second linker is a thrombin cleavable linker. The linker useful in the invention can be any length, e.g., at least 10, 50, 100, 200, 300, 400, 500, 600, or 700 amino acids. For example, the linker can be 20 amino acids, 35 amino acids, 42 amino acids, 73 amino acids, or 98 amino acids.

In certain embodiments, the VWF fragment is directly linked to the FVIII protein by a peptide bond or a linker. As one way of linking the VWF fragment and the FVIII protein directly or through a linker, an enzymatic ligation (e.g., sortase) can be employed. For example, sortase refers to a group of prokaryotic enzymes that modify surface proteins by recognizing and cleaving a carboxyl-terminal sorting signal. For most substrates of sortase enzymes, the recognition signal consists of the motif LPXTG (Leu-Pro-any-Thr-Gly (SEQ ID NO: 106), then a highly hydrophobic transmembrane sequence, then a cluster of basic residues such as arginine. Cleavage occurs between the Thr and Gly, with transient attachment through the Thr residue to the active site Cys residue of a ligation partner, followed by transpeptidation that attaches the protein covalently to the cell wall. In some embodiments, the ligation partner contains Gly(n).

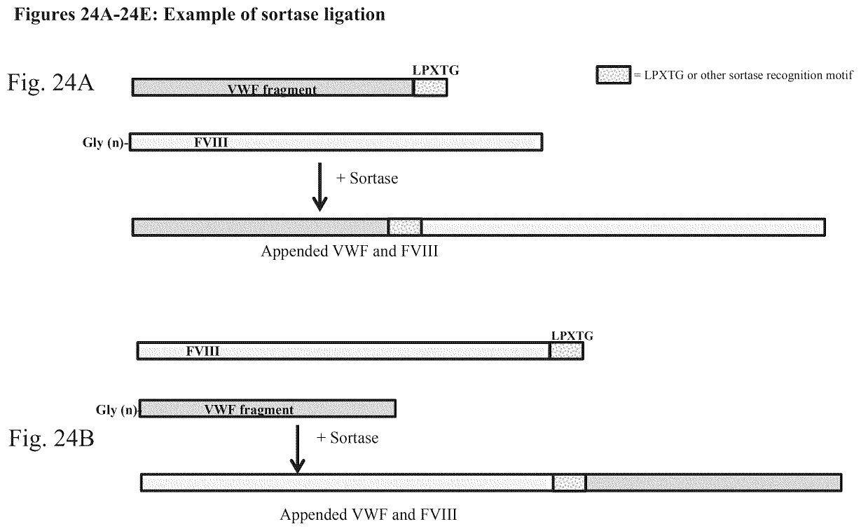

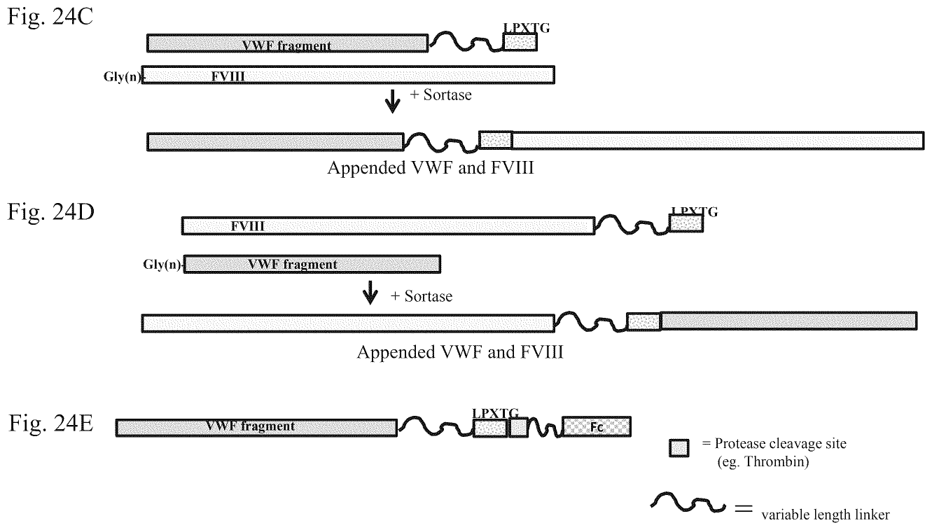

In one embodiment, a VWF fragment linked to a sortase recognition motif by an optional linker can be fused to a FVIII protein linked to Gly(n) by a sortase, wherein n can be any integer. A ligation construct comprises the VWF fragment (N-terminal portion of the construct) and the FVIII protein (C-terminal portion of the construct), wherein the sortase recognition motif is inserted in between. An exemplary construct is shown in FIG. 24(A). Another ligation construct comprises the VWF fragment (N-terminal portion of the construct, the linker, the sortase recognition motif, and the FVIII protein (C-terminal portion of the construct) (e.g., FIG. 24(C)). In another embodiment, a FVIII protein linked to a sortase recognition motif by an optional linker can be fused to a VWF fragment linked to Gly(n) by a sortase, wherein n is any integer. A resulting ligation construct comprises the FVIII protein (N-terminal portion of the construct) and the VWF fragment (C-terminal portion of the construct), wherein the sortase recognition motif is inserted in between. An exemplary construct is shown in FIG. 24(B). Another resulting ligation construct comprises the FVIII protein (N-terminal portion of the construct), the linker, the sortase recognition motif, and the VWF fragment (C-terminal portion of the construct) (e.g., FIG. 24(D)). In other embodiments, a VWF fragment linked to a sortase recognition motif by a first optional linker can be fused to a heterologous moiety, e.g., an immunoglobulin constant region or a portion thereof, e.g., an Fc region, linked to a thrombin cleavage site by a second optional linker. A resulting construct can comprise the VWF fragment (N-terminal portion), the first linker, the sortase recognition motif, the protease cleavage site, the second optional linker, and the heterologous moiety (e.g., FIG. 24(E)). In certain embodiments, this resulting construct is a part of a chimeric protein comprising the FVIII protein and a second heterologous moiety, e.g., an immunoglobulin constant region or a portion thereof, e.g., a second Fc region. In one example, In another example, a chimeric comprises three polypeptide chains, the first chain comprising a VWF fragment, the first linker, the sortase recognition motif, the protease cleavage site, the second optional linker, the first heterologous moiety, the second chain comprising the light chain of the FVIII protein and the second heterologous moiety, and the third chain comprising the heavy chain of the FVIII protein.

In still other embodiments, the chimeric protein of the invention comprising a VWF fragment and a FVIII protein, wherein the VWF fragment and the FVIII protein are covalently associated with each other or covalently linked to each other has less immunogenicity than a FVIII protein without the VWF fragment. The reduced immunogenicity includes, but is not limited to, less humoral immune response, e.g., less neutralizing antibody titer, or less cell-mediated immune response against FVIII, e.g., production of various cytokines.

In yet other embodiments, as a result of the invention the half-life of the FVIII protein (or a chimeric protein) is extended compared to a FVIII protein without the VWF fragment or wildtype FVIII. The half-life of the FVIII protein is at least about 1.5 times, at least about 2 times, at least about 2.5 times, at least about 3 times, at least about 4 times, at least about 5 times, at least about 6 times, at least about 7 times, at least about 8 times, at least about 9 times, at least about 10 times, at least about 11 times, or at least about 12 times longer than the half-life of a FVIII protein without the VWF fragment. In one embodiment, the half-life of FVIII is about 1.5-fold to about 20-fold, about 1.5 fold to about 15 fold, or about 1.5 fold to about 10 fold longer than the half-life of wild-type FVIII. In another embodiment, the half-life of the FVIII is extended about 2-fold to about 10-fold, about 2-fold to about 9-fold, about 2-fold to about 8-fold, about 2-fold to about 7-fold, about 2-fold to about 6-fold, about 2-fold to about 5-fold, about 2-fold to about 4-fold, about 2-fold to about 3-fold, about 2.5-fold to about 10-fold, about 2.5-fold to about 9-fold, about 2.5-fold to about 8-fold, about 2.5-fold to about 7-fold, about 2.5-fold to about 6-fold, about 2.5-fold to about 5-fold, about 2.5-fold to about 4-fold, about 2.5-fold to about 3-fold, about 3-fold to about 10-fold, about 3-fold to about 9-fold, about 3-fold to about 8-fold, about 3-fold to about 7-fold, about 3-fold to about 6-fold, about 3-fold to about 5-fold, about 3-fold to about 4-fold, about 4-fold to about 6 fold, about 5-fold to about 7-fold, or about 6-fold to about 8 fold as compared to wild-type FVIII or a FVIII protein without the VWF fragment. In other embodiments, the half-life of FVIII is at least about 17 hours, at least about 18 hours, at least about 19 hours, at least about 20 hours, at least about 21 hours, at least about 22 hours, at least about 23 hours, at least about 24 hours, at least about 25 hours, at least about 26 hours, at least about 27 hours, at least about 28 hours, at least about 29 hours, at least about 30 hours, at least about 31 hours, at least about 32 hours, at least about 33 hours, at least about 34 hours, at least about 35 hours, at least about 36 hours, at least about 48 hours, at least about 60 hours, at least about 72 hours, at least about 84 hours, at least about 96 hours, or at least about 108 hours. In still other embodiments, the half-life of FVIII is about 15 hours to about two weeks, about 16 hours to about one week, about 17 hours to about one week, about 18 hours to about one week, about 19 hours to about one week, about 20 hours to about one week, about 21 hours to about one week, about 22 hours to about one week, about 23 hours to about one week, about 24 hours to about one week, about 36 hours to about one week, about 48 hours to about one week, about 60 hours to about one week, about 24 hours to about six days, about 24 hours to about five days, about 24 hours to about four days, about 24 hours to about three days, or about 24 hours to about two days.

In some embodiments, the average half-life of the FVIII protein per subject is about 15 hours, about 16 hours, about 17 hours, about 18 hours, about 19 hours, about 20 hours, about 21 hours, about 22 hours, about 23 hours, about 24 hours (1 day), about 25 hours, about 26 hours, about 27 hours, about 28 hours, about 29 hours, about 30 hours, about 31 hours, about 32 hours, about 33 hours, about 34 hours, about 35 hours, about 36 hours, about 40 hours, about 44 hours, about 48 hours (2 days), about 54 hours, about 60 hours, about 72 hours (3 days), about 84 hours, about 96 hours (4 days), about 108 hours, about 120 hours (5 days), about six days, about seven days (one week), about eight days, about nine days, about 10 days, about 11 days, about 12 days, about 13 days, or about 14 days.

In certain embodiments, the half-life of the FVIII protein covalently linked to the VWF fragment is extendable in FVIII/VWF double knockout (“DKO”) mice compared to a polypeptide consisting of FVIII or a FVIII monomer-dimer hybrid.

### A) Von Willebrand Factor (VWF) Fragments

VWF (also known as F8VWF) is a large multimeric glycoprotein present in blood plasma and produced constitutively in endothelium (in the Weibel-Palade bodies), megakaryocytes (α-granules of platelets), and subendothelian connective tissue. The basic VWF monomer is a 2813 amino acid protein. Every monomer contains a number of specific domains with a specific function, the D′ and D3 domains (which together bind to Factor VIII), the A1 domain (which binds to platelet GPIb-receptor, heparin, and/or possibly collagen), the A3 domain (which binds to collagen), the C1 domain (in which the RGD domain binds to platelet integrin αIIbβ3 when this is activated), and the “cysteine knot” domain at the C-terminal end of the protein (which VWF shares with platelet-derived growth factor (PDGF), transforming growth factor-β (TGFβ) and β-human chorionic gonadotropin (βHCG)).

The 2813 monomer amino acid sequence for human VWF is reported as Accession Number _NP_000543.2_ in Genbank. The nucleotide sequence encoding the human VWF is reported as Accession Number NM_000552.3_in Genbank. The nucleotide sequence of human VWF is designated as SEQ ID NO: 1. SEQ ID NO: 2 is the amino acid sequence encoded by SEQ ID NO: 1. Each domain of VWF is listed in Table 1.

TABLE 1

| VWF domains | Amino acid Sequence | |
|---|---|---|
| VWF Signal Peptide | 1 | MIPARFAGVL LALALILPGT LC 22 |
| (Amino acids 1 to 22 of | | |
| SEQ ID NO: 2) | | |
| VWF DID2 region | 23 | AEGTRGRS STARCSLEGS |
| (Amino acids 23 to 763 | | DFVNTFDGSM |
| of SEQ ID NO: 2) | 51 | YSFAGYCSYL LAGGCQKRSF SIIGDFQNGK RVSLSVYLGE |
| | | FFDIHLFVNG |
| | 101 | TVTQGDQRVS MPYASKGLYL ETEAGYYKLS GEAYGEVARI |
| | | DGSGNFQVLL |
| | 151 | SDRYENKTCG LCGNFNIFAE DDEMTQEGTL TSDPYDFANS |
| | | WALSSGEQWC |
| | 201 | ERASPPSSSC NISSGEMQKG LWEQCQLLKS TSVFARCHPL |
| | | VDPEPEVALC |
| | 251 | EKTLCECAGG LECACPALLE YARTCAQEGM VLYGWTDHSA |
| | | CSPVCPAGME |
| | 301 | YRQCVSPCAR TCQSLHINEM CQERCVDGCS CPEGOLLDEG |
| | | LCVESTECPC |
| | 351 | VHSGKRYPPG TSLSRDCNTC ICRNSQWICS NEECPGECLV |
| | | TGQSHEKSED |
| | 401 | NRYFTESGIC QYLLARDCQD HSFSIVIETV QCADDRDAVC |
| | | TRSVTVRLPG |
| | 451 | LHNSLVKLKH GAGVAMDGQD IQLPLLKGDL RIQHTVTASV |
| | | RLSYGEDLQM |
| | 501 | DWDGRGRLLV KLSPVYAGKT CGLCGNYNGN QGDDFLTPSG |
| | | LAEPRVEDEG |
| | 551 | NAWKLHGDCQ DLQKQHSDPC ALNPRMTRFS EEACAVLTSP |
| | | TFEACHRAVS |
| | 601 | PLPYLRNCRY DVCSCSDGRE CLCGALASYA AACAGRGVRV |
| | | AWREPGRCEL |
| | 651 | NCPKGQVYLQ CGTPCNLTCR SLSYPDEECN EACLEGCFCP |
| | | PGLYMDERGD |
| | 701 | CVPKAQCPCY YDGEIFQPED IFSDHHTMCY CEDGEMHCTM |
| | | SGVPGSLLPD |
| | 751 | AVLSSPLSHR SKR 763 |
| VWF D' Domain | 764 | SLSCRPP MVKLVCPADN LRAEGLECTK |
| (Amino acids 764 to 866 | | TCQNYDLECMTCQNYDLECM |
| of SEQ ID NO: 2) | 801 | SMGCVSGCLC PPGMVRHENR CVALERCPCF HQGKEYAPGE |
| | | TVKIGCNTCV |
| | 851 | CRDRKWNCTD HVCDAT 866 |
| VWF D3 Domain | 867 | 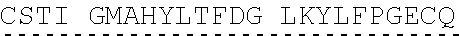 |
| (Amino acids 867 to | | 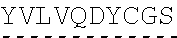 |
| 1240 of SEQ ID NO: 2) | 901 | 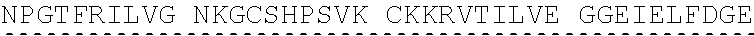 |
| | | 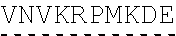 |
| | 951 | 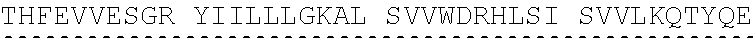 |
| | | 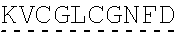 |
| | 1001 | 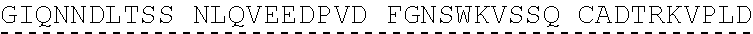 |
| | | 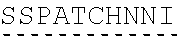 |
| | 1051 | 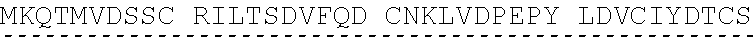 |
| | | 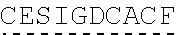 |
| | 1101 | 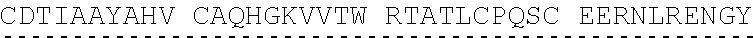 |
| | | 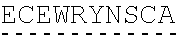 |
| | 1151 | 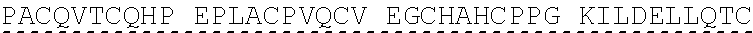 |
| | | 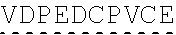 |
| | 1201 | 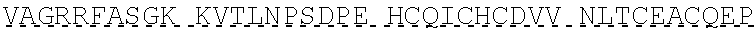 |
| | 1240 | |
| VWF A1 Domain | 1241 | GGLVVPPTDA |
| (Amino acids 1241 to | 1251 | PVSPTTLYVE DISEPPLHDF YCSRLLDLVF LLDGSSRLSE |
| 1479 of SEQ ID NO: 2) | | AEFEVLKAFV |
| | 1301 | VDMMERLRIS QKWVRVAVVE YHDGSHAYIG LKDRKRPSEL |
| | | RRIASQVKYA |
| | 1351 | GSQVASTSEV LKYTLFQIFS KIDRPEASRI ALLLMASQEP |
| | | QRMSRNFVRY |
| | 1401 | VQGLKKKKVI VIPVGIGPHA NLKQIRLIEK QAPENKAFVL |
| | | SSVDELEQQR |
| | 1451 | DEIVSYLCDL APEAPPPTLP PDMAQVTVG 1479 |
| | 1480 | P GLLGVSTLGP KRNSMVLDVA |
| | 1501 | FVLEGSDKIG EADFNRSKEF MEEVIQRMDV GQDSIHTVTVL |
| | | QYSYMVTVEY |
| | 1551 | PFSEAQSKGD ILQRVREIRY QGGNRTNTGL ALRYLSDHSF |
| | | LVSQGDREQA 1600 |
| | 1601 | PNLVYMVTGN PASDEIKRLP GDIQVVPIGV GPNANVQELE |
| | | RIGWPNAPIL |
| | 1651 | IQDFETLPRE APDLVLORCC SGEGLQIPTL SPAPDCSQPL |
| | | DVILLLDGSS |
| | 1701 | SFPASYFDEM KSFAKAFISK ANIGPRLTQV SVLQYGSITT |
| | | IDVPWNVVPE |
| | 1751 | KAHLLSLVDV MQREGGPSQI GDALGFAVRY LTSEMHGARP |
| | | GASKAVVILV |
| | 1801 | GPAGDSNVVK AADAARSNRV TVFPIGIGDR YDAAQLRILA |
| | | TDVSVDSVDA |
| | 1851 | LQRIEDLPTM VTLGNSFLHK LCSGFVRICM DEDGNEKRPG |
| | | DVWTL PDQCH |
| | 1901 | TVTCQPDGQT LLKSHRVNCD RGLRPSCPNS QSPVKVEETC |
| | | GCRWTCPCVC |
| | 1951 | TGSSTRHIVT FDGONFKLTG SCSYVLFQNK EQDLEVILHN |
| | | GACSPGARQG |
| | 2001 | CMKSIEVKHS ALSVEXHSDM EVTVNGRLVS VPYVGGNMEV |
| | | NVYGAIMHEV |
| | 2051 | RFNHLGHIFT FTPQNNEFQL QLSPKTFASK TYGLCGICDE |
| | | NGANDEMLRD |
| | 2101 | GTVTTDWKTL VQEWTVQRPG QTCQPILEEQ CLVPDSSHCQ |
| | | VLLLPLFAEC |
| | 2151 | HKVLAPATFY AICQQDSCHQ EQVCEVIASY AHLCRINGVC |
| | | VDWRT PDFCA |
| | 2201 | MSCPPSLVYN HCEHGCPRHC DGNVSSCGDH PSEGCFCPPD |
| | | KVMLEGSCVP |
| | 2251 | EEACTQCIGE DGVQHOFLEA WVPDHQPCQI CTCLSGRKVN |
| | | CTTQPCPTAK |
| | 2301 | APTCGLCEVA RLRQNADQCC PEYECVCDPV SCDLPPVPHC |
| | | ERGLQPTLTN |
| | 2351 | PGECRPNFTC ACRKEECKRV SPPSCPPHRL PTLRKTQCCD |
| | | EYECACNCVN |
| | 2401 | STVSCPLGYL ASTATNDCGC TTTTCLPDKV CVHRSTIYPV |
| | | GQFWEEGCDV |
| | 2451 | CTCTDMEDAV MGLRVAQCSQ KPCEDSCRSG FTYVLHEGEC |
| | | CGRCLPSACE |
| | 2501 | VVTGSPRGDS QSSWKSVGSQ WASPENPCLI NECVRVKEEV |
| | | FIQQRNVSCP |
| | 2551 | QLEVPVCPSG FQLSCKTSAC CPSCRCERME ACMLNGTVIG |
| | | PGKTVMIDVC |
| | 2601 | TTCRCMVQVG VISGFKLECR KTTCNPCPLG YKEENNTGEC |
| | | CGRCLPTACT |
| | 2651 | IQLRGGQIMT LKRDETLODG CDTHECKVNE RGEYFWEKRV |
| | | TGCPPEDEHK |
| | 2701 | SEVEVDIHYC IPGTCCDTCE EPECNDITAR LQYVKVGSCK |
| | | CLAEGGKIMK |
| | 2751 | QGKCASKAMY SIDINDVQDQ CSCCSPTRTE PMQVALHCTN |
| | | GSVVYHEVLN |
| | 2801 | AMECKCSPRK CSK |

| | Nucleotide Sequence |
|---|---|
| Full-length VWF | ATGATTCCTG CCAGATTTGC CGGGGTGCTG CTTGCTCTGG CCCTCATTTT |
| (SEQ ID NO: 1) | GCCAGGGACC CTTTGTGCAG AAGGAACTCG CGGCAGGTCA TCCACGGCCC |
| | TACTAAGGAC GGTCTAAACG GCCCCACGAC GAACGAGACC GGGAGTAAAA |
| | CGGTCCCTGG GAAACACGTC TTCCTTGAGC GCCGTCCAGT AGGTGCCGGG |
| | GATGCAGCCT TTTCGGAAGT GACTTCGTCA ACACCTTTGA TGGGAGCATG |
| | TACAGCTTTG CGGGATACTG CAGTTACCTC CTGGCAGGGG GCTGCCAGAA |
| | CTACGTCGGA AAAGCCTTCA CTGAAGCAGT TGTGGAAACT ACCCTCGTAC |
| | ATGTCGAAAC GCCCTATGAC GTCAATGGAG GACCGTCCCC CGACGGTCTT |
| | ACGCTCCTTC TCGATTATTG GGGACTTCCA GAATGGCAAG AGAGTGAGCC |
| | TCTCCGTGTA TCTTGGGGAA TTTTTTGACA TCCATTTGTT TGTCAATGGT |
| | TGCGAGGAAG AGCTAATAAC CCCTGAAGGT CTTACCGTTC TCTCACTCGG |
| | AGAGGCACAT AGAACCCCTT AAAAAACTGT AGGTAAACAA ACAGTTACCA |
| | ACCGTGACAC AGGGGGACCA AAGAGTCTCC ATGCCCTATG CCTCCAAAGG |
| | GCTGTATCTA GAAACTGAGG CTGGGTACTA CAAGCTGTCC GGTGAGGCCT |
| | TGGCACTGTG TCCCCCTGGT TTCTCAGAGG TACGGGATAC GGAGGTTTCC |
| | CGACATAGAT CTTTGACTCC GACCCATGAT GTTCGACAGG CCACTCCGGA |
| | ATGGCTTTGT GGCCAGGATC GATGGCAGCG GCAACTTTCA AGTCCTGCTG |
| | TCAGACAGAT ACTTCAACAA GACCTGCGGG CTGTGTGGCA ACTTTAACAT |
| | TACCGAAACA CCGGTCCTAG CTACCGTCGC CGTTGAAAGT TCAGGACGAC |
| | AGTCTGTCTA TGAAGTTGTT CTGGACGCCC GACACACCGT TGAAATTGTA |
| | CTTTGCTGAA GATGACTTTA TGACCCAAGA AGGGACCTTG ACCTCGGACC |
| | CTTATGACTT TGCCAACTCA TGGGCTCTGA GCAGTGGAGA ACAGTGGTGT |
| | GAAACGACTT CTACTGAAAT ACTGGGTTCT TCCCTGGAAC TGGAGCCTGG |
| | GAATACTGAA ACGGTTGAGT ACCCGAGACT CGTCACCTCT TGTCACCACA |
| | GAACGGGCAT CTCCTCCCAG CAGCTCATGC AACATCTCCT CTGGGGAAAT |
| | GCAGAAGGGC CTGTGGGAGC AGTGCCAGCT TCTGAAGAGC ACCTCGGTGT |
| | CTTGCCCGTA GAGGAGGGTC GTCGAGTACG TTGTAGAGGA GACCCCTTTA |
| | CGTCTTCCCG GACACCCTCG TCACGGTCGA AGACTTCTCG TGGAGCCACA |
| | TTGCCCGCTG CCACCCTCTG GTGGACCCCG AGCCTTTTGT GGCCCTGTGT |
| | GAGAAGACTT TGTGTGAGTG TGCTGGGGGG CTGGAGTGCG CCTGCCCTGC |
| | AACGGGCGAC GGTGGGAGAC CACCTGGGGC TCGGAAAACA CCGGGACACA |
| | CTCTTCTGAA ACACACTCAC ACGACCCCCC GACCTCACGC GGACGGGACG |
| | CCTCCTGGAG TACGCCCGGA CCTGTGCCCA GGAGGGAATG GTGCTGTACG |
| | GCTGGACCGA CCACAGCGCG TGCAGCCCAG TGTGCCCTGC TGGTATGGAG |
| | GGAGGACCTC ATGCGGGCCT GGACACGGGT CCTCCCTTAC CACGACATGC |
| | CGACCTGGCT GGTGTCGCGC ACGTCGGGTC ACACGGGACG ACCATACCTC |
| | TATAGGCAGT GTGTGTCCCC TTGCGCCAGG ACCTGCCAGA GCCTGCACAT |
| | CAATGAAATG TGTCAGGAGC GATGCGTGGA TGGCTGCAGC TGCCCTGAGG |
| | ATATCCGTCA CACACAGGGG AACGCGGTCC TGGACGGTCT CGGACGTGTA |
| | GTTACTTTAC ACAGTCCTCG CTACGCACCT ACCGACGTCG ACGGGACTCC |
| | GACAGCTCCT GGATGAAGGC CTCTGCGTGG AGAGCACCGA GTGTCCCTGC |
| | GTGCATTCCG GAAAGCGCTA CCCTCCCGGC ACCTCCCTCT CTCGAGACTG |
| | CTGTCGAGGA CCTACTTCCG GAGACGCACC TCTCGTGGCT CACAGGGACG |
| | CACGTAAGGC CTTTCGCGAT GGGAGGGCCG TGGAGGGAGA GAGCTCTGAC |
| | CAACACCTGC ATTTGCCGAA ACAGCCAGTG GATCTGCAGC AATGAAGAAT |
| | GTCCAGGGGA GTGCCTTGTC ACTGGTCAAT CCCACTTCAA GAGCTTTGAC |
| | GTTGTGGACG TAAACGGCTT TGTCGGTCAC CTAGACGTCG TTACTTCTTA |
| | CAGGTCCCCT CACGGAACAG TGACCAGTTA GGGTGAAGTT CTCGAAACTG |
| | AACAGATACT TCACCTTCAG TGGGATCTGC CAGTACCTGC TGGCCCGGGA |
| | TTGCCAGGAC CACTCCTTCT CCATTGTCAT TGAGACTGTC CAGTGTGCTG |
| | TTGTCTATGA AGTGGAAGTC ACCCTAGACG GTCATGGACG ACCGGGCCCT |
| | AACGGTCCTG GTGAGGAAGA GGTAACAGTA ACTCTGACAG GTCACACGAC |
| | ATGACCGCGA CGCTGTGTGC ACCCGCTCCG TCACCGTCCG GCTGCCTGGC |
| | CTGCACAACA GCCTTGTGAA ACTGAAGCAT GGGGCAGGAG TTGCCATGGA |
| | TACTGGCGCT GCGACACACG TGGGCGAGGC AGTGGCAGGC CGACGGACCG |
| | GACGTGTTGT CGGAACACTT TGACTTCGTA CCCCGTCCTC AACGGTACCT |
| | TGGCCAGGAC ATCCAGCTCC CCCTCCTGAA AGGTGACCTC CGCATCCAGC |
| | ATACAGTGAC GGCCTCCGTG CGCCTCAGCT ACGGGGAGGA CCTGCAGATG |
| | ACCGGTCCTG TAGGTCGAGG GGGAGGACTT TCCACTGGAG GCGTAGGTCG |
| | TATGTCACTG CCGGAGGCAC GCGGAGTCGA TGCCCCTCCT GGACGTCTAC |
| | GACTGGGATG GCCGCGGGAG GCTGCTGGTG AAGCTGTCCC CCGTCTATGC |
| | CGGGAAGACC TGCGGCCTGT GTGGGAATTA CAATGGCAAC CAGGGCGACG |
| | CTGACCCTAC CGGCGCCCTC CGACGACCAC TTCGACAGGG GGCAGATACG |
| | GCCCTTCTGG ACGCCGGACA CACCCTTAAT GTTACCGTTG GTCCCGCTGC |
| | ACTTCCTTAC CCCCTCTGGG CTGGCRGAGC CCCGGGTGGA GGACTTCGGG |
| | AACGCCTGGA AGCTGCACGG GGACTGCCAG GACCTGCAGA AGCAGCACAG |
| | TGAAGGAATG GGGGAGACCC GACCGYCTCG GGGCCCACCT CCTGAAGCCC |
| | TTGCGGACCT TCGACGTGCC CCTGACGGTC CTGGACGTCT TCGTCGTGTC |
| | CGATCCCTGC GCCCTCAACC CGCGCATGAC CAGGTTCTCC GAGGAGGCGT |
| | GCGCGGTCCT GACGTCCCCC ACATTCGAGG CCTGCCATCG TGCCGTCAGC |
| | GCTAGGGACG CGGGAGTTGG GCGCGTACTG GTCCAAGAGG CTCCTCCGCA |
| | CGCGCCAGGA CTGCAGGGGG TGTAAGCTCC GGACGGTAGC ACGGCAGTCG |
| | CCGCTGCCCT ACCTGCGGAA CTGCCGCTAC GACGTGTGCT CCTGCTCGGA |
| | CGGCCGCGAG TGCCTGTGCG GCGCCCTGGC CAGCTATGCC GCGGCCTGCG |
| | GGCGACGGGA TGGACGCCTT GACGGCGATG CTGCACACGA GGACGAGCCT |
| | GCCGGCGCTC ACGGACACGC CGCGGGACCG GTCGATACGG CGCCGGACGC |
| | CGGGGAGAGG CGTGCGCGTC GCGTGGCGCG AGCCAGGCCG CTGTGAGCTG |
| | AACTGCCCGA AAGGCCAGGT GTACCTGCAG TGCGGGACCC CCTGCAACCT |
| | GCCCCTCTCC GCACGCGCAG CGCACCGCGC TCGGTCCGGC GACACTCGAC |
| | TTGACGGGCT TTCCGGTCCA CATGGACGTC ACGCCCTGGG GGACGTTGGA |
| | GACCTGCCGC TCTCTCTCTT ACCCGGATGA GGAATGCAAT GAGGCCTGCC |
| | TGGAGGGCTG CTTCTGCCCC CCAGGGCTCT ACATGGATGA GAGGGGGGAC |
| | CTGGACGGCG AGAGAGAGAA TGGGCCTACT CCTTACGTTA CTCCGGACGG |
| | ACCTCCCGAC GAAGACGGGG GGTCCCGAGA TGTACCTACT CTCCCCCCTG |
| | TGCGTGCCCA AGGCCCAGTG CCCCTGTTAC TATGACGGTG AGATCTTCCA |
| | GCCAGAAGAC ATCTTCTCAG ACCATCACAC CATGTGCTAC TGTGAGGATG |
| | ACGCACGGGT TCCGGGTCAC GGGGACAATG ATACTGCCAC TCTAGAAGGT |
| | CGGTCTTCTG TAGAAGAGTC TGGTAGTGTG GTACACGATG ACACTCCTAC |
| | GCTTCATGCA CTGTACCATG AGTGGAGTCC CCGGAAGCTT GCTGCCTGAC |
| | GCTGTCCTCA GCAGTCCCCT GTCTCATCGC AGCAAAAGGA GCCTATCCTG |
| | CGAAGTACGT GACATGGTAC TCACCTCAGG GGCCTTCGAA CGACGGACTG |
| | CGACAGGAGT CGTCAGGGGA CAGAGTAGCG TCGTTTTCCT CGGATAGGAC |
| | TCGGCCCCCC ATGGTCAAGC TGGTGTGTCC CGCTGACAAC CTGCGGGCTG |
| | AAGGGCTCGA GTGTACCAAA ACGTGCCAGA ACTATGACCT GGAGTGCATG |
| | AGCCGGGGGG TACCAGTTCG ACCACACAGG GCGACTGTTG GACGCCCGAC |
| | TTCCCGAGCT CACATGGTTT TGCACGGTCT TGATACTGGA CCTCACGTAC |
| | AGCATGGGCT GTGTCTCTGG CTGCCTCTGC CCCCCGGGCA TGGTCCGGCA |
| | TGAGAACAGA TGTGTGGCCC TGGAAAGGTG TCCCTGCTTC CATCAGGGCA |
| | TCGTACCCGA CACAGAGACC GACGGAGACG GGGGGCCCGT ACCAGGCCGT |
| | ACTCTTGTCT ACACACCGGG ACCTTTCCAC AGGGACGAAG GTAGTCCCGT |
| | AGGAGTATGC CCCTGGAGAA ACAGTGAAGA TTGGCTGCAA CACTTGTGTC |
| | TGTCGGGACC GGAAGTGGAA CTGCACAGAC CATGTGTGTG ATGCCACGTG |
| | TCCTCATACG GGGACCTCTT TGTCACTTCT AACCGACGTT GTGAACACAG |
| | ACAGCCCTGG CCTTCACCTT GACGTGTCTG GTACACACAC TACGGTGCAC |
| | CTCCACGATC GGCATGGCCC ACTACCTCAC CTTCGACGGG CTCAAATACC |
| | TGTTCCCCGG GGAGTGCCAG TACGTTCTGG TGCAGGATTA CTGCGGCAGT |
| | GAGGTGCTAG CCGTACCGGG TGATGGAGTG GAAGCTGCCC GAGTTTATGG |
| | ACAAGGGGCC CCTCACGGTC ATGCAAGACC ACGTCCTAAT GACGCCGTCA |
| | AACCCTGGGA CCTTTCGGAT CCTAGTGGGG AATAAGGGAT GCAGCCACCC |
| | CTCAGTGAAA TGCAAGAAAC GGGTCACCAT CCTGGTGGAG GGAGGAGAGA |
| | TTGGGACCCT GGAAAGCCTA GGATCACCCC TTATTCCCTA CGTCGGTGGG |
| | GAGTCACTTT ACGTTCTTTG CCCAGTGGTA GGACCACCTC CCTCCTCTCT |
| | TTGAGCTGTT TGACGGGGAG GTGAATGTGA AGAGGCCCAT GAAGGATGAG |
| | ACTCACTTTG AGGTGGTGGA GTCTGGCCGG TACATCATTC TGCTGCTGGG |
| | AACTCGACAA ACTGCCCCTC CACTTACACT TCTCCGGGTA CTTCCTACTC |
| | TGAGTGAAAC TCCACCACCT CAGACCGGCC ATGTAGTAAG ACGACGACCC |
| | CAAAGCCCTC TCCGTGGTCT GGGACCGCCA CCTGAGCATC TCCGTGGTCC |
| | TGAAGCAGAC ATACCAGGAG AAAGTGTGTG GCCTGTGTGG GAATTTTGAT |
| | GTTTCGGGAG AGGCACCAGA CCCTGGCGGT GGACTCGTAG AGGCACCAGG |
| | ACTTCGTCTG TATGGTCCTC TTTCACACAC CGGACACACC CTTAAAACTA |
| | GGCATCCAGA ACAATGACCT CACCAGCAGC AACCTCCAAG TGGAGGAAGA |
| | CCCTGTGGAC TTTGGGAACT CCTGGAAAGT GAGCTCGCAG TGTGCTGACA |
| | CCGTAGGTCT TGTTACTGGA GTGGTCGTCG TTGGAGGTTC ACCTCCTTCT |
| | GGGACACCTG AAACCCTTGA GGACCTTTCA CTCGAGCGTC ACACGACTGT |
| | CCAGAAAAGT GCCTCTGGAC TCATCCCCTG CCACCTGCCA TAACAACATC |
| | ATGAAGCAGA CGATGGTGGA TTCCTCCTGT AGAATCCTTA CCAGTGACGT |
| | GGTCTTTTCA CGGAGACCTG AGTAGGGGAC GGTGGACGGT ATTGTTGTAG |
| | TACTTCGTCT GCTACCACCT AAGGAGGACA TCTTAGGAAT GGTCACTGCA |
| | CTTCCAGGAC TGCAACAAGC TGGTGGACCC CGAGCCATAT CTGGATGTCT |
| | GCATTTACGA CACCTGCTCC TGTGAGTCCA TTGGGGACTG CGCCTGCTTC |
| | GAAGGTCCTG ACGTTGTTCG ACCACCTGGG GCTCGGTATA GACCTACAGA |
| | CGTAAATGCT GTGGACGAGG ACACTCAGGT AACCCCTGAC GCGGACGAAG |
| | TGCGACACCA TTGCTGCCTA TGCCCACGTG TGTGCCCAGC ATGGCAAGGT |
| | GGTGACCTGG AGGACGGCCA CATTGTGCCC CCAGAGCTGC GAGGAGAGGA |
| | ACGCTGTGGT AACGACGGAT ACGGGTGCAC ACACGGGTCG TACCGTTCCA |
| | CCACTGGACC TCCTGCCGGT GTAACACGGG GGTCTCGACG CTCCTCTCCT |
| | ATCTCCGGGA GAACGGGTAT GAGTGTGAGT GGCGCTATAA CAGCTGTGCA |
| | CCTGCCTGTC AAGTCACGTG TCAGCACCCT GAGCCACTGG CCTGCCCTGT |
| | TAGAGGCCCT CTTGCCCATA CTCACACTCA CCGCGATATT GTCGACACGT |
| | GGACGGACAG TTCAGTGCAC AGTCGTGGGA CTCGGTGACC GGACGGGACA |
| | GCAGTGTGTG GAGGGCTGCC ATGCCCACTG CCCTCCAGGG AAAATCCTGG |
| | ATGAGCTTTT GCAGACCTGC GTTGACCCTG AAGACTGTCC AGTGTGTGAG |
| | CGTCACACAC CTCCCGACGG TACGGGTGAC GGGAGGTCCC TTTTAGGACC |
| | TACTCGAAAA CGTCTGGACG CAACTGGGAC TTCTGACAGG TCACACACTC |
| | GTGGCTGGCC GGCGTTTTGC CTCAGGAAAG AAAGTCACCT TGAATCCCAG |
| | TGACCCTGAG CACTGCCAGA TTTGCCACTG TGATGTTGTC AACCTCACCT |
| | CACCGACCGG CCGCAAAACG GAGTCCTTTC TTTCAGTGGA ACTTAGGGTC |
| | ACTGGGACTC GTGACGGTCT AAACGGTGAC ACTACAACAG TTGGAGTGGA |
| | GTGAAGCCTG CCAGGAGCCG GGAGGCCTGG TGGTGCCTCC CACAGATGCC |
| | CCGGTGAGCC CCACCACTCT GTATGTGGAG GACATCTCGG AACCGCCGTT |
| | CACTTCGGAC GGTCCTCGGC CCTCCGGACC ACCACGGAGG GTGTCTACGG |
| | GGCCACTCGG GGTGGTGAGA CATACACCTC CTGTAGAGCC TTGGCGGCAA |
| | GCACGATTTC TACTGCAGCA GGCTACTGGA CCTGGTCTTC CTGCTGGATG |
| | GCTCCTCCAG GCTGTCCGAG GCTGAGTTTG AAGTGCTGAA GGCCTTTGTG |
| | CGTGCTAAAG ATGACGTCGT CCGATGACCT GGACCAGAAG GACGACCTAC |
| | CGAGGAGGTC CGACAGGCTC CGACTCAAAC TTCACGACTT CCGGAAACAC |
| | GTGGACATGA TGGAGCGGCT GCGCATCTCC CAGAAGTGGG TCCGCGTGGC |
| | CGTGGTGGAG TACCACGACG GCTCCCACGC CTACATCGGG CTCAAGGACC |
| | CACCTGTACT ACCTCGCCGA CGCGTAGAGG GTCTTCACCC AGGCGCACCG |
| | GCACCACCTC ATGGTGCTGC CGAGGGTGCG GATGTAGCCC GAGTTCCTGG |
| | GGAAGCGACC GTCAGAGCTG CGGCGCATTG CCAGCCAGGT GAAGTATGCG |
| | GGCAGCCAGG TGGCCTCCAC CAGCGAGGTC TTGAAATACA CACTGTTCCA |
| | CCTTCGCTGG CAGTCTCGAC GCCGCGTAAC GGTCGGTCCA CTTCATACGC |
| | CCGTCGGTCC ACCGGAGGTG GTCGCTCCAG AACTTTATGT GTGACAAGGT |
| | AATCTTCAGC AAGATCGACC GCCCTGAAGC CTCCCGCATC GCCCTGCTCC |
| | TGATGGCCAG CCAGGAGCCC CAACGGATGT CCCGGAACTT TGTCCGCTAC |
| | TTAGAAGTCG TTCTAGCTGG CGGGACTTCG GAGGGCGTAG CGGGACGAGG |
| | ACTACCGGTC GGTCCTCGGG GTTGCCTACA GGGCCTTGAA ACAGGCGATG |
| | GTCCAGGGCC TGAAGAAGAA GAAGGTCATT GTGATCCCGG TGGGCATTGG |
| | GCCCCATGCC AACCTCAAGC AGATCCGCCT CATCGAGAAG CAGGCCCCTG |
| | CAGGTCCCGG ACTTCTTCTT CTTCCAGTAA CACTAGGGCC ACCCGTAACC |
| | CGGGGTACGG TTGGAGTTCG TCTAGGCGGA GTAGCTCTTC GTCCGGGGAC |
| | AGAACAAGGC CTTCGTGCTG AGCAGTGTGG ATGAGCTGGA GCAGCAAAGG |
| | GACGAGATCG TTAGCTACCT CTGTGACCTT GCCCCTGAAG CCCCTCCTCC |
| | TCTTGTTCCG GAAGCACGAC TCGTCACACC TACTCGACCT CGTCGTTTCC |
| | CTGCTCTAGC AATCGATGGA GACACTGGAA CGGGGACTTC GGGGAGGAGG |
| | TACTCTGCCC CCCGACATGG CACAAGTCAC TGTGGGCCCG GGGCTCTTGG |
| | GGGTTTCGAC CCTGGGGCCC AAGAGGAACT CCATGGTTCT GGATGTGGCG |
| | ATGAGACGGG GGGCTGTACC GTGTTCAGTG ACACCCGGGC CCCGAGAACC |
| | CCCAAAGCTG GGACCCCGGG TTCTCCTTGA GGTACCAAGA CCTACACCGC |
| | TTCGTCCTGG AAGGATCGGA CAAAATTGGT GAAGCCGACT TCAACAGGAG |
| | CAAGGAGTTC ATGGAGGAGG TGATTCAGCG GATGGATGTG GGCCAGGACA |
| | AAGCAGGACC TTCCTAGCCT GTTTTAACCA CTTCGGCTGA AGTTGTCCTC |
| | GTTCCTCAAG TACCTCCTCC ACTAAGTCGC CTACCTACAC CCGGTCCTGT |
| | GCATCCACGT CACGGTGCTG CAGTACTCCT ACATGGTGAC CGTGGAGTAC |
| | CCCTTCAGCG AGGCACAGTC CAAAGGGGAC ATCCTGCAGC GGGTGCGAGA |
| | CGTAGGTGCA GTGCCACGAC GTCATGAGGA TGTACCACTG GCACCTCATG |
| | GGGAAGTCGC TCCGTGTCAG GTTTCCCCTG TAGGACGTCG CCCACGCTCT |
| | GATCCGCTAC CAGGGCGGCA ACAGGACCAA CACTGGGCTG GCCCTGCGGT |
| | ACCTCTCTGA CCACAGCTTC TTGGTCAGCC AGGGTGACCG GGAGCAGGCG |
| | CTAGGCGATG GTCCCGCCGT TGTCCTGGTT GTGACCCGAC CGGGACGCCA |
| | TGGAGAGACT GGTGTCGAAG AACCAGTCGG TCCCACTGGC CCTCGTCCGC |
| | CCCAACCTGG TCTACATGGT CACCGGAAAT CCTGCCTCTG ATGAGATCAA |
| | GAGGCTGCCT GGAGACATCC AGGTGGTGCC CATTGGAGTG GGCCCTAATG |
| | GGGTTGGACC AGATGTACCA GTGGCCTTTA GGACGGAGAC TACTCTAGTT |
| | CTCCGACGGA CCTCTGTAGG TCCACCACGG GTAACCTCAC CCGGGATTAC |
| | CCAACGTGCA GGAGCTGGAG AGGATTGGCT GGCCCAATGC CCCTATCCTC |
| | ATCCAGGACT TTGAGACGCT CCCCCGAGAG GCTCCTGACC TGGTGCTGCA |
| | GGTTGCACGT CCTCGACCTC TCCTAACCGA CCGGGTTACG GGGATAGGAG |
| | TAGGTCCTGA AACTCTGCGA GGGGGCTCTC CGAGGACTGG ACCACGACGT |
| | GAGGTGCTGC TCCGGAGAGG GGCTGCAGAT CCCCACCCTC TCCCCTGCAC |
| | CTGACTGCAG CCAGCCCCTG GACGTGATCC TTCTCCTGGA TGGCTCCTCC |
| | CTCCACGACG AGGCCTCTCC CCGACGTCTA GGGGTGGGAG AGGGGACGTG |
| | GACTGACGTC GGTCGGGGAC CTGCACTAGG AAGAGGACCT ACCGAGGAGG |
| | AGTTTCCCAG CTTCTTATTT TGATGAAATG AAGAGTTTCG CCAAGGCTTT |
| | CATTTCAAAA GCCAATATAG GGCCTCGTCT CACTCAGGTG TCAGTGCTGC |
| | TCAAAGGGTC GAAGAATAAA ACTACTTTAC TTCTCAAAGC GGTTCCGAAA |
| | GTAAAGTTTT CGGTTATATC CCGGAGCAGA GTGAGTCCAC AGTCACGACG |
| | AGTATGGAAG CATCACCACC ATTGACGTGC CATGGAACGT GGTCCCGGAG |
| | AAAGCCCATT TGCTGAGCCT TGTGGACGTC ATGCAGCGGG AGGGAGGCCC |
| | TCATACCTTC GTAGTGGTGG TAACTGCACG GTACCTTGCA CCAGGGCCTC |
| | TTTCGGGTAA ACGACTCGGA ACACCTGCAG TACGTCGCCC TCCCTCCGGG |
| | CAGCCAAATC GGGGATGCCT TGGGCTTTGC TGTGCGATAC TTGACTTCAG |
| | AAATGCATGG TGCCAGGCCG GGAGCCTCAA AGGCGGTGGT CATCCTGGTC |
| | GTCGGTTTAG CCCCTACGGA ACCCGAAACG ACACGCTATG AACTGAAGTC |
| | TTTACGTACC ACGGTCCGGC CCTCGGAGTT TCCGCCACCA GTAGGACCAG |
| | ACGGACGTCT CTGTGGATTC AGTGGATGCA GCAGCTGATG CCGCCAGGTC |
| | CAACAGAGTG ACAGTGTTCC CTATTGGAAT TGGAGATCGC TACGATGCAG |
| | TGCCTGCAGA GACACCTAAG TCACCTACGT CGTCGACTAC GGCGGTCCAG |
| | GTTGTCTCAC TGTCACAAGG GATAACCTTA ACCTCTAGCG ATGCTACGTC |
| | CCCAGCTACG GATCTTGGCA GGCCCAGCAG GCGACTCCAA CGTGGTGAAG |
| | CTCCAGCGAA TCGAAGACCT CCCTACCATG GTCACCTTGG GCAATTCCTT |
| | GGGTCGATGC CTAGAACCGT CCGGGTCGTC CGCTGAGGTT GCACCACTTC |
| | GAGGTCGCTT AGCTTCTGGA GGGATGGTAC CAGTGGAACC CGTTAAGGAA |
| | CCTCCACAAA CTGTGCTCTG GATTTGTTAG GATTTGCATG GATGAGGATG |
| | GGAATGAGAA GAGGCCCGGG GACGTCTGGA CCTTGCCAGA CCAGTGCCAC |
| | GGAGGTGTTT GACACGAGAC CTAAACAATC CTAAACGTAC CTACTCCTAC |
| | CCTTACTCTT CTCCGGGCCC CTGCAGACCT GGAACGGTCT GGTCACGGTG |
| | ACCGTGACTT GCCAGCCAGA TGGCCAGACC TTGCTGAAGA GTCATCGGGT |
| | CAACTGTGAC CGGGGGCTGA GGCCTTCGTG CCCTAACAGC CAGTCCCCTG |
| | TGGCACTGAA CGGTCGGTCT ACCGGTCTGG AACGACTTCT CAGTAGCCCA |
| | GTTGACACTG GCCCCCGACT CCGGAAGCAC GGGATTGTCG GTCAGGGGAC |
| | TTAAAGTGGA AGAGACCTGT GGCTGCCGCT GGACCTGCCC CTGYGTGTGC |
| | ACAGGCAGCT CCACTCGGCA CATCGTGACC TTTGATGGGC AGAATTTCAA |
| | AATTTCACCT TCTCTGGACA CCGACGGCGA CCTGGACGGG GACRCACACG |
| | TGTCCGTCGA GGTGAGCCGT GTAGCACTGG AAACTACCCG TCTTAAAGTT |
| | GCTGACTGGC AGCTGTTCTT ATGTCCTATT TCAAAACAAG GAGCAGGACC |
| | TGGAGGTGAT TCTCCATAAT GGTGCCTGCA GCCCTGGAGC AAGGCAGGGC |
| | CGACTGACCG TCGACAAGAA TACAGGATAA AGTTTTGTTC CTCGTCCTGG |
| | ACCTCCACTA AGAGGTATTA CCACGGACGT CGGGACCTCG TTCCGTCCCG |
| | TGCATGAAAT CCATCGAGGT GAAGCACAGT GCCCTCTCCG TCGAGSTGCA |
| | CAGTGACATG GAGGTGACGG TGAATGGGAG ACTGGTCTCT GTTCCTTACG |
| | ACGTACTTTA GGTAGCTCCA CTTCGTGTCA CGGGAGAGGC AGCTCSACGT |
| | GTCACTGTAC CTCCACTGCC ACTTACCCTC TGACCAGAGA CAAGGAATGC |
| | TGGGTGGGAA CATGGAAGTC AACGTTTATG GTGCCATCAT GCATGAGGTC |
| | AGATTCAATC ACCTTGGTCA CATCTTCACA TTCACTCCAC AAAACAATGA |
| | ACCCACCCTT GTACCTTCAG TTGCAAATAC CACGGTAGTA CGTACTCCAG |
| | TCTAAGTTAG TGGAACCAGT GTAGAAGTGT AAGTGAGGTG TTTTGTTACT |
| | GTTCCAACTG CAGCTCAGCC CCAAGACTTT TGCTTCAAAG ACGTATGGTC |
| | TGTGTGGGAT CTGTGATGAG AACGGAGCCA ATGACTTCAT GCTGAGGGAT |
| | CAAGGTTGAC GTCGAGTCGG GGTTCTGAAA ACGAAGTTTC TGCATACCAG |
| | ACACACCCTA GACACTACTC TTGCCTCGGT TACTGAAGTA CGACTCCCTA |
| | GGCACAGTCA CCACAGACTG GAAAACACTT GTTCAGGAAT GGACTGTGCA |
| | GCGGCCAGGG CAGACGTGCC AGCCCATCCT GGAGGAGCAG TGTCTTGTCC |
| | CCGTGTCAGT GGTGTCTGAC CTTTTGTGAA CAAGTCCTTA CCTGACACGT |
| | CGCCGGTCCC GTCTGCACGG TCGGGTAGGA CCTCCTCGTC ACAGAACAGG |
| | CCGACAGCTC CCACTGCCAG GTCCTCCTCT TACCACTGTT TGCTGAATGC |
| | CACAAGGTCC TGGCTCCAGC CACATTCTAT GCCATCTGCC AGCAGGACAG |
| | GGCTGTCGAG GGTGACGGTC CAGGAGGAGA ATGGTGACAA ACGACTTACG |
| | GTGTTCCAGG ACCGAGGTCG GTGTAAGATA CGGTAGACGG TCGTCCTGTC |
| | TTGCCACCAG GAGCAAGTGT GTGAGGTGAT CGCCTCTTAT GCCCACCTCT |
| | GTCGGACCAA CGGGGTCTGC GTTGACTGGA GGACACCTGA TTTCTGTGCT |
| | AACGGTGGTC CTCGTTCACA CACTCCACTA GCGGAGAATA CGGGTGGAGA |
| | CAGCCTGGTT GCCCCAGACG CAACTGACCT CCTGTGGACT AAAGACACGA |
| | ATGTCATGCC CACCATCTCT GGTCTACAAC CACTGTGAGC ATGGCTGTCC |
| | CCGGCACTGT GATGGCAACG TGAGCTCCTG TGGGGACCAT CCCTCCGAAG |
| | TACAGTACGG GTGGTAGAGA CCAGATGTTG GTGACACTCG TACCGACAGG |
| | GGCCGTGACA CTACCGTTGC ACTCGAGGAC ACCCCTGGTA GGGAGGCTTC |
| | GCTGTTTCTG CCCTCCAGAT AAAGTCATGT TGGAAGGCAG CTGTGTCCCT |
| | GAAGAGGCCT GCACTCAGTG CATTGGTGAG GATGGAGTCC AGCACCAGTT |
| | CGACAAAGAC GGGAGGTCTA TTTCAGTACA ACCTTCCGTC GACACAGGGA |
| | CTTCTCCGGA CGTGAGTCAC GTAACCACTC CTACCTCAGG TCGTGGTCAA |
| | CCTGGAAGCC TGGGTCCCGG ACCACCAGCC CTGTCAGATC TGCACATGCC |
| | TCAGCGGGCG GAAGGTCAAC TGCACAACGC AGCCCTGCCC CACGGCCAAA |
| | GGACCTTCGG ACCCAGGGCC TGGTGGTCGG GACAGTCTAG ACGTGTACGG |
| | AGTCGCCCGC CTTCCAGTTG ACGTGTTGCG TCGGGACGGG GTGCCGGTTT |
| | GCTCCCACGT GTGGCCTGTG TGAAGTAGCC CGCCTCCGCC AGAATGCAGA |
| | CCAGTGCTGC CCCGAGTATG AGTGTGTGTG TGACCCAGTG AGCTGTGACC |
| | CGAGGGTGCA CACCGGACAC ACTTCATCGG GCGGAGGCGG TCTTACGTCT |
| | GGTCACGACG GGGCTCATAC TCACACACAC ACTGGGTCAC TCGACACTGG |
| | TGCCCCCAGT GCCTCACTGT GAACGTGGCC TCCAGCCCAC ACTGACCAAC |
| | CCTGGCGAGT GCAGACCCAA CTTCACCTGC GCCTGCAGGA AGGAGGAGTG |
| | ACGGGGGTCA CGGAGTGACA CTTGCACCGG AGGTCGGGTG TGACTGGTTG |
| | GGACCGCTCA CGTCTGGGTT GAAGTGGACG CGGACGTCCT TCCTCCTCAC |
| | CAAAAGAGTG TCCCCACCCT CCTGCCCCCC GCACCGTTTG CCCACCCTTC |
| | GGAAGACCCA GTGCTGTGAT GAGTATGAGT GTGCCTGCAA CTGTGTCAAC |
| | GTTTTCTCAC AGGGGTGGGA GGACGGGGGG CGTGGCAAAC GGGTGGGAAG |
| | CCTTCTGGGT CACGACACTA CTCATACTCA CACGGACGTT GACACAGTTG |
| | TCCACAGTGA GCTGTCCCCT TGGGTACTTG GCCTCAACCG CCACCAATGA |
| | CTGTGGCTGT ACCACAACCA CCTGCCTTCC CGACAAGGTG TGTGTCCACC |
| | AGGTGTCACT CGACAGGGGA ACCCATGAAC CGGAGTTGGC GGTGGTTACT |
| | GACACCGACA TGGTGTTGGT GGACGGAAGG GCTGTTCCAC ACACAGGTGG |
| | GAAGCACCAT CTACCCTGTG GGCCAGTTCT GGGAGGAGGG CTGCGATGTG |
| | TGCACCTGCA CCGACATGGA GGATGCCGTG ATGGGCCTCC GCGTGGCCCA |
| | CTTCGTGGTA GATGGGACAC CCGGTCAAGA CCCTCCTCCC GACGCTACAC |
| | ACGTGGACGT GGCTGTACCT CCTACGGCAC TACCCGGAGG CGCACCGGGT |
| | GTGCTCCCAG AAGCCCTGTG AGGACAGCTG TCGGTCGGGC TTCACTTACG |
| | TTCTGCATGA AGGCGAGTGC TGTGGAAGGT GCCTGCCATC TGCCTGTGAG |
| | CACGAGGGTC TTCGGGACAC TCCTGTCGAC AGCCAGCCCG AAGTGAATGC |
| | AAGACGTACT TCCGCTCACG ACACCTTCCA CGGACGGTAG ACGGACACTC |
| | GTGGTGACTG GCTCACCGCG GGGGGACTCC CAGTCTTCCT GGAAGAGTGT |
| | CGGCTCCCAG TGGGCCTCCC CGGAGAACCC CTGCCTCATC AATGAGTGTG |
| | CACCACTGAC CGAGTGGCGC CCCCCTGAGG GTCAGAAGGA CCTTCTCACA |
| | GCCGAGGGTC ACCCGGAGGG GCCTCTTGGG GACGGAGTAG TTACTCACAC |
| | TCCGAGTGAA GGAGGAGGTC TTTATACAAC AAAGGAACGT CTCCTGCCCC |
| | CAGCTGGAGG TCCCTGTCTG CCCCTCGGGC TTTCAGCTGA GCTGTAAGAC |
| | AGGCTCACTT CCTCCTCCAG AAATATGTTG TTTCCTTGCA GAGGACGGGG |
| | GTCGACCTCC AGGGACAGAC GGGGAGCCCG AAAGTCGACT CGACATTCTG |
| | CTCAGCGTGC TGCCCAAGCT GTCGCTGTGA GCGCATGGAG GCCTGCATGC |
| | TCAATGGCAC TGTCATTGGG CCCGGGAAGA CTGTGATGAT CGATGTGTGC |
| | GAGTCGCACG ACGGGTTCGA CAGCGACACT CGCGTACCTC CGGACGTACG |
| | AGTTACCGTG ACAGTAACCC GGGCCCTTCT GACACTACTA GCTACACACG |
| | ACGACCTGCC GCTGCATGGT GCAGGTGGGG GTCATCTCTG GATTCAAGCT |
| | GGAGTGCAGG AAGACCACCT GCAACCCCTG CCCCCTGGGT TACAAGGAAG |
| | TGCTGGACGG CGACGTACCA CGTCCACCCC CAGTAGAGAC CTAAGTTCGA |
| | CCTCACGTCC TTCTGGTGGA CGTTGGGGAC GGGGGACCCA ATGTTCCTTC |
| | AAAATAACAC AGGTGAATGT TGTGGGAGAT GTTTGCCTAC GGCTTGCACC |
| | ATTCAGCTAA GAGGAGGACA GATCATGACA CTGAAGCGTG ATGAGACGCT |
| | TTTTATTGTG TCCACTTACA ACACCCTCTA CAAACGGATG CCGAACGTGG |
| | TAAGTCGATT CTCCTCCTGT CTAGTACTGT GACTTCGCAC TACTCTGCGA |
| | CCAGGATGGC TGTGATACTC ACTTCTGCAA GGTCAATGAG AGAGGAGAGT |
| | ACTTCTGGGA GAAGAGGGTC ACAGGCTGCC CACCCTTTGA TGAACACAAG |
| | GGTCCTACCG ACACTATGAG TGAAGACGTT CCAGTTACTC TCTCCTCTCA |
| | TGAAGACCCT CTTCTCCCAG TGTCCGACGG GTGGGAAACT ACTTGTGTTC |
| | TGTCTTGCTG AGGGAGGTAA AATTATGAAA ATTCCAGGCA CCTGCTGTGA |
| | CACATGTGAG GAGCCTGAGT GCAACGACAT CACTGCCAGG CTGCAGTATG |
| | ACAGAACGAC TCCCTCCATT TTAATACTTT TAAGGTCCGT GGACGACACT |
| | GTGTACACTC CTCGGACTCA CGTTGCTGTA GTGACGGTCC GACGTCATAC |
| | TCAAGGTGGG AAGCTGTAAG TCTGAAGTAG AGGTGGATAT CCACTACTGC |
| | CAGGGCAAAT GTGCCAGCAA AGCCATGTAC TCCATTGACA TCAACGATGT |
| | AGTTCCACCC TTCGACATTC AGACTTCATC TCCACCTATA GGTGATGACG |
| | GTCCCGTTTA CACGGTCGTT TCGGTACATG AGGTAACTGT AGTTGCTACA |
| | GCAGGACCAG TGCTCCTGCT GCTCTCCGAC ACGGACGGAG CCCATGCAGG |
| | TGGCCCTGCA CTGCACCAAT GGCTCTGTTG TGTACCATGA GGTTCTCAAT |
| | CGTCCTGGTC ACGAGGACGA CGAGAGGCTG TGCCTGCCTC GGGTACGTCC |
| | ACCGGGACGT GACGTGGTTA CCGAGACAAC ACATGGTACT CCAAGAGTTA |
| | GCCATGGAGT GCAAATGCTC CCCCAGGAAG TGCAGCAAGT GA |

The present invention is directed to a von Willebrand Factor (VWF) fragment comprising a D′ domain and a D3 domain of VWF, wherein the VWF fragment inhibits binding of endogenous VWF (full-length VWF) to a FVIII protein. In one embodiment, the VWF fragment binds to or is associated with a FVIII protein. By binding to or associating with a FVIII protein, a VWF fragment of the invention protects FVIII from protease cleavage and FVIII activation, stabilizes the heavy chain and light chain of FVIII, and prevents clearance of FVIII by scavenger receptors. In another embodiment, the VWF fragment binds to or associates with a FVIII protein and blocks or prevents binding of the FVIII protein to phospholipid and activated Protein C. By preventing or inhibiting binding of the FVIII protein with endogenous, full-length VWF, the VWF fragment of the invention reduces the clearance of FVIII by VWF clearance receptors and thus extends the half-life of FVIII. The half-life extension of a FVIII protein is thus due to the binding of or associating with the VWF fragment lacking a VWF clearance receptor binding site to the FVIII protein and shielding or protecting of the FVIII protein by the VWF fragment from endogenous VWF which contains the VWF clearance receptor binding site. The FVIII protein bound to or protected by the VWF fragment can also allow recycling of a FVIII protein. Therefore, the VWF fragment cannot be full-length mature VWF. By eliminating the VWF clearance pathway receptor binding sites contained in the full length VWF molecule, the FVIII/VWF heterodimers of the invention are uncoupled from the VWF clearance pathway, which allows the further extending FVIII half-life.

The VWF fragment comprising the D′ domain and the D3 domain can further comprise a VWF domain selected from the group consisting of an A1 domain, an A2 domain, an A3 domain, a D1 domain, a D2 domain, a D4 domain, a B1 domain, a B2 domain, a B3 domain, a C1 domain, a C2 domain, a CK domain, one or more fragments thereof, and any combinations thereof. In one embodiment, a VWF fragment comprises, consists essentially of, or consists of: (1) the D′ and D3 domains of VWF or fragments thereof; (2) the D1, D′, and D3 domains of VWF or fragments thereof; (3) the D2, D′, and D3 domains of VWF or fragments thereof; (4) the D1, D2, D′, and D3 domains of VWF or fragments thereof; or (5) the D1, D2, D′, D3, and A1 domains of VWF or fragments thereof. The VWF fragment described herein does not contain a site binding to a VWF clearance receptor. In another embodiment, the VWF fragment described herein is not amino acids 764 to 1274 of SEQ ID NO: 2. The VWF fragment of the present invention can comprise any other sequences linked to or fused to the VWF fragment, but is not the full-length VWF. For example, a VWF fragment described herein can further comprise a signal peptide.

In one embodiment, a VWF fragment of the present invention comprises the D′ domain and the D3 domain of VWF, wherein the D′ domain is at least 60%, 70%, 80%, 85%, 90%, 95%, 96%, 97%, 98%, 99%, or 100% identical to amino acids 764 to 866 of SEQ ID NO: 2, wherein the VWF fragment binds to a FVIII protein, shields, inhibits or prevents binding of endogenous VWF fragment to a FVIII protein. In another embodiment, a VWF fragment comprises the D′ domain and the D3 domain of VWF, wherein the D3 domain is at least 60%, 70%, 80%, 85%, 90%, 95%, 96%, 97%, 98%, 99%, or 100% identical to amino acids 867 to 1240 of SEQ ID NO: 2, wherein the VWF fragment binds to a FVIII protein or inhibits or prevents binding of endogenous VWF fragment to a FVIII protein. In some embodiments, a VWF fragment described herein comprises, consists essentially of, or consists of the D′ domain and D3 domain of VWF, which are at least 60%, 70%, 80%, 85%, 90%, 95%, 96%, 97%, 98%, 99%, or 100% identical to amino acids 764 to 1240 of SEQ ID NO: 2, wherein the VWF fragment binds to a FVIII protein or inhibits or prevents binding of endogenous VWF fragment to a FVIII protein. In other embodiments, a VWF fragment comprises, consists essentially of, or consists of the D1, D2, D′, and D3 domains at least 60%, 70%, 80%, 85%, 90%, 95%, 96%, 97%, 98%, 99%, or 100% identical to amino acids 23 to 1240 of SEQ ID NO: 2, wherein the VWF fragment binds to a FVIII protein or inhibits or prevents binding of endogenous VWF fragment to a FVIII protein. In still other embodiments, the VWF fragment further comprises a signal peptide operably linked thereto.

In some embodiments, a VWF fragment of the invention consists essentially of or consists of (1) the D′D3 domain, the DID′D3 domain, D2D′D3 domain, or DID2D′D3 domain and (2) an additional VWF sequence up to about 10 amino acids (e.g., any sequences from amino acids 764 to 1240 of SEQ ID NO: 2 to amino acids 764 to 1250 of SEQ ID NO: 2), up to about 15 amino acids (e.g., any sequences from amino acids 764 to 1240 of SEQ ID NO: 2 to amino acids 764 to 1255 of SEQ ID NO: 2), up to about 20 amino acids (e.g., any sequences from amino acids 764 to 1240 of SEQ ID NO: 2 to amino acids 764 to 1260 of SEQ ID NO: 2), up to about 25 amino acids (e.g., any sequences from amino acids 764 to 1240 of SEQ ID NO: 2 to amino acids 764 to 1265 of SEQ ID NO: 2), or up to about 30 amino acids (e.g., any sequences from amino acids 764 to 1240 of SEQ ID NO: 2 to amino acids 764 to 1260 of SEQ ID NO: 2). In a particular embodiment, the VWF fragment comprising or consisting essentially of the D′ domain and the D3 domain is neither amino acids 764 to 1274 of SEQ ID NO: 2 nor the full-length mature VWF.

In other embodiments, the VWF fragment comprising the D′D3 domains linked to the DID2 domains further comprises an intracellular cleavage site, e.g., (a cleavage site by PACE or PC5), allowing cleavage of the DID2 domains from the D′D3 domains upon expression. Non-limiting examples of the intracellular cleavage site are disclosed elsewhere herein.

In yet other embodiments, a VWF fragment comprises the D′ domain and the D3 domain, but does not comprise an amino acid sequence selected from the group consisting of (1) amino acids 1241 to 2813 of SEQ ID NO: 2, (2) amino acids 1270 to amino acids 2813 of SEQ ID NO: 2, (3) amino acids 1271 to amino acids 2813 of SEQ ID NO: 2, (4) amino acids 1272 to amino acids 2813 of SEQ ID NO: 2, (5) amino acids 1273 to amino acids 2813 of SEQ ID NO: 2, and (6) amino acids 1274 to amino acids 2813 of SEQ ID NO: 2.

In still other embodiments, a VWF fragment of the present invention comprises, consists essentially of, or consists of an amino acid sequence corresponding to the D′ domain, D3 domain, and A1 domain, wherein the amino acid sequence is at least 60%, 70%, 75%, 80%, 85%, 90%, 95%, 96%, 97%, 98%, 99%, or 100% identical to amino acid 764 to 1479 of SEQ ID NO: 2, wherein the VWF binds to FVIII. In a particular embodiment, the VWF fragment is not amino acids 764 to 1274 of SEQ ID NO: 2.

In some embodiments, a VWF fragment of the invention comprises the D′ domain and the D3 domain, but does not comprise at least one VWF domain selected from the group consisting of (1) an A1 domain, (2) an A2 domain, (3) an A3 domain, (4) a D4 domain, (5) a B1 domain, (6) a B2 domain, (7) a B3 domain, (8) a C1 domain, (9) a C2 domain, (10) a CK domain, (11) a CK domain and C2 domain, (12) a CK domain, a C2 domain, and a C1 domain, (13) a CK domain, a C2 domain, a C1 domain, a B3 domain, (14) a CK domain, a C2 domain, a C1 domain, a B3 domain, a B2 domain, (15) a CK domain, a C2 domain, a C1 domain, a B3 domain, a B2 domain, and a B1 domain, (16) a CK domain, a C2 domain, a C1 domain, a B3 domain, a B2 domain, a B1 domain, and a D4 domain, (17) a CK domain, a C2 domain, a C1 domain, a B3 domain, a B2 domain, a B1 domain, a D4 domain, and an A3 domain, (18) a CK domain, a C2 domain, a C1 domain, a B3 domain, a B2 domain, a B1 domain, a D4 domain, an A3 domain, and an A2 domain, (19) a CK domain, a C2 domain, a C1 domain, a B3 domain, a B2 domain, a B1 domain, a D4 domain, an A3 domain, an A2 domain, and an A1 domain, and (20) any combinations thereof.

In yet other embodiments, the VWF fragment comprises the D′D3 domains and one or more domains or modules. Examples of such domains or modules include, but are not limited to, the domains and modules disclosed in Zhour et al., Blood published online Apr. 6, 2012: DOI 10.1182/blood-2012-01-405134. For example, the VWF fragment can comprise the D′D3 domain and one or more domains or modules selected from the group consisting of A1 domain, A2 domain, A3 domain, D4N module, VWD4 module, C8-4 module, TIL-4 module, C1 module, C2 module, C3 module, C4 module, C5 module, C5 module, C6 module, and any combinations thereof.

In still other embodiments, the VWF fragment is linked to a heterologous moiety, wherein the heterologous moiety is linked to the N-terminus or the C-terminus of the VWF fragment or inserted between two amino acids in the VWF fragment. For example, the insertion sites for the heterologous moiety in the VWF fragment can be in the D′ domain, the D3 domain, or both. The heterologous moiety can be a half-life extender.

In certain embodiments, a VWF fragment of the invention forms a multimer, e.g., dimer, trimer, tetramer, pentamer, hexamer, heptamer, or the higher order multimers. In other embodiments, the VWF fragment is a monomer having only one VWF fragment. In some embodiments, the VWF fragment of the present invention can have one or more amino acid substitutions, deletions, additions, or modifications. In one embodiment, the VWF fragment can include amino acid substitutions, deletions, additions, or modifications such that the VWF fragment is not capable of forming a disulfide bond or forming a dimer or a multimer. In another embodiment, the amino acid substitution is within the D′ domain and the D3 domain. In a particular embodiment, a VWF fragment of the invention contains at least one amino acid substitution at a residue corresponding to residue 1099, residue 1142, or both residues 1099 and 1142 of SEQ ID NO: 2. The at least one amino acid substitution can be any amino acids that are not occurring naturally in the wild type VWF. For example, the amino acid substitution can be any amino acids other than cysteine, e.g., Isoleucine, Alanine, Leucine, Asparagine, Lysine, Aspartic acid, Methionine, Phenylalanine, Glutamic acid, Threonine, Glutamine, Tryptophan, Glycine, Valine, Proline, Serine, Tyrosine, Arginine, or Histidine. In another example, the amino acid substitution has one or more amino acids that prevent or inhibit the VWF fragments from forming multimers.

In certain embodiments, the VWF fragment useful herein can be further modified to improve its interaction with FVIII, e.g., to improve binding affinity to FVIII. As a non-limiting example, the VWF fragment comprises a serine residue at the residue corresponding to amino acid 764 of SEQ ID NO: 2 and a lysine residue at the residue corresponding to amino acid 773 of SEQ ID NO: 2. Residues 764 and/or 773 can contribute to the binding affinity of the VWF fragments to FVIII. In other embodiments, the VWF fragment can have other modifications, e.g., the fragment can be pegylated, glycosylated, hesylated, or polysialylated.

### B) Heterologous Moieties

The heterologous moiety can be a heterologous polypeptide or a heterologous non-polypeptide moiety. In certain embodiments, the heterologous moiety is a half-life extending molecule which is known in the art and comprises a polypeptide, a non-polypeptide moiety, or the combination of both. The heterologous polypeptide moiety can comprise an immunoglobulin constant region or a portion thereof, albumin or a fragment thereof, an albumin binding moiety, transferrin or a fragment thereof, a PAS sequence, a HAP sequence, a derivative or variant thereof, or any combinations thereof. In some embodiments, the non-polypeptide binding moiety comprises polyethylene glycol (PEG), polysialic acid, hydroxyethyl starch (HES), a derivative thereof, or any combinations thereof. In certain embodiments, there can be one, two, three or more heterologous moieties, which can each be the same or different molecules.

### 1) Immunoglobulin Constant Region or Portion Thereof

An immunoglobulin constant region is comprised of domains denoted CH (constant heavy) domains (CH1, CH2, etc.). Depending on the isotype, (i.e. IgG, IgM, IgA IgD, or IgE), the constant region can be comprised of three or four CH domains. Some isotypes (e.g. IgG) constant regions also contain a hinge region. See Janeway et al. 2001, Immunobiology, Garland Publishing, N.Y., N.Y.

An immunoglobulin constant region or a portion thereof for producing the chimeric protein of the present invention may be obtained from a number of different sources. In preferred embodiments, an immunoglobulin constant region or a portion thereof is derived from a human immunoglobulin. It is understood, however, that the immunoglobulin constant region or a portion thereof may be derived from an immunoglobulin of another mammalian species, including for example, a rodent (e.g. a mouse, rat, rabbit, guinea pig) or non-human primate (e.g. chimpanzee, macaque) species. Moreover, the immunoglobulin constant region or a portion thereof may be derived from any immunoglobulin class, including IgM, IgG, IgD, IgA and IgE, and any immunoglobulin isotype, including IgG1, IgG2, IgG3 and IgG4. In one embodiment, the human isotype IgG1 is used.

A variety of the immunoglobulin constant region gene sequences (e.g. human constant region gene sequences) are available in the form of publicly accessible deposits. Constant region domains sequence can be selected having a particular effector function (or lacking a particular effector function) or with a particular modification to reduce immunogenicity. Many sequences of antibodies and antibody-encoding genes have been published and suitable Ig constant region sequences (e.g. hinge, CH2, and/or CH3 sequences, or portions thereof) can be derived from these sequences using art recognized techniques. The genetic material obtained using any of the foregoing methods may then be altered or synthesized to obtain polypeptides of the present invention. It will further be appreciated that the scope of this invention encompasses alleles, variants and mutations of constant region DNA sequences.

The sequences of the immunoglobulin constant region or a portion thereof can be cloned, e.g., using the polymerase chain reaction and primers which are selected to amplify the domain of interest. To clone a sequence of the immunoglobulin constant region or a portion thereof from an antibody, mRNA can be isolated from hybridoma, spleen, or lymph cells, reverse transcribed into DNA, and antibody genes amplified by PCR. PCR amplification methods are described in detail in U.S. Pat. Nos. 4,683,195; 4,683,202; 4,800,159; 4,965,188; and in, e.g., “PCR Protocols: A Guide to Methods and Applications” Innis et al. eds., Academic Press, San Diego, CA (1990); Ho et al. 1989. Gene 77:51; Horton et al. 1993. Methods Enzymol. 217:270). PCR may be initiated by consensus constant region primers or by more specific primers based on the published heavy and light chain DNA and amino acid sequences. As discussed above, PCR also may be used to isolate DNA clones encoding the antibody light and heavy chains. In this case the libraries may be screened by consensus primers or larger homologous probes, such as mouse constant region probes. Numerous primer sets suitable for amplification of antibody genes are known in the art (e.g., 5′ primers based on the N-terminal sequence of purified antibodies (Benhar and Pastan. 1994. Protein Engineering 7:1509); rapid amplification of cDNA ends (Ruberti, F. et al. 1994. J. Immunol. Methods 173:33); antibody leader sequences (Larrick et al. 1989 Biochem. Biophys. Res. Commun. 160:1250). The cloning of antibody sequences is further described in Newman et al., U.S. Pat. No. 5,658,570, filed Jan. 25, 1995, which is incorporated by reference herein.

An immunoglobulin constant region used herein can include all domains and the hinge region or portions thereof. In one embodiment, the immunoglobulin constant region or a portion thereof comprises CH2 domain, CH3 domain, and a hinge region, i.e., an Fc region or an FcRn binding partner.

As used herein, the term “Fc region” is defined as the portion of a polypeptide which corresponds to the Fc region of native immunoglobulin, i.e., as formed by the dimeric association of the respective Fc domains of its two heavy chains. A native Fc region forms a homodimer with another Fc region. In contrast, the term “genetically-fused Fc region” or “single-chain Fc region” (scFc region), as used herein, refers to a synthetic dimeric Fc region comprised of Fc domains genetically linked within a single polypeptide chain (i.e., encoded in a single contiguous genetic sequence).

In one embodiment, the “Fc region” refers to the portion of a single immunoglobulin heavy chain beginning in the hinge region just upstream of the papain cleavage site (i.e. residue 216 in IgG, taking the first residue of heavy chain constant region to be 114) and ending at the C-terminus of the antibody. Accordingly, a complete Fc domain comprises at least a hinge domain, a CH2 domain, and a CH3 domain.

The Fc region of an immunoglobulin constant region, depending on the immunoglobulin isotype can include the CH2, CH3, and CH4 domains, as well as the hinge region. Chimeric proteins comprising an Fc region of an immunoglobulin bestow several desirable properties on a chimeric protein including increased stability, increased serum half-life (see Capon et al., 1989, Nature 337:525) as well as binding to Fc receptors such as the neonatal Fc receptor (FcRn) (U.S. Pat. Nos. 6,086,875, 6,485,726, 6,030,613; WO 03/077834; US2003-0235536A1), which are incorporated herein by reference in their entireties.

An immunoglobulin constant region or a portion thereof can be an FcRn binding partner. FcRn is active in adult epithelial tissues and expressed in the lumen of the intestines, pulmonary airways, nasal surfaces, vaginal surfaces, colon and rectal surfaces (U.S. Pat. No. 6,485,726). An FcRn binding partner is a portion of an immunoglobulin that binds to FcRn.

The FcRn receptor has been isolated from several mammalian species including humans. The sequences of the human FcRn, monkey FcRn, rat FcRn, and mouse FcRn are known (Story et al. 1994, J. Exp. Med. 180:2377). The FcRn receptor binds IgG (but not other immunoglobulin classes such as IgA, IgM, IgD, and IgE) at relatively low pH, actively transports the IgG transcellularly in a luminal to serosal direction, and then releases the IgG at relatively higher pH found in the interstitial fluids. It is expressed in adult epithelial tissue (U.S. Pat. Nos. 6,485,726, 6,030,613, 6,086,875; WO 03/077834; US2003-0235536A1) including lung and intestinal epithelium (Israel et al. 1997, Immunology 92:69) renal proximal tubular epithelium (Kobayashi et al. 2002, Am. J. Physiol. Renal Physiol. 282:F358) as well as nasal epithelium, vaginal surfaces, and biliary tree surfaces.

FcRn binding partners useful in the present invention encompass molecules that can be specifically bound by the FcRn receptor including whole IgG, the Fc fragment of IgG, and other fragments that include the complete binding region of the FcRn receptor. The region of the Fc portion of IgG that binds to the FcRn receptor has been described based on X-ray crystallography (Burmeister et al. 1994, Nature 372:379). The major contact area of the Fc with the FcRn is near the junction of the CH2 and CH3 domains. Fc-FcRn contacts are all within a single Ig heavy chain. The FcRn binding partners include whole IgG, the Fc fragment of IgG, and other fragments of IgG that include the complete binding region of FcRn. The major contact sites include amino acid residues 248, 250-257, 272, 285, 288, 290-291, 308-311, and 314 of the CH2 domain and amino acid residues 385-387, 428, and 433-436 of the CH3 domain. References made to amino acid numbering of immunoglobulins or immunoglobulin fragments, or regions, are all based on Kabat et al. 1991, Sequences of Proteins of Immunological Interest, U.S. Department of Public Health, Bethesda, Md.

Fc regions or FcRn binding partners bound to FcRn can be effectively shuttled across epithelial barriers by FcRn, thus providing a non-invasive means to systemically administer a desired therapeutic molecule. Additionally, fusion proteins comprising an Fc region or an FcRn binding partner are endocytosed by cells expressing the FcRn. But instead of being marked for degradation, these fusion proteins are recycled out into circulation again, thus increasing the in vivo half-life of these proteins. In certain embodiments, the portions of immunoglobulin constant regions are an Fc region or an FcRn binding partner that typically associates, via disulfide bonds and other non-specific interactions, with another Fc region or another FcRn binding partner to form dimers and higher order multimers.

Two FcRn receptors can bind a single Fc molecule. Crystallographic data suggest that each FcRn molecule binds a single polypeptide of the Fc homodimer. In one embodiment, linking the FcRn binding partner, e.g., an Fc fragment of an IgG, to a biologically active molecule provides a means of delivering the biologically active molecule orally, buccally, sublingually, rectally, vaginally, as an aerosol administered nasally or via a pulmonary route, or via an ocular route. In another embodiment, the chimeric protein can be administered invasively, e.g., subcutaneously, intravenously.

An FcRn binding partner region is a molecule or a portion thereof that can be specifically bound by the FcRn receptor with consequent active transport by the FcRn receptor of the Fc region. Specifically bound refers to two molecules forming a complex that is relatively stable under physiologic conditions. Specific binding is characterized by a high affinity and a low to moderate capacity as distinguished from nonspecific binding which usually has a low affinity with a moderate to high capacity. Typically, binding is considered specific when the affinity constant KA is higher than 10⁶ M⁻¹, or higher than 10⁸ M⁻¹. If necessary, non-specific binding can be reduced without substantially affecting specific binding by varying the binding conditions. The appropriate binding conditions such as concentration of the molecules, ionic strength of the solution, temperature, time allowed for binding, concentration of a blocking agent (e.g. serum albumin, milk casein), etc., may be optimized by a skilled artisan using routine techniques.

In certain embodiments, a chimeric protein of the invention comprises one or more truncated Fc regions that are nonetheless sufficient to confer Fc receptor (FcR) binding properties to the Fc region. For example, the portion of an Fc region that binds to FcRn (i.e., the FcRn binding portion) comprises from about amino acids 282-438 of IgG1, EU numbering (with the primary contact sites being amino acids 248, 250-257, 272, 285, 288, 290-291, 308-311, and 314 of the CH2 domain and amino acid residues 385-387, 428, and 433-436 of the CH3 domain. Thus, an Fc region of the invention may comprise or consist of an FcRn binding portion. FcRn binding portions may be derived from heavy chains of any isotype, including IgG1, IgG2, IgG3 and IgG4. In one embodiment, an FcRn binding portion from an antibody of the human isotype IgG1 is used. In another embodiment, an FcRn binding portion from an antibody of the human isotype IgG4 is used.

In another embodiment, the “Fc region” includes an amino acid sequence of an Fc domain or derived from an Fc domain. In certain embodiments, an Fc region comprises at least one of: a hinge (e.g., upper, middle, and/or lower hinge region) domain (about amino acids 216-230 of an antibody Fc region according to EU numbering), a CH2 domain (about amino acids 231-340 of an antibody Fc region according to EU numbering), a CH3 domain (about amino acids 341-438 of an antibody Fc region according to EU numbering), a CH4 domain, or a variant, portion, or fragment thereof. In other embodiments, an Fc region comprises a complete Fc domain (i.e., a hinge domain, a CH2 domain, and a CH3 domain). In some embodiments, an Fc region comprises, consists essentially of, or consists of a hinge domain (or a portion thereof) fused to a CH3 domain (or a portion thereof), a hinge domain (or a portion thereof) fused to a CH2 domain (or a portion thereof), a CH2 domain (or a portion thereof) fused to a CH3 domain (or a portion thereof), a CH2 domain (or a portion thereof) fused to both a hinge domain (or a portion thereof) and a CH3 domain (or a portion thereof). In still other embodiments, an Fc region lacks at least a portion of a CH2 domain (e.g., all or part of a CH2 domain). In a particular embodiment, an Fc region comprises or consists of amino acids corresponding to EU numbers 221 to 447.

The Fc regions denoted as F, F1, or F2 herein may be obtained from a number of different sources. In one embodiment, an Fc region of the polypeptide is derived from a human immunoglobulin. It is understood, however, that an Fc region may be derived from an immunoglobulin of another mammalian species, including for example, a rodent (e.g. a mouse, rat, rabbit, guinea pig) or non-human primate (e.g. chimpanzee, macaque) species. Moreover, the polypeptide of the Fc domains or portions thereof may be derived from any immunoglobulin class, including IgM, IgG, IgD, IgA and IgE, and any immunoglobulin isotype, including IgG1, IgG2, IgG3 and IgG4. In another embodiment, the human isotype IgG1 is used.

In certain embodiments, the Fc variant confers a change in at least one effector function imparted by an Fc region comprising said wild-type Fc domain (e.g., an improvement or reduction in the ability of the Fc region to bind to Fc receptors (e.g. FcγRI, FcγRII, or FcγRIII) or complement proteins (e.g. C1q), or to trigger antibody-dependent cytotoxicity (ADCC), phagocytosis, or complement-dependent cytotoxicity (CDCC)). In other embodiments, the Fc variant provides an engineered cysteine residue.

The Fc regions of the invention may employ art-recognized Fc variants which are known to impart a change (e.g., an enhancement or reduction) in effector function and/or FcR or FcRn binding. Specifically, a binding molecule of the invention may include, for example, a change (e.g., a substitution) at one or more of the amino acid positions disclosed in International PCT Publications WO88/07089A1, WO96/14339A1, WO98/05787A1, WO98/23289A1, WO99/51642A1, WO99/58572A1, WO00/09560A2, WO00/32767A1, WO00/42072A2, WO02/44215A2, WO02/060919A2, WO03/074569A2, WO04/016750A2, WO04/029207A2, WO04/035752A2, WO04/063351A2, WO04/074455A2, WO04/099249A2, WO05/040217A2, WO04/044859, WO05/070963A1, WO05/077981A2, WO05/092925A2, WO05/123780A2, WO06/019447A1, WO06/047350A2, and WO06/085967A2; US Patent Publication Nos. US2007/0231329, US2007/0231329, US2007/0237765, US2007/0237766, US2007/0237767, US2007/0243188, US20070248603, US20070286859, US20080057056; or U.S. Pat. Nos. 5,648,260; 5,739,277; 5,834,250; 5,869,046; 6,096,871; 6,121,022; 6,194,551; 6,242,195; 6,277,375; 6,528,624; 6,538,124; 6,737,056; 6,821,505; 6,998,253; 7,083,784; 7,404,956, and 7,317,091, each of which is incorporated by reference herein. In one embodiment, the specific change (e.g., the specific substitution of one or more amino acids disclosed in the art) may be made at one or more of the disclosed amino acid positions. In another embodiment, a different change at one or more of the disclosed amino acid positions (e.g., the different substitution of one or more amino acid position disclosed in the art) may be made.

The Fc region or FcRn binding partner of IgG can be modified according to well recognized procedures such as site directed mutagenesis and the like to yield modified IgG or Fc fragments or portions thereof that will be bound by FcRn. Such modifications include modifications remote from the FcRn contact sites as well as modifications within the contact sites that preserve or even enhance binding to the FcRn. For example, the following single amino acid residues in human IgG1 Fc (Fc γ1) can be substituted without significant loss of Fc binding affinity for FcRn: P238A, S239A, K246A, K248A, D249A, M252A, T256A, E258A, T260A, D265A, S267A, H268A, E269A, D270A, E272A, L274A, N276A, Y278A, D280A, V282A, E283A, H285A, N286A, T289A, K290A, R292A, E293A, E294A, Q295A, Y296F, N297A, S298A, Y300F, R301A, V303A, V305A, T307A, L309A, Q311A, D312A, N315A, K317A, E318A, K320A, K322A, S324A, K326A, A327Q, P329A, A330Q, P331A, E333A, K334A, T335A, S337A, K338A, K340A, Q342A, R344A, E345A, Q347A, R355A, E356A, M358A, T359A, K360A, N361A, Q362A, Y373A, S375A, D376A, A378Q, E380A, E382A, S383A, N384A, Q386A, E388A, N389A, N390A, Y391F, K392A, L398A, S400A, D401A, D413A, K414A, R416A, Q418A, Q419A, N421A, V422A, S424A, E430A, N434A, T437A, Q438A, K439A, S440A, S444A, and K447A, where for example P238A represents wild type proline substituted by alanine at position number 238. As an example, a specific embodiment incorporates the N297A mutation, removing a highly conserved N-glycosylation site. In addition to alanine other amino acids may be substituted for the wild type amino acids at the positions specified above. Mutations may be introduced singly into Fc giving rise to more than one hundred Fc regions distinct from the native Fc. Additionally, combinations of two, three, or more of these individual mutations may be introduced together, giving rise to hundreds more Fc regions. Moreover, one of the Fc region of a construct of the invention may be mutated and the other Fc region of the construct not mutated at all, or they both may be mutated but with different mutations.

Certain of the above mutations may confer new functionality upon the Fc region or FcRn binding partner. For example, one embodiment incorporates N297A, removing a highly conserved N-glycosylation site. The effect of this mutation is to reduce immunogenicity, thereby enhancing circulating half-life of the Fc region, and to render the Fc region incapable of binding to FcγRI, FcγRIIA, FcγRIIB, and FcγRIIIA, without compromising affinity for FcRn (Routledge et al. 1995, Transplantation 60:847; Friend et al. 1999, Transplantation 68:1632; Shields et al. 1995, J. Biol. Chem. 276:6591). As a further example of new functionality arising from mutations described above affinity for FcRn may be increased beyond that of wild type in some instances. This increased affinity may reflect an increased “on” rate, a decreased “off” rate or both an increased “on” rate and a decreased “off” rate. Examples of mutations believed to impart an increased affinity for FcRn include, but not limited to, T256A, T307A, E380A, and N434A (Shields et al. 2001, J. Biol. Chem. 276:6591).

Additionally, at least three human Fc gamma receptors appear to recognize a binding site on IgG within the lower hinge region, generally amino acids 234-237. Therefore, another example of new functionality and potential decreased immunogenicity may arise from mutations of this region, as for example by replacing amino acids 233-236 of human IgG1 “ELLG” to the corresponding sequence from IgG2 “PVA” (with one amino acid deletion). It has been shown that FcγRI, FcγRII, and FcγRIII, which mediate various effector functions will not bind to IgG1 when such mutations have been introduced. Ward and Ghetie 1995, Therapeutic Immunology 2:77 and Armour et al. 1999, Eur. J. Immunol. 29:2613.

In one embodiment, the immunoglobulin constant region or a portion thereof, e.g, an Fc region, is a polypeptide including the sequence PKNSSMISNTP (SEQ ID NO: 3) and optionally further including a sequence selected from HQSLGTQ (SEQ ID NO: 4), HQNLSDGK (SEQ ID NO: 5), HQNISDGK (SEQ ID NO: 6), or VISSHLGQ (SEQ ID NO: 7) (U.S. Pat. No. 5,739,277).

In another embodiment, the immunoglobulin constant region or a portion thereof comprises an amino acid sequence in the hinge region or a portion thereof that forms one or more disulfide bonds with another immunoglobulin constant region or a portion thereof. The disulfide bond by the immunoglobulin constant region or a portion thereof places the first polypeptide comprising FVIII and the second polypeptide comprising the VWF fragment together so that endogenous VWF does not replace the VWF fragment and does not bind to the FVIII. Therefore, the disulfide bond between the first immunoglobulin constant region or a portion thereof and a second immunoglobulin constant region or a portion thereof prevents interaction between endogenous VWF and the FVIII protein. This inhibition of interaction between the VWF and the FVIII protein allows the half-life of the FVIII protein to go beyond the two fold limit. The hinge region or a portion thereof can further be linked to one or more domains of CH1, CH2, CH3, a fragment thereof, and any combinations thereof. In a particular example, an immunoglobulin constant region or a portion thereof comprises a hinge region and CH2 region (e.g., amino acids 221-340 of an Fc region).

In certain embodiments, the immunoglobulin constant region or a portion thereof is hemi-glycosylated. For example, the chimeric protein comprising two Fc regions or FcRn binding partners may contain a first, glycosylated, Fc region (e.g., a glycosylated CH2 region) or FcRn binding partner and a second, aglycosylated, Fc region (e.g., an aglycosylated CH2 region) or FcRn binding partner. In one embodiment, a linker may be interposed between the glycosylated and aglycosylated Fc regions. In another embodiment, the Fc region or FcRn binding partner is fully glycosylated, i.e., all of the Fc regions are glycosylated. In other embodiments, the Fc region may be aglycosylated, i.e., none of the Fc moieties are glycosylated.

In certain embodiments, a chimeric protein of the invention comprises an amino acid substitution to an immunoglobulin constant region or a portion thereof (e.g., Fc variants), which alters the antigen-independent effector functions of the Ig constant region, in particular the circulating half-life of the protein.

Such proteins exhibit either increased or decreased binding to FcRn when compared to proteins lacking these substitutions and, therefore, have an increased or decreased half-life in serum, respectively. Fc variants with improved affinity for FcRn are anticipated to have longer serum half-lives, and such molecules have useful applications in methods of treating mammals where long half-life of the administered polypeptide is desired, e.g., to treat a chronic disease or disorder (see, e.g, U.S. Pat. Nos. 7,348,004, 7,404,956, and 7,862,820). In contrast, Fc variants with decreased FcRn binding affinity are expected to have shorter half-lives, and such molecules are also useful, for example, for administration to a mammal where a shortened circulation time may be advantageous, e.g. for in vivo diagnostic imaging or in situations where the starting polypeptide has toxic side effects when present in the circulation for prolonged periods. Fc variants with decreased FcRn binding affinity are also less likely to cross the placenta and, thus, are also useful in the treatment of diseases or disorders in pregnant women. In addition, other applications in which reduced FcRn binding affinity may be desired include those applications in which localization the brain, kidney, and/or liver is desired. In one exemplary embodiment, the chimeric protein of the invention exhibit reduced transport across the epithelium of kidney glomeruli from the vasculature. In another embodiment, the chimeric protein of the invention exhibit reduced transport across the blood brain barrier (BBB) from the brain, into the vascular space. In one embodiment, a protein with altered FcRn binding comprises at least one Fc region or FcRn binding partner (e.g, one or two Fc regions or FcRn binding partners) having one or more amino acid substitutions within the “FcRn binding loop” of an Ig constant region. The FcRn binding loop is comprised of amino acid residues 280-299 (according to EU numbering) of a wild-type, full-length, Fc region. In other embodiments, an Ig constant region or a portion thereof in a chimeric protein of the invention having altered FcRn binding affinity comprises at least one Fc region or FcRn binding partner having one or more amino acid substitutions within the 15 Å FcRn “contact zone.” As used herein, the term 15 Å FcRn “contact zone” includes residues at the following positions of a wild-type, full-length Fc moiety: 243-261, 275-280, 282-293, 302-319, 336-348, 367, 369, 372-389, 391, 393, 408, 424, 425-440 (EU numbering). In other embodiments, a Ig constant region or a portion thereof of the invention having altered FcRn binding affinity comprises at least one Fc region or FcRn binding partner having one or more amino acid substitutions at an amino acid position corresponding to any one of the following EU positions: 256, 277-281, 283-288, 303-309, 313, 338, 342, 376, 381, 384, 385, 387, 434 (e.g., N434A or N434K), and 438. Exemplary amino acid substitutions which altered FcRn binding activity are disclosed in International PCT Publication No. WO05/047327 which is incorporated by reference herein.

An Fc region or FcRn binding partner used in the invention may also comprise an art recognized amino acid substitution which alters the glycosylation of the chimeric protein. For example, the Fc region or FcRn binding partner of the chimeric protein linked to a VWF fragment or a FVIII protein may comprise an Fc region having a mutation leading to reduced glycosylation (e.g., N- or O-linked glycosylation) or may comprise an altered glycoform of the wild-type Fc moiety (e.g., a low fucose or fucose-free glycan).

In one embodiment, an unprocessed chimeric protein of the invention may comprise a genetically fused Fc region (i.e., scFc region) having two or more of its constituent Ig constant region or a portion thereof independently selected from the Ig constant region or a portion thereof described herein. In one embodiment, the Fc regions of a dimeric Fc region are the same. In another embodiment, at least two of the Fc regions are different. For example, the Fc regions or FcRn binding partners of the proteins of the invention comprise the same number of amino acid residues or they may differ in length by one or more amino acid residues (e.g., by about 5 amino acid residues (e.g., 1, 2, 3, 4, or 5 amino acid residues), about 10 residues, about 15 residues, about 20 residues, about 30 residues, about 40 residues, or about 50 residues). In yet other embodiments, the Fc regions or FcRn binding partners of the protein of the invention may differ in sequence at one or more amino acid positions. For example, at least two of the Fc regions or FcRn binding partners may differ at about 5 amino acid positions (e.g., 1, 2, 3, 4, or 5 amino acid positions), about 10 positions, about 15 positions, about 20 positions, about 30 positions, about 40 positions, or about 50 positions).

### 2) Albumin or Fragment, or Variant Thereof

In certain embodiments, the heterologous moiety linked to the VWF fragment or linked to a FVIII protein is albumin or a functional fragment thereof. In other embodiments, a chimeric protein of the invention comprises a FVIII protein and albumin or a fragment thereof, wherein the albumin or a fragment thereof shields or protects the VWF binding site on the FVIII protein, thereby inhibiting or preventing interaction of the FVIII protein with endogenous VWF.

Human serum albumin (HSA, or HA), a protein of 609 amino acids in its full-length form, is responsible for a significant proportion of the osmotic pressure of serum and also functions as a carrier of endogenous and exogenous ligands. The term “albumin” as used herein includes full-length albumin or a functional fragment, variant, derivative, or analog thereof.

In one embodiment, the chimeric protein comprises the VWF fragment described herein and albumin, fragment, or variant thereof, wherein the VWF fragment is linked to albumin or a fragment or variant thereof. In another embodiment, the chimeric protein comprises the VWF fragment and a FVIII protein, which are bound to each other, wherein the VWF fragment is linked to albumin or a fragment or variant thereof, the protein having VIII activity is linked to albumin or a fragment or variant thereof, or both the VWF fragment and the protein having VIII activity are linked to albumin or a fragment or variant thereof. In other embodiments, the chimeric protein comprises the VWF fragment linked to albumin or a fragment or variant thereof is further linked to a heterologous moiety selected from the group consisting of an immunoglobulin constant region or a portion thereof (e.g., an Fc region), a PAS sequence, HES, and PEG. In still other embodiments, the chimeric protein comprises the VWF fragment and a FVIII protein, which are bound to each other, wherein the FVIII protein is linked to albumin or a fragment or variant thereof and further linked to a heterologous moiety selected from the group consisting of an immunoglobulin constant region or a portion thereof (e.g., an Fc region), a PAS sequence, HES, and PEG. In yet other embodiments, the chimeric protein comprises the VWF fragment linked to albumin or a fragment or variant thereof and a FVIII protein linked to albumin or a fragment or variant thereof, which are bound to each other, wherein the VWF fragment activity is further linked to a first heterologous moiety selected from the group consisting of an immunoglobulin constant region or a portion thereof (e.g., an Fc region), a PAS sequence, HES, and PEG and wherein the FVIII protein activity is further linked to a second heterologous moiety selected from the group consisting of an immunoglobulin constant region or a portion thereof (e.g., an Fc region), a PAS sequence, HES, and PEG.

In other embodiments, the heterologous moiety linked to the VWF fragment or the FVIII protein is albumin or a fragment or variant thereof, which extends (or is capable of extending) the half-life of the VWF fragment or the FVIII protein. Further examples of albumin or the fragments or variants thereof are disclosed in US Pat. Publ. Nos. 2008/0194481A1, 2008/0004206 A1, 2008/0161243 A1, 2008/0261877 A1, or 2008/0153751 A1 or PCT Appl. Publ. Nos. 2008/033413 A2, 2009/058322 A1, or 2007/021494 A2.

### 3) Albumin Binding Moiety

In certain embodiments, the heterologous moiety linked to the VWF fragment or the FVIII protein is an albumin binding moiety, which comprises an albumin binding peptide, a bacterial albumin binding domain, an albumin-binding antibody fragment, or any combinations thereof. For example, the albumin binding protein can be a bacterial albumin binding protein, an antibody or an antibody fragment including domain antibodies (see U.S. Pat. No. 6,696,245). An albumin binding protein, for example, can be a bacterial albumin binding domain, such as the one of streptococcal protein G (Konig, T. and Skerra, A. (1998) J. Immunol. Methods 218, 73-83). Other examples of albumin binding peptides that can be used as conjugation partner are, for instance, those having a Cys-Xaa ₁-Xaa ₂-Xaa ₃-Xaa ₄-Cys consensus sequence, wherein Xaa ₁ is Asp, Asn, Ser, Thr, or Trp; Xaa ₂ is Asn, Gln, H is, Ile, Leu, or Lys; Xaa ₃ is Ala, Asp, Phe, Trp, or Tyr, and Xaa ₄ is Asp, Gly, Leu, Phe, Ser, or Thr as described in US patent application 2003/0069395 or Dennis et al. (Dennis et al. (2002) J. Biol. Chem. 277, 35035-35043).

### 4) PAS Sequence

In other embodiments, the heterologous moiety linked to the VWF fragment or to the FVIII protein is a PAS sequence. In one embodiment, the chimeric protein comprises a VWF fragment described herein and a PAS sequence, wherein the VWF fragment is linked to the PAS sequence. In another embodiment, a chimeric protein of the invention comprises a FVIII protein and a PAS sequence, wherein the PAS sequence shields or protects the VWF binding site on the FVIII protein, thereby inhibiting or preventing interaction of the FVIII protein with endogenous VWF.

A PAS sequence, as used herein, means an amino acid sequence comprising mainly alanine and serine residues or comprising mainly alanine, serine, and proline residues, the amino acid sequence forming random coil conformation under physiological conditions. Accordingly, the PAS sequence is a building block, an amino acid polymer, or a sequence cassette comprising, consisting essentially of, or consisting of alanine, serine, and proline which can be used as a part of the heterologous moiety in the chimeric protein. Yet, the skilled person is aware that an amino acid polymer also may form random coil conformation when residues other than alanine, serine, and proline are added as a minor constituent in the PAS sequence. The term “minor constituent” as used herein means that amino acids other than alanine, serine, and proline may be added in the PAS sequence to a certain degree, e.g., up to about 12%, i.e., about 12 of 100 amino acids of the PAS sequence, up to about 10%, i.e. about 10 of 100 amino acids of the PAS sequence, up to about 9%, i.e., about 9 of 100 amino acids, up to about 8%, i.e., about 8 of 100 amino acids, about 6%, i.e., about 6 of 100 amino acids, about 5%, i.e., about 5 of 100 amino acids, about 4%, i.e., about 4 of 100 amino acids, about 3%, i.e., about 3 of 100 amino acids, about 2%, i.e., about 2 of 100 amino acids, about 1%, i.e., about 1 of 100 of the amino acids. The amino acids different from alanine, serine and proline may be selected from the group consisting of Arg, Asn, Asp, Cys, Gln, Glu, Gly, His, Ile, Leu, Lys, Met, Phe, Thr, Trp, Tyr, and Val.

Under physiological conditions, the PAS sequence stretch forms a random coil conformation and thereby can mediate an increased in vivo and/or in vitro stability to the VWF factor or the protein of coagulation activity. Since the random coil domain does not adopt a stable structure or function by itself, the biological activity mediated by the VWF fragment or the FVIII protein to which it is fused is essentially preserved. In other embodiments, the PAS sequences that form random coil domain are biologically inert, especially with respect to proteolysis in blood plasma, immunogenicity, isoelectric point/electrostatic behavior, binding to cell surface receptors or internalization, but are still biodegradable, which provides clear advantages over synthetic polymers such as PEG.

Non-limiting examples of the PAS sequences forming random coil conformation comprise an amino acid sequence selected from the group consisting of ASPAAPAPASPAAPAPSAPA (SEQ ID NO: 8), AAPASPAPAAPSAPAPAAPS (SEQ ID NO: 9), APSSPSPSAPSSPSPASPSS (SEQ ID NO: 10), APSSPSPSAPSSPSPASPS (SEQ ID NO: 11), SSPSAPSPSSPASPSPSSPA (SEQ ID NO: 12), AASPAAPSAPPAAASPAAPSAPPA (SEQ ID NO: 13) and ASAAAPAAASAAASAPSAAA (SEQ ID NO: 14) or any combinations thereof. Additional examples of PAS sequences are known from, e.g., US Pat. Publ. No. 2010/0292130 A1 and PCT Appl. Publ. No. WO 2008/155134 A1.

### 5) HAP Sequence

In certain embodiments, the heterologous moiety linked to the VWF fragment or the FVIII protein is a glycine-rich homo-amino-acid polymer (HAP). The HAP sequence can comprise a repetitive sequence of glycine, which has at least 50 amino acids, at least 100 amino acids, 120 amino acids, 140 amino acids, 160 amino acids, 180 amino acids, 200 amino acids, 250 amino acids, 300 amino acids, 350 amino acids, 400 amino acids, 450 amino acids, or 500 amino acids in length. In one embodiment, the HAP sequence is capable of extending half-life of a moiety fused to or linked to the HAP sequence. Non-limiting examples of the HAP sequence includes, but are not limited to (Gly)ₙ, (Gly₄Ser)ₙ or S(Gly₄Ser)ₙ, wherein n is 1, 2, 3, 4, 5, 6, 7, 8, 9, 10, 11, 12, 13, 14, 15, 16, 17, 18, 19, or 20. In one embodiment, n is 20, 21, 22, 23, 24, 25, 26, 26, 28, 29, 30, 31, 32, 33, 34, 35, 36, 37, 38, 39, or 40. In another embodiment, n is 50, 60, 70, 80, 90, 100, 110, 120, 130, 140, 150, 160, 170, 180, 190, or 200. See, e.g., Schlapschy M et al., Protein Eng. Design Selection, 20:273-284 (2007).

### 6) Transferrin or Fragment Thereof

In certain embodiments, the heterologous moiety linked to the VWF fragment or the FVIII protein is transferrin or a fragment thereof. Any transferrin may be used to make the chimeric proteins of the invention. As an example, wild-type human Tf (Tf) is a 679 amino acid protein, of approximately 75 KDa (not accounting for glycosylation), with two main domains, N (about 330 amino acids) and C (about 340 amino acids), which appear to originate from a gene duplication. See GenBank accession numbers NM001063, XM002793, M12530, XM039845, XM 039847 and S95936 (www.ncbi.nlm.nih.gov/), all of which are herein incorporated by reference in their entirety. Transferrin comprises two domains, N domain and C domain. N domain comprises two subdomains, N1 domain and N2 domain, and C domain comprises two subdomains, C1 domain and C2 domain.

In one embodiment, the transferrin portion of the chimeric protein includes a transferrin splice variant. In one example, a transferrin splice variant can be a splice variant of human transferrin, e.g., Genbank Accession AAA61140. In another embodiment, the transferrin portion of the chimeric protein includes one or more domains of the transferrin sequence, e.g., N domain, C domain, N1 domain, N2 domain, C1 domain, C2 domain or any combinations thereof.

### 7) Polymer, e.g., Polyethylene Glycol (PEG)

In other embodiments, the heterologous moiety attached to the VWF fragment or the protein having clotting activity, e.g. FVIII activity, is a soluble polymer known in the art, including, but not limited to, polyethylene glycol, ethylene glycol/propylene glycol copolymers, carboxymethylcellulose, dextran, or polyvinyl alcohol. The heterologous moiety such as soluble polymer can be attached to any positions within the VWF fragment or the FVIII protein or the N- or C-terminus. In still other embodiments, a chimeric protein of the invention comprises a FVIII protein and PEG, wherein PEG shields or protects the VWF binding site on the FVIII protein, thereby inhibiting or preventing interaction of the FVIII protein with endogenous VWF.

In certain embodiments, the chimeric protein comprises the VWF fragment described herein and PEG, wherein the VWF fragment is linked to PEG. In another embodiment, the chimeric protein comprises the VWF fragment and a FVIII protein, which are bound to each other, wherein the VWF fragment is linked to PEG, the FVIII protein is linked to PEG, or both the VWF fragment and the FVIII protein are linked to PEG. In other embodiments, the chimeric protein comprising the VWF fragment linked to PEG is further linked to a heterologous moiety selected from the group consisting of an immunoglobulin constant region or a portion thereof (e.g., an Fc region), a PAS sequence, HES, and albumin, fragment, or variant thereof. In still other embodiments, the chimeric protein comprises the VWF fragment and a FVIII protein, which are bound to each other, wherein the FVIII protein is further linked to a heterologous moiety selected from the group consisting of an immunoglobulin constant region or a portion thereof (e.g., an Fc region), a PAS sequence, HES, and albumin, fragment, or variant thereof. In yet other embodiments, the chimeric protein comprises the VWF fragment linked to PEG and a FVIII protein linked to PEG, which are bound to each other, wherein the VWF fragment activity is further linked to a first heterologous moiety selected from the group consisting of an immunoglobulin constant region or a portion thereof (e.g., an Fc region), a PAS sequence, HES, and albumin, fragment, or variant thereof and wherein the FVIII protein activity is further linked to a second heterologous moiety selected from the group consisting of an immunoglobulin constant region or a portion thereof (e.g., an Fc region), a PAS sequence, HES, and albumin, fragment, or variant thereof.

Also provided by the invention are chemically modified derivatives of the chimeric protein of the invention which may provide additional advantages such as increased solubility, stability and circulating time of the polypeptide, or decreased immunogenicity (see U.S. Pat. No. 4,179,337). The chemical moieties for modification can be selected from the group consisting of water soluble polymers including, but not limited to, polyethylene glycol, ethylene glycol/propylene glycol copolymers, carboxymethylcellulose, dextran, and polyvinyl alcohol. The chimeric protein may be modified at random positions within the molecule or at the N- or C-terminus, or at predetermined positions within the molecule and may include one, two, three or more attached chemical moieties.

The polymer can be of any molecular weight, and can be branched or unbranched. For polyethylene glycol, in one embodiment, the molecular weight is between about 1 kDa and about 100 kDa for ease in handling and manufacturing. Other sizes may be used, depending on the desired profile (e.g., the duration of sustained release desired, the effects, if any on biological activity, the ease in handling, the degree or lack of antigenicity and other known effects of the polyethylene glycol to a protein or analog). For example, the polyethylene glycol may have an average molecular weight of about 200, 500, 1000, 1500, 2000, 2500, 3000, 3500, 4000, 4500, 5000, 5500, 6000, 6500, 7000, 7500, 8000, 8500, 9000, 9500, 10,000, 10,500, 11,000, 11,500, 12,000, 12,500, 13,000, 13,500, 14,000, 14,500, 15,000, 15,500, 16,000, 16,500, 17,000, 17,500, 18,000, 18,500, 19,000, 19,500, 20,000, 25,000, 30,000, 35,000, 40,000, 45,000, 50,000, 55,000, 60,000, 65,000, 70,000, 75,000, 80,000, 85,000, 90,000, 95,000, or 100,000 kDa.

In some embodiments, the polyethylene glycol may have a branched structure. Branched polyethylene glycols are described, for example, in U.S. Pat. No. 5,643,575; Morpurgo et al., Appl. Biochem. Biotechnol. 56:59-72 (1996); Vorobjev et al., Nucleosides Nucleotides 18:2745-2750 (1999); and Caliceti et al., Bioconjug. Chem. 10:638-646 (1999), each of which is incorporated herein by reference in its entirety.

The number of polyethylene glycol moieties attached to each chimeric protein, the VWF fragment, or the FVIII protein of the invention (i.e., the degree of substitution) may also vary. For example, the pegylated proteins of the invention may be linked, on average, to 1, 2, 3, 4, 5, 6, 7, 8, 9, 10, 12, 15, 17, 20, or more polyethylene glycol molecules. Similarly, the average degree of substitution within ranges such as 1-3, 2-4, 3-5, 4-6, 5-7, 6-8, 7-9, 8-10, 9-11, 10-12, 11-13, 12-14, 13-15, 14-16, 15-17, 16-18, 17-19, or 18-20 polyethylene glycol moieties per protein molecule. Methods for determining the degree of substitution are discussed, for example, in Delgado et al., Crit. Rev. Thera. Drug Carrier Sys. 9:249-304 (1992).

In some embodiments, the FVIII protein may be PEGylated. PEGylated Factor VIII can refer to a conjugate formed between Factor VIII and at least one polyethylene glycol (PEG) molecule.

In other embodiments, a FVIII protein used in the invention is conjugated to one or more polymers. The polymer can be water-soluble and covalently or non-covalently attached to Factor VIII or other moieties conjugated to Factor VIII. Non-limiting examples of the polymer can be poly(alkylene oxide), poly(vinyl pyrrolidone), poly(vinyl alcohol), polyoxazoline, or poly(acryloylmorpholine). Additional types of polymer-conjugated FVIII are disclosed in U.S. Pat. No. 7,199,223.

### 8) Hydroxyethyl Starch (HES)

In certain embodiments, the heterologous moiety linked to the VWF fragment or the FVIII protein is a polymer, e.g., hydroxyethyl starch (HES) or a derivative thereof. In one embodiment, a chimeric protein comprises a VWF fragment described herein and HES, wherein the VWF fragment is linked to HES. In other embodiments, a chimeric protein of the invention comprises a FVIII protein fused to hydroxyethyl starch (HES), wherein the hydroxyethyl starch or a derivative thereof shields or protects the VWF binding site on the FVIII protein from endogenous VWF, thereby inhibiting or preventing interaction of the FVIII protein with endogenous VWF.

Hydroxyethyl starch (HES) is a derivative of naturally occurring amylopectin and is degraded by alpha-amylase in the body. HES is a substituted derivative of the carbohydrate polymer amylopectin, which is present in corn starch at a concentration of up to 95% by weight. HES exhibits advantageous biological properties and is used as a blood volume replacement agent and in hemodilution therapy in the clinics (Sommermeyer et al., Krankenhauspharmazie, 8 (8), 271-278 (1987); and Weidler et al., Arzneim.-Forschung/Drug Res., 41, 494-498 (1991)).

Amylopectin contains glucose moieties, wherein in the main chain alpha-1,4-glycosidic bonds are present and at the branching sites alpha-1,6-glycosidic bonds are found. The physical-chemical properties of this molecule are mainly determined by the type of glycosidic bonds. Due to the nicked alpha-1,4-glycosidic bond, helical structures with about six glucose-monomers per turn are produced. The physico-chemical as well as the biochemical properties of the polymer can be modified via substitution. The introduction of a hydroxyethyl group can be achieved via alkaline hydroxyethylation. By adapting the reaction conditions it is possible to exploit the different reactivity of the respective hydroxy group in the unsubstituted glucose monomer with respect to a hydroxyethylation. Owing to this fact, the skilled person is able to influence the substitution pattern to a limited extent.

HES is mainly characterized by the molecular weight distribution and the degree of substitution. The degree of substitution, denoted as DS, relates to the molar substitution, is known to the skilled people. See Sommermeyer et al., Krankenhauspharmazie, 8 (8), 271-278 (1987), as cited above, in particular p. 273.

In one embodiment, hydroxyethyl starch has a mean molecular weight (weight mean) of from 1 to 300 kD, from 2 to 200 kD, from 3 to 100 kD, or from 4 to 70 kD. hydroxyethyl starch can further exhibit a molar degree of substitution of from 0.1 to 3, preferably 0.1 to 2, more preferred, 0.1 to 0.9, preferably 0.1 to 0.8, and a ratio between C2:C6 substitution in the range of from 2 to 20 with respect to the hydroxyethyl groups. A non-limiting example of HES having a mean molecular weight of about 130 kD is a HES with a degree of substitution of 0.2 to 0.8 such as 0.2, 0.3, 0.4, 0.5, 0.6, 0.7, or 0.8, preferably of 0.4 to 0.7 such as 0.4, 0.5, 0.6, or 0.7. In a specific embodiment, HES with a mean molecular weight of about 130 kD is VOLUVEN® from Fresenius. VOLUVEN® is an artificial colloid, employed, e.g., for volume replacement used in the therapeutic indication for therapy and prophylaxis of hypovolaemia. The characteristics of VOLUVEN® are a mean molecular weight of 130,000+/−20,000 D, a molar substitution of 0.4 and a C2:C6 ratio of about 9:1. In other embodiments, ranges of the mean molecular weight of hydroxyethyl starch are, e.g., 4 to 70 kD or 10 to 70 kD or 12 to 70 kD or 18 to 70 kD or 50 to 70 kD or 4 to 50 kD or 10 to 50 kD or 12 to 50 kD or 18 to 50 kD or 4 to 18 kD or 10 to 18 kD or 12 to 18 kD or 4 to 12 kD or 10 to 12 kD or 4 to 10 kD. In still other embodiments, the mean molecular weight of hydroxyethyl starch employed is in the range of from more than 4 kD and below 70 kD, such as about 10 kD, or in the range of from 9 to 10 kD or from 10 to 11 kD or from 9 to 11 kD, or about 12 kD, or in the range of from 11 to 12 kD) or from 12 to 13 kD or from 11 to 13 kD, or about 18 kD, or in the range of from 17 to 18 kD or from 18 to 19 kD or from 17 to 19 kD, or about 30 kD, or in the range of from 29 to 30, or from 30 to 31 kD, or about 50 kD, or in the range of from 49 to 50 kD or from 50 to 51 kD or from 49 to 51 kD.

In certain embodiments, the heterologous moiety can be mixtures of hydroxyethyl starches having different mean molecular weights and/or different degrees of substitution and/or different ratios of C2:C6 substitution. Therefore, mixtures of hydroxyethyl starches may be employed having different mean molecular weights and different degrees of substitution and different ratios of C2:C6 substitution, or having different mean molecular weights and different degrees of substitution and the same or about the same ratio of C2:C6 substitution, or having different mean molecular weights and the same or about the same degree of substitution and different ratios of C2:C6 substitution, or having the same or about the same mean molecular weight and different degrees of substitution and different ratios of C2:C6 substitution, or having different mean molecular weights and the same or about the same degree of substitution and the same or about the same ratio of C2:C6 substitution, or having the same or about the same mean molecular weights and different degrees of substitution and the same or about the same ratio of C2:C6 substitution, or having the same or about the same mean molecular weight and the same or about the same degree of substitution and different ratios of C2:C6 substitution, or having about the same mean molecular weight and about the same degree of substitution and about the same ratio of C2:C6 substitution.

### 9) Polysialic Acids (PSA)

In certain embodiments, the non-polypeptide heterologous moiety linked to the VWF fragment or the FVIII protein is a polymer, e.g., polysialic acids (PSAs) or a derivative thereof. Polysialic acids (PSAs) are naturally occurring unbranched polymers of sialic acid produced by certain bacterial strains and in mammals in certain cells Roth J., et al. (1993) in Polysialic Acid: From Microbes to Man, eds Roth J., Rutishauser U., Troy F. A. (Birkhäuser Verlag, Basel, Switzerland), pp 335-348 . . . . They can be produced in various degrees of polymerization from n=about 80 or more sialic acid residues down to n=2 by limited acid hydrolysis or by digestion with neuraminidases, or by fractionation of the natural, bacterially derived forms of the polymer. The composition of different polysialic acids also varies such that there are homopolymeric forms i.e. the alpha-2,8-linked polysialic acid comprising the capsular polysaccharide of E. coli strain K1 and the group-B meningococci, which is also found on the embryonic form of the neuronal cell adhesion molecule (N-CAM). Heteropolymeric forms also exist-such as the alternating alpha-2,8 alpha-2,9 polysialic acid of E. coli strain K92 and group C polysaccharides of N. meningitidis. Sialic acid may also be found in alternating copolymers with monomers other than sialic acid such as group W135 or group Y of N. meningitidis. Polysialic acids have important biological functions including the evasion of the immune and complement systems by pathogenic bacteria and the regulation of glial adhesiveness of immature neurons during foetal development (wherein the polymer has an anti-adhesive function) Cho and Troy, P.N.A.S., USA, 91 (1994) 11427-11431, although there are no known receptors for polysialic acids in mammals. The alpha-2,8-linked polysialic acid of E. coli strain K1 is also known as ‘colominic acid’ and is used (in various lengths) to exemplify the present invention. Various methods of attaching or conjugating polysialic acids to a polypeptide have been described (for example, see U.S. Pat. No. 5,846,951; WO-A-0187922, and US 2007/0191597 A1, which are incorporated herein by reference in their entireties.

### C) FVIII Protein

“A FVIII protein” as used herein means a functional FVIII polypeptide in its normal role in coagulation, unless otherwise specified. The term a FVIII protein includes a functional fragment, variant, analog, or derivative thereof that retains the function of full-length wild-type Factor VIII in the coagulation pathway. “A FVIII protein” is used interchangeably with FVIII polypeptide (or protein) or FVIII. Examples of the FVIII functions include, but not limited to, an ability to activate coagulation, an ability to act as a cofactor for factor IX, or an ability to form a tenase complex with factor IX in the presence of Ca2+ and phospholipids, which then converts Factor X to the activated form Xa. The FVIII protein can be the human, porcine, canine, rat, or murine FVIII protein. In addition, comparisons between FVIII from humans and other species have identified conserved residues that are likely to be required for function (Cameron et al., Thromb. Haemost. 79:317-22 (1998); U.S. Pat. No. 6,251,632).

A number of tests are available to assess the function of the coagulation system: activated partial thromboplastin time (aPTT) test, chromogenic assay, ROTEM assay, prothrombin time (PT) test (also used to determine INR), fibrinogen testing (often by the Clauss method), platelet count, platelet function testing (often by PFA-100), TCT, bleeding time, mixing test (whether an abnormality corrects if the patient's plasma is mixed with normal plasma), coagulation factor assays, antiphosholipid antibodies, D-dimer, genetic tests (e.g. factor V Leiden, prothrombin mutation G20210A), dilute Russell's viper venom time (dRVVT), miscellaneous platelet function tests, thromboelastography (TEG or Sonoclot), thromboelastometry (TEM®, e.g, ROTEM®), or euglobulin lysis time (ELT).

The aPTT test is a performance indicator measuring the efficacy of both the “intrinsic” (also referred to the contact activation pathway) and the common coagulation pathways. This test is commonly used to measure clotting activity of commercially available recombinant clotting factors, e.g., FVIII or FIX. It is used in conjunction with prothrombin time (PT), which measures the extrinsic pathway.

ROTEM analysis provides information on the whole kinetics of haemostasis: clotting time, clot formation, clot stability and lysis. The different parameters in thromboelastometry are dependent on the activity of the plasmatic coagulation system, platelet function, fibrinolysis, or many factors which influence these interactions. This assay can provide a complete view of secondary haemostasis.

The FVIII polypeptide and polynucleotide sequences are known, as are many functional fragments, mutants and modified versions. Examples of human FVIII sequences (full-length) are shown as subsequences in SEQ ID NO: 16 or 18.

TABLE 2
Full-length FVIII (FVIII signal peptide underlined; FVIII heavy chain is
double underlined; B domain is italicized; and FVIII light chain is in
plain text)

Signal Peptide: (SEQ ID NO: 15)
MQIELSTCFFLCLLRFCFS
Mature Factor VIII (SEQ ID NO: 16)*
ATRRYYLGAVELSWDYMQSDLGELPVDARFPPRVPKSFPFNTSVVYKKTLFVEFTDHLFNIAKPRPPWMGLL
GPTIQAEVYDTVVITLKNMASHPVSLHAVGVSYWKASEGAEYDDQTSQREKEDDKVFPGGSHTYVWQVLKEN
GPMASDPLCLTYSYLSHVDLVKDLNSGLIGALLVCREGSLAKEKTQTLHKFILLFAVFDEGKSWHSETKNSL
MQDRDAASARAWPKMHTVNGYVNRSLPGLIGCHRKSVYWHVIGMGTTPEVHSIFLEGHTFLVRNHRQASLEI
SPITFLTAQTLLMDLGQFLLFCHISSHQHDGMEAYVKDSCPEEDPQLRMKNNEEAEDYDDDLTDSEMDVVRF
DDDNSPSFIQIRSVAKKHPKTWVHYIAAEEEDWDYAPLVLAPDDRSYKSQYLNNGPQRIGRKYKKVRFMAYT
DETFKTREAIQHESGILGPLLYGEVGDTLLIIFKNQASRPYNIYPHGITDVRPLYSRRLPKGVKHLKDFPIL
PGEIFKYKWTVTVEDGPTKSDPRCLTRYYSSFVNMERDLASGLIGPLLICYKESVDQRGNQIMSDKRNVILF
SVFDENRSWYLTENIQRFLPNPAGVQLEDPEFQASNIMHSINGYVFDSLQLSVCLHEVAYWYILSIGAQTDF
LSVFFSGYTFKHKMVYEDTLTLFPFSGETVFMSMENPGLWILGCHNSDFRNRGMTALLKVSSCDKNTGDYYELSVFFSGYTFKHKMVYEDTLTLFPFSGETVFMSMENPGLWILGCHNSDFRNRGMTALLKVSSCDKNTGDYYE
DSYEDISAYLLSKNNAIEPRSFSQNSRHPSTRQKQFNATTIPENDIEKTDPWFAHRTPMPKIQNVSSSDLLM
LLRQSPTPHGLSLSDLQEAKYETFSDDPSPGAIDSNNSLSEMTHFRPQLHHSGDMVFTPESGLQLRLNEKLG
TTAATELKKLDFKVSSTSNNLISTIPSDNLAAGTDNTSSLGPPSMPVHYDSQLDTTLFGKKSSPLTESGGPL
SLSEENNDSKLLESGLMNSQESSWGKNVSSTESGRLFKGKRAHGPALLTKDNALFKVSISLLKTNKTSNNSA
TNRKTHIDGPSLLIENSPSVWQNILESDTEFKKVTPLIHDRMLMDKNATALRLNHMSNKTTSSKNMEMVQQK
KEGPIPPDAQNPDMSFFKMLFLPESARWIQRTHGKNSLNSGQGPSPKQLVSLGPEKSVEGQNFLSEKNKVVV
GKGEFTKDVGLKEMVFPSSRNLFLTNLDNLHENNTHNQEKKIQEEIEKKETLIQENVVLPQIHTVTGTKNFM
KNLFLLSTRQNVEGSYDGAYAPVLQDFRSLNDSTNRTKKHTAHFSKKGEEENLEGLGNQTKQIVEKYACTTR
ISPNTSQQNFVTQRSKRALKQFRLPLEETELEKRIIVDDTSTQWSKNMKHLTPSTLTQIDYNEKEKGAITQS
PLSDCLTRSHSIPQANRSPLPIAKVSSFPSIRPIYLTRVLFQDNSSHLPAASYRKKDSGVQESSHFLQGAKK
NNLSLAILTLEMTGDQREVGSLGTSATNSVTYKKVENTVLPKPDLPKTSGKVELLPKVHIYQKDLFPTETSN
GSPGHLDLVEGSLLQGTEGAIKWNEANRPGKVPFLRVATESSAKTPSKLLDPLAWDNHYGTQIPKEEWKSQE
KSPEKTAFKKKDTILSLNACESNHAIAAINEGQNKPEIEVTWAKQGRTERLCSQNPPVLKRHQREITRTTLQ
SDQEEIDYDDTISVEMKKEDFDIYDEDENQSPRSFQKKTRHYFIAAVERLWDYGMSSSPHVLRNRAQSGSVP
QFKKVVFQEFTDGSFTQPLYRGELNEHLGLLGPYIRAEVEDNIMVTFRNQASRPYSFYSSLISYEEDQRQGA
EPRKNFVKPNETKTYFWKVQHHMAPTKDEFDCKAWAYFSDVDLEKDVHSGLIGPLLVCHTNTLNPAHGRQVT
VQEFALFFTIFDETKSWYFTENMERNCRAPCNIQMEDPTFKENYRFHAINGYIMDTLPGLVMAQDQRIRWYL
LSMGSNENIHSIHFSGHVFTVRKKEEYKMALYNLYPGVFETVEMLPSKAGIWRVECLIGEHLHAGMSTLFLV
YSNKCQTPLGMASGHIRDFQITASGQYGQWAPKLARLHYSGSINAWSTKEPFSWIKVDLLAPMIIHGIKTQG
ARQKFSSLYISQFIIMYSLDGKKWQTYRGNSTGTLMVFFGNVDSSGIKHNIFNPPIIARYIRLHPTHYSIRS
TLRMELMGCDLNSCSMPLGMESKAISDAQITASSYFTNMFATWSPSKARLHLQGRSNAWRPQVNNPKEWLQV
DFQKTMKVTGVTTQGVKSLLTSMYVKEFLISSSQDGHQWTLFFQNGKVKVFQGNQDSFTPVVNSLDPPLLTR
YLRIHPQSWVHQIALRMEVLGCEAQDLY

TABLE 3
Nucleotide Sequence Encoding Full-Length FVIII (SEQ ID NO: 17)*

| | |
|---|---|
| 661 | ATG CAAATAGAGC TCTCCACCTG |
| 721 | CTTCTTTCTG TGCCTTTTGC GATTCTGCTT TAGTGCCACC AGAAGATACT ACCTGGGTGC |
| 781 | AGTGGAACTG TCATGGGACT ATATGCAAAG TGATCTCGGT GAGCTGCCTG TGGACGCAAG |
| 841 | ATTTCCTCCT AGAGTGCCAA AATCTTTTCC ATTCAACACC TCAGTCGTGT ACAAAAAGAC |
| 901 | TCTGTTTGTA GAATTCACGG ATCACCTTTT CAACATCGCT AAGCCAAGGC CACCCTGGAT |
| 961 | GGGTCTGCTA GGTCCTACCA TCCAGGCTGA GGTTTATGAT ACAGTGGTCA TTACACTTAA |
| 1021 | GAACATGGCT TCCCATCCTG TCAGTCTTCA TGCTGTTGGT GTATCCTACT GGAAAGCTTC |
| 1081 | TGAGGGAGCT GAATATGATG ATCAGACCAG TCAAAGGGAG AAAGAAGATG ATAAAGTCTT |
| 1141 | CCCTGGTGGA AGCCATACAT ATGTCTGGCA GGTCCTGAAA GAGAATGGTC CAATGGCCTC |
| 1201 | TGACCCACTG TGCCTTACCT ACTCATATCT TTCTCATGTG GACCTGGTAA AAGACTTGAA |
| 1261 | TTCAGGCCTC ATTGGAGCCC TACTAGTATG TAGAGAAGGG AGTCTGGCCA AGGAAAAGAC |
| 1321 | ACAGACCTTG CACAAATTTA TACTACTTTT TGCTGTATTT GATGAAGGGA AAAGTTGGCA |
| 1381 | CTCAGAAACA AAGAACTCCT TGATGCAGGA TAGGGATGCT GCATCTGCTC GGGCCTGGCC |
| 1441 | TAAAATGCAC ACAGTCAATG GTTATGTAAA CAGGTCTCTG CCAGGTCTGA TTGGATGCCA |
| 1501 | CAGGAAATCA GTCTATTGGC ATGTGATTGG AATGGGCACC ACTCCTGAAG TGCACTCAAT |
| 1561 | ATTCCTCGAA GGTCACACAT TTCTTGTGAG GAACCATCGC CAGGCGTCCT TGGAAATCTC |
| 1621 | GCCAATAACT TTCCTTACTG CTCAAACACT CTTGATGGAC CTTGGACAGT TTCTACTGTT |
| 1681 | TTGTCATATC TCTTCCCACC AACATGATGG CATGGAAGCT TATGTCAAAG TAGACAGCTG |
| 1741 | TCCAGAGGAA CCCCAACTAC GAATGAAAAA TAATGAAGAA GCGGAAGACT ATGATGATGA |
| 1801 | TCTTACTGAT TCTGAAATGG ATGTGGTCAG GTTTGATGAT GACAACTCTC CTTCCTTTAT |
| 1861 | CCAAATTCGC TCAGTTGCCA AGAAGCATCC TAAAACTTGG GTACATTACA TTGCTGCTGA |
| 1921 | AGAGGAGGAC TGGGACTATG CTCCCTTAGT CCTCGCCCCC GATGACAGAA GTTATAAAAG |
| 1981 | TCAATATTTG AACAATGGCC CTCAGCGGAT TGGTAGGAAG TACAAAAAAG TCCGATTTAT |
| 2041 | GGCATACACA GATGAAACCT TTAAGACTCG TGAAGCTATT CAGCATGAAT CAGGAATCTT |
| 2101 | GGGACCTTTA CTTTATGGGG AAGTTGGAGA CACACTGTTG ATTATATTTA AGAATCAAGC |
| 2161 | AAGCAGACCA TATAACATCT ACCCTCACGG AATCACTGAT GTCCGTCCTT TGTATTCAAG |
| 2221 | GAGATTACCA AAAGGTGTAA AACATTTGAA GGATTTTCCA ATTCTGCCAG GAGAAATATT |
| 2281 | CAAATATAAA TGGACAGTGA CTGTAGAAGA TGGGCCAACT AAATCAGATC CTCGGTGCCT |
| 2341 | GACCCGCTAT TACTCTAGTT TCGTTAATAT GGAGAGAGAT CTAGCTTCAG GACTCATTGG |
| 2401 | CCCTCTCCTC ATCTGCTACA AAGAATCTGT AGATCAAAGA GGAAACCAGA TAATGTCAGA |
| 2461 | CAAGAGGAAT GTCATCCTGT TTTCTGTATT TGATGAGAAC CGAAGCTGGT ACCTCACAGA |
| 2521 | GAATATACAA CGCTTTCTCC CCAATCCAGC TGGAGTGCAG CTTGAGGATC CAGAGTTCCA |
| 2581 | AGCCTCCAAC ATCATGCACA GCATCAATGG CTATGTTTTT GATAGTTTGC AGTTGTCAGT |
| 2641 | TTGTTTGCAT GAGGTGGCAT ACTGGTACAT TCTAAGCATT GGAGCACAGA CTGACTTCCT |
| 2701 | TTCTGTCTTC TTCTCTGGAT ATACCTTCAA ACACAAAATG GTCTATGAAG ACACACTCAC |
| 2761 | CCTATTCCCA TTCTCAGGAG AAACTGTCTT CATGTCGATG GAAAACCCAG GTCTATGGAT |
| 2821 | TCTGGGGTGC CACAACTCAG ACTTTCGGAA CAGAGGCATG ACCGCCTTAC TGAAGGTTTC |
| 2881 | TAGTTGTGAC AAGAACACTG GTGATTATTA CGAGGACAGT TATGAAGATA TTTCAGCATA |
| 2941 | CTTGCTGAGT AAAAACAATG CCATTGAACC AAGAAGCTTC TCCCAGAATT CAAGACACCC |
| 3001 | TAGCACTAGG CAAAAGCAAT TTAATGCCAC CACAATTCCA GAAAATGACA TAGAGAAGAC |
| 3061 | TGACCCTTGG TTTGCACACA GAACACCTAT GCCTAAAATA CAAAATGTCT CCTCTAGTGA |
| 3121 | TTTGTTGATG CTCTTGCGAC AGAGTCCTAC TCCACATGGG CTATCCTTAT CTGATCTCCA |
| 3181 | AGAAGCCAAA TATGAGACTT TTTCTGATGA TCCATCACCT GGAGCAATAG ACAGTAATAA |
| 3241 | CAGCCTGTCT GAAATGACAC ACTTCAGGCC ACAGCTCCAT CACAGTGGGG ACATGGTATT |
| 3301 | TACCCCTGAG TCAGGCCTCC AATTAAGATT AAATGAGAAA CTGGGGACAA CTGCAGCAAC |
| 3361 | AGAGTTGAAG AAACTTGATT TCAAAGTTTC TAGTACATCA AATAATCTGA TTTCAACAAT |
| 3421 | TCCATCAGAC AATTTGGCAG CAGGTACTGA TAATACAAGT TCCTTAGGAC CCCCAAGTAT |
| 3481 | GCCAGTTCAT TATGATAGTC AATTAGATAC CACTCTATTT GGCAAAAAGT CATCTCCCCT |
| 3541 | TACTGAGTCT GGTGGACCTC TGAGCTTGAG TGAAGAAAAT AATGATTCAA AGTTGTTAGA |
| 3601 | ATCAGGTTTA ATGAATAGCC AAGAAAGTTC ATGGGGAAAA AATGTATCGT CAACAGAGAG |
| 3661 | TGGTAGGTTA TTTAAAGGGA AAAGAGCTCA TGGACCTGCT TTGTTGACTA AAGATAATGC |
| 3721 | CTTATTCAAA GTTAGCATCT CTTTGTTAAA GACAAACAAA ACTTCCAATA ATTCAGCAAC |
| 3781 | TAATAGAAAG ACTCACATTG ATGGCCCATC ATTATTAATT GAGAATAGTC CATCAGTCTG |
| 3841 | GCAAAATATA TTAGAAAGTG ACACTGAGTT TAAAAAAGTG ACACCTTTGA TTCATGACAG |
| 3901 | AATGCTTATG GACAAAAATG CTACAGCTTT GAGGCTAAAT CATATGTCAA ATAAAACTAC |
| 3961 | TTCATCAAAA AACATGGAAA TGGTCCAACA GAAAAAAGAG GGCCCCATTC CACCAGATGC |
| 4021 | ACAAAATCCA GATATGTCGT TCTTTAAGAT GCTATTCTTG CCAGAATCAG CAAGGTGGAT |
| 4081 | ACAAAGGACT CATGGAAAGA ACTCTCTGAA CTCTGGGCAA GGCCCCAGTC CAAAGCAATT |
| 4141 | AGTATCCTTA GGACCAGAAA AAGTTGGAGA AGGTCAGAAT TTCTTGTCTG AGAAAAACAA |
| 4201 | AGTGGTAGTA GGAAAGGGTG AATTTACAAA GGACGTAGGA CTCAAAGAGA TGGTTTTTCC |
| 4261 | AAGCAGCAGA AACCTATTTC TTACTAACTT GGATAATTTA CATGAAAATA ATACACACAA |
| 4321 | TCAAGAAAAA AAAATTCAGG AAGAAATAGA AAAGAAGGAA ACATTAATCC AAGAGAATGT |
| 4381 | AGTTTTGCCT CAGATACATA CAGTGACTGG CACTAAGAAT TTCATGAAGA ACCTTTTCTT |
| 4441 | ACTGAGCACT AGGCAAAATG TAGAAGGTTC ATATGACGGG GCATATGCTC CAGTACTTCA |
| 4501 | AGATTTTAGG TCATTAAATG ATTCAACAAA TAGAACAAAG AAACACACAG CTCATTTCTC |
| 4561 | AAAAAAAGGG GAGGAAGAAA ACTTGGAAGG CTTGGGAAAT CAAACCAAGC AAATTGTAGA |
| 4621 | GAAATATGCA TGCACCACAA GGATATCTCC TAATACAAGC CAGCAGAATT TTGTCACGCA |
| 4681 | ACGTAGTAAG AGAGCTTTGA AACAATTCAG ACTCCCACTA GAAGAAACAG AACTTGAAAA |
| 4741 | AAGGATAATT GTGGATGACA CCTCAACCCA GTGGTCCAAA AACATGAAAC ATTTGACCCC |
| 4801 | GAGCACCCTC ACACAGATAG ACTACAATGA GAAGGAGAAA GGGGCCATTA CTCAGTCTCC |
| 4861 | CTTATCAGAT TGCCTTACGA GGAGTCATAG CATCCCTCAA GCAAATAGAT CTCCATTACC |
| 4921 | CATTGCAAAG GTATCATCAT TTCCATCTAT TAGACCTATA TATCTGACCA GGGTCCTATT |
| 4981 | CCAAGACAAC TCTTCTCATC TTCCAGCAGC ATCTTATAGA AAGAAAGATT CTGGGGTCCA |
| 5041 | AGAAAGCAGT CATTTCTTAC AAGGAGCCAA AAAAAATAAC CTTTCTTTAG CCATTCTAAC |
| 5101 | CTTGGAGATG ACTGGTGATC AAAGAGAGGT TGGCTCCCTG GGGACAAGTG CCACAAATTC |
| 5161 | AGTCACATAC AAGAAAGTTG AGAACACTGT TCTCCCGAAA CCAGACTTGC CCAAAACATC |
| 5221 | TGGCAAAGTT GAATTGCTTC CAAAAGTTCA CATTTATCAG AAGGACCTAT TCCCTACGGA |
| 5281 | AACTAGCAAT GGGTCTCCTG GCCATCTGGA TTTCAGGGAA GGGAGCCTTC TTCAGGGAAC |
| 5341 | AGAGGGAGCG ATTAAGTGGA ATGAAGCAAA CAGACCTGGA AAAGTTCCCT TTCTGAGAGT |
| 5401 | AGCAACAGAA AGCTCTGCAA AGACTCCCTC CAAGCTATTG GATCCTCTTG CTTGGGATAA |
| 5461 | CCACTATGGT ACTCAGATAC CAAAAGAAGA GTGGAAATCC CAAGAGAAGT CACCAGAAAA |
| 5521 | AACAGCTTTT AAGAAAAAGG ATACCATTTT GTCCCTGAAC GCTTGTGAAA GCAATCATGC |
| 5581 | AATAGCAGCA ATAAATGAGG GACAAAATAA GCCCGAAATA GAAGTCACCT GGGCAAAGCA |
| 5641 | AGGTAGGACT GAAAGGCTGT GCTCTCAAAA CCCACCAGTC TTGAAACGCC ATCAACGGGA |
| 5701 | AATAACTCGT ACTACTCTTC AGTCAGATCA AGAGGAAATT GACTATGATG ATACCATATC |
| 5761 | AGTTGAAATG AAGAAGGAAG ATTTTGACAT TTATGATGAG GATGAAAATC AGAGCCCCCG |
| 5821 | CAGCTTTCAA AAGAAAACAC GACACTATTT TATTGCTGCA GTGGAGAGGC TCTGGGATTA |
| 5881 | TGGGATGAGT AGCTCCCCAC ATGTTCTAAG AAACAGGGCT CAGAGTGGCA GTGTCCCTCA |
| 5941 | GTTCAAGAAA GTTGTTTTCC AGGAATTTAC TGATGGCTCC TTTACTCAGC CCTTATACCG |
| 6001 | TGGAGAACTA AATGAACATT TGGGACTCCT GGGGCCATAT ATAAGAGCAG AAGTTGAAGA |
| 6061 | TAATATCATG GTAACTTTCA GAAATCAGGC CTCTCGTCCC TATTCCTTCT ATTCTAGCCT |
| 6121 | TATTTCTTAT GAGGAAGATC AGAGGCAAGG AGCAGAACCT AGAAAAAACT TTCTCCAGCC |
| 6181 | TAATGAAACC AAAACTTACT TTTGGAAAGT GCAACATCAT ATGGCACCCA CTAAAGATGA |
| 6241 | GTTTGACTGC AAAGCCTGGG CTTATTTCTC TGATGTTGAC CTGGAAAAAG ATGTGCACTC |
| 6301 | AGGCCTGATT GGACCCCTTC TGGTCTGCCA CACTAACACA CTGAACCCTG CTCATGGGAG |
| 6361 | ACAAGTGACA GTACAGGAAT TTGCTCTGTT TTTCACCATC TTTGATGAGA CCAAAAGCTG |
| 6421 | GTACTTCACT GAAAATATGG AAAGAAACTG CAGGGCTCCC TGCAATATCC AGATGGAAGA |
| 6481 | TCCCACTTTT AAAGAGAATT ATCGCTTCCA TGCAATCAAT GGCTACATAA TGGATACACT |
| 6541 | ACCTGGCTTA GTAATGGCTC AGGATCAAAG GATTCGATGG TATCTGCTCA GCATGGGCAG |
| 6601 | CAATGAAAAC ATCCATTCTA TTCATTTCAG TGGACATGTG TTCACTGTAC GAAAAAAAGA |
| 6661 | GGAGTATAAA ATGGCACTGT ACAATCTCTA TCCAGGTGTT TTTGAGACAG TGGAAATGTT |
| 6721 | ACCATCCAAA GCTGGAATTT GGCGGGTGGA ATGCCTTATT GGCGAGCATC TACATGCTGG |
| 6781 | GATGAGCACA CTTTTTCTGG TGTACAGCAA TAAGTGTCAG ACTCCCCTGG GAATGGCTTC |
| 6841 | TGGACACATT AGAGATTTTC AGATTACAGC TTCAGGACAA TATGGACAGT GGGCCCCAAA |
| 6901 | GCTGGCCAGA CTTCATTATT CCGGATCAAT CAATGCCTGG AGCACCAAGG AGCCCTTTTC |
| 6961 | TTGGATCAAG GTGGATCTGT TGGCACCAAT GATTATTCAC GGCATCAAGA CCCAGGGTGC |
| 7021 | CCGTCAGAAG TTCTCCAGCC TCTACATCTC TCAGTTTATC ATCATGTATA GTCTTGATGG |
| 7081 | GAAGAAGTGG CAGACTTATC GAGGAAATTC CACTGGAACC TTAATGGTCT TCTTTGGCAA |
| 7141 | TGTGGATTCA TCTGGGATTA AACACAATAT TTTTAACCCT CCAATTATTG CTCGATACAT |
| 7201 | CCGTTTGCAC CCAACTCATT ATAGCATTCG CAGCACTCTT CGCATGGAGT TGATGGGCTG |
| 7261 | TGATTTAAAT AGTTGCAGCA TGCCATTGGG AATGGAGAGT AAAGCAATAT CAGATGCACA |
| 7321 | GATTACTGCT TCATCCTACT TTACCAATAT GTTTGCCACC TGGTCTCCTT CAAAAGCTCG |
| 7381 | ACTTCACCTC CAAGGGAGGA GTAATGCCTG GAGACCTCAG GTGAATAATC CAAAAGAGTG |
| 7441 | GCTGCAAGTG GACTTCCAGA AGACAATGAA AGTCACAGGA GTAACTACTC AGGGAGTAAA |
| 7501 | ATCTCTGCTT ACCAGCATGT ATGTGAAGGA GTTCCTCATC TCCAGCAGTC AAGATGGCCA |
| 7561 | TCAGTGGACT CTCTTTTTTC AGAATGGCAA AGTAAAGGTT TTTCAGGGAA ATCAAGACTC |
| 7621 | CTTCACACCT GTGGTGAACT CTCTAGACCC ACCGTTACTG ACTCGCTACC TTCGAATTCA |
| 7681 | CCCCCAGAGT TGGGTGCACC AGATTGCCCT GAGGATGGAG GTTCTGGGCT GCGAGGCACA |
| 7741 | GGACCTCTAC |

*The underlined nucleic acids encode a signal peptide.

FVIII polypeptides include full-length FVIII, full-length FVIII minus Met at the N-terminus, mature FVIII (minus the signal sequence), mature FVIII with an additional Met at the N-terminus, and/or FVIII with a full or partial deletion of the B domain. In certain embodiments, FVIII variants include B domain deletions, whether partial or full deletions.

The human FVIII gene was isolated and expressed in mammalian cells (Toole, J. J., et al., Nature 312:342-347 (1984); Gitschier, J., et al., Nature 312:326-330 (1984); Wood, W. I., et al., Nature 312:330-337 (1984); Vehar, G. A., et al., Nature 312:337-342 (1984); WO 87/04187; WO 88/08035; WO 88/03558; and U.S. Pat. No. 4,757,006). The FVIII amino acid sequence was deduced from cDNA as shown in U.S. Pat. No. 4,965,199. In addition, partially or fully B-domain deleted FVIII is shown in U.S. Pat. Nos. 4,994,371 and 4,868,112. In some embodiments, the human FVIII B-domain is replaced with the human Factor V B-domain as shown in U.S. Pat. No. 5,004,803. The cDNA sequence encoding human Factor VIII and amino acid sequence are shown in SEQ ID NOs: 17 and 16, respectively, of US Application Publ. No. 2005/0100990.

The porcine FVIII sequence is published in Toole, J. J., et al., Proc. Natl. Acad. Sci. USA 83:5939-5942 (1986). Further, the complete porcine cDNA sequence obtained from PCR amplification of FVIII sequences from a pig spleen cDNA library has been reported in Healey, J. F., et al., Blood 88:4209-4214 (1996). Hybrid human/porcine FVIII having substitutions of all domains, all subunits, and specific amino acid sequences were disclosed in U.S. Pat. No. 5,364,771 by Lollar and Runge, and in WO 93/20093. More recently, the nucleotide and corresponding amino acid sequences of the A1 and A2 domains of porcine FVIII and a chimeric FVIII with porcine A1 and/or A2 domains substituted for the corresponding human domains were reported in WO 94/11503. U.S. Pat. No. 5,859,204, Lollar, J. S., also discloses the porcine cDNA and deduced amino acid sequences. U.S. Pat. No. 6,458,563 discloses a B-domain-deleted porcine FVIII.

U.S. Pat. No. 5,859,204 to Lollar, J. S. reports functional mutants of FVIII having reduced antigenicity and reduced immunoreactivity. U.S. Pat. No. 6,376,463 to Lollar, J. S. also reports mutants of FVIII having reduced immunoreactivity. US Appl. Publ. No. 2005/0100990 to Saenko et al. reports functional mutations in the A2 domain of FVIII.

In one embodiment, the FVIII (or FVIII portion of a chimeric protein) may be at least 50%, 60%, 70%, 80%, 90%, 95%, 96%, 97%, 98%, 99%, or 100% identical to a FVIII amino acid sequence of amino acids 1 to 1438 of SEQ ID NO: 18 or amino acids 1 to 2332 of SEQ ID NO: 16 (without a signal sequence) or a FVIII amino acid sequence of amino acids -19 to 1438 of SEQ ID NO: 15 and SEQ ID NO: 18 or amino acids -19 to 2332 of SEQ ID NO: 15 and SEQ ID NO: 16 (with a signal sequence), wherein the FVIII has a clotting activity, e.g., activates Factor IX as a cofactor to convert Factor X to activated Factor X. The FVIII (or FVIII portion of a chimeric protein) may be identical to a FVIII amino acid sequence of amino acids 1 to 1438 of SEQ ID NO: 18 or amino acids 1 to 2332 of SEQ ID NO: 16 (without a signal sequence). The FVIII may further comprise a signal sequence.

The “B-domain” of FVIII, as used herein, is the same as the B-domain known in the art that is defined by internal amino acid sequence identity and sites of proteolytic cleavage, e.g., residues Ser741-Arg1648 of full-length human FVIII. The other human FVIII domains are defined by the following amino acid residues: A1, residues Ala1-Arg372; A2, residues Ser373-Arg740; A3, residues Ser1690-Asn2019; C1, residues Lys2020-Asn2172; C2, residues Ser2173-Tyr2332. The A3-C1-C2 sequence includes residues Ser1690-Tyr2332. The remaining sequence, residues Glu1649-Arg1689, is usually referred to as the a3 acidic region. The locations of the boundaries for all of the domains, including the B-domains, for porcine, mouse and canine FVIII are also known in the art. In one embodiment, the B domain of FVIII is deleted (“B-domain-deleted factor VIII” or “BDD FVIII”). An example of a BDD FVIII is REFACTOR (recombinant BDD FVIII), which has the same sequence as the Factor VIII portion of the sequence in Table 4. (BDD FVIII heavy chain is double underlined; B domain is italicized; and BDD FVIII light chain is in plain text).

TABLE 4
BDD FVIII (SEQ ID NO: 18)

ATRRYYLGAVELSWDYMQSDLGELPVDARFPPRVPKSFPFNTSVVYKKTLFVEFTDHLFNIAKPRPPWMGLL
GPTIQAEVYDTVVITLKNMASHPVSLHAVGVSYWKASEGAEYDDQTSQREKEDDKVFPGGSHTYVWQVLKEN
GPMASDPLCLTYSYLSHVDLVKDLNSGLIGALLVCREGSLAKEKTQTLHKFILLFAVFDEGKSWHSETKNSL
MQDRDAASARAWPKMHTVNGYVNRSLPGLIGCHRKSVYWHVIGMGTTPEVHSIFLEGHTFLVRNHRQASLEI
SPITFLTAQTLLMDLGQFLLFCHISSHQHDGMEAYVKVDSCPEEPQLRMKNNEEAEDYDDDLTDSEMDVVRF
DDDNSPSFIQIRSVAKKHPKTWVHYIAAEEEDWDYAPLVLAPDDRSYKSQYLNNGPQRIGRKYKKVRFMAYT
DETFKTREAIQHESGILGPLLYGEVGDTLLIIFKNQASRPYNIYPHGITDVRPLYSRRLPKGVKHLKDRPIL
PGEIFKYKWTVTVEDGPTKSDPRCLTRYYSSFVNMERDLASGLIGPLLICYKESVDQRGNQIMSDKRNVILF
SVFDENRSWYLTENIQRFLPNPAGVQLEDPEFQASNIMHSINGYVFDSLQLSVCLHEVAYWYILSIGAQTDF
LSVFFSGYTFKHKMVYEDTLTLFPFSGETVFMSMENPGLWILGCHNSDFRNRGMTALLKVSSCDKNTGDYYE
DSYEDISAYLLSKNNAIEPRSFSQNPPVLKRHQREITRTTLQSDQEEIDYDDTISVEMKKEDFDIYDEDENQ
SPRSFQKKTRHYFIAAVERLWDYGMSSSPHVLRNRAQSGSVPQFKKVVFQEFTDGSFTQPLYRGELNEHLGL
LGPYIRAEVEDNIMVTFRNQASRPYSFYSSLISYEEDQRQGAEPRKNFVKPNETKTYFWKVQHHMAPTKDEF
DCKAWAYFSDVDLEKDVHSGLIGPLLVCHTNTLNPAHGRQVTVQEFALFFTIFDETKSWYFTENMERNCRAP
CNIQMEDPTFKENYRFHAINGYIMDTLPGLVMAQDQRIRWYLLSMGSNENIHSIHFSGHVFTVRKKEEYKMA
LYNLYPGVFETVEMLPSKAGIWRVECLIGEHLHAGMSTLFLVYSNKCQTPLGMASGHIRDFQITASGQYGQW
APKLARLHYSGSINAWSTKEPFSWIKVDLLAPMIIHGIKTQGARQKFSSLYISQFIIMYSLDGKKWQTYRGN
STGTLMVFFGNVDSSGIKHNIFNPPIIARYIRLHPTHYSIRSTLRMELMGCDLNSCSMPLGMESKAISDAQI
TASSYFTNMFATWSPSKARLHLQGRSNAWRPQVNNPKEWLQVDFQKTMKVTGVTTQGVKSLLTSMYVKEFLI
SSSQDGHQWTLFFQNGKVKVFQGNQDSFTPVVNSLDPPLLTRYLRIHPQSWVHQIALRMEVLGCEAQDLY

TABLE 5
Nucleotide Sequence Encoding BDD FVIII (SEQ ID NO: 19)*

| | |
|---|---|
| 661 | A TGCAAATAGA GCTCTCCACC TGCTTCTTTC |
| 721 | TGTGCCTTTT GCGATTCTGC TTTAGTGCCA CCAGAAGATA CTACCTGGGT GCAGTGGAAC |
| 781 | TGTCATGGGA CTATATGCAA AGTGATCTCG GTGAGCTGCC TGTGGACGCA AGATTTCCTC |
| 841 | CTAGAGTGCC AAAATCTTTT CCATTCAACA CCTCAGTCGT GTACAAAAAG ACTCTGTTTG |
| 901 | TAGAATTCAC GGATCACCTT TTCAACATCG CTAAGCCAAG GCCACCCTGG ATGGGTCTGC |
| 961 | TAGGTCCTAC CATCCAGGCT GAGGTTTATG ATACAGTGGT CATTACACTT AAGAACATGG |
| 1021 | CTTCCCATCC TGTCAGTCTT CATGCTGTTG GTGTATCCTA CTGGAAAGCT TCTGAGGGAG |
| 1081 | CTGAATATGA TGATCAGACC AGTCAAAGGG AGAAAGAAGA TGATAAAGTC TTCCCTGGTG |
| 1141 | GAAGCCATAC ATATGTCTGG CAGGTCCTGA AAGAGAATGG TCCAATGGCC TCTGACCCAC |
| 1201 | TGTGCCTTAC CTACTCATAT CTTTCTCATG TGGACCTGGT AAAAGACTTG AATTCAGGCC |
| 1261 | TCATTGGAGC CCTACTAGTA TGTAGAGAAG GGAGTCTGGC CAAGGAAAAG ACACAGACCT |
| 1321 | TGCACAAATT TATACTACTT TTTGCTGTAT TTGATGAAGG GAAAAGTTGG CACTCAGAAA |
| 1381 | CAAAGAACTC CTTGATGCAG GATAGGGATG CTGCATCTGC TCGGGCCTGG CCTAAAATGC |
| 1441 | ACACAGTCAA TGGTTATGTA AACAGGTCTC TGCCAGGTCT GATTGGATGC CACAGGAAAT |
| 1501 | CAGTCTATTG GCATGTGATT GGAATGGGCA CCACTCCTGA AGTGCACTCA ATATTCCTCG |
| 1561 | AAGGTCACAC ATTTCTTGTG AGGAACCATC GCCAGGCGTC CTTGGAAATC TCGCCAATAA |
| 1621 | CTTTCCTTAC TGCTCAAACA CTCTTGATGG ACCTTGGACA GTTTCTACTG TTTTGTCATA |
| 1681 | TCTCTTCCCA CCAACATGAT GGCATGGAAG CTTATGTCAA AGTAGACAGC TGTCCAGAGG |
| 1741 | AACCCCAACT ACGAATGAAA AATAATGAAG AAGCGGAAGA CTATGATGAT GATCTTACTG |
| 1801 | ATTCTGAAAT GGATGTGGTC AGGTTTGATG ATGACAACTC TCCTTCCTTT ATCCAAATTC |
| 1861 | GCTCAGTTGC CAAGAAGCAT CCTAAAACTT GGGTACATTA CATTGCTGCT GAAGAGGAGG |
| 1921 | ACTGGGACTA TGCTCCCTTA GTCCTCGCCC CCGATGACAG AAGTTATAAA AGTCAATATT |
| 1981 | TGAACAATGG CCCTCAGCGG ATTGGTAGGA AGTACAAAAA AGTCCGATTT ATGGCATACA |
| 2041 | CAGATGAAAC CTTTAAGACT CGTGAAGCTA TTCAGCATGA ATCAGGAATC TTGGGACCTT |
| 2101 | TACTTTATGG GGAAGTTGGA GACACACTGT TGATTATATT TAAGAATCAA GCAAGCAGAC |
| 2161 | CATATAACAT CTACCCTCAC GGAATCACTG ATGTCCGTCC TTTGTATTCA AGGAGATTAC |
| 2221 | CAAAAGGTGT AAAACATTTG AAGGATTTTC CAATTCTGCC AGGAGAAATA TTCAAATATA |
| 2281 | AATGGACAGT GACTGTAGAA GATGGGCCAA CTAAATCAGA TCCTCGGTGC CTGACCCGCT |
| 2341 | ATTACTCTAG TTTCGTTAAT ATGGAGAGAG ATCTAGCTTC AGGACTCATT GGCCCTCTCC |
| 2401 | TCATCTGCTA CAAAGAATCT GTAGATCAAA GAGGAAACCA GATAATGTCA GACAAGAGGA |
| 2461 | ATGTCATCCT GTTTTCTGTA TTTGATGAGA ACCGAAGCTG GTACCTCACA GAGAATATAC |
| 2521 | AACGCTTTCT CCCCAATCCA GCTGGAGTGC AGCTTGAGGA TCCAGAGTTC CAAGCCTCCA |
| 2581 | ACATCATGCA CAGCATCAAT GGCTATGTTT TTGATAGTTT GCAGTTGTCA GTTTGTTTGC |
| 2641 | ATGAGGTGGC ATACTGGTAC ATTCTAAGCA TTGGAGCACA GACTGACTTC CTTTCTGTCT |
| 2701 | TCTTCTCTGG ATATACCTTC AAACACAAAA TGGTCTATGA AGACACACTC ACCCTATTCC |
| 2761 | CATTCTCAGG AGAAACTGTC TTCATGTCGA TGGAAAACCC AGGTCTATGG ATTCTGGGGT |
| 2821 | GCCACAACTC AGACTTTCGG AACAGAGGCA TGACCGCCTT ACTGAAGGTT TCTAGTTGTG |
| 2881 | ACAAGAACAC TGGTGATTAT TACGAGGACA GTTATGAAGA TATTTCAGCA TACTTGCTGA |
| 2941 | GTAAAAACAA TGCCATTGAA CCAAGAAGCT TCTCTCAAAA CCCACCAGTC TTGAAACGCC |
| 3001 | ATCAACGGGA AATAACTCGT ACTACTCTTC AGTCAGATCA AGAGGAAATT GACTATGATG |
| 3061 | ATACCATATC AGTTGAAATG AAGAAGGAAG ATTTTGACAT TTATGATGAG GATGAAAATC |
| 3121 | AGAGCCCCCG CAGCTTTCAA AAGAAAACAC GACACTATTT TATTGCTGCA GTGGAGAGGC |
| 3181 | TCTGGGATTA TGGGATGAGT AGCTCCCCAC ATGTTCTAAG AAACAGGGCT CAGAGTGGCA |
| 3241 | GTGTCCCTCA GTTCAAGAAA GTTGTTTTCC AGGAATTTAC TGATGGCTCC TTTACTCAGC |
| 3301 | CCTTATACCG TGGAGAACTA AATGAACATT TGGGACTCCT GGGGCCATAT ATAAGAGCAG |
| 3361 | AAGTTGAAGA TAATATCATG GTAACTTTCA GAAATCAGGC CTCTCGTCCC TATTCCTTCT |
| 3421 | ATTCTAGCCT TATTTCTTAT GAGGAAGATC AGAGGCAAGG AGCAGAACCT AGAAAAAACT |
| 3481 | TTGTCAAGCC TAATGAAACC AAAACTTACT TTTGGAAAGT GCAACATCAT ATGGCACCCA |
| 3541 | CTAAAGATGA GTTTGACTGC AAAGCCTGGG CTTATTTCTC TGATGTTGAC CTGGAAAAAG |
| 3601 | ATGTGCACTC AGGCCTGATT GGACCCCTTC TGGTCTGCCA CACTAACACA CTGAACCCTG |
| 3661 | CTCATGGGAG ACAAGTGACA GTACAGGAAT TTGCTCTGTT TTTCACCATC TTTGATGAGA |
| 3721 | CCAAAAGCTG GTACTTCACT GAAAATATGG AAAGAAACTG CAGGGCTCCC TGCAATATCC |
| 3781 | AGATGGAAGA TCCCACTTTT AAAGAGAATT ATCGCTTCCA TGCAATCAAT GGCTACATAA |
| 3841 | TGGATACACT ACCTGGCTTA GTAATGGCTC AGGATCAAAG GATTCGATGG TATCTGCTCA |
| 3901 | GCATGGGCAG CAATGAAAAC ATCCATTCTA TTCATTTCAG TGGACATGTG TTCACTGTAC |
| 3961 | GAAAAAAAGA GGAGTATAAA ATGGCACTGT ACAATCTCTA TCCAGGTGTT TTTGAGACAG |
| 4021 | TGGAAATGTT ACCATCCAAA GCTGGAATTT GGCGGGTGGA ATGCCTTATT GGCGAGCATC |
| 4081 | TACATGCTGG GATGAGCACA CTTTTTCTGG TGTACAGCAA TAAGTGTCAG ACTCCCCTGG |
| 4141 | GAATGGCTTC TGGACACATT AGAGATTTTC AGATTACAGC TTCAGGACAA TATGGACAGT |
| 4201 | GGGCCCCAAA GCTGGCCAGA CTTCATTATT CCGGATCAAT CAATGCCTGG AGCACCAAGG |
| 4261 | AGCCCTTTTC TTGGATCAAG GTGGATCTGT TGGCACCAAT GATTATTCAC GGCATCAAGA |
| 4321 | CCCAGGGTGC CCGTCAGAAG TTCTCCAGCC TCTACATCTC TCAGTTTATC ATCATGTATA |
| 4381 | GTCTTGATGG GAAGAAGTGG CAGACTTATC GAGGAAATTC CACTGGAACC TTAATGGTCT |
| 4441 | TCTTTGGCAA TGTGGATTCA TCTGGGATAA AACACAATAT TTTTAACCCT CCAATTATTG |
| 4501 | CTCGATACAT CCGTTTGCAC CCAACTCATT ATAGCATTCG CAGCACTCTT CGCATGGAGT |
| 4561 | TGATGGGCTG TGATTTAAAT AGTTGCAGCA TGCCATTGGG AATGGAGAGT AAAGCAATAT |
| 4621 | CAGATGCACA GATTACTGCT TCATCCTACT TTACCAATAT GTTTGCCACC TGGTCTCCTT |
| 4681 | CAAAAGCTCG ACTTCACCTC CAAGGGAGGA GTAATGCCTG GAGACCTCAG GTGAATAATC |
| 4741 | CAAAAGAGTG GCTGCAAGTG GACTTCCAGA AGACAATGAA AGTCACAGGA GTAACTACTC |
| 4801 | AGGGAGTAAA ATCTCTGCTT ACCAGCATGT ATGTGAAGGA GTTCCTCATC TCCAGCAGTC |
| 4861 | AAGATGGCCA TCAGTGGACT CTCTTTTTTC AGAATGGCAA AGTAAAGGTT TTTCAGGGAA |
| 4921 | ATCAAGACTC CTTCACACCT GTGGTGAACT CTCTAGACCC ACCGTTACTG ACTCGCTACC |
| 4981 | TTCGAATTCA CCCCCAGAGT TGGGTGCACC AGATTGCCCT GAGGATGGAG GTTCTGGGCT |
| 5041 | GCGAGGCACA GGACCTCTAC |

*The underlined nucleic acids encode a signal peptide.

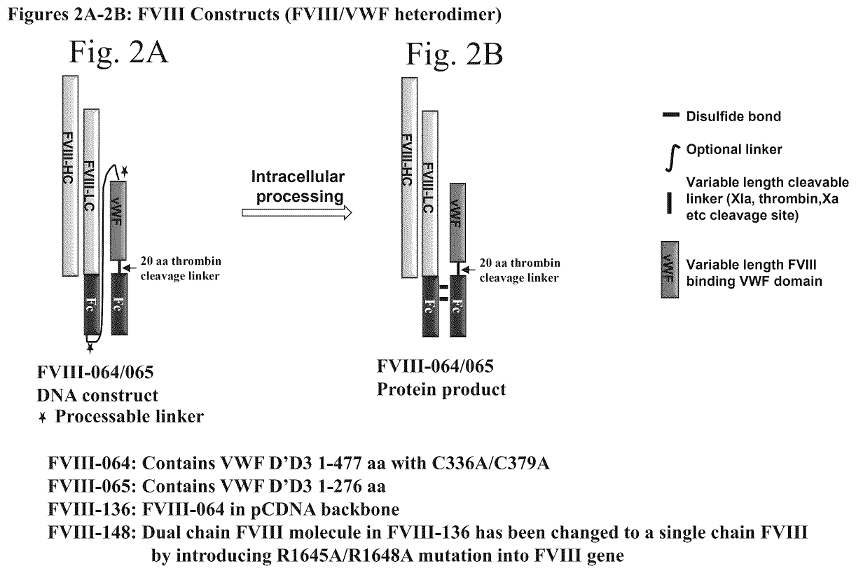

A “B-domain-deleted FVIII” may have the full or partial deletions disclosed in U.S. Pat. Nos. 6,316,226, 6,346,513, 7,041,635, 5,789,203, 6,060,447, 5,595,886, 6,228,620, 5,972,885, 6,048,720, 5,543,502, 5,610,278, 5,171,844, 5,112,950, 4,868,112, and 6,458,563. In some embodiments, a B-domain-deleted FVIII sequence of the present invention comprises any one of the deletions disclosed at col. 4, line 4 to col. 5, line 28 and Examples 1-5 of U.S. Pat. No. 6,316,226 (also in U.S. Pat. No. 6,346,513). In another embodiment, a B-domain deleted Factor VIII is the S743/Q1638 B-domain deleted Factor VIII (SQ BDD FVIII) (e.g., Factor VIII having a deletion from amino acid 744 to amino acid 1637, e.g., Factor VIII having amino acids 1-743 and amino acids 1638-2332 of SEQ ID NO: 16, i.e., SEQ ID NO: 18). In some embodiments, a B-domain-deleted FVIII of the present invention has a deletion disclosed at col. 2, lines 26-51 and examples 5-8 of U.S. Pat. No. 5,789,203 (also U.S. Pat. Nos. 6,060,447, 5,595,886, and 6,228,620). In some embodiments, a B-domain-deleted Factor VIII has a deletion described in col. 1, lines 25 to col. 2, line 40 of U.S. Pat. No. 5,972,885; col. 6, lines 1-22 and example 1 of U.S. Pat. No. 6,048,720; col. 2, lines 17-46 of U.S. Pat. No. 5,543,502; col. 4, line 22 to col. 5, line 36 of U.S. Pat. No. 5,171,844; col. 2, lines 55-68, FIG. 2, and example 1 of U.S. Pat. No. 5,112,950; col. 2, line 2 to col. 19, line 21 and table 2 of U.S. Pat. No. 4,868,112; col. 2, line 1 to col. 3, line 19, col. 3, line 40 to col. 4, line 67, col. 7, line 43 to col. 8, line 26, and col. 11, line 5 to col. 13, line 39 of U.S. Pat. No. 7,041,635; or col. 4, lines 25-53, of U.S. Pat. No. 6,458,563.

In some embodiments, a B-domain-deleted FVIII has a deletion of most of the B domain, but still contains amino-terminal sequences of the B domain that are essential for in vivo proteolytic processing of the primary translation product into two polypeptide chain, as disclosed in WO 91/09122. In some embodiments, a B-domain-deleted FVIII is constructed with a deletion of amino acids 747-1638, i.e., virtually a complete deletion of the B domain. Hoeben R. C., et al. J. Biol. Chem. 265 (13): 7318-7323 (1990). A B-domain-deleted Factor VIII may also contain a deletion of amino acids 771-1666 or amino acids 868-1562 of FVIII. Meulien P., et al. Protein Eng. 2 (4): 301-6 (1988). Additional B domain deletions that are part of the invention include: deletion of amino acids 982 through 1562 or 760 through 1639 (Toole et al., Proc. Natl. Acad. Sci. U.S.A. (1986) 83, 5939-5942)), 797 through 1562 (Eaton, et al. Biochemistry (1986) 25:8343-8347)), 741 through 1646 (Kaufman (PCT published application No. WO 87/04187)), 747-1560 (Sarver, et al., DNA (1987) 6:553-564)), 741 through 1648 (Pasek (PCT application No.88/00831)), or 816 through 1598 or 741 through 1648 (Lagner (Behring Inst. Mitt. (1988) No 82:16-25, EP 295597)). In other embodiments, BDD FVIII includes a FVIII polypeptide containing fragments of the B-domain that retain one or more N-linked glycosylation sites, e.g., residues 757, 784, 828, 900, 963, or optionally 943, which correspond to the amino acid sequence of the full-length FVIII sequence. Examples of the B-domain fragments include 226 amino acids or 163 amino acids of the B-domain as disclosed in Miao, H. Z., et al., Blood 103(a): 3412-3419 (2004), Kasuda, A, et al., J. Thromb. Haemost. 6:1352-1359 (2008), and Pipe, S. W., et al., J. Thromb. Haemost. 9:2235-2242 (2011) (i.e., the first 226 amino acids or 163 amino acids of the B domain are retained). In some embodiments, the FVIII with a partial B-domain is FVIII198 (SEQ ID NO: 105). FVIII198 is a partial B-domain containing single chain FVIIIFc molecule-226N6. 226 represents the N-terminus 226 amino acid of the FVIII B-domain, and N6 represents six N-glycosylation sites in the B-domain. In still other embodiments, BDD FVIII further comprises a point mutation at residue 309 (from Phe to Ser) to improve expression of the BDD FVIII protein. See Miao, H. Z., et al., Blood 103(a): 3412-3419 (2004). In still other embodiments, the BDD FVIII includes a FVIII polypeptide containing a portion of the B-domain, but not containing one or more furin cleavage sites (e.g., Arg1313 and Arg 1648). See Pipe, S. W., et al., J. Thromb. Haemost. 9:2235-2242 (2011). Each of the foregoing deletions may be made in any FVIII sequence.

A FVIII protein useful in the present invention can include FVIII having one or more additional heterologous sequences or chemical or physical modifications therein, which do not affect the FVIII coagulation activity. Such heterologous sequences or chemical or physical modifications can be fused to the C-terminus or N-terminus of the FVIII protein or inserted between one or more of the two amino acid residues in the FVIII protein. Such insertions in the FVIII protein do not affect the FVIII coagulation activity or FVIII function. In one embodiment, the insertions improve pharmacokinetic properties of the FVIII protein (e.g., half-life). In another embodiment, the insertions can be more than two, three, four, five, or six sites.

In one embodiment, FVIII is cleaved right after Arginine at amino acid 1648 (in full-length Factor VIII or SEQ ID NO: 16), amino acid 754 (in the S743/Q1638 B-domain deleted Factor VIII or SEQ ID NO: 16), or the corresponding Arginine residue (in other variants), thereby resulting in a heavy chain and a light chain. In another embodiment, FVIII comprises a heavy chain and a light chain, which are linked or associated by a metal ion-mediated non-covalent bond.

In other embodiments, FVIII is a single chain FVIII that has not been cleaved right after Arginine at amino acid 1648 (in full-length FVIII or SEQ ID NO: 16), amino acid 754 (in the S743/Q1638 B-domain-deleted FVIII or SEQ ID NO: 18), or the corresponding Arginine residue (in other variants). A single chain FVIII may comprise one or more amino acid substitutions. In one embodiment, the amino acid substitution is at a residue corresponding to residue 1648, residue 1645, or both of full-length mature Factor VIII polypeptide (SEQ ID NO: 16) or residue 754, residue 751, or both of SQ BDD Factor VIII (SEQ ID NO: 18). The amino acid substitution can be any amino acids other than Arginine, e.g., isoleucine, leucine, lysine, methionine, phenylalanine, threonine, tryptophan, valine, alanine, asparagine, aspartic acid, cysteine, glutamic acid, glutamine, glycine, proline, selenocysteine, serine, tyrosine, histidine, ornithine, pyrrolysine, or taurine.

FVIII can further be cleaved by thrombin and then activated as FVIIIa, serving as a cofactor for activated Factor IX (FIXa). And the activated FIX together with activated FVIII forms a Xase complex and converts Factor X to activated Factor X (FXa). For activation, FVIII is cleaved by thrombin after three Arginine residues, at amino acids 372, 740, and 1689 (corresponding to amino acids 372, 740, and 795 in the B-domain deleted FVIII sequence), the cleavage generating FVIIIa having the 50 kDa A1, 43 kDa A2, and 73 kDa A3-C1-C2 chains. In one embodiment, the FVIII protein useful for the present invention is non-active FVIII. In another embodiment, the FVIII protein is an activated FVIII.

The protein having FVIII polypeptide linked to or associated with the VWF fragment can comprise a sequence at least 50%, 60%, 70%, 80%, 90%, 95%, 96%, 97%, 98%, 99%, or 100% identical to SEQ ID NO: 16 or 18, wherein the sequence has the FVIII clotting activity, e.g., activating Factor IX as a cofactor to convert Factor X to activated Factor X (FXa).

“Hybrid” polypeptides and proteins, as used herein, means a combination of a first polypeptide chain, e.g., the VWF fragment, optionally fused to a first heterologous moiety, with a second polypeptide chain, e.g., a FVIII protein, optionally fused to a second heterologous moiety, thereby forming a heterodimer. In one embodiment, the first polypeptide and the second polypeptide in a hybrid are associated with each other via protein-protein interactions, such as charge-charge or hydrophobic interactions. In another embodiment, the first polypeptide and the second polypeptide in a hybrid are associated with each other via disulfide or other covalent bond(s). Hybrids are described, for example, in US 2004/101740 and US 2006/074199. The second polypeptide may be an identical copy of the first polypeptide or a non-identical polypeptide. In one embodiment, the first polypeptide is a VWF fragment-Fc fusion protein, and the second polypeptide is a polypeptide comprising, consisting essentially of, or consisting of an FcRn binding domain, wherein the first polypeptide and the second polypeptide are associated with each other. In another embodiment, the first polypeptide comprises a VWF fragment-Fc fusion protein, and the second polypeptide comprises FVIII-Fc fusion protein, making the hybrid a heterodimer. The first polypeptide and the second polypeptide can be associated through a covalent bond, e.g., a disulfide bond, between the first Fc region and the second Fc region. The first polypeptide and the second polypeptide can further be associated with each other by binding between the VWF fragment and the FVIII protein.

### D) Linkers

The chimeric protein of the present invention further comprises a linker. One or more linkers can be present between any two proteins, e.g., between the adjunct moiety and the FVIII protein (sometimes also referred to as “FVIII/AM linker”), between the VWF fragment and a first heterologous moiety (sometime also referred to as “VWF linker”), e.g., a first Fc region, between a FVIII protein and a second heterologous moiety (sometimes also referred to as “FVIII linker”), e.g., a second Fc region, between the VWF fragment and a FVIII protein (e.g., FVIII/AM linker), between the VWF fragment and a second heterologous moiety, and/or between a FVIII protein and a first heterologous moiety. Each of the linkers can have the same or different sequence. In one embodiment, the linker is a polypeptide linker. In another embodiment, the linker is a non-polypeptide linker.

The linker useful in the present invention can comprise any organic molecule. In one embodiment, the linker is a polymer, e.g., polyethylene glycol (PEG) or hydroxyethyl starch (HES). In another embodiment, the linker is an amino acid sequence (e.g., a polypeptide linker). The polypeptide linker can comprise at least about 10, 20, 30, 40, 50, 60, 70, 80, 90, 100, 150, 200, 300, 400, 500, 600, 700, 800, 900, 1000, 1100, 1200, 1300, 1400, 1500, 1600, 1700, 1800, 1900, or 2000 amino acids. The linker can comprise 1-5 amino acids, 1-10 amino acids, 1-20 amino acids, 10-50 amino acids, 50-100 amino acids, 100-200 amino acids, 200-300 amino acids, 300-400 amino acids, 400-500 amino acids, 500-600 amino acids, 600-700 amino acids, 700-800 amino acids, 800-900 amino acids, or 900-1000 amino acids.

Examples of polypeptide linkers are well known in the art. In one embodiment, the linker comprises the sequence Gₙ. The linker can comprise the sequence (GA)ₙ. The linker can comprise the sequence (GGS)ₙ. In other embodiments, the linker comprises (GGGS)ₙ (SEQ ID NO: 20). In still other embodiments, the linker comprises the sequence (GGS)ₙ(GGGGS)ₙ (SEQ ID NO: 21). In these instances, n may be an integer from 1-100. In other instances, n may be an integer from 1-20, i.e., 1, 2, 3, 4, 5, 6, 7, 8, 9, 10, 11, 12, 13, 14, 15, 16, 17, 18, 19, or 20. Examples of linkers include, but are not limited to, GGG, SGGSGGS (SEQ ID NO: 22), GGSGGSGGSGGSGGG (SEQ ID NO: 23), GGSGGSGGGGSGGGGS (SEQ ID NO: 24), GGSGGSGGSGGSGGSGGS (SEQ ID NO: 25), GGGGSGGGGSGGGGS (SEQ ID NO: 26), the linkers in Table 13 (SEQ ID NOs: 92, 93, and 94), and the linkers in Table 14A (SEQ ID NOs: 95, 96 and 97). The linker does not eliminate or diminish the VWF fragment activity or the clotting activity of Factor VIII. Optionally, the linker enhances the VWF fragment activity or the clotting activity of Factor VIII protein, e.g., by further diminishing the effects of steric hindrance and making the VWF fragment or Factor VIII portion more accessible to its target binding site.

In one embodiment, the linker useful for the chimeric protein is 15-25 amino acids long. In another embodiment, the linker useful for the chimeric protein is 15-20 amino acids long. In some embodiments, the linker for the chimeric protein is 10-25 amino acids long. In other embodiments, the linker for the chimeric protein is 15 amino acids long. In still other embodiments, the linker for the chimeric protein is (GGGGS)ₙ (SEQ ID NO: 27) where G represents glycine, S represents serine and n is an integer from 1-20.

### E) Cleavage Sites

The linker may also incorporate a moiety capable of being cleaved either chemically (e.g., hydrolysis of an ester bond), enzymatically (i.e., incorporation of a protease cleavage sequence), or photolytically (e.g., a chromophore such as 3-amino-3-(2-nitrophenyl) proprionic acid (ANP)) in order to release one molecule from another.

In one embodiment, the linker is a cleavable linker. The cleavable linkers can comprise one or more cleavage sites at the N-terminus or C-terminus or both. In another embodiment, the cleavable linker consists essentially of or consists of one or more cleavable sites. In other embodiments, the cleavable linker comprises heterologous amino acid linker sequences described herein or polymers and one or more cleavable sites.

In certain embodiments, a cleavable linker comprises one or more cleavage sites that can be cleaved in a host cell (i.e., intracellular processing sites). Non limiting examples of the cleavage site include RRRR (SEQ ID NO: 52), RKRRKR (SEQ ID NO: 53), and RRRRS (SEQ ID NO: 54).

In other embodiments, a cleavable linker comprises one or more cleavage sites that are cleaved by a protease after a chimeric protein comprising the cleavable linker is administered to a subject. In one embodiment, the cleavage site is cleaved by a protease selected from the group consisting of factor XIa, factor XIIa, kallikrein, factor VIIa, factor IXa, factor Xa, factor IIa (thrombin), Elastase-2, MMP-12, MMP-13, MMP-17, and MMP-20. In another embodiment, the cleavage site is selected from the group consisting of a FXIa cleavage site (e.g., KLTR↓AET (SEQ ID NO: 29)), a FXIa cleavage site (e.g, DFTR ↓VVG (SEQ ID NO: 30)), a FXIIa cleavage site (e.g., TMTR↓IVGG (SEQ ID NO: 31)), a Kallikrein cleavage site (e.g., SPFR↓STGG (SEQ ID NO: 32)), a FVIIa cleavage site (e.g., LQVR↓IVGG (SEQ ID NO: 33)), a FIXa cleavage site (e.g., PLGR↓IVGG (SEQ ID NO: 34)), a FXa cleavage site (e.g., IEGR↓TVGG (SEQ ID NO: 35)), a FIIa (thrombin) cleavage site (e.g, LTPR↓SLLV (SEQ ID NO: 36)), a Elastase-2 cleavage site (e.g, LGPV ↓SGVP (SEQ ID NO: 37)), a Granzyme-B cleavage (e.g, VAGD↓SLEE (SEQ ID NO: 38)), a MMP-12 cleavage site (e.g., GPAG↓LGGA (SEQ ID NO: 39)), a MMP-13 cleavage site (e.g., GPAG↓LRGA (SEQ ID NO: 40)), a MMP-17 cleavage site (e.g., APLG↓LRLR (SEQ ID NO: 41)), a MMP-20 cleavage site (e.g., PALP↓LVAQ (SEQ ID NO: 42)), a TEV cleavage site (e.g., ENLYFQ↓G (SEQ ID NO: 43)), a Enterokinase cleavage site (e.g., DDDK↓IVGG (SEQ ID NO: 44)), a Protease 3C (PRESCISSION™) cleavage site (e.g., LEVLFQ↓GP (SEQ ID NO: 45)), and a Sortase A cleavage site (e.g., LPKT↓GSES) (SEQ ID NO: 46). In certain embodiments, the FXIa cleavage sites include, but are not limited to, e.g., TQSFNDFTR (SEQ ID NO: 47) and SVSQTSKLTR (SEQ ID NO: 48). Non-limiting exemplary thrombin cleavage sites include, e.g., DFLAEGGGVR (SEQ ID NO: 49), TTKIKPR (SEQ ID NO: 50), or LVPRG (SEQ ID NO: 55), and a sequence comprising, consisting essentially of, or consisting of ALRPR (e.g., ALRPRVVGGA (SEQ ID NO: 51)).

In a specific embodiment, the cleavage site is TLDPRSFLLRNPNDKYEPFWEDEEK (SEQ ID NO: 56).

Polynucleotides, Vectors, Host cells, and Methods of Making

Also provided in the invention is a polynucleotide encoding a VWF fragment described herein, a chimeric protein comprising the VWF fragment and a heterologous moiety, a chimeric protein comprising a FVIII protein and an adjunct moiety, or a chimeric protein comprising a VWF fragment and a FVIII protein. When a VWF fragment is linked to a heterologous moiety or a FVIII protein in a chimeric protein as a single polypeptide chain, the invention is drawn to a polynucleotide encoding the VWF fragment linked to the heterologous moiety or the FVIII protein. When the chimeric protein comprises a first and a second polypeptide chains, the first polypeptide chain comprising a VWF fragment and a first heterologous moiety (e.g., a first Fc region) and the second polypeptide chain comprising a second heterologous moiety (e.g., a second Fc region), wherein the first polypeptide chain and the second polypeptide chain are associated with each other, a polynucleotide can comprise the first nucleotide sequence and the second nucleotide sequence. In one embodiment, the first nucleotide sequence and the second nucleotide sequence are on the same polynucleotide. In another embodiment, the first nucleotide sequence and the second nucleotide sequence are on two different polynucleotides (e.g., different vectors). In certain embodiments, the present invention is directed to a set of polynucleotides comprising a first nucleotide chain and a second nucleotide chain, wherein the first nucleotide chain encodes the VWF fragment of the chimeric protein and the second nucleotide chain encodes the FVIII protein.

In other embodiments, the set of the polynucleotides further comprises an additional nucleotide chain (e.g., a second nucleotide chain when the chimeric polypeptide is encoded by a single polynucleotide chain or a third nucleotide chain when the chimeric protein is encoded by two polynucleotide chains) which encodes a protein convertase. The protein convertase can be selected from the group consisting of proprotein convertase subtilisin/kexin type 5 (PCSK5 or PC5), proprotein convertase subtilisin/kexin type 7 (PCSK7 or PC5), a yeast Kex 2, proprotein convertase subtilisin/kexin type 3 (PACE or PCSK3), and two or more combinations thereof. In some embodiments, the protein convertase is PACE, PC5, or PC7. In a specific embodiment, the protein convertase is PC5 or PC7. See International Application no. PCT/US2011/043568, which is incorporated herein by reference. In another embodiment, the protein convertase is PACE/Furin.

In certain embodiments, the invention includes a set of the polynucleotides comprising a first nucleotide sequence encoding a VWF fragment comprising a D′ domain and a D3 domain of VWF, a second nucleotide sequence encoding a FVIII protein, and a third nucleotide sequence encoding a D1 domain and D2 domain of VWF. In this embodiment, the D1 domain and D2 domain are separately expressed (not linked to the D′D3 domain of the VWF fragment) in order for the proper disulfide bond formation and folding of the D′D3 domains. The DID2 domain expression can either be in cis or trans.

As used herein, an expression vector refers to any nucleic acid construct which contains the necessary elements for the transcription and translation of an inserted coding sequence, or in the case of an RNA viral vector, the necessary elements for replication and translation, when introduced into an appropriate host cell. Expression vectors can include plasmids, phagemids, viruses, and derivatives thereof.

Expression vectors of the invention will include polynucleotides encoding the VWF fragment or the chimeric protein comprising the VWF fragment.

In one embodiment, a coding sequence for the VWF fragment, the second heterologous moiety (e.g., a second Fc region), or the FVIII protein is operably linked to an expression control sequence. As used herein, two nucleic acid sequences are operably linked when they are covalently linked in such a way as to permit each component nucleic acid sequence to retain its functionality. A coding sequence and a gene expression control sequence are said to be operably linked when they are covalently linked in such a way as to place the expression or transcription and/or translation of the coding sequence under the influence or control of the gene expression control sequence. Two DNA sequences are said to be operably linked if induction of a promoter in the 5′ gene expression sequence results in the transcription of the coding sequence and if the nature of the linkage between the two DNA sequences does not (1) result in the introduction of a frame-shift mutation, (2) interfere with the ability of the promoter region to direct the transcription of the coding sequence, or (3) interfere with the ability of the corresponding RNA transcript to be translated into a protein. Thus, a gene expression sequence would be operably linked to a coding nucleic acid sequence if the gene expression sequence were capable of effecting transcription of that coding nucleic acid sequence such that the resulting transcript is translated into the desired protein or polypeptide.

A gene expression control sequence as used herein is any regulatory nucleotide sequence, such as a promoter sequence or promoter-enhancer combination, which facilitates the efficient transcription and translation of the coding nucleic acid to which it is operably linked. The gene expression control sequence may, for example, be a mammalian or viral promoter, such as a constitutive or inducible promoter. Constitutive mammalian promoters include, but are not limited to, the promoters for the following genes: hypoxanthine phosphoribosyl transferase (HPRT), adenosine deaminase, pyruvate kinase, beta-actin promoter, and other constitutive promoters. Exemplary viral promoters which function constitutively in eukaryotic cells include, for example, promoters from the cytomegalovirus (CMV), simian virus (e.g., SV40), papilloma virus, adenovirus, human immunodeficiency virus (HIV), Rous sarcoma virus, cytomegalovirus, the long terminal repeats (LTR) of Moloney leukemia virus, and other retroviruses, and the thymidine kinase promoter of herpes simplex virus. Other constitutive promoters are known to those of ordinary skill in the art. The promoters useful as gene expression sequences of the invention also include inducible promoters. Inducible promoters are expressed in the presence of an inducing agent. For example, the metallothionein promoter is induced to promote transcription and translation in the presence of certain metal ions. Other inducible promoters are known to those of ordinary skill in the art.

In general, the gene expression control sequence shall include, as necessary, 5′ non-transcribing and 5′ non-translating sequences involved with the initiation of transcription and translation, respectively, such as a TATA box, capping sequence, CAAT sequence, and the like. Especially, such 5′ non-transcribing sequences will include a promoter region which includes a promoter sequence for transcriptional control of the operably joined coding nucleic acid. The gene expression sequences optionally include enhancer sequences or upstream activator sequences as desired.

Viral vectors include, but are not limited to, nucleic acid sequences from the following viruses: retrovirus, such as Moloney murine leukemia virus, Harvey murine sarcoma virus, murine mammary tumor virus, and Rous sarcoma virus; adenovirus, adeno-associated virus; SV40-type viruses; polyomaviruses; Epstein-Barr viruses; papilloma viruses; herpes virus; vaccinia virus; polio virus; and RNA virus such as a retrovirus. One can readily employ other vectors well-known in the art. Certain viral vectors are based on non-cytopathic eukaryotic viruses in which non-essential genes have been replaced with the gene of interest. Non-cytopathic viruses include retroviruses, the life cycle of which involves reverse transcription of genomic viral RNA into DNA with subsequent proviral integration into host cellular DNA. Retroviruses have been approved for human gene therapy trials. Most useful are those retroviruses that are replication-deficient (i.e., capable of directing synthesis of the desired proteins, but incapable of manufacturing an infectious particle). Such genetically altered retroviral expression vectors have general utility for the high-efficiency transduction of genes in vivo. Standard protocols for producing replication-deficient retroviruses (including the steps of incorporation of exogenous genetic material into a plasmid, transfection of a packaging cell line with plasmid, production of recombinant retroviruses by the packaging cell line, collection of viral particles from tissue culture media, and infection of the target cells with viral particles) are provided in Kriegler, M., Gene Transfer and Expression, A Laboratory Manual, W.H. Freeman Co., New York (1990) and Murry, E. J., Methods in Molecular Biology, Vol. 7, Humana Press, Inc., Cliffton, N.J. (1991).

In one embodiment, the virus is an adeno-associated virus, a double-stranded DNA virus. The adeno-associated virus can be engineered to be replication-deficient and is capable of infecting a wide range of cell types and species. It further has advantages such as heat and lipid solvent stability; high transduction frequencies in cells of diverse lineages, including hemopoietic cells; and lack of superinfection inhibition thus allowing multiple series of transductions. Reportedly, the adeno-associated virus can integrate into human cellular DNA in a site-specific manner, thereby minimizing the possibility of insertional mutagenesis and variability of inserted gene expression characteristic of retroviral infection. In addition, wild-type adeno-associated virus infections have been followed in tissue culture for greater than 100 passages in the absence of selective pressure, implying that the adeno-associated virus genomic integration is a relatively stable event. The adeno-associated virus can also function in an extrachromosomal fashion.

Other vectors include plasmid vectors. Plasmid vectors have been extensively described in the art and are well-known to those of skill in the art. See, e.g., Sambrook et al., Molecular Cloning: A Laboratory Manual, Second Edition, Cold Spring Harbor Laboratory Press, 1989. In the last few years, plasmid vectors have been found to be particularly advantageous for delivering genes to cells in vivo because of their inability to replicate within and integrate into a host genome. These plasmids, however, having a promoter compatible with the host cell, can express a peptide from a gene operably encoded within the plasmid. Some commonly used plasmids available from commercial suppliers include pBR322, pUC18, pUC19, various pcDNA plasmids, pRC/CMV, various pCMV plasmids, pSV40, and pBlueScript. Additional examples of specific plasmids include pcDNA3.1, catalog number V79020; pcDNA3.1/hygro, catalog number V87020; pcDNA4/myc-His, catalog number V86320; and pBudCE4.1, catalog number V53220, all from Invitrogen (Carlsbad, CA.). Other plasmids are well-known to those of ordinary skill in the art. Additionally, plasmids may be custom designed using standard molecular biology techniques to remove and/or add specific fragments of DNA.

In one insect expression system that may be used to produce the proteins of the invention, Autographa californica nuclear polyhidrosis virus (AcNPV) is used as a vector to express the foreign genes. The virus grows in Spodoptera frugiperda cells. A coding sequence may be cloned into non-essential regions (for example, the polyhedron gene) of the virus and placed under control of an ACNPV promoter (for example, the polyhedron promoter). Successful insertion of a coding sequence will result in inactivation of the polyhedron gene and production of non-occluded recombinant virus (i.e., virus lacking the proteinaceous coat coded for by the polyhedron gene). These recombinant viruses are then used to infect Spodoptera frugiperda cells in which the inserted gene is expressed. (see, e.g., Smith et al. (1983) J Virol 46:584; U.S. Pat. No. 4,215,051). Further examples of this expression system may be found in Ausubel et al., eds. (1989) Current Protocols in Molecular Biology, Vol. 2, Greene Publish. Assoc. & Wiley Interscience.

Another system which can be used to express the proteins of the invention is the glutamine synthetase gene expression system, also referred to as the “GS expression system” (Lonza Biologics PLC, Berkshire UK). This expression system is described in detail in U.S. Pat. No. 5,981,216.

In mammalian host cells, a number of viral based expression systems may be utilized. In cases where an adenovirus is used as an expression vector, a coding sequence may be ligated to an adenovirus transcription/translation control complex, e.g., the late promoter and tripartite leader sequence. This chimeric gene may then be inserted in the adenovirus genome by in vitro or in vivo recombination. Insertion in a non-essential region of the viral genome (e.g., region E1 or E3) will result in a recombinant virus that is viable and capable of expressing peptide in infected hosts. See, e.g., Logan & Shenk (1984) Proc Natl Acad Sci USA 81:3655). Alternatively, the vaccinia 7.5 K promoter may be used. See, e.g., Mackett et al. (1982) Proc Natl Acad Sci USA 79:7415; Mackett et al. (1984) J Virol 49:857; Panicali et al. (1982) Proc Natl Acad Sci USA 79:4927.

To increase efficiency of production, the polynucleotides can be designed to encode multiple units of the protein of the invention separated by enzymatic cleavage sites. The resulting polypeptide can be cleaved (e.g., by treatment with the appropriate enzyme) in order to recover the polypeptide units. This can increase the yield of polypeptides driven by a single promoter. When used in appropriate viral expression systems, the translation of each polypeptide encoded by the mRNA is directed internally in the transcript; e.g., by an internal ribosome entry site, IRES. Thus, the polycistronic construct directs the transcription of a single, large polycistronic mRNA which, in turn, directs the translation of multiple, individual polypeptides. This approach eliminates the production and enzymatic processing of polyproteins and may significantly increase the yield of polypeptides driven by a single promoter.

Vectors used in transformation will usually contain a selectable marker used to identify transformants. In bacterial systems, this can include an antibiotic resistance gene such as ampicillin or kanamycin. Selectable markers for use in cultured mammalian cells include genes that confer resistance to drugs, such as neomycin, hygromycin, and methotrexate. The selectable marker may be an amplifiable selectable marker. One amplifiable selectable marker is the dihydrofolate reductase (DHFR) gene. Simonsen C C et al. (1983) Proc Natl Acad Sci USA 80:2495-9. Selectable markers are reviewed by Thilly (1986) Mammalian Cell Technology, Butterworth Publishers, Stoneham, Mass., and the choice of selectable markers is well within the level of ordinary skill in the art.

Selectable markers may be introduced into the cell on a separate plasmid at the same time as the gene of interest, or they may be introduced on the same plasmid. If on the same plasmid, the selectable marker and the gene of interest may be under the control of different promoters or the same promoter, the latter arrangement producing a dicistronic message. Constructs of this type are known in the art (for example, U.S. Pat. No. 4,713,339).

The expression vectors can encode for tags that permit easy purification of the recombinantly produced protein. Examples include, but are not limited to, vector pUR278 (Ruther et al. (1983) EMBO J 2:1791), in which coding sequences for the protein to be expressed may be ligated into the vector in frame with the lac z coding region so that a tagged fusion protein is produced; pGEX vectors may be used to express proteins of the invention with a glutathione S-transferase (GST) tag. These proteins are usually soluble and can easily be purified from cells by adsorption to glutathione-agarose beads followed by elution in the presence of free glutathione. The vectors include cleavage sites (thrombin or Factor Xa protease or PRESCISSION PROTEASE™ (Pharmacia, Peapack, N.J.)) for easy removal of the tag after purification.

The expression vector or vectors are then transfected or co-transfected into a suitable target cell, which will express the polypeptides. Transfection techniques known in the art include, but are not limited to, calcium phosphate precipitation (Wigler et al. (1978) Cell 14:725), electroporation (Neumann et al. (1982) EMBO J 1:841), and liposome-based reagents. A variety of host-expression vector systems may be utilized to express the proteins described herein including both prokaryotic and eukaryotic cells. These include, but are not limited to, microorganisms such as bacteria (e.g., E. coli) transformed with recombinant bacteriophage DNA or plasmid DNA expression vectors containing an appropriate coding sequence; yeast or filamentous fungi transformed with recombinant yeast or fungi expression vectors containing an appropriate coding sequence; insect cell systems infected with recombinant virus expression vectors (e.g., baculovirus) containing an appropriate coding sequence; plant cell systems infected with recombinant virus expression vectors (e.g., cauliflower mosaic virus or tobacco mosaic virus) or transformed with recombinant plasmid expression vectors (e.g., Ti plasmid) containing an appropriate coding sequence; or animal cell systems, including mammalian cells (e.g., HEK 293, CHO, Cos, HeLa, HKB11, and BHK cells).

In one embodiment, the host cell is a eukaryotic cell. As used herein, a eukaryotic cell refers to any animal or plant cell having a definitive nucleus. Eukaryotic cells of animals include cells of vertebrates, e.g., mammals, and cells of invertebrates, e.g., insects. Eukaryotic cells of plants specifically can include, without limitation, yeast cells. A eukaryotic cell is distinct from a prokaryotic cell, e.g., bacteria.

In certain embodiments, the eukaryotic cell is a mammalian cell. A mammalian cell is any cell derived from a mammal. Mammalian cells specifically include, but are not limited to, mammalian cell lines. In one embodiment, the mammalian cell is a human cell. In another embodiment, the mammalian cell is a HEK 293 cell, which is a human embryonic kidney cell line. HEK 293 cells are available as CRL-1533 from American Type Culture Collection, Manassas, VA, and as 293-H cells, Catalog No. 11631-017 or 293-F cells, Catalog No. 11625-019 from Invitrogen (Carlsbad, Calif.). In some embodiments, the mammalian cell is a PER.C6® cell, which is a human cell line derived from retina. PER.C6® cells are available from Crucell (Leiden, The Netherlands). In other embodiments, the mammalian cell is a Chinese hamster ovary (CHO) cell. CHO cells are available from American Type Culture Collection, Manassas, VA. (e.g., CHO-K1; CCL-61). In still other embodiments, the mammalian cell is a baby hamster kidney (BHK) cell. BHK cells are available from American Type Culture Collection, Manassas, Va. (e.g., CRL-1632). In some embodiments, the mammalian cell is a HKB11 cell, which is a hybrid cell line of a HEK293 cell and a human B cell line. Mei et al., Mol. Biotechnol. 34(2): 165-78 (2006).

In one embodiment, a plasmid encoding the VWF fragment or the chimeric protein of the invention further includes a selectable marker, e.g., zeocin resistance, and is transfected into HEK 293 cells, for production of the VWF fragment or the chimeric protein.

In another embodiment, a first plasmid comprising a Factor VIII-Fc fusion coding sequence and a first selectable marker, e.g., a zeocin resistance gene, and a second plasmid comprising a VWF fragment-Fc coding sequence and a second selectable marker, e.g., a neomycin resistance gene, are cotransfected into HEK 293 cells, for production of Factor VIII-Fc and VWF-Fc hybrid. The first and second plasmids can be introduced in equal amounts (i.e., 1:1 ratio), or they can be introduced in unequal amounts.

In some embodiments, a first plasmid including a Factor VIII-Fc fusion coding sequence and a first selectable marker, e.g., a zeocin resistance gene, and a second plasmid including a VWF fragment-Fc coding sequence and a second selectable marker, e.g., a neomycin resistance gene, and a third plasmid including a protein convertase coding sequence (e.g., PC5 or Furin) and a third selectable marker, e.g., a hygromycin resistance gene, are cotransfected into HEK 293 cells, for production of Factor VIII-VWF fragment hybrid. The first and second plasmids can be introduced in equal amounts (i.e., 1:1 molar ratio), or they can be introduced in unequal amounts. In certain embodiments, a first plasmid, including a Factor VIII-Fc fusion coding sequence, a VWF fragment-Fc coding sequence, and a first selectable marker, e.g., a zeocin resistance gene, and a second plasmid including a protein convertase coding sequence (e.g., PC5 or Furin) and a second selectable marker, e.g., a hygromycin resistance gene, are cotransfected into HEK 293 cells, for production of Factor VIII-VWF-fragment hybrid. In one embodiment, the nucleotide sequence encoding the FVIII-Fc sequence and the VWF fragment-Fc sequence can be connected to encode one single polypeptide. In another embodiment, the nucleotide sequence encoding the FVIII-Fc sequence and the VWF fragment-Fc sequence can be encoded as two polypeptide chains. The promoters for the Factor VIII-Fc fusion coding sequence and the VWF fragment-Fc coding sequence can be different or they can be the same.

In some embodiments, a plasmid comprising Furin is co-transfected with the plasmid containing the Factor VIII-Fc coding sequence and/or VWF fragment-Fc coding sequence. In some embodiments, the Furin protein is on the same plasmid comprising the Factor VIII-Fc fusion coding sequence. In some embodiments, the Furin protein is on the same plasmid comprising the VWF fragment-Fc coding sequence. In some embodiments, the Furin protein is on a separate plasmid.

In still other embodiments, transfected cells are stably transfected. These cells can be selected and maintained as a stable cell line, using conventional techniques known to those of skill in the art.

Host cells containing DNA constructs of the protein are grown in an appropriate growth medium. As used herein, the term “appropriate growth medium” means a medium containing nutrients required for the growth of cells. Nutrients required for cell growth may include a carbon source, a nitrogen source, essential amino acids, vitamins, minerals, and growth factors. Optionally, the media can contain one or more selection factors. Optionally the media can contain bovine calf serum or fetal calf serum (FCS). In one embodiment, the media contains substantially no IgG. The growth medium will generally select for cells containing the DNA construct by, for example, drug selection or deficiency in an essential nutrient which is complemented by the selectable marker on the DNA construct or co-transfected with the DNA construct. Cultured mammalian cells are generally grown in commercially available serum-containing or serum-free media (e.g., MEM, DMEM, DMEM/F12). In one embodiment, the medium is CD293 (Invitrogen, Carlsbad, CA.). In another embodiment, the medium is CD17 (Invitrogen, Carlsbad, CA.). Selection of a medium appropriate for the particular cell line used is within the level of those ordinary skilled in the art.

In order to co-express the VWF fragment and a second heterologous moiety or a FVIII protein, the host cells are cultured under conditions that allow expression of both the VWF fragment and a second heterologous moiety or a FVIII protein. As used herein, culturing refers to maintaining living cells in vitro for at least a definite time. Maintaining can, but need not include, an increase in population of living cells. For example, cells maintained in culture can be static in population, but still viable and capable of producing a desired product, e.g., a recombinant protein or recombinant fusion protein. Suitable conditions for culturing eukaryotic cells are well known in the art and include appropriate selection of culture media, media supplements, temperature, pH, oxygen saturation, and the like. For commercial purposes, culturing can include the use of any of various types of scale-up systems including shaker flasks, roller bottles, hollow fiber bioreactors, stirred-tank bioreactors, airlift bioreactors, Wave bioreactors, and others.

The cell culture conditions are also selected to allow association of the VWF fragment with the second heterologous moiety or a FVIII protein. Conditions that allow expression of the VWF fragment and/or the FVIII protein, may include the presence of a source of vitamin K. For example, in one embodiment, stably transfected HEK 293 cells are cultured in CD293 media (Invitrogen, Carlsbad, CA) or OptiCHO media (Invitrogen, Carlsbad, CA) supplemented with 4 mM glutamine.

In one aspect, the present invention is directed to a method of expressing, making, or producing the VWF fragment of the invention comprising a) transfecting a host cell with a polynucleotide encoding the VWF fragment and b) culturing the host cell in a culture medium under a condition suitable for expressing the VWF fragment, wherein the VWF fragment is expressed. In one embodiment, the invention is drawn to a method of producing a mature VWF protein or a fragment thereof comprising a) transfecting a host cell with a first polynucleotide encoding the VWF protein or a fragment thereof, which is fused to a propeptide of VWF, and a second polynucleotide encoding a protein convertase, e.g., PC5, PC7, or Furin and b) culturing the host cell in a culture medium under a condition suitable for expressing the mature VWF protein or fragment thereof. The polynucleotide encoding the VWF protein or a fragment thereof can also be fused to a prepeptide of VWF. The prepeptide sequence can be cleaved during insertion to the endoplasmic reticulum before secretion.

In another aspect, the invention is directed to a method of expressing, making, or producing a chimeric protein comprising the VWF fragment linked to or associated with a heterologous moiety or a FVIII protein comprising a) transfecting one or more host cells with a polynucleotide or a set of polynucleotides encoding the chimeric protein and b) culturing the host cell in a culture medium under conditions suitable for expressing the chimeric protein. In one embodiment, the invention is drawn to a method of expressing, making, or producing a chimeric protein comprising a) transfecting a host cell with a first polynucleotide encoding a VWF fragment linked to a heterologous moiety and a second polynucleotide encoding a FVIII protein linked to a heterologous moiety and b) culturing the host cell in a culture medium under conditions suitable for expressing the chimeric protein. The first polynucleotide and the second polynucleotide can be in one vector or two vectors. In another embodiment, the invention is drawn to a method of expressing, making, or producing a chimeric protein comprising a) transfecting a host cell with a first polynucleotide encoding a VWF fragment linked to a heterologous moiety, a second polynucleotide encoding a FVIII protein linked to a heterologous moiety, and a third polynucleotide encoding a protein convertase, and b) culturing the host cell in a culture medium under conditions suitable for expressing the chimeric protein. In other embodiments, the invention is drawn to a method of expressing, making, or producing a chimeric protein comprising a) transfecting a host cell with a first polynucleotide encoding a VWF fragment comprising a D′ domain and a D3 domain linked to a heterologous moiety, a second polynucleotide encoding a FVIII protein linked to a heterologous moiety, and a third polynucleotide encoding a D1 domain and a D2 domain of VWF, and b) culturing the host cell in a culture medium under conditions suitable for expressing the chimeric protein. In one embodiment, the first polynucleotide, the second polynucleotide, and the third polynucleotide can be in one vector or separate vectors. In another embodiment, the first polynucleotide and the second polynucleotide can be in one vector, and the third polynucleotide can be another vector. In other embodiments, the first polynucleotide and the third polynucleotide can be in one vector, and the second polynucleotide can be another vector. In some embodiments, the second polynucleotide and the third polynucleotide can be in one vector and the first polynucleotide can be in another vector.

In further embodiments, the protein product containing the VWF fragment or the chimeric protein comprising the VWF fragment is secreted into the media. Media is separated from the cells, concentrated, filtered, and then passed over two or three affinity columns, e.g., a protein A column and one or two anion exchange columns.

In certain aspects, the present invention relates to the VWF fragment or the chimeric polypeptide produced by the methods described herein.

In vitro production allows scale-up to give large amounts of the desired altered polypeptides of the invention. Techniques for mammalian cell cultivation under tissue culture conditions are known in the art and include homogeneous suspension culture, e.g. in an airlift reactor or in a continuous stirrer reactor, or immobilized or entrapped cell culture, e.g. in hollow fibers, microcapsules, on agarose microbeads or ceramic cartridges. If necessary and/or desired, the solutions of polypeptides can be purified by the customary chromatography methods, for example gel filtration, ion-exchange chromatography, hydrophobic interaction chromatography (HIC, chromatography over DEAE-cellulose or affinity chromatography.

### Pharmaceutical Composition

Compositions containing the VWF fragment or the chimeric protein of the present invention may contain a suitable pharmaceutically acceptable carrier. For example, they may contain excipients and/or auxiliaries that facilitate processing of the active compounds into preparations designed for delivery to the site of action.

The pharmaceutical composition can be formulated for parenteral administration (i.e. intravenous, subcutaneous, or intramuscular) by bolus injection. Formulations for injection can be presented in unit dosage form, e.g., in ampoules or in multidose containers with an added preservative. The compositions can take such forms as suspensions, solutions, or emulsions in oily or aqueous vehicles, and contain formulatory agents such as suspending, stabilizing and/or dispersing agents. Alternatively, the active ingredient can be in powder form for constitution with a suitable vehicle, e.g., pyrogen free water.

Suitable formulations for parenteral administration also include aqueous solutions of the active compounds in water-soluble form, for example, water-soluble salts. In addition, suspensions of the active compounds as appropriate oily injection suspensions may be administered. Suitable lipophilic solvents or vehicles include fatty oils, for example, sesame oil, or synthetic fatty acid esters, for example, ethyl oleate or triglycerides. Aqueous injection suspensions may contain substances, which increase the viscosity of the suspension, including, for example, sodium carboxymethyl cellulose, sorbitol and dextran. Optionally, the suspension may also contain stabilizers. Liposomes also can be used to encapsulate the molecules of the invention for delivery into cells or interstitial spaces. Exemplary pharmaceutically acceptable carriers are physiologically compatible solvents, dispersion media, coatings, antibacterial and antifungal agents, isotonic and absorption delaying agents, water, saline, phosphate buffered saline, dextrose, glycerol, ethanol and the like. In some embodiments, the composition comprises isotonic agents, for example, sugars, polyalcohols such as mannitol, sorbitol, or sodium chloride. In other embodiments, the compositions comprise pharmaceutically acceptable substances such as wetting agents or minor amounts of auxiliary substances such as wetting or emulsifying agents, preservatives or buffers, which enhance the shelf life or effectiveness of the active ingredients.

Compositions of the invention may be in a variety of forms, including, for example, liquid (e.g., injectable and infusible solutions), dispersions, suspensions, semi-solid and solid dosage forms. The preferred form depends on the mode of administration and therapeutic application.

The composition can be formulated as a solution, micro emulsion, dispersion, liposome, or other ordered structure suitable to high drug concentration. Sterile injectable solutions can be prepared by incorporating the active ingredient in the required amount in an appropriate solvent with one or a combination of ingredients enumerated above, as required, followed by filtered sterilization. Generally, dispersions are prepared by incorporating the active ingredient into a sterile vehicle that contains a basic dispersion medium and the required other ingredients from those enumerated above. In the case of sterile powders for the preparation of sterile injectable solutions, the preferred methods of preparation are vacuum drying and freeze-drying that yields a powder of the active ingredient plus any additional desired ingredient from a previously sterile-filtered solution. The proper fluidity of a solution can be maintained, for example, by the use of a coating such as lecithin, by the maintenance of the required particle size in the case of dispersion and by the use of surfactants. Prolonged absorption of injectable compositions can be brought about by including in the composition an agent that delays absorption, for example, monostearate salts and gelatin.

The active ingredient can be formulated with a controlled-release formulation or device. Examples of such formulations and devices include implants, transdermal patches, and microencapsulated delivery systems. Biodegradable, biocompatible polymers can be used, for example, ethylene vinyl acetate, polyanhydrides, polyglycolic acid, collagen, polyorthoesters, and polylactic acid. Methods for the preparation of such formulations and devices are known in the art. See e.g., Sustained and Controlled Release Drug Delivery Systems, J. R. Robinson, ed., Marcel Dekker, Inc., New York, 1978.

Injectable depot formulations can be made by forming microencapsulated matrices of the drug in biodegradable polymers such as polylactide-polyglycolide. Depending on the ratio of drug to polymer, and the nature of the polymer employed, the rate of drug release can be controlled. Other exemplary biodegradable polymers are polyorthoesters and polyanhydrides. Depot injectable formulations also can be prepared by entrapping the drug in liposomes or microemulsions.

Supplementary active compounds can be incorporated into the compositions. In one embodiment, the VWF fragment or the chimeric protein of the invention is formulated with another clotting factor, or a variant, fragment, analogue, or derivative thereof. For example, the clotting factor includes, but is not limited to, factor V, factor VII, factor VIII, factor IX, factor X, factor XI, factor XII, factor XIII, prothrombin, fibrinogen, von Willebrand factor or recombinant soluble tissue factor (rsTF) or activated forms of any of the preceding. The clotting factor of hemostatic agent can also include anti-fibrinolytic drugs, e.g., epsilon-amino-caproic acid, tranexamic acid.

Dosage regimens may be adjusted to provide the optimum desired response. For example, a single bolus may be administered, several divided doses may be administered over time, or the dose may be proportionally reduced or increased as indicated by the exigencies of the therapeutic situation. It is advantageous to formulate parenteral compositions in dosage unit form for ease of administration and uniformity of dosage. See, e.g., Remington's Pharmaceutical Sciences (Mack Pub. Co., Easton, Pa. 1980).

In addition to the active compound, the liquid dosage form may contain inert ingredients such as water, ethyl alcohol, ethyl carbonate, ethyl acetate, benzyl alcohol, benzyl benzoate, propylene glycol, 1,3-butylene glycol, dimethylformamide, oils, glycerol, tetrahydrofurfuryl alcohol, polyethylene glycols, and fatty acid esters of sorbitan.

Non-limiting examples of suitable pharmaceutical carriers are also described in Remington's Pharmaceutical Sciences by E. W. Martin. Some examples of excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol, and the like. The composition can also contain pH buffering reagents, and wetting or emulsifying agents.

For oral administration, the pharmaceutical composition can take the form of tablets or capsules prepared by conventional means. The composition can also be prepared as a liquid for example a syrup or a suspension. The liquid can include suspending agents (e.g., sorbitol syrup, cellulose derivatives or hydrogenated edible fats), emulsifying agents (lecithin or acacia), non-aqueous vehicles (e.g., almond oil, oily esters, ethyl alcohol, or fractionated vegetable oils), and preservatives (e.g., methyl or propyl-p-hydroxybenzoates or sorbic acid). The preparations can also include flavoring, coloring and sweetening agents. Alternatively, the composition can be presented as a dry product for constitution with water or another suitable vehicle.

For buccal administration, the composition may take the form of tablets or lozenges according to conventional protocols.

For administration by inhalation, the compounds for use according to the present invention are conveniently delivered in the form of a nebulized aerosol with or without excipients or in the form of an aerosol spray from a pressurized pack or nebulizer, with optionally a propellant, e.g., dichlorodifluoromethane, trichlorofluoromethane, dichlorotetrafluoromethane, carbon dioxide or other suitable gas. In the case of a pressurized aerosol the dosage unit can be determined by providing a valve to deliver a metered amount. Capsules and cartridges of, e.g., gelatin for use in an inhaler or insufflator can be formulated containing a powder mix of the compound and a suitable powder base such as lactose or starch.

The pharmaceutical composition can also be formulated for rectal administration as a suppository or retention enema, e.g., containing conventional suppository bases such as cocoa butter or other glycerides.

### Gene Therapy

A VWF fragment or chimeric protein thereof of the invention can be produced in vivo in a mammal, e.g., a human patient, using a gene therapy approach to treatment of a bleeding disease or disorder selected from the group consisting of a bleeding coagulation disorder, hemarthrosis, muscle bleed, oral bleed, hemorrhage, hemorrhage into muscles, oral hemorrhage, trauma, trauma capitis, gastrointestinal bleeding, intracranial hemorrhage, intra-abdominal hemorrhage, intrathoracic hemorrhage, bone fracture, central nervous system bleeding, bleeding in the retropharyngeal space, bleeding in the retroperitoneal space, and bleeding in the illiopsoas sheath would be therapeutically beneficial. In one embodiment, the bleeding disease or disorder is hemophilia. In another embodiment, the bleeding disease or disorder is hemophilia A. This involves administration of a suitable VWF fragment or chimeric protein-encoding nucleic acid operably linked to suitable expression control sequences. In certain embodiment, these sequences are incorporated into a viral vector. Suitable viral vectors for such gene therapy include adenoviral vectors, lentiviral vectors, baculoviral vectors, Epstein Barr viral vectors, papovaviral vectors, vaccinia viral vectors, herpes simplex viral vectors, and adeno associated virus (AAV) vectors. The viral vector can be a replication-defective viral vector. In other embodiments, a adenoviral vector has a deletion in its E1 gene or E3 gene. When an adenoviral vector is used, the mammal may not be exposed to a nucleic acid encoding a selectable marker gene. In other embodiments, the sequences are incorporated into a non-viral vector known to those skilled in the art.

### Methods of Using VWF Fragment or Chimeric Protein

One aspect of the present invention is directed to preventing or inhibiting FVIII interaction with endogenous VWF by blocking or shielding the VWF binding site on the FVIII from endogenous VWF. In one embodiment, the invention is directed to a method of constructing a FVIII protein having half-life longer than wild-type FVIII or a FVIII monomer-dimer hybrid, the method comprising covalently associating an adjunct moiety with the FVIII protein, thereby making a chimeric protein comprising the FVIII protein and the adjunct moiety, wherein the adjunct moiety shields or prevents the FVIII protein interaction with endogenous VWF. The chimeric protein useful in the method includes any one or more chimeric protein described herein.

Another aspect of the invention includes a method of administering to a subject in need thereof a FVIII protein having half-life longer than wild-type FVIII or a FVIII monomer-dimer hybrid, which consists of two polypeptide chains, a first chain consisting of an amino acid sequence encoding FVIII and an Fc region and a second chain consisting of an Fc region, wherein the method comprises administering the VWF fragment described herein or the chimeric protein described herein to the subject. The FVIII amino acid sequence in the monomer-dimer hybrid can be SQ FVIII or wild-type FVIII.

In one embodiment, the invention is directed to a method of using an adjunct moiety, e.g., a VWF fragment described herein or a chimeric protein comprising the VWF fragment, to prevent or inhibit endogenous VWF interaction with a FVIII protein. In another embodiment, a FVIII protein that is capable of interacting with the VWF fragment is endogenous FVIII. In other embodiments, a FVIII protein that is capable of interacting with the VWF fragment is a FVIII composition separately administered to a subject before or after or simultaneously with the VWF fragment or the chimeric protein comprising the VWF fragment. In other embodiments, a FVIII protein that is capable of binding to the VWF fragment is a FVIII composition administered to a subject together with the VWF fragment or the chimeric protein. In still other embodiments, a FVIII protein that is capable of binding to the VWF fragment is FVIII present with the VWF fragment or associated with the VWF fragment in the chimeric protein. The VWF fragment or the chimeric protein comprising the VWF fragment binds to, or is associated with, the FVIII protein and thus extends the half-life of the FVIII protein bound to the VWF fragment or the chimeric protein. The FVIII protein bound to the VWF fragment or the chimeric protein is shielded or protected from the clearance pathway of VWF and thus has reduced clearance compared to the FVIII protein not bound to the VWF fragment or the chimeric protein. The shielded FVIII protein thus has a longer half-life than a FVIII protein not bound to or associated with the VWF fragment or the chimeric protein. In certain embodiments, the FVIII protein associated with or protected by a VWF fragment or a chimeric protein of the invention is not cleared by a VWF clearance receptor. In other embodiments, the FVIII protein associated with or protected by a VWF fragment or a chimeric protein is cleared from the system slower than the FVIII protein that is not associated with or protected by the VWF fragment.

In one aspect, the VWF fragment of this invention or the chimeric protein comprising the same has reduced clearance from circulation as the VWF fragment or the chimeric protein does not contain a VWF clearance receptor binding site. The VWF fragment prevents or inhibits clearance of FVIII bound to or associated with the VWF fragment from the system through the VWF clearance pathway. The VWF fragments useful for the present invention can also provide at least one or more VWF-like FVIII protection properties that are provided by endogenous VWF. In certain embodiments, the VWF fragments can also mask one or more FVIII clearance receptor binding site, thereby preventing clearance of FVIII by its own clearance pathway.

In another aspect, the VWF fragment or chimeric protein of the invention can be used to treat or prevent a disease or disorder associated with a Type 2N von Willebrand disease (VWD). Type 2N VWD is a qualitative VWF defect resulting from defective VWF binding to FVIII and consequently resulting in low levels of circulating FVIII. Therefore, the VWF fragment or chimeric protein of the invention by binding to or being bound to the FVIII protein not only stabilizes the FVIII protein, but also prevents clearance of the FVIII protein from the circulation.

In some embodiments, the prevention or inhibition of a FVIII protein binding to endogenous VWF by the VWF fragment or chimeric protein can be in vitro or in vivo.

Also provided is a method of increasing the half-life of a FVIII protein comprising administering the VWF fragment or the chimeric protein comprising the VWF fragment and a FVIII protein to a subject in need thereof. The half-life of non-activated FVIII bound to or associated with full-length VWF is about 12 to 14 hours in plasma. In VWD type 3, wherein there is almost no VWF in circulation, the half-life of FVIII is only about six hours, leading to symptoms of mild to moderate hemophilia A in such patients due to decreased concentrations of FVIII. The half-life of the FVIII protein linked to or associated with the VWF fragment of the present invention can increase at least about 1.5 times, 1.6 times, 1.7 times, 1.8 times, 1.9 times, 2.0 times, 2.1 times, 2.2 times, 2.3 times, 2.4 times, 2.6 times, 2.7. times, 2.8 times, 2.9 times, 3.0 times, 3.1 times, 3.2 times, 3.3 times, 3.4 times, 3.5 times, 3.6 times, 3.7 times, 3.8 times, 3.9 times, or 4.0 times higher than the half-life of the non-activated FVIII bound to or associated with full-length VWF. In one embodiment, the half-life of the FVIII protein linked to or associated with the VWF fragment in the chimeric protein increases at least about 2 times, 2.5 times, 3.0 times, 3.5 times, 4.0 times, 4.5 times, 5.0 times, 5.5 times, 6.0 times, 7 times, 8 times, 9 times, or 10 times higher than the half-life of the non-activated FVIII bound to or associated with full-length VWF. In another embodiment, the half-life of the FVIII protein linked to or associated with the VWF fragment in the chimeric protein increases about 2 to about 5 times, about 3 to about 10 times, about 5 to about 15 times, about 10 to about 20 times, about 15 to about 25 times, about 20 to about 30 times, about 25 to about 35 times, about 30 to about 40 times, about 35 to about 45 times higher than the half-life of the non-activated FVIII bound to or associated with full-length VWF. In a specific embodiment, the half-life of the FVIII protein linked to or associated with the VWF fragment in the chimeric protein increases at least about 30, 31, 32, 33, 34, 35, 36, 37, 38, 39, or 40 times higher than the half-life of the wild type FVIII in a FVIII and VWF double knockout mouse. In some embodiments, the half-life of the chimeric protein comprising the VWF fragment fused to a first heterologous moiety, e.g., a first Fc region, and a FVIII protein linked to a second heterologous moiety, e.g., a second Fc region is longer than the half-life of a chimeric protein comprising a FVIII protein and two Fc regions, wherein the FVIII protein is linked to one of the two Fc regions (i.e., FVIII monomer-dimer hybrid). In other embodiments, the half-life of the chimeric protein comprising the VWF fragment fused to a first heterologous moiety, e.g., a first Fc region, and a FVIII protein linked to a second heterologous moiety, e.g., a second Fc region is at least about 1.5 times, 2 times, 2.5 times, 3.5 times, 3.6 times, 3.7 times, 3.8 times, 3.9 times, 4.0 times, 4.5 times, or 5.0 times the half-life of a chimeric protein comprising a FVIII protein and two Fc regions, wherein the FVIII protein is linked to one of the two Fc regions (i.e., FVIII monomer-dimer hybrid).

In some embodiments, as a result of the invention the half-life of the FVIII protein is extended compared to a FVIII protein without the VWF fragment or wildtype FVIII. The half-life of the FVIII protein is at least about 1.5 times, at least about 2 times, at least about 2.5 times, at least about 3 times, at least about 4 times, at least about 5 times, at least about 6 times, at least about 7 times, at least about 8 times, at least about 9 times, at least about 10 times, at least about 11 times, or at least about 12 times longer than the half-life of a FVIII protein without the VWF fragment. In one embodiment, the half-life of FVIII is about 1.5-fold to about 20-fold, about 1.5 fold to about 15 fold, or about 1.5 fold to about 10 fold longer than the half-life of wild-type FVIII. In another embodiment, the half-life of the FVIII is extended about 2-fold to about 10-fold, about 2-fold to about 9-fold, about 2-fold to about 8-fold, about 2-fold to about 7-fold, about 2-fold to about 6-fold, about 2-fold to about 5-fold, about 2-fold to about 4-fold, about 2-fold to about 3-fold, about 2.5-fold to about 10-fold, about 2.5-fold to about 9-fold, about 2.5-fold to about 8-fold, about 2.5-fold to about 7-fold, about 2.5-fold to about 6-fold, about 2.5-fold to about 5-fold, about 2.5-fold to about 4-fold, about 2.5-fold to about 3-fold, about 3-fold to about 10-fold, about 3-fold to about 9-fold, about 3-fold to about 8-fold, about 3-fold to about 7-fold, about 3-fold to about 6-fold, about 3-fold to about 5-fold, about 3-fold to about 4-fold, about 4-fold to about 6 fold, about 5-fold to about 7-fold, or about 6-fold to about 8 fold as compared to wild-type FVIII or a FVIII protein without the VWF fragment. In other embodiments, the half-life of FVIII is at least about 17 hours, at least about 18 hours, at least about 19 hours, at least about 20 hours, at least about 21 hours, at least about 22 hours, at least about 23 hours, at least about 24 hours, at least about 25 hours, at least about 26 hours, at least about 27 hours, at least about 28 hours, at least about 29 hours, at least about 30 hours, at least about 31 hours, at least about 32 hours, at least about 33 hours, at least about 34 hours, at least about 35 hours, at least about 36 hours, at least about 48 hours, at least about 60 hours, at least about 72 hours, at least about 84 hours, at least about 96 hours, or at least about 108 hours. In still other embodiments, the half-life of FVIII is about 15 hours to about two weeks, about 16 hours to about one week, about 17 hours to about one week, about 18 hours to about one week, about 19 hours to about one week, about 20 hours to about one week, about 21 hours to about one week, about 22 hours to about one week, about 23 hours to about one week, about 24 hours to about one week, about 36 hours to about one week, about 48 hours to about one week, about 60 hours to about one week, about 24 hours to about six days, about 24 hours to about five days, about 24 hours to about four days, about 24 hours to about three days, or about 24 hours to about two days.

In some embodiments, the average half-life of the FVIII protein per subject is about 15 hours, about 16 hours, about 17 hours, about 18 hours, about 19 hours, about 20 hours, about 21 hours, about 22 hours, about 23 hours, about 24 hours (1 day), about 25 hours, about 26 hours, about 27 hours, about 28 hours, about 29 hours, about 30 hours, about 31 hours, about 32 hours, about 33 hours, about 34 hours, about 35 hours, about 36 hours, about 40 hours, about 44 hours, about 48 hours (2 days), about 54 hours, about 60 hours, about 72 hours (3 days), about 84 hours, about 96 hours (4 days), about 108 hours, about 120 hours (5 days), about six days, about seven days (one week), about eight days, about nine days, about 10 days, about 11 days, about 12 days, about 13 days, or about 14 days.

In a specific embodiment, a half-life of the chimeric protein of the invention is about two fold longer than the half-life of wild-type FVIII or BDD FVIII. In another embodiment, a half-life of the chimeric protein is about three fold longer than the half-life of wild-type FVIII or BDD FVIII.

In addition, the invention provides a method of treating or preventing a bleeding disease or disorder comprising administering an effective amount of the VWF fragment or the chimeric protein (e.g., a chimeric protein comprising the VWF fragment linked to a first heterologous moiety, e.g., a first Fc region, and a FVIII protein linked to a second heterologous moiety, e.g., a second Fc region, wherein the VWF fragment is bound to or associated with the FVIII protein). In one embodiment, the bleeding disease or disorder is selected from the group consisting of a bleeding coagulation disorder, hemarthrosis, muscle bleed, oral bleed, hemorrhage, hemorrhage into muscles, oral hemorrhage, trauma, trauma capitis, gastrointestinal bleeding, intracranial hemorrhage, intra-abdominal hemorrhage, intrathoracic hemorrhage, bone fracture, central nervous system bleeding, bleeding in the retropharyngeal space, bleeding in the retroperitoneal space, and bleeding in the illiopsoas sheath. In a specific embodiment, the bleeding disease or disorder is hemophilia A.

The VWF fragment and the chimeric protein comprising an adjunct moiety, e.g., the VWF fragment described herein and a FVIII protein prepared by the invention has many uses as will be recognized by one skilled in the art, including, but not limited to methods of treating a subject having a hemostatic disorder and methods of treating a subject in need of a general hemostatic agent. In one embodiment, the invention relates to a method of treating a subject having a hemostatic disorder comprising administering a therapeutically effective amount of the VWF fragment or the chimeric protein.

The FVIII protein portion in the chimeric protein treats or prevents a hemostatic disorder by serving as a cofactor to Factor IX on a negatively charged phospholipid surface, thereby forming a Xase complex. The binding of activated coagulation factors to a phospholipid surface localizes this process to sites of vascular damage. On a phospholipid surface, Factor VIIIa increases the maximum velocity of Factor X activation by Factor IXa, by approximately 200,000-fold, leading to the large second burst of thrombin generation.

The chimeric protein comprising an adjunct moiety, e.g., a VWF fragment, and a FVIII protein can be used to treat any hemostatic disorder. The hemostatic disorders that may be treated by administration of the chimeric protein of the invention include, but are not limited to, hemophilia A, as well as deficiencies or structural abnormalities relating to Factor VIII. In one embodiment, the hemostatic disorder is hemophilia A.

The chimeric protein comprising an adjunct moiety, e.g., a VWF fragment, and a FVIII protein can be used prophylactically to treat a subject with a hemostatic disorder. The chimeric protein of the invention can be used to treat an acute bleeding episode in a subject with a hemostatic disorder. In another embodiment, the hemostatic disorder can be the result of a defective clotting factor, e.g., von Willebrand's factor. In one embodiment, the hemostatic disorder is an inherited disorder. In another embodiment, the hemostatic disorder is an acquired disorder. The acquired disorder can result from an underlying secondary disease or condition. The unrelated condition can be, as an example, but not as a limitation, cancer, an auto-immune disease, or pregnancy. The acquired disorder can result from old age or from medication to treat an underlying secondary disorder (e.g. cancer chemotherapy).

The invention also relates to methods of treating a subject that does not have a congenital hemostatic disorder, but has a secondary disease or condition resulting in acquisition of a hemostatic disorder, e.g., due to development of an anti-FVIII antibody or a surgery. The invention thus relates to a method of treating a subject in need of a general hemostatic agent comprising administering a therapeutically effective amount of the chimeric protein comprising the VWF fragment and a FVIII protein prepared by the present methods.

The present invention is also related to methods of reducing immunogenicity of FVIII or inducing less immunogenicity against FVIII comprising administering an effective amount of the VWF fragment, the chimeric proteins described herein, or the polynucleotides encoding the same.

In one embodiment, the subject in need of a general hemostatic agent is undergoing, or is about to undergo, surgery. The chimeric protein comprising the VWF fragment and a FVIII protein can be administered prior to, during, or after surgery as a prophylactic regimen. The chimeric protein comprising the VWF fragment and a FVIII protein can be administered prior to, during, or after surgery to control an acute bleeding episode.

The chimeric protein comprising the VWF fragment and a FVIII protein can be used to treat a subject having an acute bleeding episode who does not have a hemostatic disorder. The acute bleeding episode can result from severe trauma, e.g., surgery, an automobile accident, wound, laceration gun shot, or any other traumatic event resulting in uncontrolled bleeding. Non limiting examples of bleeding episodes include a bleeding coagulation disorder, hemarthrosis, muscle bleed, oral bleed, hemorrhage, hemorrhage into muscles, oral hemorrhage, trauma, trauma capitis, gastrointestinal bleeding, intracranial hemorrhage, intra-abdominal hemorrhage, intrathoracic hemorrhage, bone fracture, central nervous system bleeding, bleeding in the retropharyngeal space, bleeding in the retroperitoneal space, bleeding in the illiopsoas sheath, and any combinations thereof.

In prophylactic applications, one or more compositions containing the chimeric protein or the VWF fragment of the invention or a cocktail thereof are administered to a patient not already in the disease state to enhance the patient's resistance or reduce symptoms associated with a disease or disorder. Such an amount is defined to be a “prophylactic effective dose.” In therapeutic applications, a relatively high dosage (e.g., from about 1 to 400 mg/kg of polypeptide per dose, with dosages of from 5 to 25 mg being more commonly used for radioimmunoconjugates and higher doses for cytotoxin-drug modified polypeptides) at relatively short intervals is sometimes required until progression of the disease is reduced or terminated, and until the patient shows partial or complete amelioration of symptoms of disease. Thereafter, the patient can be administered a prophylactic regime.

In some embodiments, a chimeric protein, a VWF fragment, or a composition of the invention is used for on-demand treatment, which includes treatment for a bleeding episode, hemarthrosis, muscle bleed, oral bleed, hemorrhage, hemorrhage into muscles, oral hemorrhage, trauma, trauma capitis (head trauma), gastrointestinal bleeding, intracranial hemorrhage, intra-abdominal hemorrhage, intrathoracic hemorrhage, bone fracture, central nervous system bleeding, bleeding in the retropharyngeal space, bleeding in the retroperitoneal space, or bleeding in the illiopsoas sheath. The subject may be in need of surgical prophylaxis, peri-operative management, or treatment for surgery. Such surgeries include, e.g., minor surgery, major surgery, tooth extraction, tonsillectomy, inguinal herniotomy, synovectomy, total knee replacement, craniotomy, osteosynthesis, trauma surgery, intracranial surgery, intra-abdominal surgery, intrathoracic surgery, or joint replacement surgery.

In one embodiment, the chimeric protein comprising the VWF fragment and a FVIII protein is administered intravenously, subcutaneously, intramuscularly, or via any mucosal surface, e.g., orally, sublingually, buccally, nasally, rectally, vaginally or via pulmonary route. The chimeric protein comprising the VWF fragment and a FVIII protein can be implanted within or linked to a biopolymer solid support that allows for the slow release of the chimeric protein to the site of bleeding or implanted into bandage/dressing. The dose of the chimeric protein comprising the VWF fragment and a FVIII protein will vary depending on the subject and upon the particular route of administration used. Dosages can range from 0.1 to 100,000 μg/kg body weight. In one embodiment, the dosing range is 0.1-1,000 μg/kg. In another embodiment, the dosing range is 0.1-500 μg/kg. The protein can be administered continuously or at specific timed intervals. In vitro assays may be employed to determine optimal dose ranges and/or schedules for administration. In vitro assays that measure clotting factor activity are known in the art, e.g., STA-CLOT VIIa-rTF clotting assay or ROTEM clotting assay. Additionally, effective doses may be extrapolated from dose-response curves obtained from animal models, e.g., a hemophiliac dog (Mount et al. 2002, Blood 99 (8): 2670).

Having now described the present invention in detail, the same will be more clearly understood by reference to the following examples, which are included herewith for purposes of illustration only and are not intended to be limiting of the invention. All patents and publications referred to herein are expressly incorporated by reference.

### EXAMPLES

Throughout the examples, the following materials and methods were used unless otherwise stated.

### Materials and Methods

In general, the practice of the present invention employs, unless otherwise indicated, conventional techniques of chemistry, biophysics, molecular biology, recombinant DNA technology, immunology (especially, e.g., antibody technology), and standard techniques in electrophoresis. See, e.g., Sambrook, Fritsch and Maniatis, Molecular Cloning: Cold Spring Harbor Laboratory Press (1989); Antibody Engineering Protocols (Methods in Molecular Biology), 510, Paul, S., Humana Pr (1996); Antibody Engineering: A Practical Approach (Practical Approach Series, 169), McCafferty, Ed., Irl Pr (1996); Antibodies: A Laboratory Manual, Harlow et al., CS.H.L. Press, Pub. (1999); and Current Protocols in Molecular Biology, eds. Ausubel et al., John Wiley & Sons (1992).

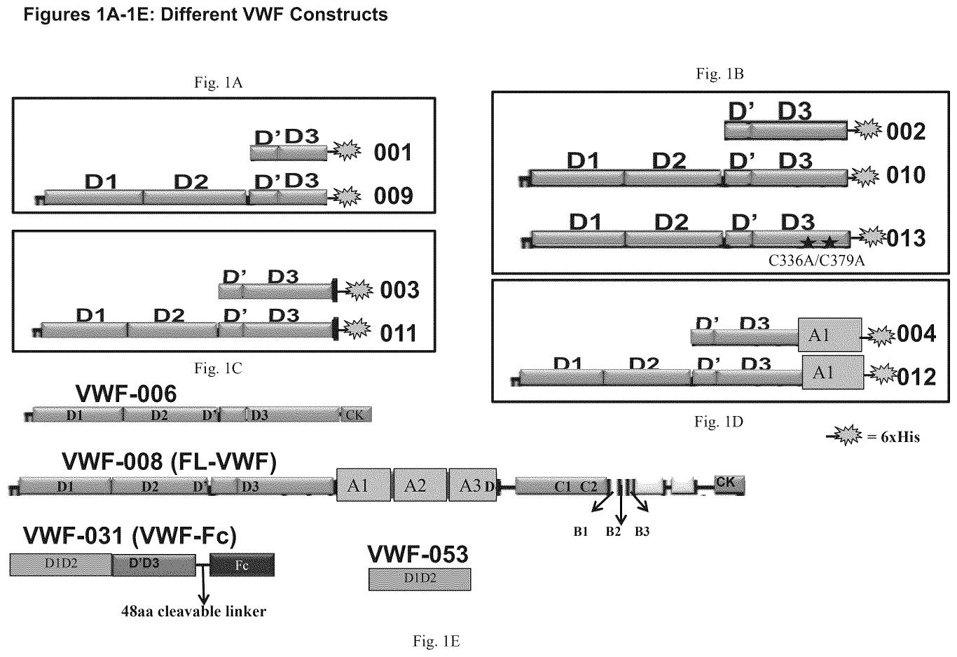

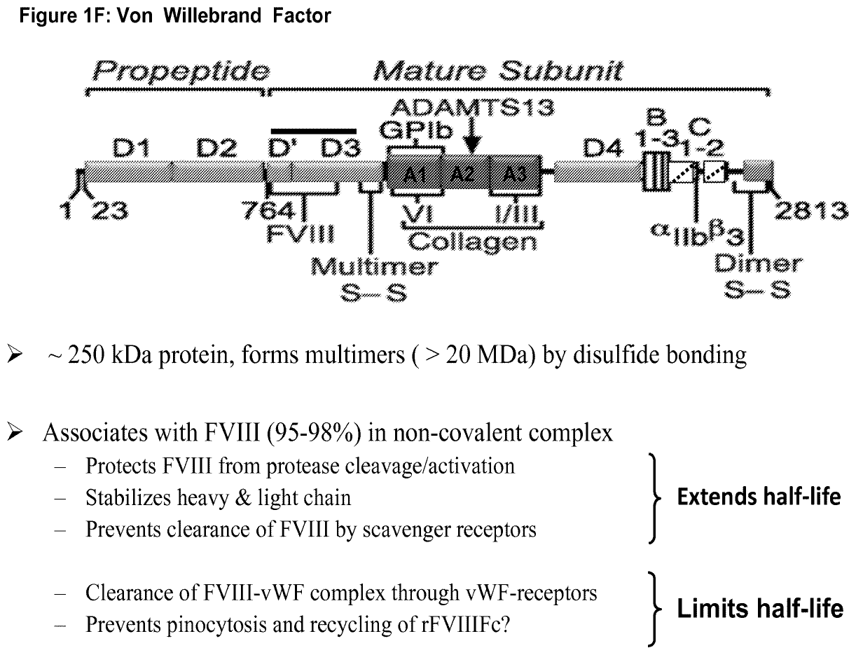

### Example 1: Cloning Different VWF Domains (FIG. 1)

(a) Cloning pSYN-VWF-001, 002, 003 and 004

pSYN-VWF-001 through 004 contain nucleotide sequences encoding VWF fragments, which are amino acids 1-276 (001), amino acids 1-477 (002), amino acids 1-511 (003) and amino acids 1-716 (004) VWF-D′D3A protein sequence. Amino acid numbering represents the mature VWF sequence without propeptide and corresponds to amino acids 764-1039 (001), amino acids 764-1240 (002), amino acids 764-1274 (003), and amino acids 764-1479 (004) of SEQ ID NO: 2, respectively. All four constructs have the FVIII signal peptide at N-terminus, which allows proper secretion of the synthesized protein and followed by a 6×His tag at C-terminus, which is used for protein purification. Above constructs were synthesized by using following primer combinations:

pSYN VWF-001:
ESC48-Fwd-VWF-D′D3 with VIII signal and BsiW1 site
(SEQ ID NO: 57)
TCGCGACGTACGGCCGCCACCATGCAAATAGAGCTCTCCACCTGCTTCTT
TCTGTGCCTTTTGCGATTCTGCTTTAGCCTATCCTGTCGGCCCCCCATG
ESC50-Rev-VWF-partial D′D3 (1-276 amino acid) with
6 His and Not1 site
(SEQ ID NO: 58)
TGACCTCGAGCGGCCGCTCAGTGGTGATGGTGATGATGCAGAGGCACTTT
TCTGGTGTCAGCACACTG
pSYN VWF-002:
ESC48-Fwd-VWF-D′D3 with VIII signal and BsiW1 site
(SEQ ID NO: 59)
TCGCGACGTACGGCCGCCACCATGCAAATAGAGCTCTCCACCTGCTTCTT
TCTGTGCCTTTTGCGATTCTGCTTTAGCCTATCCTGTCGGCCCCCCATG
ESC51-Rev-VWF D′D3 (1-477 amino acid) with 6His
and Not 1 site
(SEQ ID NO: 60)
TGACCTCGAGCGGCCGCTCAGTGGTGATGGTGATGATGCGGCTCCTGGCA
GGCTTCACAGGTGAGGTTGACAAC
pSYN VWF-003:
ESC48-Fwd-VWF-D′D3 with VIII signal and BsiW1 site
(SEQ ID NO: 61)
TCGCGACGTACGGCCGCCACCATGCAAATAGAGCTCTCCACCTGCTTCTT
TCTGTGCCTTTTGCGATTCTGCTTTAGCCTATCCTGTCGGCCCCCCATG
ESC52-Rev-VWF-D′D3 Partial A1 (1-511 amino acids)
with 6His and Not1 site
(SEQ ID NO: 62)
TGACCTCGAGCGGCCGCTCAGTGGTGATGGTGATGATGCCTGCTGCAGTA
GAAATCGTGCAACGGCGGTTC
pSYN VWF-004:
ESC48-Fwd-VWF-D′D3 with VIII signal and BsiW1 site
(SEQ ID NO: 63)
TCGCGACGTACGGCCGCCACCATGCAAATAGAGCTCTCCACCTGCTTCTT
TCTGTGCCTTTTGCGATTCTGCTTTAGCCTATCCTGTCGGCCCCCCATG
ESC53-Rev-VWF-D′D3A1 (1-716 amino acids) with 6His
and Not1 site
(SEQ ID NO: 64)
TGACCTCGAGCGGCCGCTCAGTGGTGATGGTGATGATGGCCCACAGTGAC
TTGTGCCATGTGGGG

Proteins from VWF-001, 002, 003 and 004 Constructs are Supposed to Exists as a Monomer.

A 50 μl PCR reaction was carried out with ESC 48/ESC50, ESC 48/ESC 51, ESC 48/ESC52, ESC48/ESC53 primer combinations and full length VWF plasmid as the template, using the 2 step PCR amplification cycle: 94° C. 2 minutes; 21 cycles of (96° C. 30 seconds, 68° C. 2 minute). Correct sized bands (~960 bp for VWF 001; 1460 for VWF 002, 1520 bp for VWF 003; and 2150 bp for VWF 004) were gel purified with a Gel Extraction kit (Qiagen, Valencia, Calif.) and cloned into the BsiWI and Notl restriction sites of pcDNA 4 to generate pSYN-VWF 001,002,003 and 004, respectively.

(b) Cloning pSYN-VWF-006

pSYN-VWF-006 contains DID2D′D3-CK (cysteine knot) domain of VWF. To clone this construct, synthesis of DNA fragment containing a portion of D3 domain and CK domain was outsourced (Genscript-sequence id number 122026, shown below). A fragment of Genscript construct was sub-cloned into the BamH1/EcoRV digested pSYN-VWF 008, i.e., the vector coding full-length VWF.

Genscript-Sequence number-122026
(SEQ ID NO: 65)
GGATCCTAGTGGGGAATAAGGGATGCAGCCACCCCTCAGTGAAATGCAAG
AAACGGGTCACCATCCTGGTGGAGGGAGGAGAGATTGAGCTGTTTGACGG
GGAGGTGAATGTGAAGAGGCCCATGAAGGATGAGACTCACTTTGAGGTGG
TGGAGTCTGGCCGGTACATCATTCTGCTGCTGGGCAAAGCCCTCTCCGTG
GTCTGGGACCGCCACCTGAGCATCTCCGTGGTCCTGAAGCAGACATACCA
GGAGAAAGTGTGTGGCCTGTGTGGGAATTTTGATGGCATCCAGAACAATG
ACCTCACCAGCAGCAACCTCCAAGTGGAGGAAGACCCTGTGGACTTTGGG
AACTCCTGGAAAGTGAGCTCGCAGTGTGCTGACACCAGAAAAGTGCCTCT
GGACTCATCCCCTGCCACCTGCCATAACAACATCATGAAGCAGACGATGG
TGGATTCCTCCTGTAGAATCCTTACCAGTGACGTCTTCCAGGACTGCAAC
AAGCTGGTGGACCCCGAGCCATATCTGGATGTCTGCATTTACGACACCTG
CTCCTGTGAGTCCATTGGGGACTGCGCCTGCTTCTGCGACACCATTGCTG
CCTATGCCCACGTGTGTGCCCAGCATGGCAAGGTGGTGACCTGGAGGACG
GCCACATTGTGCCCCCAGAGCTGCGAGGAGAGGAATCTCCGGGAGAACGG
GTATGAGTGTGAGTGGCGCTATAACAGCTGTGCACCTGCCTGTCAAGTCA
CGTGTCAGCACCCTGAGCCACTGGCCTGCCCTGTGCAGTGTGTGGAGGGC
TGCCATGCCCACTGCCCTCCAGGGAAAATCCTGGATGAGCTTTTGCAGAC
CTGCGTTGACCCTGAAGACTGTCCAGTGTGTGAGGTGGCTGGCCGGCGTT
TTGCCTCAGGAAAGAAAGTCACCTTGAATCCCAGTGACCCTGAGCACTGC
CAGATTTGCCACTGTGATGTTGTCAACCTCACCTGTGAAGCCTGCCAGGA
GCCGGGAGGCCTGGTGGTGCCTCCCACAGATGCCCCGGTGAGCCCCACCA
CTCTGTATGTGGATGAGACGCTCCAGGATGGCTGTGATACTCACTTCTGC
AAGGTCAATGAGAGAGGAGAGTACTTCTGGGAGAAGAGGGTCACAGGCTG
CCCACCCTTTGATGAACACAAGTGTCTTGCTGAGGGAGGTAAAATTATGA
AAATTCCAGGCACCTGCTGTGACACATGTGAGGAGCCTGAGTGCAACGAC
ATCACTGCCAGGCTGCAGTATGTCAAGGTGGGAAGCTGTAAGTCTGAAGT
AGAGGTGGATATC

(c) Cloning pSYN-VWF-009, 010, 011, 012 and 013

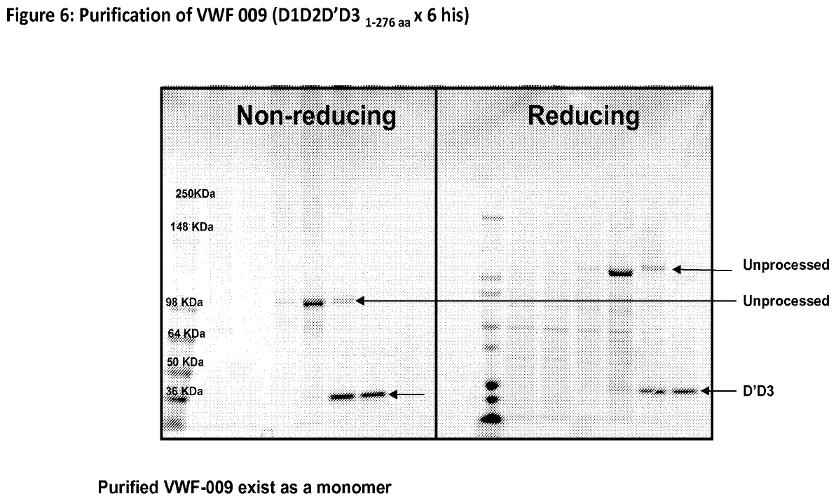

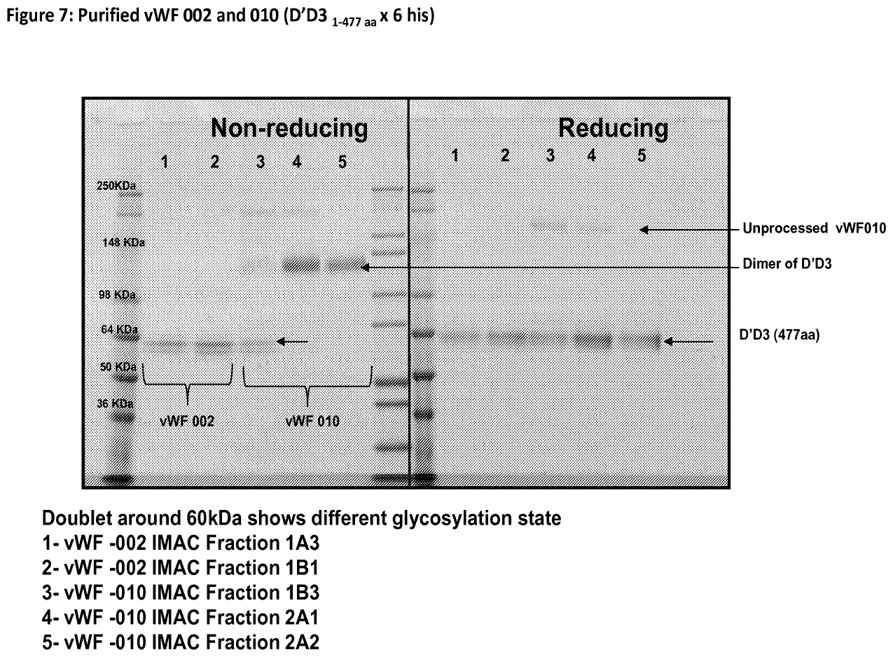

pSYN VWF 008 construct contains the full-length VWF sequence in pcDNA 3.1 (amino acids 1-2813 of SEQ ID NO: 2). It includes 763 amino acid propeptide (i.e., DID2 domains) followed by remaining 2050 amino acids sequence of mature VWF. pSYN-VWF-009, 010, 011 and 012 contain the same coding sequences as VWF 001, 002, 003 and 004, respectively, but additionally has DID2 domains (VWF propeptide) at the N-terminus instead of the FVIII signal peptide. pSYN-VWF-008 has a BamH1 site at Arg907 and Not1 site at the end of coding region (after stop codon). pSYN-VWF-008, 001, 002, 003 and 004 were digested with BamH1 and Not1 restriction enzymes. Inserts from pSYN-VWF-001 (423 bp), pSYN-VWF-002 (1026 bp), pSYN-VWF-003 (1128 bp) and pSYN-VWF-004 (1743 bp) were ligated into bamH1/Not1 digested pSYN-VWF-008 (8242 bp) to obtain pSYN-VWF-009 (D1D2D′D3: amino acid 1-1039 of SEQ ID NO: 2); pSYN-VWF-010 (D1D2D′D3: amino acid 1-1240 of SEQ ID NO: 2); pSYN-VWF-011 (DID2D′D3: amino acid 1-1274 of SEQ ID NO: 2); pSYN-VWF-012 (D1D2D′D3: amino acid 1-1479). All 4 constructs have 6×His tag at the C-terminus. In transfected cells, pSYN-VWF-009, 010, 011, and 012 are synthesized with propeptide, but due to intracellular processing, the secreted products do not contain any propeptide (DID2). The protein expressed from the VWF-009 construct exists as a monomer and the proteins expressed from the VWF-010, 011, and 012 constructs are supposed to exist as dimers, as shown in FIG. 6 and FIG. 7 using VWF-009 and VWF-010 as examples, respectively.

pSYN-VWF-010 was used to generate pSYN-VWF-013, which has two point mutations at C336A and C379A corresponding to SEQ ID NO: 73 (amino acid numbering represents mature VWF sequence without DID2 domain-VWF sequence 2). These mutations are predicted to prevent dimerization of VWF D′D3 domain.

(d) Cloning pSYN-VWF-025 and 029

pSYN-VWF-025 contains wild type D1D2D′D3 sequences of full-length VWF in pLIVE vector while pSYN-VWF-029 contains DID2D′D3 domains with C336A/C379A mutations in pLIVE vector. For cloning pSYN-VWF-025 and 029, the following primer combination was used:

ESC 89-fwd with Nhe1 site =
(SEQ ID NO: 66)
CTCACTATAGGGAGACCCAAGCTGGCTAGCCG
ESC 91-rev with Sal1 =
(SEQ ID NO: 67)
CTGGATCCCGGGAGTCGACTCGTCAGTGGTGATGGTGATGATG

A 50 μl PCR reaction was carried out with ESC 89/ESC91 primer combinations and either pSYN-VWF-010 (for pSYN-VWF-025) or pSYN-VWF-013 (for pSYN-VWF-029) plasmid as the template using the 3 step PCR amplification cycle: 94° C.-2 minutes; 21 cycles of (96° C.-30 seconds, 55° C.-30 second, 68° C.-4 minutes). The expected sized band (~3800 bp) was gel purified with a Gel Extraction kit (Qiagen, Valencia, Calif.) and cloned into the Nhe1 and Sal1 restriction sites of pLIVE-Mirus vector (Invitrogen, Carlsbad, Calif.) to generate pSYN-VWF-025 and 029.

(e) Cloning pSYN-VWF-031

pSYN-VWF-031 is a DID2D′D3(C336A/C379A)-Fc construct which has a 48 amino acid long thrombin cleavable linker (8×GGGGS (SEQ ID NO: 110)+thrombin site) in between the VWF DID2D′D3 (C336A/C379A) and the Fc sequences. To make this construct, VWF-Fc region was amplified from construct pSYN-FVIII-064 (refer FVIII-VWF construct below). pSYN-FVIII-VWF was digested with Xba1 and Nhe1. The resulting insert region of 4165 bp, containing the VWF fragment and Fc region, was used as a template for amplifying the VWF and Fc region by primer combinations LW 22/LW23.

| LW 22-FWD-VWF-D′D3 with FVIII signal sequence and BsiW1 site | |
|---|---|
| (SEQ ID NO: 68) | |
| GCGCCGGCCGTACGATGCAAATAGAGCTCTCCACCTGCTTCTTTCTGTGCCTTTTGC | |
| GATTCTGCTTTAGCCTATCCTGTCGGCCCCCCATG | |
| LW 23-Rev- Fc with stop codon and Not1 site | |
| (SEQ ID NO: 69) | |
| TCATCAATGTATCTTATCATGTCTGAATTCGCGGCCGCTCATTTACC | |
| Nucleotide sequence of VWF 031 | |
| (SEQ ID NO: 108) | |
| 1 | ATGATTCCTG CCAGATTTGC CGGGGTGCTG CTTGCTCTGG CCCTCATTTT |
| 51 | GCCAGGGACC CTTTGTGCAG AAGGAACTCG CGGCAGGTCA TCCACGGCCC |
| 101 | GATGCAGCCT TTTCGGAAGT GACTTCGTCA ACACCTTTGA TGGGAGCATG |
| 151 | TACAGCTTTG CGGGATACTG CAGTTACCTC CTGGCAGGGG GCTGCCAGAA |
| 201 | ACGCTCCTTC TCGATTATTG GGGACTTCCA GAATGGCAAG AGAGTGAGCC |
| 251 | TCTCCGTGTA TCTTGGGGAA TTTTTTGACA TCCATTTGTT TGTCAATGGT |
| 301 | ACCGTGACAC AGGGGGACCA AAGAGTCTCC ATGCCCTATG CCTCCAAAGG |
| 351 | GCTGTATCTA GAAACTGAGG CTGGGTACTA CAAGCTGTCC GGTGAGGCCT |
| 401 | ATGGCTTTGT GGCCAGGATC GATGGCAGCG GCAACTTTCA AGTCCTGCTG |
| 451 | TCAGACAGAT ACTTCAACAA GACCTGCGGG CTGTGTGGCA ACTTTAACAT |
| 501 | CTTTGCTGAA GATGACTTTA TGACCCAAGA AGGGACCTTG ACCTCGGACC |
| 551 | CTTATGACTT TGCCAACTCA TGGGCTCTGA GCAGTGGAGA ACAGTGGTGT |
| 601 | GAACGGGCAT CTCCTCCCAG CAGCTCATGC AACATCTCCT CTGGGGAAAT |
| 651 | GCAGAAGGGC CTGTGGGAGC AGTGCCAGCT TCTGAAGAGC ACCTCGGTGT |
| 701 | TTGCCCGCTG CCACCCTCTG GTGGACCCCG AGCCTTTTGT GGCCCTGTGT |
| 751 | GAGAAGACTT TGTGTGAGTG TGCTGGGGGG CTGGAGTGCG CCTGCCCTGC |
| 801 | CCTCCTGGAG TACGCCCGGA CCTGTGCCCA GGAGGGAATG GTGCTGTACG |
| 851 | GCTGGACCGA CCACAGCGCG TGCAGCCCAG TGTGCCCTGC TGGTATGGAG |
| 901 | TATAGGCAGT GTGTGTCCCC TTGCGCCAGG ACCTGCCAGA GCCTGCACAT |
| 951 | CAATGAAATG TGTCAGGAGC GATGCGTGGA TGGCTGCAGC TGCCCTGAGG |
| 1001 | GACAGCTCCT GGATGAAGGC CTCTGCGTGG AGAGCACCGA GTGTCCCTGC |
| 1051 | GTGCATTCCG GAAAGCGCTA CCCTCCCGGC ACCTCCCTCT CTCGAGACTG |
| 1101 | CAACACCTGC ATTTGCCGAA ACAGCCAGTG GATCTGCAGC AATGAAGAAT |
| 1151 | GTCCAGGGGA GTGCCTTGTC ACTGGTCAAT CCCACTTCAA GAGCTTTGAC |
| 1201 | AACAGATACT TCACCTTCAG TGGGATCTGC CAGTACCTGC TGGCCCGGGA |
| 1251 | TTGCCAGGAC CACTCCTTCT CCATTGTCAT TGAGACTGTC CAGTGTGCTG |
| 1301 | ATGACCGCGA CGCTGTGTGC ACCCGCTCCG TCACCGTCCG GCTGCCTGGC |
| 1351 | CTGCACAACA GCCTTGTGAA ACTGAAGCAT GGGGCAGGAG TTGCCATGGA |
| 1401 | TGGCCAGGAC ATCCAGCTCC CCCTCCTGAA AGGTGACCTC CGCATCCAGC |
| 1451 | ATACAGTGAC GGCCTCCGTG CGCCTCAGCT ACGGGGAGGA CCTGCAGATG |
| 1501 | GACTGGGATG GCCGCGGGAG GCTGCTGGTG AAGCTGTCCC CCGTCTATGC |
| 1551 | CGGGAAGACC TGCGGCCTGT GTGGGAATTA CAATGGCAAC CAGGGCGACG |
| 1601 | ACTTCCTTAC CCCCTCTGGG CTGGCGGAGC CCCGGGTGGA GGACTTCGGG |
| 1651 | AACGCCTGGA AGCTGCACGG GGACTGCCAG GACCTGCAGA AGCAGCACAG |
| 1701 | CGATCCCTGC GCCCTCAACC CGCGCATGAC CAGGTTCTCC GAGGAGGCGT |
| 1751 | GCGCGGTCCT GACGTCCCCC ACATTCGAGG CCTGCCATCG TGCCGTCAGC |
| 1801 | CCGCTGCCCT ACCTGCGGAA CTGCCGCTAC GACGTGTGCT CCTGCTCGGA |
| 1851 | CGGCCGCGAG TGCCTGTGCG GCGCCCTGGC CAGCTATGCC GCGGCCTGCG |
| 1901 | CGGGGAGAGG CGTGCGCGTC GCGTGGCGCG AGCCAGGCCG CTGTGAGCTG |
| 1951 | AACTGCCCGA AAGGCCAGGT GTACCTGCAG TGCGGGACCC CCTGCAACCT |
| 2001 | GACCTGCCGC TCTCTCTCTT ACCCGGATGA GGAATGCAAT GAGGCCTGCC |
| 2051 | TGGAGGGCTG CTTCTGCCCC CCAGGGCTCT ACATGGATGA GAGGGGGGAC |
| 2101 | TGCGTGCCCA AGGCCCAGTG CCCCTGTTAC TATGACGGTG AGATCTTCCA |
| 2151 | GCCAGAAGAC ATCTTCTCAG ACCATCACAC CATGTGCTAC TGTGAGGATG |
| 2201 | GCTTCATGCA CTGTACCATG AGTGGAGTCC CCGGAAGCTT GCTGCCTGAC |
| 2251 | GCTGTCCTCA GCAGTCCCCT GTCTCATCGC AGCAAAAGGA GCCTATCCTG |
| 2301 | TCGGCCCCCC ATGGTCAAGC TGGTGTGTCC CGCTGACAAC CTGCGGGCTG |
| 2351 | AAGGGCTCGA GTGTACCAAA ACGTGCCAGA ACTATGACCT GGAGTGCATG |
| 2401 | AGCATGGGCT GTGTCTCTGG CTGCCTCTGC CCCCCGGGCA TGGTCCGGCA |
| 2451 | TGAGAACAGA TGTGTGGCCC TGGAAAGGTG TCCCTGCTTC CATCAGGGCA |
| 2501 | AGGAGTATGC CCCTGGAGAA ACAGTGAAGA TTGGCTGCAA CACTTGTGTC |
| 2551 | TGTCGGGACC GGAAGTGGAA CTGCACAGAC CATGTGTGTG ATGCCACGTG |
| 2601 | CTCCACGATC GGCATGGCCC ACTACCTCAC CTTCGACGGG CTCAAATACC |
| 2651 | TGTTCCCCGG GGAGTGCCAG TACGTTCTGG TGCAGGATTA CTGCGGCAGT |
| 2701 | AACCCTGGGA CCTTTCGGAT CCTAGTGGGG AATAAGGGAT GCAGCCACCC |
| 2751 | CTCAGTGAAA TGCAAGAAAC GGGTCACCAT CCTGGTGGAG GGAGGAGAGA |
| 2801 | TTGAGCTGTT TGACGGGGAG GTGAATGTGA AGAGGCCCAT GAAGGATGAG |
| 2851 | ACTCACTTTG AGGTGGTGGA GTCTGGCCGG TACATCATTC TGCTGCTGGG |
| 2901 | CAAAGCCCTC TCCGTGGTCT GGGACCGCCA CCTGAGCATC TCCGTGGTCC |
| 2951 | TGAAGCAGAC ATACCAGGAG AAAGTGTGTG GCCTGTGTGG GAATTTTGAT |
| 3001 | GGCATCCAGA ACAATGACCT CACCAGCAGC AACCTCCAAG TGGAGGAAGA |
| 3051 | CCCTGTGGAC TTTGGGAACT CCTGGAAAGT GAGCTCGCAG TGTGCTGACA |
| 3101 | CCAGAAAAGT GCCTCTGGAC TCATCCCCTG CCACCTGCCA TAACAACATC |
| 3151 | ATGAAGCAGA CGATGGTGGA TTCCTCCTGT AGAATCCTTA CCAGTGACGT |
| 3201 | CTTCCAGGAC TGCAACAAGC TGGTGGACCC CGAGCCATAT CTGGATGTCT |
| 3251 | GCATTTACGA CACCTGCTCC TGTGAGTCCA TTGGGGACTG CGCCGCATTC |
| 3301 | TGCGACACCA TTGCTGCCTA TGCCCACGTG TGTGCCCAGC ATGGCAAGGT |
| 3351 | GGTGACCTGG AGGACGGCCA CATTGTGCCC CCAGAGCTGC GAGGAGAGGA |
| 3401 | ATCTCCGGGA GAACGGGTAT GAGGCTGAGT GGCGCTATAA CAGCTGTGCA |
| 3451 | CCTGCCTGTC AAGTCACGTG TCAGCACCCT GAGCCACTGG CCTGCCCTGT |
| 3501 | GCAGTGTGTG GAGGGCTGCC ATGCCCACTG CCCTCCAGGG AAAATCCTGG |
| 3551 | ATGAGCTTTT GCAGACCTGC GTTGACCCTG AAGACTGTCC AGTGTGTGAG |
| 3601 | GTGGCTGGCC GGCGTTTTGC CTCAGGAAAG AAAGTCACCT TGAATCCCAG |
| 3651 | TGACCCTGAG CACTGCCAGA TTTGCCACTG TGATGTTGTC AACCTCACCT |
| 3701 | GTGAAGCCTG CCAGGAGCCG ATATCTGGCG GTGGAGGTTC CGGTGGCGGG |
| 3751 | GGATCCGGCG GTGGAGGTTC CGGCGGTGGA GGTTCCGGTG GCGGGGGATC |
| 3801 | CGGTGGCGGG GGATCCCTGG TCCCCCGGGG CAGCGGCGGT GGAGGTTCCG |
| 3851 | GTGGCGGGGG ATCCGACAAA ACTCACACAT GCCCACCGTG CCCAGCTCCA |
| 3901 | GAACTCCTGG GCGGACCGTC AGTCTTCCTC TTCCCCCCAA AACCCAAGGA |
| 3951 | CACCCTCATG ATCTCCCGGA CCCCTGAGGT CACATGCGTG GTGGTGGACG |
| 4001 | TGAGCCACGA AGACCCTGAG GTCAAGTTCA ACTGGTACGT GGACGGCGTG |
| 4051 | GAGGTGCATA ATGCCAAGAC AAAGCCGCGG GAGGAGCAGT ACAACAGCAC |
| 4101 | GTACCGTGTG GTCAGCGTCC TCACCGTCCT GCACCAGGAC TGGCTGAATG |
| 4151 | GCAAGGAGTA CAAGTGCAAG GTCTCCAACA AAGCCCTCCC AGCCCCCATC |
| 4201 | GAGAAAACCA TCTCCAAAGC CAAAGGGCAG CCCCGAGAAC CACAGGTGTA |
| 4251 | CACCCTGCCC CCATCCCGGG ATGAGCTGAC CAAGAACCAG GTCAGCCTGA |
| 4301 | CCTGCCTGGT CAAAGGCTTC TATCCCAGCG ACATCGCCGT GGAGTGGGAG |
| 4351 | AGCAATGGGC AGCCGGAGAA CAACTACAAG ACCACGCCTC CCGTGTTGGA |
| 4401 | CTCCGACGGC TCCTTCTTCC TCTACAGCAA GCTCACCGTG GACAAGAGCA |
| 4451 | GGTGGCAGCA GGGGAACGTC TTCTCATGCT CCGTGATGCA TGAGGCTCTG |
| 4501 | CACAACCACT ACACGCAGAA GAGCCTCTCC CTGTCTCCGG GTAAATGA |
| Protein sequence of VWF 031 | |
| (SEQ ID NO: 109) | |
| 1 | MIPARFAGVL LALALILPGT LCAEGTRGRS STARCSLFGS DFVNTFDGSM |
| 51 | YSFAGYCSYL LAGGCQKRSF SIIGDFQNGK RVSLSVYLGE FFDIHLFVNG |
| 101 | TVTQGDQRVS MPYASKGLYL ETEAGYYKLS GEAYGFVARI DGSGNFQVLL |
| 151 | SDRYFNKTCG LCGNFNIFAE DDFMTQEGTL TSDPYDFANS WALSSGEQWC |
| 201 | ERASPPSSSC NISSGEMQKG LWEQCQLLKS TSVFARCHPL VDPEPFVALC |
| 251 | EKTLCECAGG LECACPALLE YARTCAQEGM VLYGWTDHSA CSPVCPAGME |
| 301 | YRQCVSPCAR TCQSLHINEM CQERCVDGCS CPEGQLLDEG LCVESTECPC |
| 351 | VHSGKRYPPG TSLSRDCNTC ICRNSQWICS NEECPGECLV TGQSHFKSFD |
| 401 | NRYFTFSGIC QYLLARDCQD HSFSIVIETV QCADDRDAVC TRSVTVRLPG |
| 451 | LHNSLVKLKH GAGVAMDGQD IQLPLLKGDL RIQHTVTASV RLSYGEDLQM |
| 501 | DWDGRGRLLV KLSPVYAGKT CGLCGNYNGN QGDDFLTPSG LAEPRVEDFG |
| 551 | NAWKLHGDCQ DLQKQHSDPC ALNPRMTRFS EEACAVLTSP TFEACHRAVS |
| 601 | PLPYLRNCRY DVCSCSDGRE CLCGALASYA AACAGRGVRV AWREPGRCEL |
| 651 | NCPKGQVYLQ CGTPCNLTCR SLSYPDEECN EACLEGCFCP PGLYMDERGD |
| 701 | CVPKAQCPCY YDGEIFQPED IFSDHHTMCY CEDGFMHCTM SGVPGSLLPD |
| 751 | AVLSSPLSHR SKRSLSCRPP MVKLVCPADN LRAEGLECTK TCQNYDLECM |
| 801 | SMGCVSGCLC PPGMVRHENR CVALERCPCF HQGKEYAPGE TVKIGCNTCV |
| 851 | CRDRKWNCTD HVCDATCSTI GMAHYLTFDG LKYLFPGECQ YVLVQDYCGS |
| 901 | NPGTFRILVG NKGCSHPSVK CKKRVTILVE GGEIELFDGE VNVKRPMKDE |
| 951 | THFEVVESGR YIILLLGKAL SVVWDRHLSI SVVLKQTYQE KVCGLCGNFD |
| 1001 | GIQNNDLTSS NLQVEEDPVD FGNSWKVSSQ CADTRKVPLD SSPATCHNNI |
| 1051 | MKQTMVDSSC RILTSDVFQD CNKLVDPEPY LDVCIYDTCS CESIGDCAAF |
| 1101 | CDTIAAYAHV CAQHGKVVTW RTATLCPQSC EERNLRENGY EAEWRYNSCA |
| 1151 | PACQVTCQHP EPLACPVQCV EGCHAHCPPG KILDELLQTC VDPEDCPVCE |
| 1201 | VAGRRFASGK KVTLNPSDPE HCQICHCDVV NLTCEACQEP ISGGGGSGGG |
| 1251 | GSGGGGSGGG GSGGGGSGGG GSLVPRGSGG GGSGGGGSDK THTCPPCPAP |
| 1301 | ELLGGPSVFL FPPKPKDTLM ISRTPEVTCV VVDVSHEDPE VKFNWYVDGV |
| 1351 | EVHNAKTKPR EEQYNSTYRV VSVLTVLHQD WLNGKEYKCK VSNKALPAPI |
| 1401 | EKTISKAKGQ PREPQVYTLP PSRDELTKNQ VSLTCLVKGF YPSDIAVEWE |
| 1451 | SNGQPENNYK TTPPVLDSDG SFFLYSKLTV DKSRWQQGNV FSCSVMHEAL |
| 1501 | HNHYTQKSLS LSPGK* |

| DNA | |
|---|---|
| construct | Linker between VWF and Fc |
| VWF035 | 73 aa = IS{11X(GGGGS)}LVPRGSGGGGSGGGGS |
| | (SEQ ID NO: 96) |
| VWF036 | 98 aa = IS{16X(GGGGS)}LVPRGSGGGGSGGGGS |
| | (SEQ ID NO: 97) |

VWF = D′D3 (1-477 aa with C336A/C379A)

The PCR product obtained from LW22/LW23 amplification (~2300 bp) was cloned in BsiW1/Not1 digested pSYN-VWF-002 to obtain pSYN-VWF-014 intermediate. pSYN-VWF-014 contains FVIII signal peptide-D′D3-20 amino acid thrombin cleavable linker followed by the Fc region.

To generate the DID2D′D3-Fc construct, the DID2D′D3 region was amplified from pSYN-VWF-013 using primer combination LW24/LW27 by standard PCR method.

LW24-Fwd-VWF D1D2D′D3 cloning oligo with BsiW1 site
(SEQ ID NO: 70)
GCGCCGGCCGTACGATGATTCCTGCCAGATTTGCCGGGGTG
LW27-Rev-VWF D′D3 oligo with EcoRV
(SEQ ID NO: 71)
CCACCGCCAGATATCGGCTCCTGGCAGGCTTCACAGGTGAG

The PCR product obtained from LW22/LW23 amplification (~3750 bp) was cloned in BsiW1/EcoRV digested pSYN-VWF-014 to obtain pSYN-VWF-015 intermediate. The linker length between the VWF fragment and Fc region was changed to obtain pSYN-VWF-031.

Full length VWF protein sequence is shown at Table 1.

| VWF-DID2D′D3 protein sequence 1b | |
|---|---|
| (SEQ ID NO: 72) | |
| 1 | MIPARFAGVL LALALILPGT LCAEGTRGRS STARCSLFGS DFVNTFDGSM |
| 51 | YSFAGYCSYL LAGGCQKRSF SIIGDFQNGK RVSLSVYLGE FFDIHLFVNG |
| 101 | TVTQGDQRVS MPYASKGLYL ETEAGYYKLS GEAYGFVARI DGSGNFQVLL |
| 151 | SDRYFNKTCG LCGNFNIFAE DDFMTQEGTL TSDPYDFANS WALSSGEQWC |
| 201 | ERASPPSSSC NISSGEMQKG LWEQCQLLKS TSVFARCHPL VDPEPFVALC |
| 251 | EKTLCECAGG LECACPALLE YARTCAQEGM VLYGWTDHSA CSPVCPAGME |
| 301 | YRQCVSPCAR TCQSLHINEM CQERCVDGCS CPEGQLLDEG LCVESTECPC |
| 351 | VHSGKRYPPG TSLSRDCNTC ICRNSQWICS NEECPGECLV TGQSHFKSFD |
| 401 | NRYFTFSGIC QYLLARDCQD HSFSIVIETV QCADDRDAVC TRSVTVRLPG |
| 451 | LHNSLVKLKH GAGVAMDGQD IQLPLLKGDL RIQHTVTASV RLSYGEDLQM |
| 501 | DWDGRGRLLV KLSPVYAGKT CGLCGNYNGN QGDDFLTPSG LAEPRVEDFG |
| 551 | NAWKLHGDCQ DLQKQHSDPC ALNPRMTRFS EEACAVLTSP TFEACHRAVS |
| 601 | PLPYLRNCRY DVCSCSDGRE CLCGALASYA AACAGRGVRV AWREPGRCEL |
| 651 | NCPKGQVYLQ CGTPCNLTCR SLSYPDEECN EACLEGCFCP PGLYMDERGD |
| 701 | CVPKAQCPCY YDGEIFQPED IFSDHHTMCY CEDGFMHCTM SGVPGSLLPD |
| 751 | AVLSSPLSHR SKRSLSCRPP MVKLVCPADN LRAEGLECTK TCQNYDLECM |
| 801 | SMGCVSGCLC PPGMVRHENR CVALERCPCF HQGKEYAPGE TVKIGCNTCV |
| 851 | CRDRKWNCTD HVCDATCSTI GMAHYLTFDG LKYLFPGECQ YVLVQDYCGS |
| 901 | NPGTFRILVG NKGCSHPSVK CKKRVTILVE GGEIELFDGE VNVKRPMKDE |
| 951 | THFEVVESGR YIILLLGKAL SVVWDRHLSI SVVLKQTYQE KVCGLCGNFD |
| 1001 | GIQNNDLTSS NLQVEEDPVD FGNSWKVSSQ CADTRKVPLD SSPATCHNNI |
| 1051 | MKQTMVDSSC RILTSDVFQD CNKLVDPEPY LDVCIYDTCS CESIGDCACF |
| 1101 | CDTIAAYAHV CAQHGKVVTW RTATLCPQSC EERNLRENGY ECEWRYNSCA |
| 1151 | PACQVTCQHP EPLACPVQCV EGCHAHCPPG KILDELLQTC VDPEDCPVCE |
| 1201 | VAGRRFASGK KVTLNPSDPE HCQICHCDVV NLTCEACQEP* |
| VWF-D′D3 protein sequence 2 | |
| (SEQ ID NO: 73) | |
| 1 | SLSCRPPMVK LVCPADNLRA EGLECTKTCQ NYDLECMSMG CVSGCLCPPG |
| 51 | MVRHENRCVA LERCPCFHQG KEYAPGETVK IGCNTCVCRD RKWNCTDHVC |
| 101 | DATCSTIGMA HYLTFDGLKY LFPGECQYVL VQDYCGSNPG TFRILVGNKG |
| 151 | CSHPSVKCKK RVTILVEGGE IELFDGEVNV KRPMKDETHF EVVESGRYII |
| 201 | LLLGKALSVV WDRHLSISVV LKQTYQEKVC GLCGNFDGIQ NNDLTSSNLQ |
| 251 | VEEDPVDFGN SWKVSSQCAD TRKVPLDSSP ATCHNNIMKQ TMVDSSCRIL |
| 301 | TSDVFQDCNK LVDPEPYLDV CIYDTCSCES IGDCACFCDT IAAYAHVCAQ |
| 351 | HGKVVTWRTA TLCPQSCEER NLRENGYECE WRYNSCAPAC QVTCQHPEPL |
| 401 | ACPVQCVEGC HAHCPPGKIL DELLQTCVDP EDCPVCEVAG RRFASGKKVT |
| 451 | LNPSDPEHCQ ICHCDVVNLT CEACQEP |

### Example 2: Heterodimeric Constructs Comprising FVIII-Fc and VWF-D′D3 Domain at the Amino Terminus of the Second Fc Chain (FVIII-VWF-Fc Heterodimer, FIG. 2)

(a) Cloning of pSYN-FVIII-064

The FVIII-064 plasmid comprises a single chain FC (scFc) scaffold with enzyme cleavage sites which are processed during synthesis in a cell. The construct has a FVIII binding domain of full-length VWF (D′D3).

Plasmid (pSYN-FVIII-064) was designed for the expression FVIII-Fc and VWF-Fc heterodimer, where the D′D3 domains to bind FVIII and prevents FVIII interaction with phospholipids and activated protein C and/or preventing or inhibiting binding to endogenous VWF. Protein from pSYN-FVIII-064 is expressed in the cell as a single polypeptide where the C-terminus of the FVIII-Fc subunit is linked to the N-terminus of the VWF D′D3-Fc subunit by a 6× (GGGGS) polypeptide linker (SEQ ID NO: 74). In addition, RRRRS (SEQ ID NO: 75) and RKRRKR (SEQ ID NO: 76) sequences were inserted at the 5′ and 3′ end of the polypeptide linker, respectively, for intracellular cleavage by proprotein convertases following the last Arg at each sequence. Hence, the cells can express a double chain FVIII-Fc/D′D3-Fc heterodimer where the FVIII-Fc chain has a RRRRS sequence (SEQ ID NO: 75) at the C-terminus, but the remainder of the linker sequence has been removed. Another 3× (GGGGS) polypeptide linker (SEQ ID NO: 28) along with a thrombin cleavage site is introduced in between the VWF domains and the Fc region to facilitate release of the VWF fragment from FVIII once the FVIII-VWF hetero-dimeric protein is activated by thrombin allowing interaction of FVIII with other clotting factors.

Synthesis of the DNA fragments containing a portion of the first Fc region followed by a 6× (GGGGS) (SEQ ID NO: 74), the VWF-D′D3 domain (1-477aa; C336A/C379A mutation), 3× (GGGGS) (SEQ ID NO:28), the thrombin cleavage site and a portion of the second Fc was outsourced (Genscript-sequence number 103069, shown below). A fragment of Genscript construct was sub cloned into the SalI/RsRII digested pSYN-FVIII-049, which is FVIII-Fc construct with a cleavable linker in between two Fc domains.

Genscript-Sequence number 103069 (SEQ ID NO: 82):
CCGTCGACAAGAGCAGGTGGCAGCAGGGGAACGTCTTCTCATGCTCCGT
GATGCATGAGGCTCTGCACAACCACTACACGCAGAAGAGCCTCTCCCTG
TCTCCGGGTAAACGGCGCCGCCGGAGCGGTGGCGGCGGATCAGGTGGGG
GTGGATCAGGCGGTGGAGGTTCCGGTGGCGGGGGATCCGGCGGTGGAGG
TTCCGGTGGGGGTGGATCAAGGAAGAGGAGGAAGAGAAGCCTATCCTGT
CGGCCCCCCATGGTCAAGCTGGTGTGTCCCGCTGACAACCTGCGGGCTG
AAGGGCTCGAGTGTACCAAAACGTGCCAGAACTATGACCTGGAGTGCAT
GAGCATGGGCTGTGTCTCTGGCTGCCTCTGCCCCCCGGGCATGGTCCGG
CATGAGAATCGATGTGTGGCCCTGGAAAGGTGTCCCTGCTTCCATCAGG
GCAAGGAGTATGCCCCTGGAGAAACAGTGAAGATTGGCTGCAACACTTG
TGTCTGTCGGGACCGGAAGTGGAACTGCACAGACCATGTGTGTGATGCC
ACGTGCTCCACGATCGGCATGGCCCACTACCTCACCTTCGACGGGCTCA
AATACCTGTTCCCCGGGGAGTGCCAGTACGTTCTGGTGCAGGATTACTG
CGGCAGTAACCCTGGGACCTTTCGGATCCTAGTGGGGAATAAGGGATGC
AGCCACCCCTCAGTGAAATGCAAGAAACGGGTCACCATCCTGGTGGAGG
GAGGAGAGATTGAGCTGTTTGACGGGGAGGTGAATGTGAAGAGGCCCAT
GAAGGATGAGACTCACTTTGAGGTGGTGGAGTCTGGCCGGTACATCATT
CTGCTGCTGGGCAAAGCCCTCTCCGTGGTCTGGGACCGCCACCTGAGCA
TCTCCGTGGTCCTGAAGCAGACATACCAGGAGAAAGTGTGTGGCCTGTG
TGGGAATTTTGATGGCATCCAGAACAATGACCTCACCAGCAGCAACCTC
CAAGTGGAGGAAGACCCTGTGGACTTTGGGAACTCCTGGAAAGTGAGCT
CGCAGTGTGCTGACACCAGAAAAGTGCCTCTGGACTCATCCCCTGCCAC
CTGCCATAACAACATCATGAAGCAGACGATGGTGGATTCCTCCTGTAGA
ATCCTTACCAGTGACGTCTTCCAGGACTGCAACAAGCTGGTGGACCCCG
AGCCATATCTGGATGTCTGCATTTACGACACCTGCTCCTGTGAGTCCAT
TGGGGACTGCGCCGCATTCTGCGACACCATTGCTGCCTATGCCCACGTG
TGTGCCCAGCATGGCAAGGTGGTGACCTGGAGGACGGCCACATTGTGCC
CCCAGAGCTGCGAGGAGAGGAATCTCCGGGAGAACGGGTATGAGGCTGA
GTGGCGCTATAACAGCTGTGCACCTGCCTGTCAAGTCACGTGTCAGCAC
CCTGAGCCACTGGCCTGCCCTGTGCAGTGTGTGGAGGGCTGCCATGCCC
ACTGCCCTCCAGGGAAAATCCTGGATGAGCTTTTGCAGACCTGCGTTGA
CCCTGAAGACTGTCCAGTGTGTGAGGTGGCTGGCCGGCGTTTTGCCTCA
GGAAAGAAAGTCACCTTGAATCCCAGTGACCCTGAGCACTGCCAGATTT
GCCACTGTGATGTTGTCAACCTCACCTGTGAAGCCTGCCAGGAGCCGAT
CGATGGCGGTGGAGGTTCCGGTGGCGGGGGATCCCTGGTCCCCCGGGGC
AGCGGAGGCGACAAAACTCACACATGCCCACCGTGCCCAGCTCCAGAAC
TCCTGGGCGGACCGTCA

(b) Cloning of pSYN-FVIII-065

The FVIII-065 plasmid comprises the first 276 amino acids of the D′D3 domain of VWF attached to a second Fc region. The VWF fragment was PCR amplified from full-length VWF plasmid pSYN-VWF-008 by using primer combinations ESC17 and ESC41.

ESC17-Fwd-VWF cloning oligo with Cla1
(SEQ ID NO: 77)
GTCCGGCATGAGAATCGATGTGTG
ESC41-Rev-VWF with EcoRV
(SEQ ID NO: 78)
CCTCCACCGCCAGATATCAGAGGCACTTTTC

The expected sized band (~692 bp) was gel purified with a Gel Extraction kit (Qiagen, Valencia, Calif.) and cloned into the Cla1 and EcoRV sites of pSYN-FVIII-064 to generate pSYN-FVIII-065.

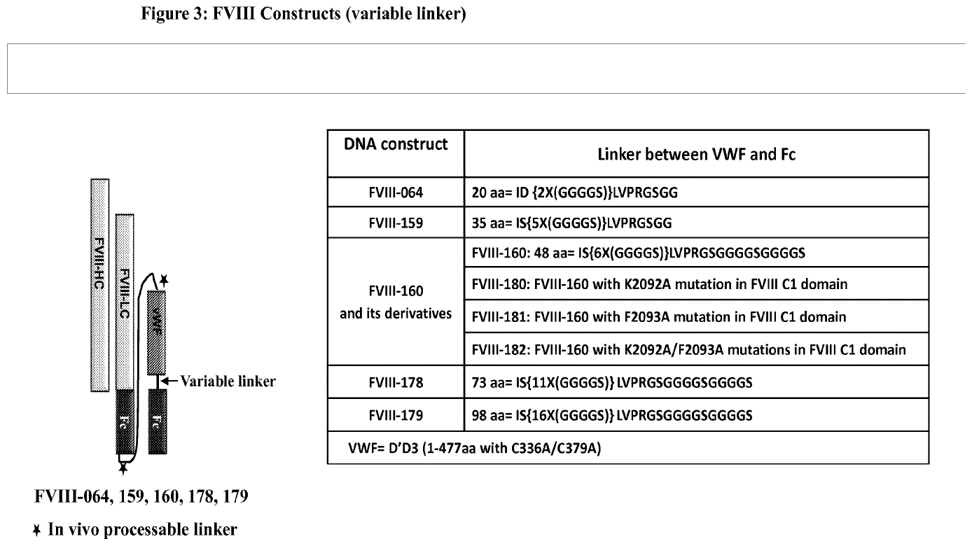

### Example 3: Cloning of pSYN-FVIII-159, 160, 178, 179 (FIG. 3)

In order to vary the linker length between the VWF fragment and Fc region, an EcoRV site was introduced at the junction of VWF and the beginning of 20 amino acid linker in pSYN-FVIII-064, variable size linkers were then used to replace the 20 aa linker in PSYN-FVIII-064. The new DNA constructs are: pSYN-FVIII-159, 160, 178, and 179 which contains 35 aa, 48 aa, 73 aa and 98 aa linkers, respectively.

To insert a 35 amino acid linker in pSYN-FVIII-159, two oligos (ESC78-105 bp and ESC79-107 bp) were ordered from Integrated DNA Technologies, Inc (Coralville, IA). Oligos were annealed and extended using a standard PCR method:

### Primers:

ESC78-Fwd with EcoRV site
(SEQ ID NO: 79)
AAAGTGCCTCTGATATCTGGCGGTGGAGGTTCCGGTGGCGGGGGATCCG
GTGGCGGGGGATCCGGTGGCGGGGGATCCGGTGGCGGGGGATCCCTGGT
CCCCCGG
ESC79-Rev with RsRII site
(SEQ ID NO: 80)
GAAGAGGAAGACTGACGGTCCGCCCAGGAGTTCTGGAGCTGGGCACGGT
GGGCATGTGTGAGTTTTGTCGCCTCCGCTGCCCCGGGGGACCAGGGATC
CCCCGCCAC

A 50 μl PCR oligo annealing and extension reaction was carried out with ESC78/ESC79 primer combo using the 3 step PCR amplification cycle: 25 cycles of (96° C. 30 seconds, 55° C. 30 seconds, 68° C. 30 seconds). The expected sized band (~186 bp) was gel purified with a Gel Extraction kit (Qiagen, Valencia, Calif.) and cloned into the EcoRV and RsRII restriction sites of pSYN-FVIII-064 to generate pSYN-FVIII-159.

(b) Cloning pSYN-FVIII-160, 178, and 179

pSYN-VIII-160 has a 48 amino acids linker in between the VWF fragment and the Fc region. Synthesis of DNA fragment coding for 48 amino acids linker (ISGG GGSGGGGGGGGSGGGGSGGGGSGGGGSLVPRGSGGGGSGGGGS) (SEQ ID NO: 81) and a portion of the Fc region was outsourced (Genscript-Sequence no-132601, shown below). A fragment of the Genscript construct was sub cloned into the EcoRV/RsRII digested pSYN-FVIII-0159 (mentioned above).

AAAGTGCCTCTGATATCTGGCGGTGGAGGTTCCGGTGGCGGGGGATCCG
GCGGTGGAGGTTCCGGCGGTGGAGGTTCCGGTGGCGGGGGATCCGGTGG
CGGGGGATCCCTGGTCCCCCGGGGCAGCGGCGGTGGAGGTTCCGGTGGC
GGGGGATCCGACAAAACTCACACATGCCCACCGTGCCCAGCTCCAGAAC
TCCTGGGCGGACCGTCAGTCTTCC

pSYN-VIII-178 has a 73 amino acids linker in between the VWF fragment and the Fc region. Synthesis of DNA fragment coding for 73 amino acids linker (ISGGGGSGGGGSGGGGSGGGGSGGGGSGGGGSGGGGGGGGSGGGGSGGGGSGGGGSLVP RGSGGGGSGGGGS) (SEQ ID NO: 84) and a portion of Fc region was outsourced (Genscript-Sequence no-144849, shown below). A fragment of Genscript construct was sub cloned into the EcoRV/RsRII digested pSYN-FVIII-0159 (mentioned above).

GCCTGCCAGGAGCCGATATCTGGCGGTGGAGGTTCCGGTGGCGGGGGAT
CCGGCGGTGGAGGTTCCGGCGGTGGAGGTTCCGGTGGCGGGGGATCCGG
CGGTGGAGGTTCCGGTGGCGGGGGATCCGGCGGTGGAGGTTCCGGCGGT
GGAGGTTCCGGTGGCGGGGGATCCGGTGGCGGGGGATCCCTGGTCCCCC
GGGGCAGCGGCGGTGGAGGTTCCGGTGGCGGGGGATCCGACAAAACTCA
CACATGCCCCCGTGCCCAGCTCCAGAACTCCTGGGCGGACCGTCAGTCT
TCCTC

pSYN-VIII-179 has a 98 amino acids linker in between the VWF fragment and the Fc region. Synthesis of DNA fragment coding for 98 amino acids linker (ISGGGGSGGGGSGGGGSGGGGSGGGGSGGGGSGGGGSGGGGSGGGGSGGGGSGGGGSGG GGSGGGGSGGGGSGGGGSGGGGSLVPRGSGGGGSGGGGS) (SEQ ID NO: 86) and a portion of Fc region was outsourced (Genscript-Sequence no-144849shown below). A fragment of Genscript construct was sub cloned into the EcoRV/RsRII digested pSYN-FVIII-0159 (mentioned above).

GCCTGCCAGGAGCCGATATCTGGCGGTGGAGGTTCCGGTGGCGGGGGAT
CCGGCGGTGGAGGTTCCGGCGGTGGAGGTTCCGGTGGCGGGGGATCCGG
CGGTGGAGGTTCCGGTGGCGGGGGATCCGGCGGTGGAGGTTCCGGCGGT
GGAGGTTCCGGTGGCGGGGGATCCGGCGGTGGAGGTTCCGGTGGCGGGG
GATCCGGCGGTGGAGGTTCCGGCGGTGGAGGTTCCGGTGGCGGGGGATC
CGGTGGCGGGGGATCCCTGGTCCCCCGGGGCAGCGGCGGTGGAGGTTCC
GGTGGCGGGGGATCCGACAAAACTCACACATGCCCACCGTGCCCAGCTC
CAGAACTCCTGGGCGGACCGTCAGTCTTCCTCTTCCC

Cloning of pSYN-FVIII-180, 181, and 182

pSYN-FVIII-180, 181, and 182 were constructed from pSYN-FVIII-160. K2093A or F2093A or K2093A/F2093A mutations were introduced into the C1 domain of FVIII in pSYN-FVIII-160 to form pSYN-FVIII-180, pSYN-FVIII-181 and pSYN-FVIII-182 respectively.

### FVIII-VWF-Fc Heterodimer Protein Sequence (SEQ ID NO: 88)

(FVIII sequence amino acid position 1-1457; underlined region represents Fc region; curvy underline represents cleavable linker in between first Fc and VWF fragment; double underlined region represents VWF fragment; bold region represents variable length cleavable linker in between VWF fragment and Fc. The linker length varies in FVIII-064, 159, 160, 178, and 179 constructs).

| | |
|---|---|
| 1 | MQIELSTCFF LCLLRFCFSA TRRYYLGAVE LSWDYMQSDL GELPVDAREP |
| 51 | PRVPKSFPFN TSVVYKKTLF VEFTDHLENI AKPRPPWMGL LGPTIQAEVY |
| 101 | DTVVITLKNM ASHPVSLHAV GVSYWKASEG AEYDDQTSQR EKEDDKVFPG |
| 151 | GSHTYVWQVL KENGPMASDP LCLTYSYLSH VDLVKDLNSG LIGALLVCRE |
| 201 | GSLAKEKTQT LHKFILLFAV FDEGKSWHSE TKNSLMQDRD AASARAWPKM |
| 251 | HTVNGYVNRS LPGLIGCHRK SVYWHVIGMG TTPEVHSIFL EGHTFLVRNH |
| 301 | RQASLEISPI TELTAQTLLM DLGQFLLFCH ISSHQHDGME AYVKVDSCPE |
| 351 | EPQLRMKNNE EAEDYDDDLT DSEMDVVRED DDNSPSFIQI RSVAKKHPKT |
| 401 | WVHYIAAEEE DWDYAPLVLA PDDRSYKSQY LNNGPORIGR KYKKVREMAY |
| 451 | TDETFKTREA IQHESGILGP LLYGEVGDTL LIIFKNQASR PYNIYPHGIT |
| 501 | DVRPLYSRRL PKGVKHLKDF PILPGEIFKY KWTVTVEDGP TKSDPRCLTR |
| 551 | YYSSFVNMER DLASGLIGPL LICYKESVDQ RGNQIMSDKR NVILESVEDE |
| 601 | NRSWYLTENI QRFLPNPAGV QLEDPEFQAS NIMHSINGYV FDSLQLSVCL |
| 651 | HEVAYWYILS IGAQTDFLSV FFSGYTFKHK MVYEDTLTLF PESGETVEMS |
| 701 | MENPGLWILG CHNSDERNRG MTALLKVSSC DKNTGDYYED SYEDISAYLL |
| 751 | SKNNAIEPRS FSQNPPVLKR HQREITRTTL QSDQEEIDYD DTISVEMKKE |
| 801 | DEDIYDEDEN QSPRSFQKKT RHYFIAAVER LWDYGMSSSP HVLRNRAQSG |
| 851 | SVPQFKKVVF QEFTDGSFTQ PLYRGELNEH LGLLGPYIRA EVEDNIMVTE |
| 901 | RNQASRPYSF YSSLISYEED QRQGAEPRKN FVKPNETKTY FWKVQHHMAP |
| 951 | TKDEFDCKAW AYFSDVDLEK DVHSGLIGPL LVCHTNTLNP AHGRQVTVQE |
| 1001 | FALFFTIFDE TKSWYFTENM ERNCRAPCNI QMEDPTEKEN YRFHAINGYI |
| 1051 | MDTLPGLVMA QDQRIRWYLL SMGSNENIHS IHFSGHVETV RKKEEYKMAL |
| 1101 | YNLYPGVFET VEMLPSKAGI WRVECLIGEH LHAGMSTLEL VYSNKCQTPL |
| 1151 | GMASGHIRDE QITASGQYGQ WAPKLARLHY SGSINAWSTK EPFSWIKVDL |
| 1201 | LAPMIIHGIK TQGARQKESS LYISQFIIMY SLDGKKWOTY RGNSTGTLMV |
| 1251 | FFGNVDSSGI KHNIENPPII ARYIRLHPTH YSIRSTLRME LMGCDLNSCS |
| 1301 | MPLGMESKAI SDAQITASSY FTNMFATWSP SKARLHLQGR SNAWRPQVNN |
| 1351 | PKEWLQVDFQ KTMKVTGVTT QGVKSLLTSM YVKEFLISSS QDGHQWTLFF |
| 1401 | QNGKVKVFQG NQDSFTPVVN SLDPPLLTRY LRIHPQSWVH QIALRMEVLG |
| 1451 | CEAQDLYDKT HTCPPCPAPE LLGGPSVFLF PPKPKDTLMI SRTPEVTCVV |
| 1501 | VDVSHEDPEV KFNWYVDGVE FHNAKTKPRE EQYNSTYRVV SVLTVLHQDW |
| 1551 | LNGKEYKCKV SNKALPAPIE KTISKAKGQP REPQVYTLPP SRDELTKNQV |
| 1601 | SLTCLVKGFY PSDIAVEWES NGQPENNYKT TPPVLDSDGS FFLYSKLTVD |
| 1651 | 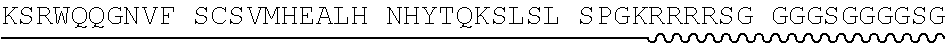 |
| 1701 | 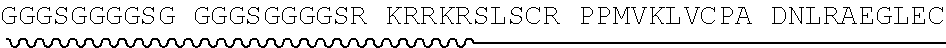 |
| 1751 | TKTCQNYDLE DMSMGCVSGC LCPPGMVRHE NRCVALERCP CFHQGKEYAP |
| 1801 | GETVKIGCNT CVCRDRKWNC TDHVCDATCS TIGMAHYLTE DGLKYLEPGE |
| 1851 | CQYVLVQDYC GSNPGTFRIL VGNKGCSHPS VKCKKRVTIL VEGGEIELED |
| 1901 | GEVNVKRPMK DETHFEVVES GRYIILLLGK ALSVVWDRHL SISVVLKQTY |
| 1951 | QEKVCGLCGN FDGIQNNDLT SSNLQVEEDP VDEGNSWKVS SQCADTRKVP |
| 2001 | LDSSPATCHN NIMKQTMVDS SCRILTSDVE QDCNKLVDPE PYLDVCIYDT |
| 2051 | CSCESIGDCA AFCDTIAAYA HVCAQHGKVV TWRTATLCPQ SCEERNLREN |
| 2101 | GYEAEWRYNS CAPACQVTCQ HPEPLACPVQ CVEGCHAHCP PGKILDELLQ |
| 2151 | TCVDPEDCPV CEVAGRRFAS GKKVTLNPSD PEHCQICHCD VVNLTCEACQ |
| 2201 | EPIDGGGGSG GGGSLVPRGS GGDKTHTCPP CPAPELLGGP SVELFPPKPK |
| 2251 | DTLMISRTPE VTCVVVDVSH EDPEVKENWY VDGVEVHNAK TKPREEQYNS |
| 2301 | TYRVVSVLTV LHQDWLNGKE YKCKVSNKAL PAPIEKTISK AKGQPREPQV |
| 2351 | YTLPPSRDEL TKNQVSLTCL VKGFYPSDIA VEWESNGQPE NNYKTTPPVL |
| 2401 | DSDGSFFLYS KLTVDKSRWQ QGNVFSCSVM HEALHNHYTQ KSLSLSPGK |

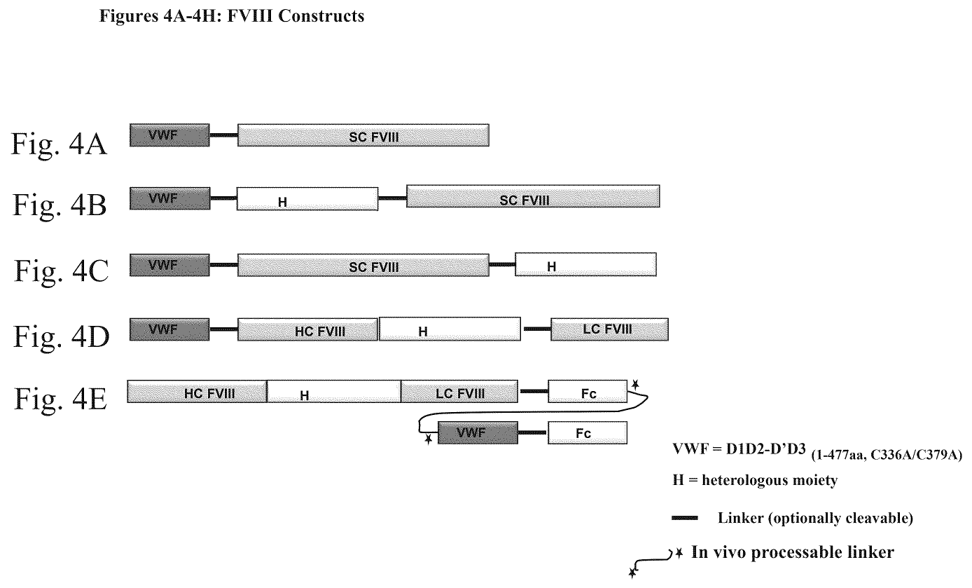

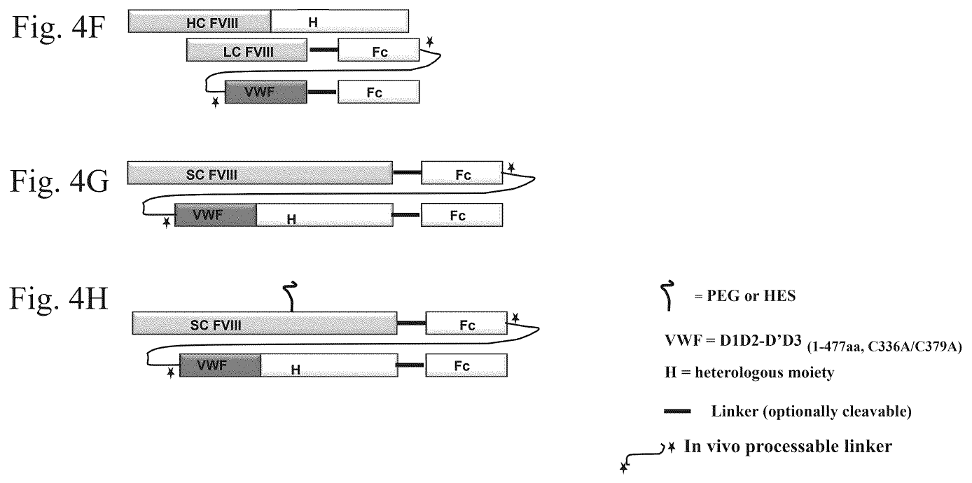

### Example 4: Example of FVIII-VWF DNA Constructs (FIG. 4)

The VWF fragment and FVIII protein can be linked together by a linker or another protein or a polypeptide using conventional recombinant DNA techniques, as show in FIG. 4. In FIG. 4A, the DID2D′D3 domains of VWF is linked to the FVIII protein by a 48aa linker-ISGGGGGGGGSGGGGSGGGGSGGGGSGGGGSLVPRGSGGGGSGGGGS (SEQ ID NO: 89) and protects FVIII from premature clearance. To further enhance the FVIII protecting activity of D′D3, another protein or polypeptide that has half-life extension potential such as albumin or a PAS sequence (heterologous moieties) can be incorporated into the construct. The heterologous moiety, e.g., albumin protein or PAS sequence, can be incorporated into different positions of the FVIII molecule; a few examples were shown in FIG. 4B-4D: at the N-termini of FVIII (4B), at the C-termini of FVIII (4C), or in the B region (4D). In those constructs, the additional protein sequences could enhance the D′D3 protecting activity and further extend FVIII half-life.

In addition, a heterologous moiety, e.g., albumin or PAS sequence, can also be incorporated into the FVIII/VWF heterodimer constructs as shown in FIG. 4E-4G. In FIG. 4E, a heterologous moiety, e.g., albumin or PAS sequence, is incorporated into the FVIII B domain region of FVIII-148; In FIG. 4F, a heterologous moiety, e.g., albumin or PAS sequence, is incorporated into the FVIII B domain region of FVIII-136; In FIG. 4G, a heterologous moiety, e.g., albumin or PAS sequence, is used as a linker to connect D′D3 fragment and Fc. In those configurations, a synergetic effect of D′D3, Fc, and heterologous moiety that is a half-life extender (e.g., albumin/PAS sequence) is expected on FVIII half-life extension.

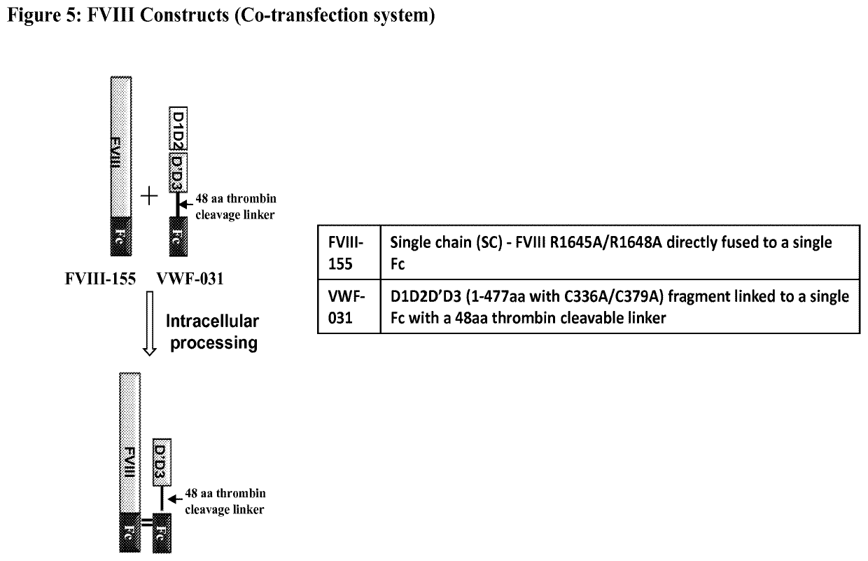

### Example 5: Plasmid Construction of Co-Transfection System for FVIIIFc-VWF Heterodimer (FIG. 5)

A co-transfection system was generated for FVIIIFc-VWF heterodimer production, which contains three DNA constructs. The first DNA construct-pSYN-FVIII-155 is encoding a FVIII-Fc fusion protein in which a single chain FVIII protein was directly fused to a single Fc fragment, and the second DNA construct is pSYN-VWF-031, which encodes a D′D3-Fc fusion protein (mentioned above in example 1). HEK293F cells were transfected with the two plasmids along with a third plasmid (PC5) at a 80:15:5 ratio. Co-transfection with PC5 is to ensure full propeptide processing of the D1 and D2 regions so that we have mature D′D3 domains. The synthesized proteins were secreted as FVIIIFc/D′D3Fc heterodimer and D′D3Fc homodimer and the FVIIIFc/D′D3Fc heterodimer was separated from the D′D3Fc homodimer by protein purification.

pSYN-FVIII-155 mature Protein sequencing (SEQ ID NO: 90):
ATRRYYLGAVELSWDYMQSDLGELPVDARFPPRVPKSFPFNTSVVYKKTLFVEFTDHLFNIAKPRPPWMGLL
GPTIQAEVYDTVVITLKNMASHPVSLHAVGVSYWKASEGAEYDDQTSQREKEDDKVFPGGSHTYVWQVLKEN
GPMASDPLCLTYSYLSHVDLVKDLNSGLIGALLVCREGSLAKEKTQTLHKFILLFAVFDEGKSWHSETKNSL
MQDRDAASARAWPKMHTVNGYVNRSLPGLIGCHRKSVYWHVIGMGTTPEVHSIFLEGHTFLVRNHRQASLEI
SPITFLTAQTLLMDLGQFLLFCHISSHQHDGMEAYVKVDSCPEEPQLRMKNNEEAEDYDDDLTDSEMDVVRF
DDDNSPSFIQIRSVAKKHPKTWVHYIAAEEEDWDYAPLVLAPDDRSYKSQYLNNGPQRIGRKYKKVRFMAYT
DETFKTREAIQHESGILGPLLYGEVGDTLLIIFKNQASRPYNIYPHGITDVRPLYSRRLPKGVKHLKDFPIL
PGEIFKYKWTVTVEDGPTKSDPRCLTRYYSSFVNMERDLASGLIGPLLICYKESVDQRGNQIMSDKRNVILF
SVFDENRSWYLTENIQRFLPNPAGVQLEDPEFQASNIMHSINGYVFDSLQLSVCLHEVAYWYILSIGAQTDF
LSVFFSGYTFKHKMVYEDTLTLFPFSGETVFMSMENPGLWILGCHNSDFRNRGMTALLKVSSCDKNTGDYYE
DSYEDISAYLLSKNNAIEPRSFSQNPPVLKAHQAEITRTTLQSDQEEIDYDDTISVEMKKEDFDIYDEDENQ
SPRSFQKKTRHYFIAAVERLWDYGMSSSPHVLRNRAQSGSVPQFKKVVFQEFTDGSFTQPLYRGELNEHLGL
LGPYIRAEVEDNIMVTFRNQASRPYSFYSSLISYEEDQRQGAEPRKNFVKPNETKTYFWKVQHHMAPTKDEF
DCKAWAYFSDVDLEKDVHSGLIGPLLVCHTNTLNPAHGRQVTVQEFALFFTIFDETKSWYFTENMERNCRAP
CNIQMEDPTFKENYRFHAINGYIMDTLPGLVMAQDQRIRWYLLSMGSNENIHSIHFSGHVFTVRKKEEYKMA
LYNLYPGVFETVEMLPSKAGIWRVECLIGEHLHAGMSTLFLVYSNKCQTPLGMASGHIRDFQITASGQYGQW
APKLARLHYSGSINAWSTKEPFSWIKVDLLAPMIIHGIKTQGARQKFSSLYISQFIIMYSLDGKKWQTYRGN
STGTLMVFFGNVDSSGIKHNIFNPPIIARYIRLHPTHYSIRSTLRMELMGCDLNSCSMPLGMESKAISDAQI
TASSYFTNMFATWSPSKARLHLQGRSNAWRPQVNNPKEWLQVDFQKTMKVTGVTTQGVKSLLTSMYVKEFLI
SSSQDGHQWTLFFQNGKVKVFQGNQDSFTPVVNSLDPPLLTRYLRIHPQSWVHQIALRMEVLGCEAQDLYDK
THTCPPCPAPELLGGPSVFLFPPKPKDTLMISRTPEVTCVVVDVSHEDPEVKFNWYVDGVEVHNAKTKPREE
QYNSTYRVVSVLTVLHQDWLNGKEYKCKVSNKALPAPIEKTISKAKGQPREPQVYTLPPSRDELTKNQVSLT
CLVKGFYPSDIAVEWESNGQPENNYKTTPPVLDSDGSFFLYSKLTVDKSRWQQGNVFSCSVMHEALHNHYTQ
KSLSLSPGK
pSYN-FVIII-155 DNA sequencing (SEQ ID NO: 91):
ATGCAAATAGAGCTCTCCACCTGCTTCTTTCTGTGCCTTTTGCGATTCTGCTTTAGTGCCACCAGAAGATAC
TACCTGGGTGCAGTGGAACTGTCATGGGACTATATGCAAAGTGATCTCGGTGAGCTGCCTGTGGACGCAAGA
TTTCCTCCTAGAGTGCCAAAATCTTTTCCATTCAACACCTCAGTCGTGTACAAAAAGACTCTGTTTGTAGAA
TTCACGGATCACCTTTTCAACATCGCTAAGCCAAGGCCACCCTGGATGGGTCTGCTAGGTCCTACCATCCAG
GCTGAGGTTTATGATACAGTGGTCATTACACTTAAGAACATGGCTTCCCATCCTGTCAGTCTTCATGCTGTT
GGTGTATCCTACTGGAAAGCTTCTGAGGGAGCTGAATATGATGATCAGACCAGTCAAAGGGAGAAAGAAGAT
GATAAAGTCTTCCCTGGTGGAAGCCATACATATGTCTGGCAGGTCCTGAAAGAGAATGGTCCAATGGCCTCT
GACCCACTGTGCCTTACCTACTCATATCTTTCTCATGTGGACCTGGTAAAAGACTTGAATTCAGGCCTCATT
GGAGCCCTACTAGTATGTAGAGAAGGGAGTCTGGCCAAGGAAAAGACACAGACCTTGCACAAATTTATACTA
CTTTTTGCTGTATTTGATGAAGGGAAAAGTTGGCACTCAGAAACAAAGAACTCCTTGATGCAGGATAGGGAT
GCTGCATCTGCTCGGGCCTGGCCTAAAATGCACACAGTCAATGGTTATGTAAACAGGTCTCTGCCAGGTCTG
ATTGGATGCCACAGGAAATCAGTCTATTGGCATGTGATTGGAATGGGCACCACTCCTGAAGTGCACTCAATA
TTCCTCGAAGGTCACACATTTCTTGTGAGGAACCATCGCCAGGCGTCCTTGGAAATCTCGCCAATAACTTTC
CTTACTGCTCAAACACTCTTGATGGACCTTGGACAGTTTCTACTGTTTTGTCATATCTCTTCCCACCAACAT
GATGGCATGGAAGCTTATGTCAAAGTAGACAGCTGTCCAGAGGAACCCCAACTACGAATGAAAAATAATGAA
GAAGCGGAAGACTATGATGATGATCTTACTGATTCTGAAATGGATGTGGTCAGGTTTGATGATGACAACTCT
CCTTCCTTTATCCAAATTCGCTCAGTTGCCAAGAAGCATCCTAAAACTTGGGTACATTACATTGCTGCTGAA
GAGGAGGACTGGGACTATGCTCCCTTAGTCCTCGCCCCCGATGACAGAAGTTATAAAAGTCAATATTTGAAC
AATGGCCCTCAGCGGATTGGTAGGAAGTACAAAAAAGTCCGATTTATGGCATACACAGATGAAACCTTTAAG
ACTCGTGAAGCTATTCAGCATGAATCAGGAATCTTGGGACCTTTACTTTATGGGGAAGTTGGAGACACACTG
TTGATTATATTTAAGAATCAAGCAAGCAGACCATATAACATCTACCCTCACGGAATCACTGATGTCCGTCCT
TTGTATTCAAGGAGATTACCAAAAGGTGTAAAACATTTGAAGGATTTTCCAATTCTGCCAGGAGAAATATTC
AAATATAAATGGACAGTGACTGTAGAAGATGGGCCAACTAAATCAGATCCTCGGTGCCTGACCCGCTATTAC
TCTAGTTTCGTTAATATGGAGAGAGATCTAGCTTCAGGACTCATTGGCCCTCTCCTCATCTGCTACAAAGAA
TCTGTAGATCAAAGAGGAAACCAGATAATGTCAGACAAGAGGAATGTCATCCTGTTTTCTGTATTTGATGAG
AACCGAAGCTGGTACCTCACAGAGAATATACAACGCTTTCTCCCCAATCCAGCTGGAGTGCAGCTTGAGGAT
CCAGAGTTCCAAGCCTCCAACATCATGCACAGCATCAATGGCTATGTTTTTGATAGTTTGCAGTTGTCAGTT
TGTTTGCATGAGGTGGCATACTGGTACATTCTAAGCATTGGAGCACAGACTGACTTCCTTTCTGTCTTCTTC
TCTGGATATACCTTCAAACACAAAATGGTCTATGAAGACACACTCACCCTATTCCCATTCTCAGGAGAAACT
GTCTTCATGTCGATGGAAAACCCAGGTCTATGGATTCTGGGGTGCCACAACTCAGACTTTCGGAACAGAGGC
ATGACCGCCTTACTGAAGGTTTCTAGTTGTGACAAGAACACTGGTGATTATTACGAGGACAGTTATGAAGAT
ATTTCAGCATACTTGCTGAGTAAAAACAATGCCATTGAACCAAGAAGCTTCTCTCAAAACCCACCAGTCTTG
AAAGCCCATCAGGCGGAAATAACTCGTACTACTCTTCAGTCAGATCAAGAGGAAATTGACTATGATGATACC
ATATCAGTTGAAATGAAGAAGGAAGATTTTGACATTTATGATGAGGATGAAAATCAGAGCCCCCGCAGCTTT
CAAAAGAAAACACGACACTATTTTATTGCTGCAGTGGAGAGGCTCTGGGATTATGGGATGAGTAGCTCCCCA
CATGTTCTAAGAAACAGGGCTCAGAGTGGCAGTGTCCCTCAGTTCAAGAAAGTTGTTTTCCAGGAATTTACT
GATGGCTCCTTTACTCAGCCCTTATACCGTGGAGAACTAAATGAACATTTGGGACTCCTGGGGCCATATATA
AGAGCAGAAGTTGAAGATAATATCATGGTAACTTTCAGAAATCAGGCCTCTCGTCCCTATTCCTTCTATTCT
AGCCTTATTTCTTATGAGGAAGATCAGAGGCAAGGAGCAGAACCTAGAAAAAACTTTGTCAAGCCTAATGAA
ACCAAAACTTACTTTTGGAAAGTGCAACATCATATGGCACCCACTAAAGATGAGTTTGACTGCAAAGCCTGG
GCTTATTTCTCTGATGTTGACCTGGAAAAAGATGTGCACTCAGGCCTGATTGGACCCCTTCTGGTCTGCCAC
ACTAACACACTGAACCCTGCTCATGGGAGACAAGTGACAGTACAGGAATTTGCTCTGTTTTTCACCATCTTT
GATGAGACCAAAAGCTGGTACTTCACTGAAAATATGGAAAGAAACTGCAGGGCTCCCTGCAATATCCAGATG
GAAGATCCCACTTTTAAAGAGAATTATCGCTTCCATGCAATCAATGGCTACATAATGGATACACTACCTGGC
TTAGTAATGGCTCAGGATCAAAGGATTCGATGGTATCTGCTCAGCATGGGCAGCAATGAAAACATCCATTCT
ATTCATTTCAGTGGACATGTGTTCACTGTACGAAAAAAAGAGGAGTATAAAATGGCACTGTACAATCTCTAT
CCAGGTGTTTTTGAGACAGTGGAAATGTTACCATCCAAAGCTGGAATTTGGCGGGTGGAATGCCTTATTGGC
GAGCATCTACATGCTGGGATGAGCACACTTTTTCTGGTGTACAGCAATAAGTGTCAGACTCCCCTGGGAATG
GCTTCTGGACACATTAGAGATTTTCAGATTACAGCTTCAGGACAATATGGACAGTGGGCCCCAAAGCTGGCC
AGACTTCATTATTCCGGATCAATCAATGCCTGGAGCACCAAGGAGCCCTTTTCTTGGATCAAGGTGGATCTG
TTGGCACCAATGATTATTCACGGCATCAAGACCCAGGGTGCCCGTCAGAAGTTCTCCAGCCTCTACATCTCT
CAGTTTATCATCATGTATAGTCTTGATGGGAAGAAGTGGCAGACTTATCGAGGAAATTCCACTGGAACCTTA
ATGGTCTTCTTTGGCAATGTGGATTCATCTGGGATAAAACACAATATTTTTAACCCTCCAATTATTGCTCGA
TACATCCGTTTGCACCCAACTCATTATAGCATTCGCAGCACTCTTCGCATGGAGTTGATGGGCTGTGATTTA
AATAGTTGCAGCATGCCATTGGGAATGGAGAGTAAAGCAATATCAGATGCACAGATTACTGCTTCATCCTAC
TTTACCAATATGTTTGCCACCTGGTCTCCTTCAAAAGCTCGACTTCACCTCCAAGGGAGGAGTAATGCCTGG
AGACCTCAGGTGAATAATCCAAAAGAGTGGCTGCAAGTGGACTTCCAGAAGACAATGAAAGTCACAGGAGTA
ACTACTCAGGGAGTAAAATCTCTGCTTACCAGCATGTATGTGAAGGAGTTCCTCATCTCCAGCAGTCAAGAT
GGCCATCAGTGGACTCTCTTTTTTCAGAATGGCAAAGTAAAGGTTTTTCAGGGAAATCAAGACTCCTTCACA
CCTGTGGTGAACTCTCTAGACCCACCGTTACTGACTCGCTACCTTCGAATTCACCCCCAGAGTTGGGTGCAC
CAGATTGCCCTGAGGATGGAGGTTCTGGGCTGCGAGGCACAGGACCTCTACGACAAAACTCACACATGCCCA
CCGTGCCCAGCTCCAGAACTCCTGGGCGGACCGTCAGTCTTCCTCTTCCCCCCAAAACCCAAGGACACCCTC
ATGATCTCCCGGACCCCTGAGGTCACATGCGTGGTGGTGGACGTGAGCCACGAAGACCCTGAGGTCAAGTTC
AACTGGTACGTGGACGGCGTGGAGGTGCATAATGCCAAGACAAAGCCGCGGGAGGAGCAGTACAACAGCACG
TACCGTGTGGTCAGCGTCCTCACCGTCCTGCACCAGGACTGGCTGAATGGCAAGGAGTACAAGTGCAAGGTC
TCCAACAAAGCCCTCCCAGCCCCCATCGAGAAAACCATCTCCAAAGCCAAAGGGCAGCCCCGAGAACCACAG
GTGTACACCCTGCCCCCATCCCGGGATGAGCTGACCAAGAACCAGGTCAGCCTGACCTGCCTGGTCAAAGGC
TTCTATCCCAGCGACATCGCCGTGGAGTGGGAGAGCAATGGGCAGCCGGAGAACAACTACAAGACCACGCCT
CCCGTGTTGGACTCCGACGGCTCCTTCTTCCTCTACAGCAAGCTCACCGTGGACAAGAGCAGGTGGCAGCAG
GGGAACGTCTTCTCATGCTCCGTGATGCATGAGGCTCTGCACAACCACTACACGCAGAAGAGCCTCTCCCTG
TCTCCGGGTAAA

Additional VWF fragments and FVIIIFC-VWF heterodimers that have been constructed are listed below.

TABLE 6
VWF Fragments and FVIII/VWF Heterodimer Constructs

| Construct | Description | Vector |
|---|---|---|
| VWF | | |
| pSYN-VWF-001 | FVIII signal peptide D′D3 region (1-276 amino acids | pcDNA 4 |
| | long 6x His) | |
| pSYN-VWF-002 | FVIII signal peptide D′D3 region (1-477 amino acids | pcDNA 4 |
| | long 6x His) | |
| pSYN-VWF-003 | FVIII signal peptide D′D3 region partial A1 (1-511 | pcDNA 4 |
| | amino acids long 6x His) | |
| pSYN-VWF-004 | FVIII signal peptide D′D3A1 region (1-716 amino | pcDNA 4 |
| | acids long 6x His) | |
| pSYN-VWF-006 | D1D2D′D3-linker-CK1 | pcDNA 3.1 |
| pSYN-VWF-008 | Full length WT-VWF | pcDNA 3.1 |
| pSYN-VWF-009 | D1D2D′D3 region (1-276 aa, 6x His) | pcDNA 3.1 |
| pSYN-VWF-010 | D1D2D′D3 region (1-477 aa, 6x His) | pcDNA 3.1 |
| pSYN-VWF-011 | D1D2D′D3 region partial A1 (1-511 aa, 6x His) | pcDNA 3.1 |
| pSYN-VWF-012 | D1D2D′D3A1 region (1-716 aa, 6x His) | pcDNA 3.1 |
| pSYN-VWF-013 | D1D2D′D3 region (1-477 aa, C336A/C379A, 6x His) | pcDNA 3.1 |
| pSYN-VWF-014 | FVIII signal peptide-D′D3 (1-477aa, C336A/C379A)-single | pcDNA 4 |
| | Fc with 20aa linker containing thrombin site | |
| pSYN-VWF-015 | D1D2D′D3 (1-477aa, C336A/C379A)-single Fc with 20aa | pcDNA 4 |
| | linker containing thrombin site | |
| pSYN-VWF-016 | FVIII signal peptide-D′D3 (1-477aa, WT)-single Fc with 20aa | pcDNA 4 |
| | linker containing thrombin site | |
| pSYN-VWF-017 | D1D2D′D3 (1-477aa, WT)-single Fc with 20aa linker | pcDNA 4 |
| | containing thrombin site | |
| pSYN-VWF-025 | D1D2D′D3 region (1-477 aa, 6x His) in pLIVE | pLIVE |
| pSYN-VWF-029 | D1D2D′D3 region (1-477 aa, C336A/C379A, 6x His) in pLIVE | pLIVE |
| pSYN-VWF-030 | FVIII signal peptide-D′D3 (1-477aa, C336A/C379A)-single Fc | pcDNA 4 |
| | with 48aa linker containing thrombin site | |
| pSYN-VWF-031 | D1D2D′D3 (1-477aa, C336A/C379A)-single Fc with 48aa | pcDNA 4 |
| | linker containing thrombin site | |
| pSYN-VWF-032 | FVIII signal peptide-D′D3 (1-477aa, WT)-single Fc with 48aa | pcDNA 4 |
| | linker containing thrombin site | |
| pSYN-VWF-033 | FVIII signal peptide-D′D3 (1-477aa, WT)-single Fc with 35 aa | pcDNA 4 |
| | linker containing thrombin site | |
| FVIII | | |
| pSYN-FVIII-055 | BDD-FVIII scFc with R336I and Y1680F | pBUD |
| pSYN-FVIII-056 | BDD-FVIII scFc with R336I, R562 and Y1680F | pBUD |
| pSYN-FVIII-057 | BDD-FVIII scFc with Y1680F | pBUD |
| pSYN-FVIII-058 | BDD-FVIII scFc with S488A | pBUD |
| pSYN-FVIII-059 | BDD-FVIII scFc with R336I, R562K, S488A | pBUD |
| pSYN-FVIII-060 | BDD-FVIII scFc with R336I, R562K, Y1680F | pBUD |
| pSYN-FVIII-061 | BDD-FVIII scFc with R336I, R562K, S488A, Y1680F | pBUD |
| pSYN-FVIII-064 | BDD-FVIII cleavable scFc with VWF D′D3 (1-477aa, | pBUD |
| | C336A/C379A) on second Fc & 20aa thrombin cleavable | |
| | linker in between | |
| pSYN-FVIII-065 | BDD-FVIII cleavable scFc with VWF D′D3 (1-276aa) on | pBUD |
| | second Fc & 20aa thrombin cleavable linker in between | |
| pSYN-FVIII-083 | BDD-FVIII scFc with R336I, S488A, R562K, Y1680F, E1984V | pBUD |
| pSYN-FVIII-086 | BDD-FVIII scFc with 6x(GGGGS) linker in between C2 of | pBUD |
| | FVIII and Fc | |
| pSYN-FVIII-095 | BDD-FVIII scFc with S104C, R562K, Y1680F, G1960C | pBUD |
| pSYN-FVIII-101 | BDD-FVIII scFc from FVIII-041 into pcDNA 3.3. Topo | pcDNA 3.3 |
| | | Topo |
| pSYN-FVIII-102 | BDD-FVIII (M662C/D1828C for disulfide binding; APC | pBUD |
| | cleavage mutations R336I/R562K; along with Y1680F | |
| | mutation for VWF binding) | |
| pSYN-FVIII-103 | BDD-FVIII scFc (Y662C/T1828C) | pBUD |
| pSYN-FVIII-104 | BDD-FVIII scFc (G655C/ST1788C) | pBUD |
| pSYN-FVIII-113 | BDD-FVIII (R490A/H497A) cleavable scFc with VWF D′D3 | pBUD |
| | (1-477aa, C336A/C379A) on second Fc & 20aa thrombin | |
| | cleavable linker in between | |
| pSYN-FVIII-114 | BDD-FVIII (R490A/H497A) cleavable scFc with VWF D′D3 | pBUD |
| | (1-276) on second Fc & 20aa thrombin cleavable linker in | |
| | between | |
| pSYN-FVIII-126 | BDD-FVIII scFc (M662C/D1828C) | pcDNA 3.3 |
| | | Topo |
| pSYN-FVIII-127 | BDD-FVIII scFc (M662C/D1828C for disulfide binding; APC | Topo |
| | cleavage mutations R336I/R562K; along with Y1680F mutation | pcDNA 3.3 |
| | for VWF binding) | |
| pSYN-FVIII-128 | BDD-FVIII scFc (Y664C/T1826C) | pcDNA 3.3 |
| | | Topo |
| pSYN-FVIII-129 | mutation of R336I R562K R490A H497A N1224A in the | pBUD |
| | background of pSYN-VIII-64 | |
| pSYN-FVIII-130 | mutation of R336I R562K R490A H497A N1224A in the | pBUD |
| | background of pSYN-VIII-65 | |
| pSYN-FVIII-131 | mutation of R471A Y487A R490A H497A N1224A in the | pBUD |
| | background of p SYN-VIII-64 | |
| pSYN-FVIII-132 | mutation of R471A Y487A R490A H497A N1224A in the | pBUD |
| | background of pSYN-VIII-65 | |
| pSYN-FVIII-135 | BDD-FVIII scFc with R1645A/R1648A | pcDNA 3.3 |
| | | Topo |
| pSYN-FVIII-136 | BDD-FVIII cleavable scFc with VWF D′D3 (1-477aa, | pcDNA 3.3 |
| | C336A/C379A) on second Fc & 20aa thrombin cleavable linker | Topo |
| | in between | |
| pSYN-FVIII-137 | BDD-FVIII cleavable scFc with VWF D′D3 (1-276aa) on | pcDNA 3.3 |
| | second Fc & 20aa thrombin cleavable linker in between | Topo |
| pSYN-FVIII-145 | BDD-FVIII scFc with R471A/Y487A, R490A/H497A | pcDNA 3.3 |
| | | Topo |
| pSYN-FVIII-146 | BDD-FVIII cleavable scFc (R471A/Y487A) with VWF D′D3 | pcDNA 3.3 |
| | (1-477aa, C336A/C379A) on second Fc & 20aa thrombin | Topo |
| | cleavable linker | |
| pSYN-FVIII-147 | BDD-FVIII cleavable scFc (R471A/Y487A) with VWF D′D3 | pcDNA 3.3 |
| | (1-276aa) on second Fc & 20aa thrombin cleavable linker in | Topo |
| | between | |
| pSYN-FVIII-148 | BDD-FVIII cleavable scFc (R1645A/R1648A) with VWF | pcDNA 3.3 |
| | D′D3 (1-477aa, C336A/C379A) on second Fc & 20aa | Topo |
| | thrombin cleavable linker | |
| pSYN-FVIII-149 | BDD-FVIII cleavable scFc (R1645A/R1648A) with VWF | pcDNA 3.3 |
| | D′D3 (1-276aa) on second Fc & 20aa thrombin cleavable linker | Topo |
| pSYN-FVIII-155 | BDD-FVIII fused to single Fc (R1645A/R1648A) | pcDNA 4 |
| pSYN-FVIII-159 | BDD-FVIII cleavable scFc with VWF D′D3 (1-477aa, | pBUD |
| | C336A/C379A) on second Fc & 35 aa thrombin cleavable | |
| | linker in between | |
| pSYN-FVIII-160 | BDD-FVIII cleavable scFc with VWF D′D3 (1-477aa, | pBUD |
| | C336A/C379A) on second Fc & 48 aa thrombin cleavable | |
| | linker in between | |
| pSYN-FVIII-164 | BDD-FVIII cleavable scFc (R490A/H497A, R1645A/R1648A) | pcDNA 3.3 |
| | with VWF D′D3 (1-477aa, C336A/C379A) on second Fc & | Topo |
| | 20aa thrombin cleavable linker | |
| pSYN-FVIII-165 | BDD-FVIII cleavable scFc (R336I/R562K, R490A/H497A, | pcDNA 3.3 |
| | R1645A/R1648A) with VWF D′D3 (1-477aa, C336A/C379A) | Topo |
| | on second Fc & 20aa thrombin cleavable linker | |
| pSYN-FVIII-178 | BDD-FVIII cleavable scFc with VWF D′D3 (1-477aa, | pBUD |
| | C336A/C379A) on second Fc & 73 aa thrombin cleavable | |
| | linker in between | |
| pSYN-FVIII-179 | BDD-FVIII cleavable scFc with VWF D′D3 (1-477aa, | pBUD |
| | C336A/C379A) on second Fc & 98 aa thrombin cleavable | |
| | linker in between | |
| pSYN-FVIII-180 | BDD-FVIII (K2092A) cleavable scFc with VWF D′D3 (1- | pBUD |
| | 477aa, C336A/C379A) on second Fc & 48 aa thrombin | |
| | cleavable linker in between | |
| pSYN-FVIII-181 | BDD-FVIII (F2093A) cleavable scFc with VWF D′D3 (1- | pBUD |
| | 477aa, C336A/C379A) on second Fc & 48 aa thrombin | |
| | cleavable linker in between | |
| pSYN-FVIII-182 | BDD-FVIII (K2092A/F2093A) cleavable scFc with VWF | pBUD |
| | D′D3 (1-477aa, C336A/C379A) on second Fc & 48 aa | |
| | thrombin cleavable linker in between | |

### Example 6: Protein Purification

### Protein Purification of VWF Fragments

The VWF fragments were purified through a two-step purification method. A Nickel Sulfate charged IMAC (Immobilized Metal Affinity Chromatography) column was used for the primary purification, a Fractogel DEAE ion exchange column was used for the final purification. The detail purification method is described below.

### (a) Primary Purification of VWF Fragment on Nickel IMAC

A 14 mL Nickel IMAC Sepharose HP column [XK26/3] was equilibrated with 25 mM HEPES, 500 mM NaCl, 10 mM Imidazole, and 0.05% Tween-20 @pH 7.5. Approximately 7.2 L of VWF conditioned media was adjusted with 100 mL of 1M HEPES @pH 7.5 and 600 mL of 5M NaCl. Then 80 mL of 1M Imidazole (@pH 7.5) was added to a final concentration of 10 mM. The 7.8 L of the adjusted VWF conditioned media was then loaded onto the column at 2-8° C. at 10 mL/min [113 cm/hour]. The wash steps were performed at 13.3 mL/minute [150 cm/hour]. First, a 2×Column Volume (CV) wash was performed with 25 mM HEPES, 500 mM NaCl, 10 mM Imidazole, and 0.05% Tween-20 @pH 7.5 in normal flow {“DownFlow”}. Next, a 3×CV wash was performed with 25 mM HEPES, 500 mM NaCl, 10 mM Imidazole, and 0.05% Tween-20 @pH 7.5 in reverse flow {“UpFlow”}. Lastly, A 3×CV wash was performed with 25 mM HEPES, 500 mM NaCl, 10 mM Imidazole, and 0.05% Tween-20 @pH 7.5 in normal flow {“DownFlow”}. The elution was performed as a 10×CV gradient to 50% B1 (25 mM HEPES, 500 mM NaCl, 500 mM Imidazole, and 0.05% Tween-20 @pH 7.5). The fraction volume was set to 10 mL. Then, the column was stripped with 100% B1. This was followed by a wash with 25 mM HEPES, 500 mM NaCl, 10 mM Imidazole, and 0.05% Tween-20 @pH 7.5. A second Strip was performed with 1N NaOH. Then the column was flushed with 1M TRIS, 1M NaCl@pH 7.8, followed by 25 mM HEPES, 500 mM NaCl, 10 mM Imidazole, and 0.05% Tween-20 @pH 7.5. Finally, the column was flushed with 5 CV's of DPBS+20% Ethanol and stored at 4° C.

### (b) Secondary Purification of VWF Fragment on Fractogel DEAE

Secondary purification of VWF fragment was performed on Fractogel DEAE@pH 7.5. Firstly, 20 mL of VWF Nickel IMAC eluate (corresponding to the VWF fragment peak) was adjusted with 200 mg of Zwittergent 3-14 zwitterionic detergent in an attempt to disrupt aggregated species without using denaturing or reducing excipients. After the detergent was dissolved, the protein was left at RT for approximately 15 minutes. Then, the protein was adjusted with 4 grams of trehalose, 1 mL of 10% Tween-20, 5 mL of 1M HEPES@pH 7.5 and 174 mL of “Milli-Q” water. The equilibration buffer “A12” was 25 mM HEPES, 50 mM NaCl, 1% Trehalose, 0.05% Tween-20 @pH 7.5. The elution buffer “B1” was 25 mM HEPES, 1000 mM NaCl, 1% Trehalose, 0.05% Tween-20 @pH 7.5. The elution was performed as a 10 CV gradient to 50% B1, with a 5+ CV hold followed by a step to 100% B1. Then the column was stripped with 0.85% Phosphoric Acid, followed by 1M TRIS, 1M NaCl@pH 7.5. Then the column was stripped with 1N NaOH, 2M NaCl followed by 1M TRIS, 1M NaCl@pH 7.5. Then the column was flushed with 25 mM HEPES, 100 mM NaCl+20% Ethanol@pH 7.5 for storage.

### (c) Protein Purification of FVIII-VWF Heterodimer

The FVIII-VWF heterodimer was first purified by an affinity column (GE VIIISelect), then followed by a Fractogal TMAE ion exchange column. (McCue J T, Selvitelli K, Walker J, J Chromatogr A. 2009 Nov. 6; 1216(45): 7824-30. Epub 2009 Sep. 23.)

For the purification of FVIII-155/VWF-31, a tangential flow filtration (TFF) step was used to buffer exchange the clarified conditioned media. The targeted proteins in the filtrate were then captured using affinity chromatography. A weak anion exchange chromatography step was followed to reduce HMW species. Both the purity and size of the molecule were accessed by HPLC-SEC and SDS-PAGE. The presence of different domains of FVIII-155/VWF-31 was further confirmed by western blotting. The specific activity of the molecule was comparable to B-domain deleted FVIII.

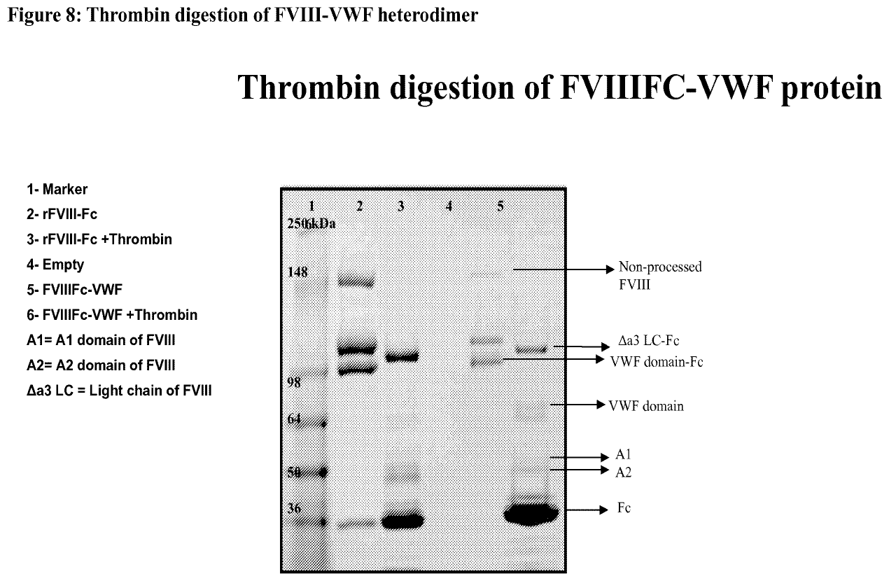

### (d) Thrombin Digestion of FVIII-VWF Heterodimer (FIG. 8)

FVIII-VWF-Fc heterodimer or FVIII-Fc (control) was mixed with thrombin in 1:10 ratio in thrombin cleavage buffer (50 mM Tris, pH 7.4, 150 mM NaCl, 2 mM CaCl2, 5% Glycerol). The reaction was incubated at 37° C. for 20 minutes. Digested product was run on 4-12% reducing tris-glycine gel. Undigested protein was used as a control. Bands were visualized by coomassie stain.

### (e) Evaluation of the VWF Binding Ability of FVIII-155/VWF-031 by Octet Assay

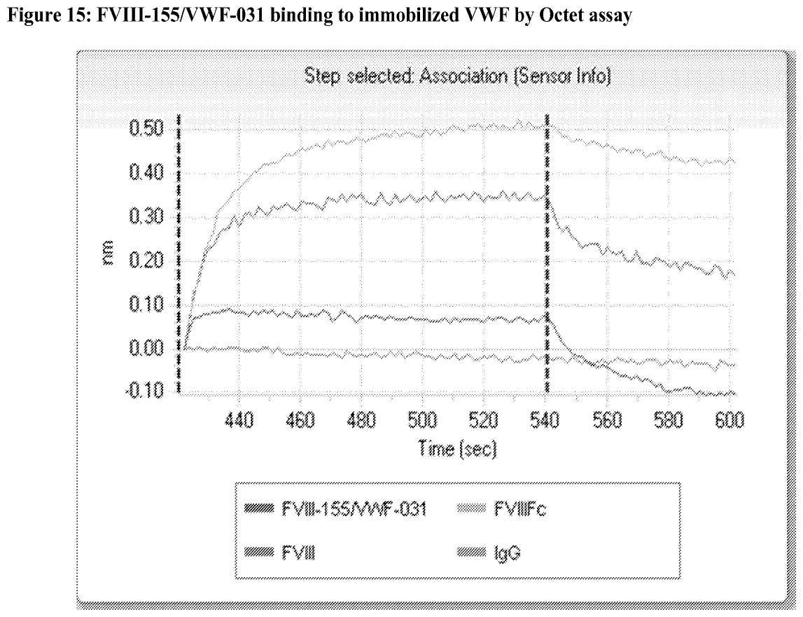

The VWF binding ability of FVIII-155/VWF-031 was determined by Bio-Layer Interferometry (BLI) based measurements (Octet assay) at 25° C. with a ForteBio Octet 384 instrument using Tris binding buffer (50 mM Tris, pH 7.2, 150 mM NaCl, 5 mM CaCl₂)). The Octet assay for determining FVIII binding was based on the hydrophobic immobilization of human von Willebrand Factor (hVWF) (Haematologic Technologies Catalog No. HCVWF-0191) onto the APS Biosensor, followed by binding of 1.0% Bovine Serum Albumin (Jackson ImmunoResearch Catalog No. 001-000-161). Briefly, hVWF (38.5 nM) was diluted in Tris buffer and loaded across APS Biosensors for 600 sec, yielding approximately 3.0-3.5 nm binding on the reaction probes. Control APS probes were loaded with 1.0% BSA in the absence of hVWF for reference subtraction. After loading, all probes were incubated in Tris buffer for 300 sec to establish a new baseline. Subsequently, biosensor probes were incubated in solutions of FVIII-155/VWF-031, FVIIIFc Drug Substance, or IFVIII (60 nM) for 5 min at room temperature, followed by a 5 min dissociation step. Using the Octet data analysis software, the binding response (nm) was derived from the subtracted data (Reaction probe minus Reference probe). As shown in FIG. 15, compared to the VWF binding affinity of rFVIIIFc and IFVIII, the VWF binding affinity of FVIII-155/VWF-031 was severely impaired. This indicates successful shielding of FVIII from full length VWF by the D′D3 fragment within the FVIIIFc/VWF heterodimer.

### Example 7. VWF-FVIII Interaction is a Limiting Factor for FVIII Half-Life Extension

The majority of the circulating FVIII exists as a FVIII-VWF complex (>95% of plasma FVIII). This FVIII-VWF interaction promotes FVIII clearance through the VWF clearance pathway, thus making the VWF half-life (T1/2) a limitation of the FVIII half-life extension. To evaluate this hypothesis, the limitation of FVIII half-life extension by Fc technology was tested in FVIII deficient mice (HemA mice, which have intact VWF gene) and FVIII/VWF deficient (FVIII-VWF Double Knockout (DKO)) mice.

The HemA mice or FVIII-VWF DKO mice were treated with a single intravenous dose of rFVIII or rFVIIIFc at 125 IU/kg in HemA mice or 200 IU/kg in DKO mice. Blood samples were collected up to 72 hrs in the HemA mice or up to 8 hrs in the FVIII/VWF DKO mice. Plasma sample's FVIII activity was then measured by a FVIII chromogenic assay. The pharmacokinetic (PK) profile of the two rFVIII variance was analyzed using WinNonline program.

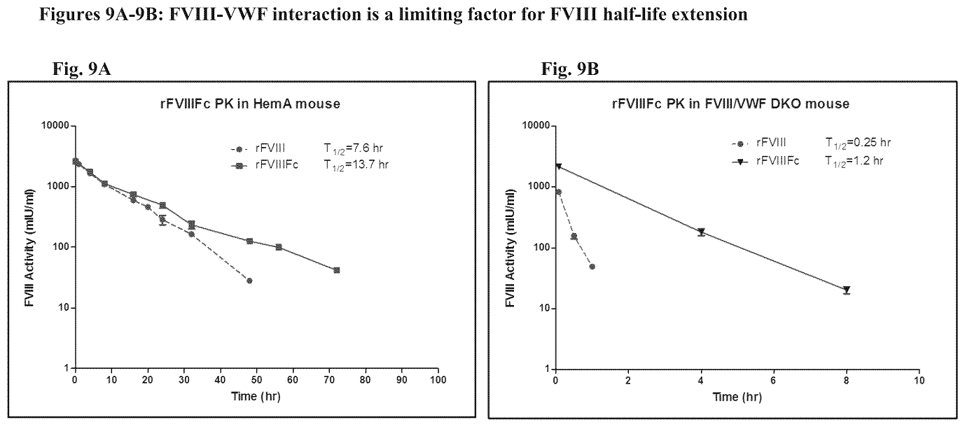

As shown in Table 7 and FIG. 9, in the FVIII/VWF DKO mice, IFVIIIFc showed about 4.8 fold longer T_{1/2} (i.e., T_{1/2} of 1.2 hr) compared to T_{1/2} of rFVIII (i.e., T_{1/2} of 0.25 hr). In contrast, when tested in HemA mice, rFVIIIFc only had 1.8 fold longer T_{1/2} compare to rFVIII. The T_{1/2} of rFVIIIFc was 13.7 hr, which is in line with the endogenous murine VWF half-life. This indicates that the FVIII-VWF interaction is a limiting factor for FVIII half-life extension. In order to achieve more than 2 fold FVIII half-life extension, the FVIII-VWF interaction will have to be eliminated.

TABLE 7
FVIII PK in HemA and FVIIII/VWF DKO mice

| | FVIII- | | FVIII/VWF- | |
|---|---|---|---|---|
| | deficient Mice | | deficient Mice | |
| Test Molecule | T_{1/2} (hr) | T_{1/2} Ratio vs rFVIII | T_{1/2} (hr) | T_{1/2} Ratio |
| rFVIII | 7.6 | 1 | 0.25 | 1 |
| rFVIIIFc | 13.7 | 1.8 | 1.2 | 4.8 |

The FVIII activity was measured using the COATEST SP FVIII kit from DiaPharma (lot #N089019) and all incubations were performed on a 37° C. plate heater with shaking.

The range of rFVIII standard was from 100 mIU/mL to 0.78 mIU/mL. A pooled normal human plasma assay control and plasma samples (diluted with 1×Coatest buffer) were added into Immulon 2HB 96-well plates in duplicate (25 μL/well). Freshly prepared IXa/FX/Phospholipid mix (50 μL), 25 μL of 25 mM CaCl₂), and 50 μL of FXa substrate were added sequentially into each well with 5 minutes incubation between each addition. After incubating with the substrate, 25 μL of 20% Acetic Acid was added to terminate the color reaction, and the absorbance of OD405 was measured with a SpectraMAX plus (Molecular Devices) instrument. Data were analyzed with SoftMax Pro software (version 5.2). The Lowest Level of Quantification (LLOQ) is 7.8 mIU/mL.

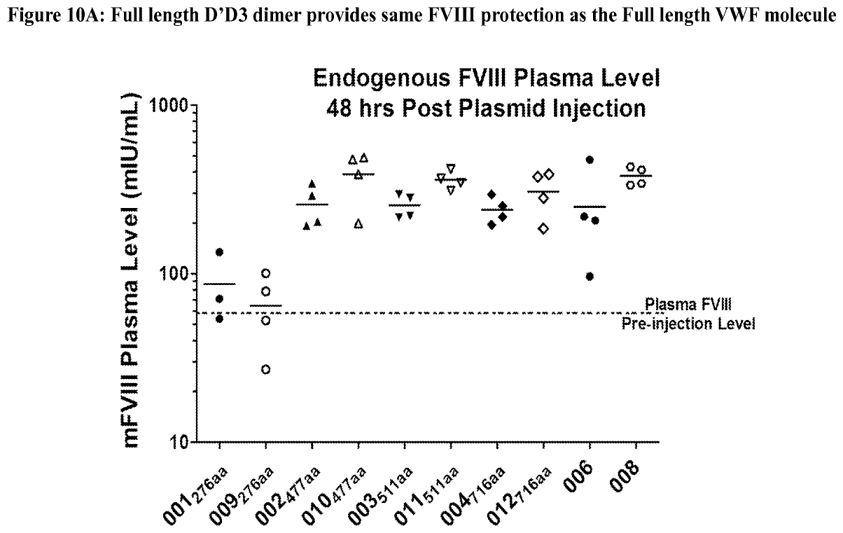

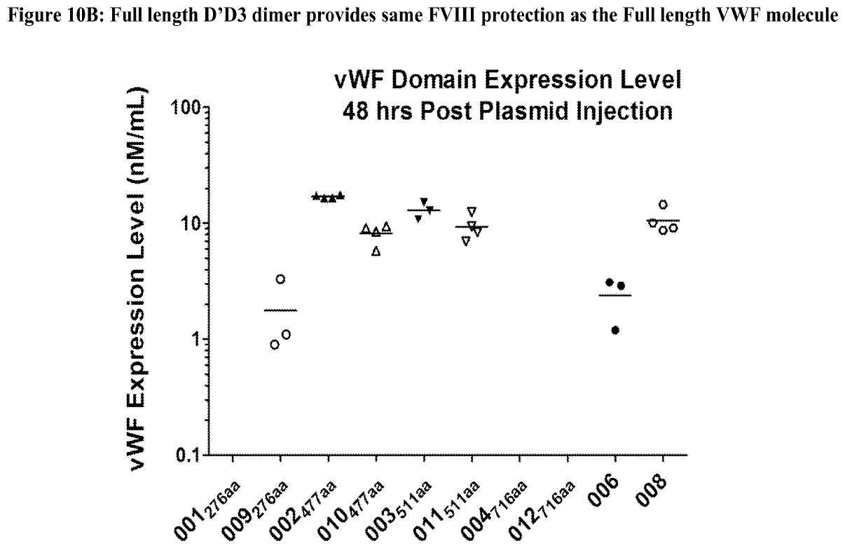

### Example 8. VWF D′D3 Dimer Protects FVIII from FVIII Proteolysis and Clearance (FIG. 10)

The FVIII protection activity of the VWF fragments was evaluated by their ability to protect endogenous murine FVIII from its clearance in VWF deficient mice. Different VWF fragment as listed in Table 8 Column 1 (FIG. 1, Example 1) were introduced into the blood circulation of the VWF deficient mice by Hydrodynamic injection of their corresponding DNA constructs at 100 μg/mouse. The plasma samples were collected at 48 hrs post injection, and murine FVIII plasma activity was measured by a FVIII chromogenic assay. VWF expression level was measured by VWF ELISA.

Four different lengths of the VWF fragments that have been tested are 276, 477, 511, and 716 amino acids. The 276 to 716 amino acid range was tested to find out the length of the VWF fragments required for FVIII binding (276aa) without VWF's clearance receptor's binding domain (716aa). The full length VWF and the D1D2D′D3CK multimer were used as the positive control for FVIII protection. In blood circulation, the VWF fragments synthesized with the DID2 domain exist as a dimer and exist as monomers when they are synthesized without the DID2 domain.

The increase of murine FVIII activity in plasma post hydrodynamic injection measures the FVIII protection effect of the VWF fragments. As shown in Table 8 and FIG. 10A-B, the first 276aa of the D′D3 fragment had no FVIII protection activity as demonstrated by the similar pre/post injection FVIII plasma level (FIG. 10A). However, the introduction of the other VWF fragments induced a significant increase on FVIII plasma level, indicating that those VWF fragments can protect FVIII from its clearance pathway.

TABLE 8
FVIII/VWF DKO mice murine FVIII plasma level
Pre/post introduction of VWF fragments (DNA
constructs were illustrated in FIG. 1)

| | | FVIII | | FVIII | | VWF | |
|---|---|---|---|---|---|---|---|
| | | Activity- | | Activity- | | Antigen- | |
| | | Pre | | 48 hr | | 48 hr | |
| DNA | Encoding | (mIU/mL) | | (mIU/mL) | | (nM/mL) | |
| CONSTRUCT | VWF Fragment | Avg. | SD | Avg. | SD | Avg. | SD |
| VWF-001 | D′D3₂₇₆ₐₐ | 53 | 31 | 86 | 16 | 2.8 | 1.9 |
| VWF 009 | D1D2D′D3₂₇₆ₐₐ | 45 | 20 | 65 | 17 | 1.8 | 1.3 |
| VWF-002 | D′D3₄₇₇ₐₐ | 56 | 3 | 257 | 38 | 17.0 | 0.5 |
| VWF 010 | D1D2D′D3₄₇₇ₐₐ | 42 | 11 | 387 | 22 | 8.2 | 1.6 |
| VWF-003 | D′D3A1₅₁₁ₐₐ | 88 | 21 | 253 | 47 | 12.9 | 2.2 |
| VWF-011 | D1D2D′D3A1₅₁₁ₐₐ | 63 | 42 | 360 | 15 | 9.3 | 2.3 |
| VWF-004 | D′D3A1₇₁₆ₐₐ | 87 | 8 | 239 | 56 | | |
| VWF-012 | D1D2D′D3A1₇₁₆ₐₐ | 64 | 22 | 307 | 29 | | |
| VWF-006 | D1D2D′D3CK | 38 | 10 | 249 | 20 | 2.4 | 1.0 |
| VWF-008 | Full length VWF | 51 | 8 | 380 | 41 | 10.6 | 2.3 |

The ratio of post injection plasma FVIII activity and the plasma antigen level of the VWF fragments that contain the D′D3 domain of full-length VWF were listed in Table 8. Similar post injection FVIII/VWF ratio was observed from the full length VWF and the two dimer forms of the VWF fragments, meaning that those two VWF fragment dimers provide the same FVIII protection as the full length VWF. In addition, threefold higher FVIII/VWF ratio was observed from the VWF fragment dimer isoforms compare to their corresponding monomers: the D′D3 (477aa) dimer has the FVIII/VWF ratio of 38.7 mIU/nmol; the D′D3 (477aa) monomer has the FVIII/VWF ration of 11.6 mIU/nmol: the D′D3A1 (511aa) dimer has the FVIII/VWF ratio of 32.9 mIU/nmol; and the D′D3 (511aa) monomer has the FVIII/VWF ratio of 13.8 mIU/nmol, indicating the dimer isoforms of the VWF fragments provides better FVIII protections compare to their corresponding monomers.

TABLE 9
FVIII protection effect of full length D′D3 fragment

| DNA | Encoding | Multimer | FVIII/VWF (mIU/nmol) | |
|---|---|---|---|---|
| Construct | VWF Fragment | State | Mean | (SD) |
| VWF-002 | D′D3₄₇₇ₐₐ | Monomer | 11.6 | (4.4) |
| VWF-010 | D1D2D′D3₄₇₇ₐₐ | Dimer | 38.7 | (11.7) |
| VWF-003 | D′D3A1₅₁₁ₐₐ | Monomer | 13.8 | (1.3) |
| VWF-011 | D1D2D′D3A1₅₁₁ₐₐ | Dimer | 32.9 | (5.5) |
| VWF-008 | Full length VWF | Multimer | 31.1 | (6.7) |

### Hydrodynamic Injection:

Hydrodynamic Injection is an efficient and safe non-viral gene delivery method to the liver in small animals, such as mice and rats. It was originally described as a rapid injection of a naked plasmid DNA/saline solution free of endotoxin at a tenth volume of the animal's body weight in about 5-7 seconds. The naked plasmid DNA contains the gene of interest and the liver produced targeted protein from the injected DNA can be detected within 24 hours post-injection. Plasma samples were then collected to study the therapeutic property of the expressed protein.

For all the hydrodynamic injections that were performed herein in this patent application, 2 ml of plasmid DNA in 0.9% sterile saline solution was delivered via intravenous tail vein injection within about 4-7 seconds to mice weighing 20-35 grams. The mice were closely monitored for the first couple of hours until the normal activity resumed. After the blood samples were collected via retro orbital blood collection, plasma samples were then obtained and stored at −80° C. for further analysis.

### VWF ELISA:

Goat anti-human VWF antibody (Affinity purified, affinity biological, GAVWF-AP) was used as the capture antibody at 0.5 ug/well and VWF-EIA-D (Affinity Biologicals, VWF-EIA-D, 1:100 dilution) was used as the detecting antibody for the VWF ELISA. ELISA assay was performed following the standard ELISA procedure, TMB was used as the HRP substrate, PBST/1.5% BSA/0.5M NaCl buffer was used as blocking and binding buffer. The assay standard range is 100 ng to 0.78 ng, and the assay's lowest limit of quantification (LLOQ) is 7.8 ng/ml.

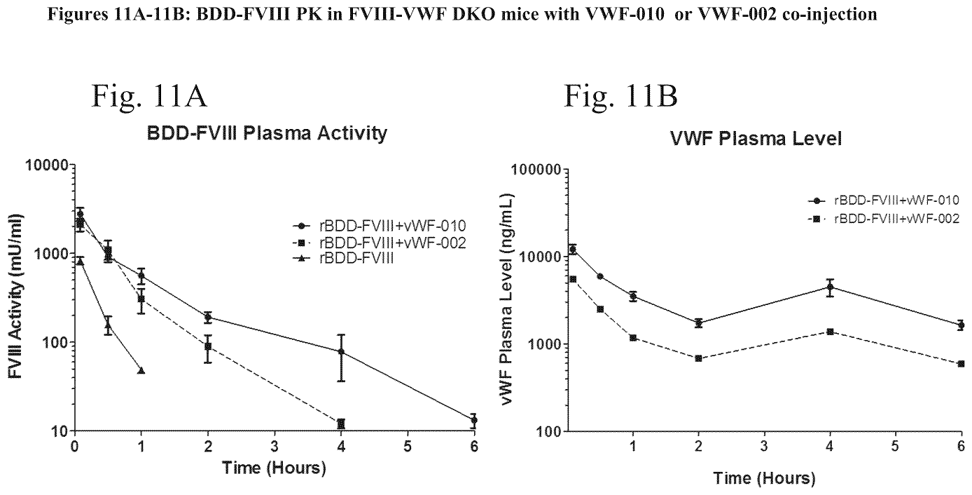

### Example 9: Co-Administration of Full Length VWF D′D3 Fragment Extend rBDD-FVIII Half-Life in FVIII-VWF DKO Mice (FIG. 11)

Example 8 has demonstrated that full length D′D3 fragment can protect endogenous FVIII from its clearance pathway. In order to further evaluate the FVIII protection activity of D′D3 protein, FVIII-VWF DKO mice were co-administered with B domain deleted FVIII (rBDD-FVIII) and D′D3 dimer (VWF-010) or rBDD-FVIII and D′D3 monomer (VWF-002), via intravenous injection at 200 IU/kg for rBDD-FVIII, 770 μg/kg for D′D3 dimer and 590 μg/kg for D′D3 monomer. The PK profile of rBDD-FVIII was then monitored by its post injection plasma activity. Due to the short in vivo half-life of the D′D3 fragments, at three hour post the initial co-injection, another dose of D′D3 was administered through the same route to maintain a desirable D′D3 plasma level.

For PK analysis, plasma sample was obtained via retro-orbital blood collection at 5 min, 30 min, 1 hour, 2 hour, 4 hour and 6 hour post injection, plasma FVIII activity and D′D3 antigen level was analyzed by FVIII chromogenic assay and VWF ELISA.

As shown in FIG. 11 and Table 10, the D′D3 monomer prolonged rBDD-FVIII half-life by 2.5 fold and improved its recovery by 1.8 fold. The D′D3 dimer prolonged rBDD-FVIII half-life by 4.1 fold and improved its recovery by 3.5 fold. Improved mean residency time, clearance and AUC were also observed from both of the D′D3 isoforms. The D′D3 dimer, however, achieved better results in all the PK parameters compared to its monomer form.

In summary, co-injection of full length D′D3 protects FVIII from its clearance pathway, as show in the improved PK profile of rBDD-FVIII. The potential clinical value of this finding needs to be further evaluated.

TABLE 10
BDD-FVIII PK parameter in FVIII-VWF DKO mice when co-administered
with D′D3 fragments

| | 5 min | | | | | AUC_D | T_{1/2} | Recovery |
|---|---|---|---|---|---|---|---|---|
| | Recovery | T_{1/2} | MRT | Cl | Vss | (hr * kg * mIU/ | Fold | Fold |
| Treatment | (%) | (hr) | (hr) | (mL/hr/kg) | (mL/kg) | mL/mIU) | Increase | Increase |
| rBDD-FVIII | 25 | 0.23 | 0.24 | 407.72 | 133.14 | 0.0025 | | |
| rBDD-FVIII | 44 | 0.57 | 0.58 | 151.93 | 124.63 | 0.0066 | 2.5 | 1.8 |
| VWF-002 | | | | | | | | |
| rBDD-FVIII | 87 | 0.95 | 0.98 | 71.48 | 97.54 | 0.014 | 4.1 | 3.5 |
| VWF-010 | | | | | | | | |

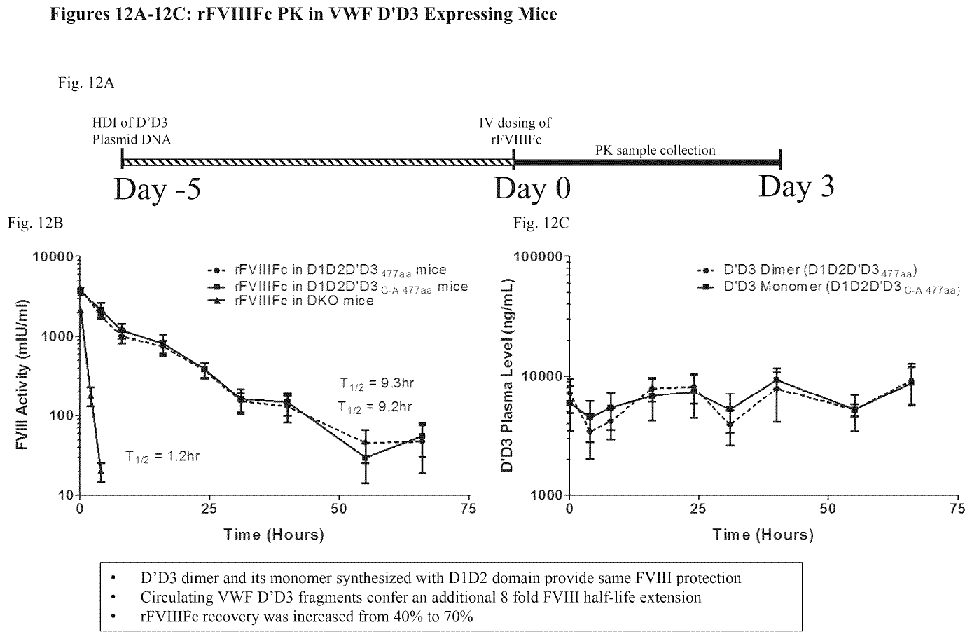

### Example 10. The D′D3 Monomer Synthesized with DID2 Domain and its Dimer Isoform have Same FVIII Protection Activity and Further Extended FVIIIFc's Half-Life by ~4 Fold in FVIII-VWF DKO Mice (FIG. 12)

In order to quantify the FVIII protection ability of the D′D3 domains and determine if the D′D3 dimerization is necessary for its FVIII protection activity, each of two DNA constructs (i.e., VWF-025 (containing DNA sequence encoding DID2D′D3) and VWF-029 (containing DID2D′D3 codon DNA with C336A and C379A mutation)) was administered into FVIII/VWF DKO mice by hydrodynamic injection. This injection resulted in D′D3 dimer (VWF-025) or monomer expression (VWF-029) in the FVIII/VWF DKO mice. At day5 post hydrodynamic injection, a single intravenous dose of rFVIIIFc was administered at 200 IU/kg, and plasma samples was collected at 5 min, 4, 8, 16, 24, 31, 40, 55, 66 hrs post rFVIIIFc IV injection. An rFVIIIFc PK study that was performed in naïve FVIII-VWF DKO mice at the same dose was used as the rFVIIIFc half-life base line. Plasma FVIII activity was analyzed by a FVIII chromogenic assay. Plasma D′D3 level was measured by VWF ELISA, and rFVIIIFc PK profile was analyzed using WinNonlin program.

As shown in Table 11 and FIG. 12, with the VWF D′D3 fragments in the circulation, rFVIIIFc's initial recovery increased from 42% to 75% with D′D3 dimer and 60% with D′D3 monomer. rFVIIIFc's T_{1/2} was also increased from 2.5 hrs to 9.3 hrs and 9.2 hrs, respectively. Similar to T_{1/2}, improved mean residency time, clearance, and volume distribution were also observed from the D′D3 monomer and dimer expressing mice. Overall, we see about 8 fold improvements on the rFVIIIFc's half-life and 6 fold improvements on AUC in both D′D3 monomer and dimer expressing mice. Same as its dimer isoform, the D′D3 monomer of full-length VWF that was synthesized with propeptide (DID2) of VWF is sufficient to provide the full FVIII protection effect as the full length VWF molecule.

In FVIII/VWF DKO mice, WT-FVIII has a 0.25 hr T_{1/2}. The Fc fusion technology increased FVIII T_{1/2} to 1.2 hour, which is about 4.8 fold increase. When the Fc fusion technology was combined with the D′D3 domains, the FVIII T_{1/2} was increased to 9.3 hr (D′D3 dimer) and 9.2 hr (D′D3 monomer), which are about 37fold increases in total. (Table 10) This result demonstrated the synergistic effect of the Fc fusion and D′D3 VWF fragment on the FVIII half-life extension.

TABLE 11
rFVIIIFc PK parameter with/without D′D3 fragment in blood circulation

| | 5 min | | | | | AUC_D | T_{1/2} | AUC D |
|---|---|---|---|---|---|---|---|---|
| | Recovery | T_{1/2} | MRT | Cl | Vss | (hr * kg * mIU/ | Fold | Fold |
| Treatment | (%) | (hr) | (hr) | (mL/hr/kg) | (mL/kg) | mL/mIU) | Increase | Increase |
| rFVIIIFc | 43 | 1.2 | 0.76 | 39.5 | 67.0 | 0.025 | | |
| rFVIIIFc | 75 | 9.3 | 11.1 | 6.1 | 67.6 | 0.164 | 7.8 | 6.6 |
| VWF-025 | | | | | | | | |
| rFVIIIFc | 60 | 9.2 | 11.3 | 6.7 | 75.7 | 0.149 | 7.7 | 6.0 |
| VWF-029 | | | | | | | | |

### Example 11: FVIII-VWF Heterodimer PK in HemA Mice

The PK profile of the lead candidates of FVIII-VWF heterodimer (such as FVIII-155/VWF-031) will be tested in HemA mice to evaluate their ability of shielding FVIII from the endogenous VWF and their ability for FVIII half-life extension.

HemA mice will be treated with a single intravenous dose of the lead candidates at 200 IU/kg, plasma samples will then be collected at 5 min, 4 hr, 8 hr, 24 hr, 48 hr, 72 hr, 96 hr and 120 hr, plasma activity will be tested by FVIII chromogenic assay, and FVIII variance half-life will be calculated by WinNonlin program.

In an optimal FVIII/VWF heterodimer configuration, the FVIII binding to the endogenous VWF will be completely inhibited, therefor the base line half-life of rFVIII will be decreased from 7.6 hr to 0.25 hr as shown in example 7. When D′D3 fragment non-covalently associated with FVIII, about 8 fold of half-life benefit was observed (example 9). In the lead candidates of the FVIII/VWF heterodimer, the VWF fragment is covalently associated with the FVIII molecule, better FVIII protection might be able to be achieved. The invention of this application opened the door to further extend FVIII half-life beyond the two fold ceiling, with the combination of the available half-life extension technologies, HemA patients could expect a better long acting FVIII variance in the near future.

The PK profile of FVIII-155/VWF-031 was tested in HemA and FVIII/VWF DKO mice to evaluate the ability of the D′D3 fragment to shield the FVIII moiety from the endogenous VWF. HemA or FVIII/VWF DKO mice were treated with a single intravenous dose of FVIII-155/VWF-031 at 200 IU/kg, plasma samples were then collected at 5 min, 8 hrs, 24 hrs, and 48 hours post dosing. The FVIII activity of the plasma sample was tested by a FVIII chromogenic assay, and the half-life of FVIII-155/VWF-031 was calculated using WinNonlin program.

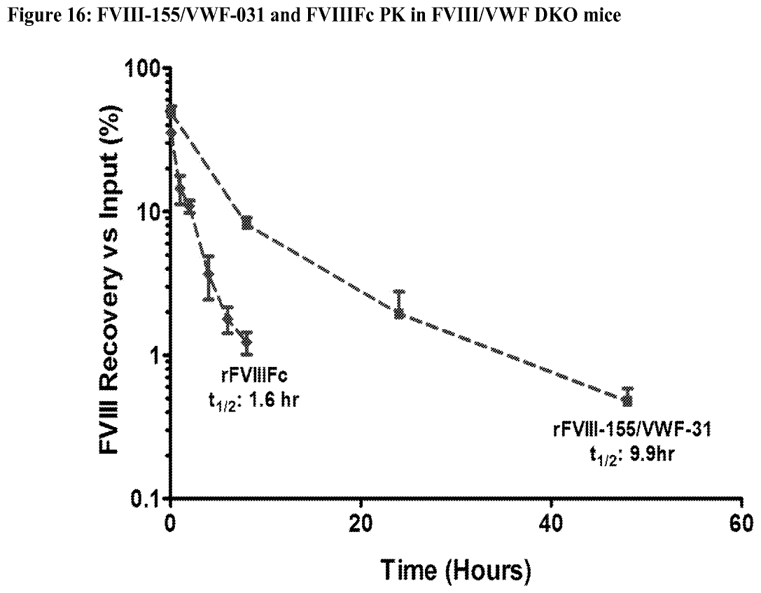

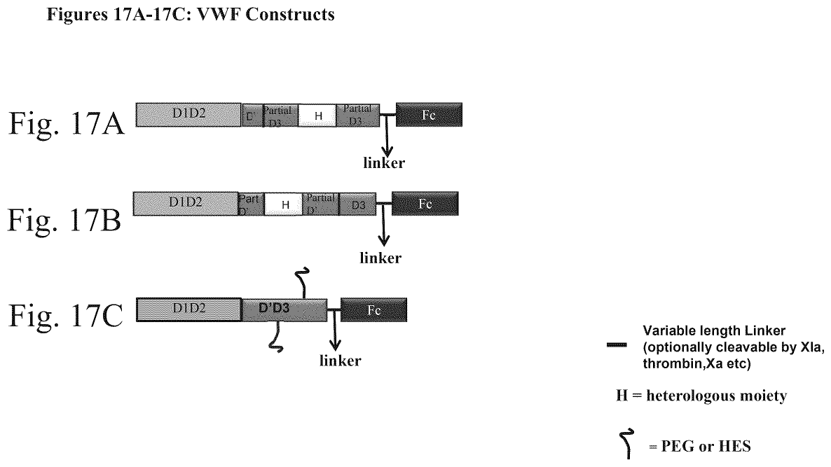

Severely impaired binding to immobilized VWF was detected by biolayer interferometry (FIG. 15, Octet; ForteBio Inc., Menlo Park, CA) for FVIII-155/VWF-031 compared to rFVIIIFc and rFVIII. This shows the D′D3 domain in the molecule had successfully blocked the FVIII binding to native VWF molecules. Therefore, similar half-life of rFVIII-155/VWF-031 was expected in the two different mouse strains. Study results are listed in FIG. 16 and Table 12A. As predicted, rFVIII-155/VWF-031 had comparable PK profile in both HemA and FVIII/VWF DKO mice, indicating that the half-life of FVIIIFc/VWF heterodimer is independent from the half-life of endogenous VWF. The results show that inhibition of the interaction between the rFVIIIFc with endogenous VWF by the VWF D′D3 domains allows elimination of the FVIII half-life ceiling and opens up the possibility of extending FVIII half-life beyond the half-life achievable without the VWF D′D3 domains (about two fold of the wild type FVIII).

TABLE 12A
FVIII-155/VWF-031 PK in FVIII/VWF DKO mice and HemA mice

| | 5 min | | | Cl | Vss | AUC_D |
|---|---|---|---|---|---|---|
| | Recovery | T_{1/2} | MRT | (mL/ | (mL/ | (hr * kg * mIU/ |
| Treatment | (%) | (hr) | (hr) | hr/kg) | kg) | mL/mIU) |
| FVIII- | 49 | 9.9 | 6.9 | 11.6 | 80.5 | 0.09 |
| 155/VWF- | | | | | | |
| 031 DKO | | | | | | |
| FVIII- | 69 | 10.8 | 707 | 11.9 | 92.1 | 0.08 |
| 155/VWF- | | | | | | |
| 031 HemA | | | | | | |

The FVIII protecting ability of the D′D3 domains was evaluated by comparing the t_{1/2} of FVIII-155/VWF-031 with FVIIIFc in FVIII/VWF DKO mice. After a single IV administration, blood samples were collected at 5 min, 8 hrs, 24 hrs and 48 hrs for FVIII-155/VWF-031, and at 5 min, 1 hrs, 2 hrs, 4 hrs, 6 hrs and 8 hrs for FVIIIFc. The FVIII activity of plasma sample was tested by a FVIII chromogenic assay, and the half-life of FVIII-155/VWF-031 was calculated using WinNonlin program.

FIG. 16B and Table 12B show a significantly improved PK profile for FVIII-155/VWF-031 compared to FFVIIIFc in DKO mice: about 6 fold increases on t_{1/2}; and about 5 fold increases in clearance and AUC. This result demonstrates that the D′D3 domain in FVIIIFc/VWF heterodimer protects the FVIII moiety from some clearance pathways, thus providing some of the protection normally provided by full length VWF. This conclusion is also confirmed in HemA mice. When compared to rFVIIIFc in HemA mice, rFVIII-155/VWF-031 has shown shorter t_{1/2} and lesser AUC, meaning in this configuration, the D′D3 domains (VWF-031) successfully prevents binding of the FVIII protein (rFVIII-155) to endogenous VWF, which has half-life extending properties to some degree, as well as a FVIII half-life limiting property. Full length VWF is 250 kDa, and forms multimers such that endogenous VWF can be up to 2 MDa, and therefore it is consistent with this hypothesis that the 55 kDa D′D3 region of VWF does not provide the same protection normally afforded by the much large endogenous VWF in this context. Since the VWF fragment prevents endogenous VWF from binding rFVIII-155/VWF-031, in this particular construct the half-life is decreased in the HemA mouse. Therefore, the results in Table 12B indicate that the rFVIII-155/VWF-031 molecule is capable of preventing the FVIII half-life extender (endogenous VWF) from binding the rFVIII-155/VWF-031. However, the experiment shows that removing the FVIII half-life limiting factor has opened up the possibility of extending a half-life of the FVIII protein beyond 1.5 fold or 2 fold shown previously. When FVIII is combined with other half-life extension elements as shown in FIG. 4, a breakthrough of the 2 fold half-life extension ceiling of FVIII could be achieved.

TABLE 12B
FVIII-155/VWF-031 and FVIIIFc PK in FVIII/VWF DKO mice

| | | | | | | AUC_D |
|---|---|---|---|---|---|---|
| | 5 min | | | Cl | Vss | (hr * |
| | Recovery | T_{1/2} | MRT | (mL/ | (mL/ | kg * mIU/ |
| Treatment | (%) | (hr) | (hr) | hr/kg) | kg) | mL/mIU) |
| FVIIIFc DKO | 43 | 1.6 | 1.9 | 63.9 | 123.2 | 0.02 |
| FVIII-155/ | 49 | 9.9 | 6.9 | 11.6 | 80.5 | 0.09 |
| VWF-031 | | | | | | |
| DKO | | | | | | |
| Fold Increase | | 6.2 | 3.6 | 5.5 | | 4.5 |
| FVIII-155/ | 69 | 10.8 | 7.7 | 11.9 | 92.1 | 0.08 |
| VWF-031 | | | | | | |
| HemA | | | | | | |
| FVIIIFc HemA | 86 | 16.4 | 20.3 | 2.9 | 57.7 | 0.35 |

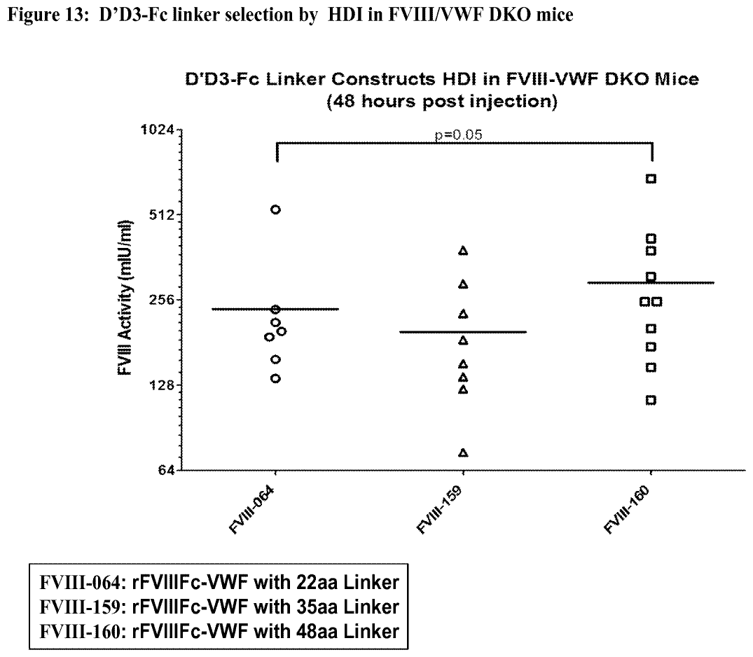

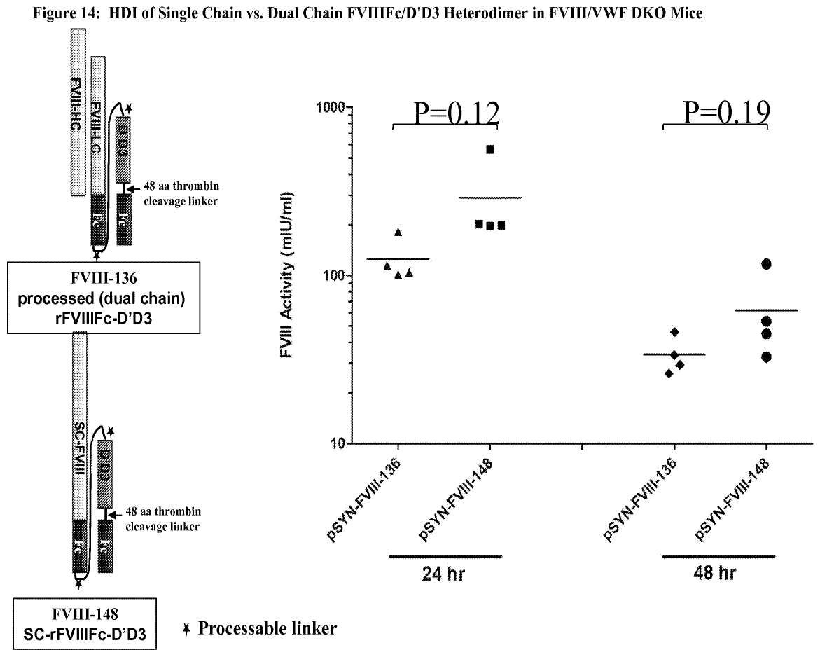

### Example 12: Optimization of the D′D3-Fc Linker of FVIII/D′D3 Heterodimer (FIG. 13)

To allow rFVIIIFc to escape the VWF clearance pathway and eliminate the 2 fold FVIII half-life extension ceiling, the VWF D′D3 fragment has been incorporated into the rFVIIIFc molecule (FIG. 2), resulting in an FVIIIFc/VWF heterodimer. In order to eliminate the interaction between rFVIIIFc and endogenous VWF and maximize the D′D3 FVIII protection potential, the linker between the D′D3 domain and the Fc region was adjusted to allow the optimal FVIII/D′D3 binding. A more optimal linker will allow the D′D3 domain to have greater FVIII protection than a less optimal linker construct does. This can be tested by hydrodynamic injection of the DNA constructs in FVIII/VWF DKO mice. A more optimal construct will yield higher steady state protein expression of the FVIIIFc/D′D3 heterodimer.

Three different FVIIIFc/D′D3 heterodimers (FIG. 3, Example 3) were engineered for optimal linker selection. The possible linkers between the D′D3 domains and the Fc region were listed in Table 13. Those DNA constructs were administered into FVIII/VWF DKO mice by hydrodynamic injection (“HDI”) at 100 μg/mouse, and plasma samples were collected 48 hr post HDI. Circulating FVIIIFc/D′D3 heterodimer activity was analyzed by a FVIII chromogenic assay.

The study result was shown in FIG. 13. 48 hours post HDI, similar expression level were reached by FVIII-064 and FVIII-159, indicating the 20aa linker and the 35aa linker promote similar level of FVIII/D′D3 interaction. In another hand, FVIII-160 showed significantly higher expression than FVIII-064, meaning that the 48aa linker allows better FVIII/D′D3 binding compare to the 20aa and 35aa linkers.

An optimal linker between the VWF fragment and the Fc region is one of the key elements of the FVIIIFc/VWF heterodimer. Finding the best linker will allow the optimal interaction between FVIII and the VWF fragment, prevent FVIII binding to endogenous VWF, enable FVIII to escape the VWF clearance pathway, and extend the FVIII half-life beyond the plasma VWF half-life.

TABLE 13
Different linkers between D′D3 and Fc fragment

| DNA | |
|---|---|
| construct | Linker between D′D3 and Fc |
| FVIII-064 | 20 aa = I D G G G G S G G G G S L V P R G S |
| (SEQ ID NO: 92) | G G |
| FVIII-159 | 35 aa = I S G G G G S G G G G S G G G G S G |
| (SEQ ID NO: 93) | G G G S G G G G S L V P R G S G G |
| FVIII-160 | 48 aa = I S G G G G S G G G G S G G G G S G |
| (SEQ ID NO: 94) | G G S G G G G S G G G G S L V P R G S G G G |
| | G G S G G G G S |

### Example 13: Single Chain FVIII Stability

The Single chain FVIII protein might be more stable than its dual chain isoform. To test this hypothesis, two DNA constructs were made: FVIII-136 (processable FVIIIFc with the D′D3 domain) and FVIII-148 (Single Chain (SC) FVIIIFc with the D′D3 domain, which contains R1645A/R1648A mutation to prevent cleavage between FVIII heavy chain and light chain).

Both plasmids were administered into FVIII/VWF DKO mice by hydrodynamic injection. Plasma samples were collected 24 hr and 48 hr post injections to measure the expression level of the two FVIIIFc/D′D3 isoforms. As shown in FIG. 14, at both time points, a trend of better expression was observed by the SC-FVIIIFc/D′D3 construct (FVIII-148) (p=0.12, p=0.19), indicating single chain FVIII might be more stable or better expressed than its dual chain isoform (FVIII-136). The PK profile of the two FVIII isoforms and their cell culture expression levels will be further investigated. The single chain FVIII isoform could be potentially used to replace the conventional dual chain isoform to achieve better protein production and better in vivo FVIII half-life.

### Example 14. PEGylation

One or more polyethylene glycol (PEG) molecules can be attached within any regions of the FVIII protein, the VWF fragment, or both. As FVIII does not have a free cysteine at its surface based on crystal structure (PDB: 2R7E, Shen et al., Blood 111:1240 (2008); PDB: 3CDZ, Ngo, Structure, 16:597-606 (2008)), one approach is to insert a cysteine containing peptide (e.g., GGGSGCGGGS) (SEQ ID NO: 107) into or link it to the FVIII protein, the VWF fragment, or both. PEG molecules containing maleimide can then be conjugated specifically to the cysteine introduced on the recombinant FVIII protein. Briefly, the recombinant FVIII protein containing the Cys insertion can be constructed by standard molecular technology, and the recombinant FVIII protein expressed in mammalian expression system (e.g., HEK293, CHO, BHK21, PER. C6, and CAP cells) can be purified via affinity and ion exchange chromatography. The purified recombinant FVIII protein is reduced by Tris(2-carboxyethyl)phosphine (TCEP) to expose the thiol group of the introduced cysteine and then reacted with maleimide PEG. The resulting recombinant FVIII protein is tested for procoagulant activity and extended half-life.

PEG is attached to at least one of the locations disclosed in U.S. Appl. No. 61/670,553, which is incorporated herein by reference in its entirety, or other suitable insertion sites. The FVIII activity of the PEGylated recombinant FVIII protein is analyzed using a FVIII chromogenic assay. The PK of the PEGylated recombinant FVIII protein is analyzed in HemA mice and FVIII-VWF DKO Mice as described above.

### Example 15: FVIII Stability in HemA and FVIII/VWF Double Knockout (DKO) Plasma

Plasma stability of different FVIIIFc fusions was tested in HemA or FVIII/VWF double knockout (DKO) plasma. For stability assay, 5 IU/ml of various FVIIIFc proteins were incubated with either mouse HemA or DKO plasma at 37° C. Aliquots were collected at different time points to measure activity by FVIII chromogenic assay. Activity at each time point was measured in duplicate and average activity was plotted as a function of time.

For the FVIIIFc immuno-precipitation assay, 5 μg FVIIIFc was incubated with either 250 μl of PBS or mouse DKO plasma for 24 hrs at 37° C. FVIIIFc was immuno-precipitated by adding 5 μg sheep anti-FVIII polyclonal antibody (ab61370) for 1 hr at room temperature and 100 μl protein A beads. After 4×1 ml PBS washes, beads were re-suspended in 50 μl 1× reducing SDS-PAGE buffer. After boiling, 20 μl sample (i.e. ~1 μg FVIIIFc) was loaded on to 4-15% Bio-Rad stain free gel. Gel was imaged by Bio-rad system followed by western analysis with FVIII anti heavy chain antibody (GMA012).

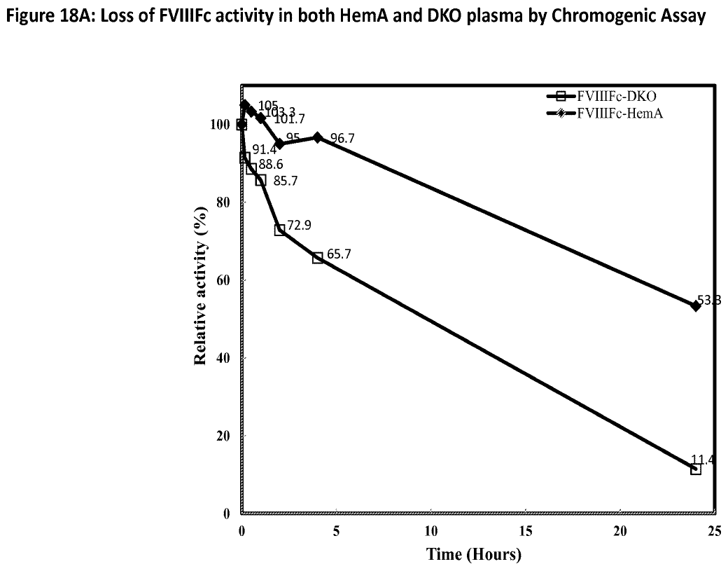

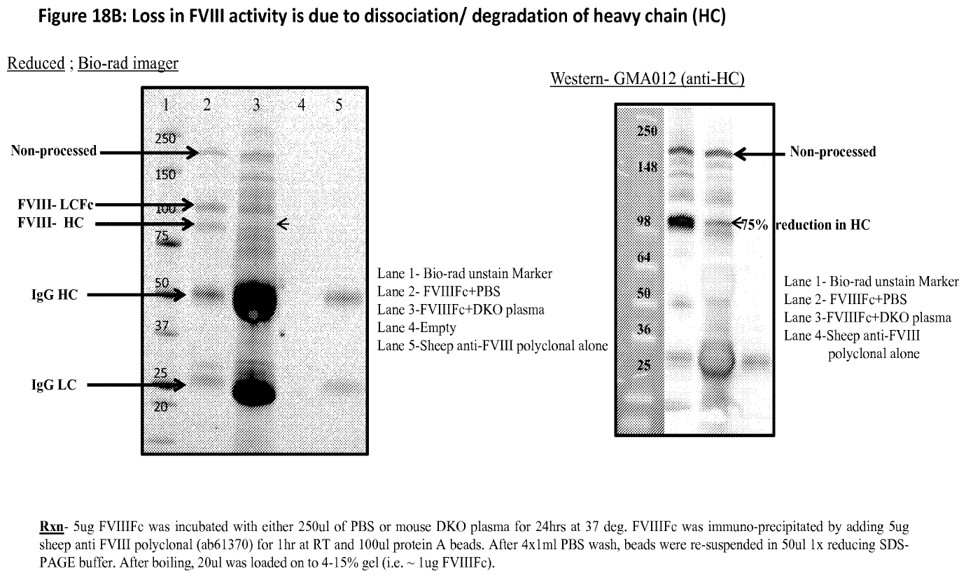

Activity of FVIIIFc (dual chain FVIII molecule, which has separate FVIII heavy and light chains, held together by non-covalent interactions) decreases with time in both HemA and DKO plasma (FIG. 18A). Due to lack of VWF mediated protection, loss in FVIIIFc activity was more pronounced in DKO plasma. This loss in FVIII activity was mainly due to dissociation or degradation of FVIII heavy chain (HC). About a 75% reduction in FVIIIFc heavy chain was observed after a 24 hr incubation in DKO plasma (FIG. 18B). No significant reduction was observed for either light chain (LC) (data not shown) or non-processed/single chain FVIIIFc (i.e. FVIII molecule in which light chain and heavy chain are still held together covalently-top band in the gel picture) (FIG. 18B).

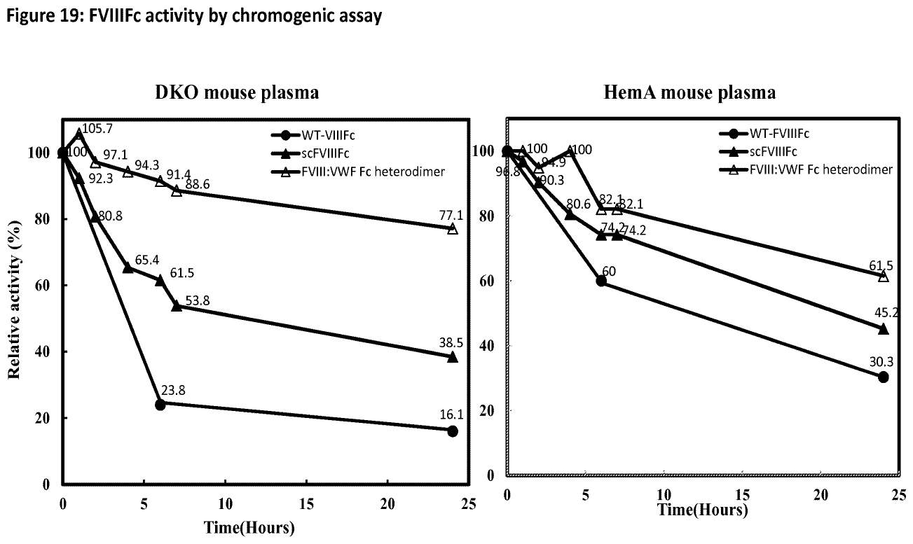

As VWF is proposed to increase the stability of FVIII in vivo, we tested if chimeric protein-FVIII-VWF heterodimer (FVIII155:VWF31, which has VWF D′D3 covalently, attached to FVIII through Fc) was more stable in Hem A and DKO plasma. From plasma stability data shown in FIG. 19, the presence of D′D3 increased the stability of FVIIIFc, both in HemA and DKO plasma. Single chain FVIIIFc without D′D3 was used as control in these experiments (scFVIII). From FIG. 19, single chain FVIII was more stable than dual chain FVIIIFc; however the presence of D′D3 significantly increased the plasma stability of single chain FVIIIFc molecule further. This suggests that D′D3 stabilizes FVIII, not just by holding heavy and light chain together but also through some other unknown mechanisms.

### Example 16: Use of Furin/PACE for VWF Processing

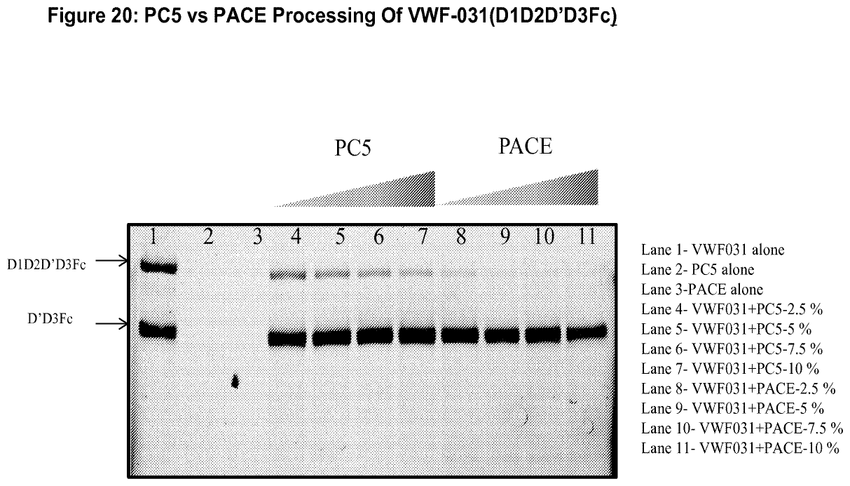

VWF is a unique protein in the sense that it contains a very large pro-peptide (i.e.D1D2 domain of VWF, ~85 kDa). The VWF pro-peptide serves as an internal chaperone for proper folding of VWF molecule. Two enzymes were tested for VWF processing-PC5 and Furin (PACE). VWF031 construct (D1D2D′D3Fc) was transiently co-transfected in HEK293 cells with various concentrations of either PC5 or PACE. After four days, the tissue culture media was collected and subjected to protein A pull down. Even at a lower concentration (2.5%), furin (PACE) was more efficient than 10% PC5, in removing the pro-peptide (DID2) from D′D3Fc (FIG. 20). Removal of DID2 is important, as the presence of DID2 has been implicated in preventing interaction of D′D3 with FVIII.

### Example 17: VWF Fragment in FVIII-VWF Heterodimer Prevents FVIII Interaction with Full Length VWF

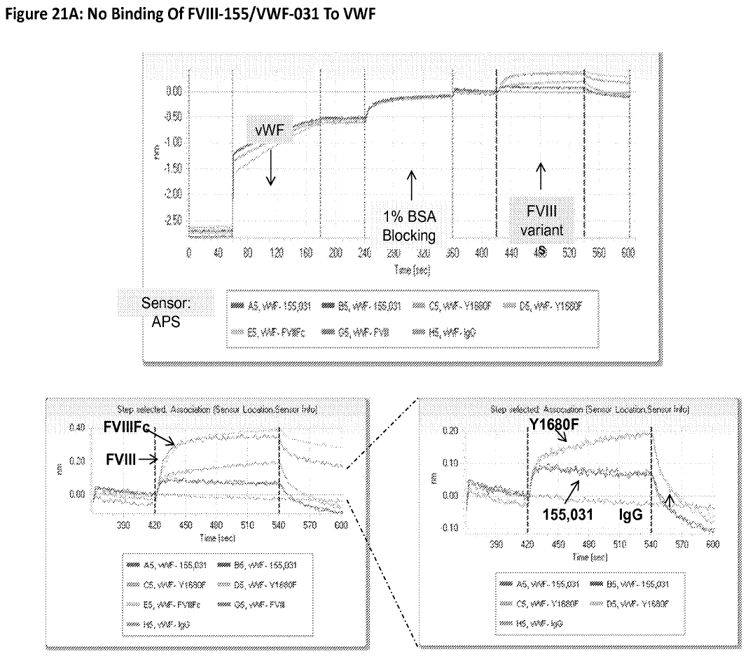

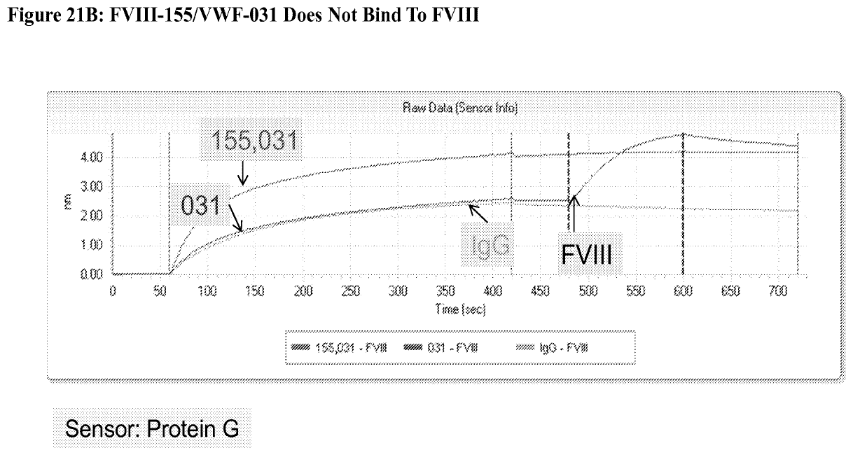

A ForteBio octet instrument was used to test FVIII construct 155/VWF31 heterodimer binding to full length VWF (FIG. 21A). For the binding assay, full length VWF was captured by using APS sensor, followed by blocking with 1% BSA. After blocking, different FVIII constructs were tested for VWF binding. As predicted, wild type FVIII and FVIIIFc bound strongly to the VWF sensors. FVIII Y1680F mutant, which is known to have low or no affinity for VWF showed significantly reduced VWF binding. FVIII155/VWF31 heterodimer did not bind at all to full length VWF, confirming shielding of FVIII with D′D3 in FVIII-VWF heterodimer.

The same experiment was performed in reverse orientation to determine if the D′D3 portion in the FVIII-VWF heterodimer can interact with other FVIII molecules not covalently attached to D′D3. As shown in FIG. 21B, the VWF31 (D′D3Fc) construct alone when immobilized on protein G sensor can bind strongly to FVIII, however the D′D3 in FVIII155:VWF31 heterodimer did not show any binding to FVIII. Protein G alone with FVIII was used as control. These binding experiments confirmed that D′D3 in heterodimer can interact with only one FVIII molecule which is covalently attached to it and prevent FVIII from interacting with full length wild type VWF molecules.

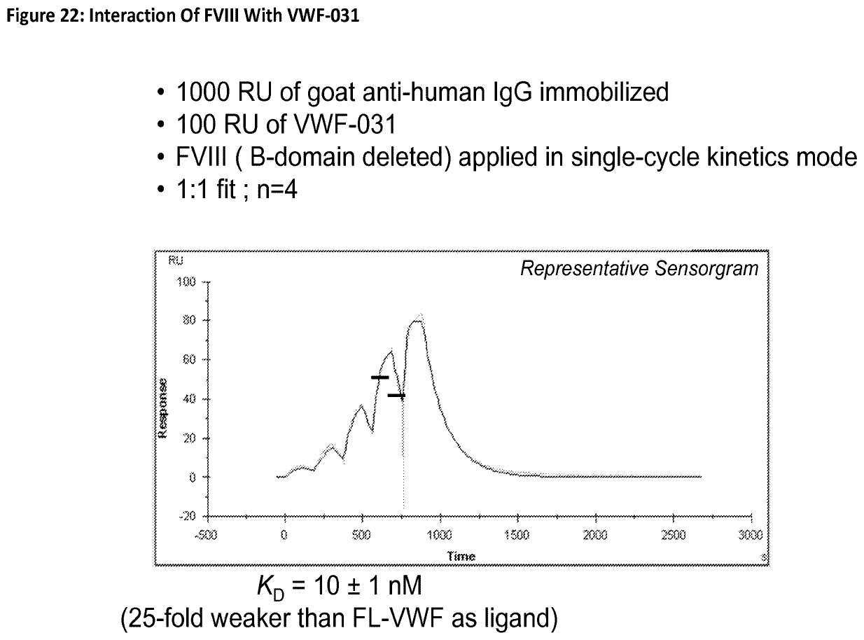

To determine the exact binding affinity of VWF D′D3 for FVIII molecule, surface plasma resonance experiments were performed with VWF031 (FIG. 22). VWF031 construct (D′D3Fc) was captured by using anti-human IgG and B-domain deleted FVIII was passed over D′D3Fc containing chip. A K_{D} of about 10 nM was observed for FVIII. This affinity is about 25-fold lower compare to full length wild type VWF molecule and is similar to what is reported previously in literature.

### Example 18: Effect of Different Linker Length in Between D′D3 and Fc on Heterodimer Activity and PK

To check if varying the length of thrombin cleavable linker in between D′D3 and Fc has any effect on the PK and activity of FVIII-VWF heterodimer, different VWF constructs were co-expressed along with FVIII 155. Three different linker lengths constructs listed in Table 14A were tested (VWF031, VWF035 and VWF036). Each plasmid was mixed with FVIII155 plasmid (Example 5) and transfected into HEK293 cells. At day four post transfection, cell culture media was harvested and concentrated to 10 IU/ml FVIII chromogenic activity.

Concentrated cell media was then administered into 8-12 weeks old FVIII/VWF DKO mice at 100 IU/10 mL/kg dose. Plasma samples were collected at 5 min, 8 hr, 16 hr, 24 hr, 32 hr and 48 hr post dosing. FVIII activity of the plasma samples were analyzed by FVIII chromogenic assay and half-life was calculated using WinNonlin-Phoenix program.

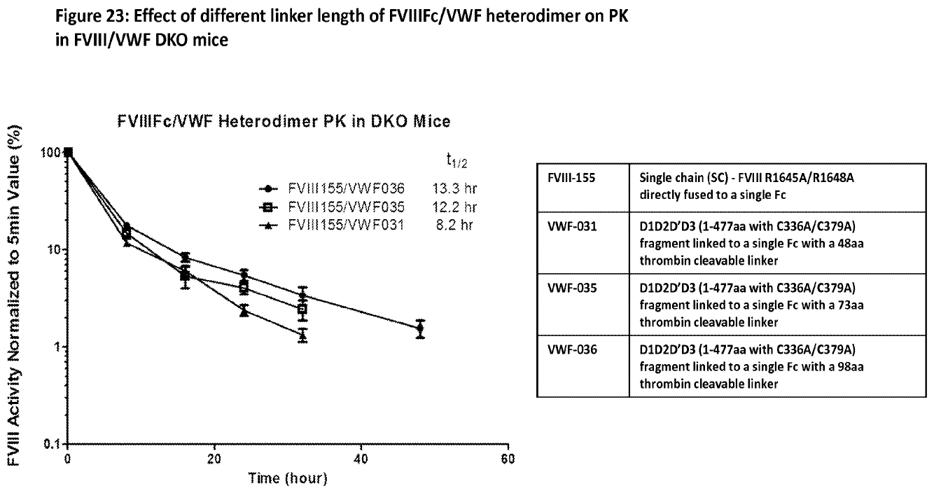

As shown in FIG. 23, when the linker length between D′D3 and Fc fragment was increased from 48 aa to 73aa or 98aa, the half-life of the corresponding FVIIIFc/VWF heterodimer increased and reached 12.2 hr and 13.3 hr respectively. This represents a 1.5 to 1.6 fold increase over 48aa long variant. To date, the 98aa linker is the most optimal linker to utilize the FVIII protection activity of the D′D3 fragment, and it will be incorporated into FVIIIFc/VWF heterodimer to further improve its half-life.

To compare the effect of linker on FVIII activity, FVIII chromogenic and aPTT assay were performed on tissue culture media from cells expressing different FVIII-VWF heterodimers. Though aPTT activity was 2-fold reduced compare to chromogenic activity for heterodimer constructs, no significant difference was seen between various linkers, except when the linker also contain a PARI site next to thrombin site (Table 14B).

TABLE 14A
Sequence of Variable Linker in between VWF D′D3 and Fc

| DNA construct | Linker between D′D3 and Fc |
|---|---|
| VWF031 | 48 aa = I S G G G G S G G G G S G G G G S G |
| | G G G S G G G G S G G G G S L V P R G S G G |
| | G G S G G G G S (SEQ ID NO: 95) |
| VWF035 | 73 aa = I S G G G G S G G G G S G G G G S G G |
| | G G S G G G G S G G G G S G G G G S G G G G S |
| | G G G G S G G G G S G G G G S L V P R G S G G |
| | G G S G G G G S (SEQ ID NO: 96) |
| VWF036 | 98 aa = I G G G G S G G G G S G G G G S G G G |
| | G S G G G G S G G G G S G G G G S G G G G S G |
| | G G G S G G G G S G G G G S G G G G S G G G G |
| | S G G G G S G G G G S G G G G S L V P R G S G |
| | G G G S G G G G S (SEQ ID NO: 97) |

TABLE 14B
Heterodimer activity with different linker length

| | | Linker | | | |
|---|---|---|---|---|---|
| | | length | Chro- | | Chro- |
| | | between | mo- | aPTT | mo- |
| Sample | Sample | D′D3 | genic | IU/ | genic/ |
| ID | description | and Fc (aa) | IU/mL | mL | aPTT |
| 1 | FVIII Fc 155 + VWF15 | 20 | 1.81 | 0.85 | 2.14 |
| 2 | FVIII Fc 155 + VWF31 | 48 | 2.32 | 1.05 | 2.21 |
| 3 | FVIII Fc 155 + VWF33 | 35 | 2.21 | 1.02 | 2.16 |
| 4 | FVIII Fc 155 + VWF35 | 73 | 2.65 | 1.24 | 2.14 |
| 5 | FVIII Fc 155 + VWF36 | 98 | 2.75 | 1.11 | 2.47 |
| 6 | FVIII Fc 155 + VWF39 | 26 | 1.85 | 1.21 | 1.53 |
| | | (thrombin + | | | |
| | | PAR1) | | | |

### Example 19: Linking FVIII with VWF Fragment Using Sortase Enzyme

In another aspect, a VWF fragment (e.g. D1D2D′D3 or D′D3 domain) is attached to FVIII by using sortase mediated in vitro protein ligation method. In one example, Staphylococcus aureus sortase A (LPXTG) recognition motif was introduced at the C-terminus of VWF fragment and Gly(n) residue at the N-terminus of FVIII (where the number of glycine residues is variable). The FVIII molecule used can be either single chain or dual chain. The sortase catalyzed trans-peptidation reaction will covalently attach the VWF fragment to FVIII. Reverse orientation of recognition motif can also be used to link these two proteins, where we have FVIII at the N-terminus with LPXTG motif and VWF fragment at the C-terminus with Gly(n) (See FIG. 24—example of sortase ligation for reference). The LPXTG motif and Glycine residues can be replaced with other sortase recognition sequences.

VWF fragment containing sortase A recognition sequence Fc fusion protein was also made. For Fc fusion constructs, VWF DID2D′D3 fragment was fused with Fc region of IgG through a GS linker that contains a sortase recognition sequence and a thrombin cleavage site (Table 15 and 16). Once protein is expressed and purified on Protein A column, the Fc region can be removed by thrombin cleavage. Resulting VWF fragment with sortase A recognition site can then be used for ligation with FVIII molecule (FIG. 24—Example of sortase ligation for reference-row E).

pSYN-VWF-051 has a 54 amino acids linker with sortase and thrombin site in between the VWF fragment and the Fc region. Synthesis of DNA fragment coding for 54 amino acids linker (ISGGGGSGGG GSGGGGSGGG GSGGGGSGGG GSLPETGALR PRVVGGGGSG GGGS) (SEQ ID NO: 98) and a portion of the Fc region was outsourced (Genewiz Sequence no-10-210746313, shown below). A fragment of the Genewiz construct was sub cloned into the EcoRV/RsRII digested pSYN-VWF-031.

Genewiz-Sequence no-10-210746313
(SEQ ID NO: 99)
AGGAGCCGATATCTGGCGGTGGAGGTTCCGGTGGCGGGGGATCCGGCGGT
GGAGGTTCCGGCGGTGGAGGTTCCGGTGGCGGGGGATCCGGTGGCGGGGG
ATCCTTACCTGAAACTGGAGCCCTGCGGCCCCGGGTCGTCGGCGGTGGAG
GTTCCGGTGGCGGGGGATCCGACAAAACTCACACATGCCCACCGTGCCCA
GCTCCAGAACTCCTGGGCGGACCGTCAGTCTT

The sequence of N-terminus pentaglycine containing single chain FVIII is shown in Table 17 and 18.

TABLE 15
Nucleotide sequence of pSYN-VWF051 (VWF D1D2D′D3Fc with sortase
A recognition motif and thrombin cleavable linker in between
VWF fragment and Fc) (SEQ ID NO: 100)

| | |
|---|---|
| 1 | ATGATTCCTG CCAGATTTGC CGGGGTGCTG CTTGCTCTGG CCCTCATTTT |
| 51 | GCCAGGGACC CTTTGTGCAG AAGGAACTCG CGGCAGGTCA TCCACGGCCC |
| 101 | GATGCAGCCT TTTCGGAAGT GACTTCGTCA ACACCTTTGA TGGGAGCATG |
| 151 | TACAGCTTTG CGGGATACTG CAGTTACCTC CTGGCAGGGG GCTGCCAGAA |
| 201 | ACGCTCCTTC TCGATTATTG GGGACTTCCA GAATGGCAAG AGAGTGAGCC |
| 251 | TCTCCGTGTA TCTTGGGGAA TTTTTTGACA TCCATTTGTT TGTCAATGGT |
| 301 | ACCGTGACAC AGGGGGACCA AAGAGTCTCC ATGCCCTATG CCTCCAAAGG |
| 351 | GCTGTATCTA GAAACTGAGG CTGGGTACTA CAAGCTGTCC GGTGAGGCCT |
| 401 | ATGGCTTTGT GGCCAGGATC GATGGCAGCG GCAACTTTCA AGTCCTGCTG |
| 451 | TCAGACAGAT ACTTCAACAA GACCTGCGGG CTGTGTGGCA ACTTTAACAT |
| 501 | CTTTGCTGAA GATGACTTTA TGACCCAAGA AGGGACCTTG ACCTCGGACC |
| 551 | CTTATGACTT TGCCAACTCA TGGGCTCTGA GCAGTGGAGA ACAGTGGTGT |
| 601 | GAACGGGCAT CTCCTCCCAG CAGCTCATGC AACATCTCCT CTGGGGAAAT |
| 651 | GCAGAAGGGC CTGTGGGAGC AGTGCCAGCT TCTGAAGAGC ACCTCGGTGT |
| 701 | TTGCCCGCTG CCACCCTCTG GTGGACCCCG AGCCTTTTGT GGCCCTGTGT |
| 751 | GAGAAGACTT TGTGTGAGTG TGCTGGGGGG CTGGAGTGCG CCTGCCCTGC |
| 801 | CCTCCTGGAG TACGCCCGGA CCTGTGCCCA GGAGGGAATG GTGCTGTACG |
| 851 | GCTGGACCGA CCACAGCGCG TGCAGCCCAG TGTGCCCTGC TGGTATGGAG |
| 901 | TATAGGCAGT GTGTGTCCCC TTGCGCCAGG ACCTGCCAGA GCCTGCACAT |
| 951 | CAATGAAATG TGTCAGGAGC GATGCGTGGA TGGCTGCAGC TGCCCTGAGG |
| 1001 | GACAGCTCCT GGATGAAGGC CTCTGCGTGG AGAGCACCGA GTGTCCCTGC |
| 1051 | GTGCATTCCG GAAAGCGCTA CCCTCCCGGC ACCTCCCTCT CTCGAGACTG |
| 1101 | CAACACCTGC ATTTGCCGAA ACAGCCAGTG GATCTGCAGC AATGAAGAAT |
| 1151 | GTCCAGGGGA GTGCCTTGTC ACTGGTCAAT CCCACTTCAA GAGCTTTGAC |
| 1201 | AACAGATACT TCACCTTCAG TGGGATCTGC CAGTACCTGC TGGCCCGGGA |
| 1251 | TTGCCAGGAC CACTCCTTCT CCATTGTCAT TGAGACTGTC CAGTGTGCTG |
| 1301 | ATGACCGCGA CGCTGTGTGC ACCCGCTCCG TCACCGTCCG GCTGCCTGGC |
| 1351 | CTGCACAACA GCCTTGTGAA ACTGAAGCAT GGGGCAGGAG TTGCCATGGA |
| 1401 | TGGCCAGGAC ATCCAGCTCC CCCTCCTGAA AGGTGACCTC CGCATCCAGC |
| 1451 | ATACAGTGAC GGCCTCCGTG CGCCTCAGCT ACGGGGAGGA CCTGCAGATG |
| 1501 | GACTGGGATG GCCGCGGGAG GCTGCTGGTG AAGCTGTCCC CCGTCTATGC |
| 1551 | CGGGAAGACC TGCGGCCTGT GTGGGAATTA CAATGGCAAC CAGGGCGACG |
| 1601 | ACTTCCTTAC CCCCTCTGGG CTGGCGGAGC CCCGGGTGGA GGACTTCGGG |
| 1651 | AACGCCTGGA AGCTGCACGG GGACTGCCAG GACCTGCAGA AGCAGCACAG |
| 1701 | CGATCCCTGC GCCCTCAACC CGCGCATGAC CAGGTTCTCC GAGGAGGCGT |
| 1751 | GCGCGGTCCT GACGTCCCCC ACATTCGAGG CCTGCCATCG TGCCGTCAGC |
| 1801 | CCGCTGCCCT ACCTGCGGAA CTGCCGCTAC GACGTGTGCT CCTGCTCGGA |
| 1851 | CGGCCGCGAG TGCCTGTGCG GCGCCCTGGC CAGCTATGCC GCGGCCTGCG |
| 1901 | CGGGGAGAGG CGTGCGCGTC GCGTGGCGCG AGCCAGGCCG CTGTGAGCTG |
| 1951 | AACTGCCCGA AAGGCCAGGT GTACCTGCAG TGCGGGACCC CCTGCAACCT |
| 2001 | GACCTGCCGC TCTCTCTCTT ACCCGGATGA GGAATGCAAT GAGGCCTGCC |
| 2051 | TGGAGGGCTG CTTCTGCCCC CCAGGGCTCT ACATGGATGA GAGGGGGGAC |
| 2101 | TGCGTGCCCA AGGCCCAGTG CCCCTGTTAC TATGACGGTG AGATCTTCCA |
| 2151 | GCCAGAAGAC ATCTTCTCAG ACCATCACAC CATGTGCTAC TGTGAGGATG |
| 2201 | GCTTCATGCA CTGTACCATG AGTGGAGTCC CCGGAAGCTT GCTGCCTGAC |
| 2251 | GCTGTCCTCA GCAGTCCCCT GTCTCATCGC AGCAAAAGGA GCCTATCCTG |
| 2301 | TCGGCCCCCC ATGGTCAAGC TGGTGTGTCC CGCTGACAAC CTGCGGGCTG |
| 2351 | AAGGGCTCGA GTGTACCAAA ACGTGCCAGA ACTATGACCT GGAGTGCATG |
| 2401 | AGCATGGGCT GTGTCTCTGG CTGCCTCTGC CCCCCGGGCA TGGTCCGGCA |
| 2451 | TGAGAACAGA TGTGTGGCCC TGGAAAGGTG TCCCTGCTTC CATCAGGGCA |
| 2501 | AGGAGTATGC CCCTGGAGAA ACAGTGAAGA TTGGCTGCAA CACTTGTGTC |
| 2551 | TGTCGGGACC GGAAGTGGAA CTGCACAGAC CATGTGTGTG ATGCCACGTG |
| 2601 | CTCCACGATC GGCATGGCCC ACTACCTCAC CTTCGACGGG CTCAAATACC |
| 2651 | TGTTCCCCGG GGAGTGCCAG TACGTTCTGG TGCAGGATTA CTGCGGCAGT |
| 2701 | AACCCTGGGA CCTTTCGGAT CCTAGTGGGG AATAAGGGAT GCAGCCACCC |
| 2751 | CTCAGTGAAA TGCAAGAAAC GGGTCACCAT CCTGGTGGAG GGAGGAGAGA |
| 2801 | TTGAGCTGTT TGACGGGGAG GTGAATGTGA AGAGGCCCAT GAAGGATGAG |
| 2851 | ACTCACTTTG AGGTGGTGGA GTCTGGCCGG TACATCATTC TGCTGCTGGG |
| 2901 | CAAAGCCCTC TCCGTGGTCT GGGACCGCCA CCTGAGCATC TCCGTGGTCC |
| 2951 | TGAAGCAGAC ATACCAGGAG AAAGTGTGTG GCCTGTGTGG GAATTTTGAT |
| 3001 | GGCATCCAGA ACAATGACCT CACCAGCAGC AACCTCCAAG TGGAGGAAGA |
| 3051 | CCCTGTGGAC TTTGGGAACT CCTGGAAAGT GAGCTCGCAG TGTGCTGACA |
| 3101 | CCAGAAAAGT GCCTCTGGAC TCATCCCCTG CCACCTGCCA TAACAACATC |
| 3151 | ATGAAGCAGA CGATGGTGGA TTCCTCCTGT AGAATCCTTA CCAGTGACGT |
| 3201 | CTTCCAGGAC TGCAACAAGC TGGTGGACCC CGAGCCATAT CTGGATGTCT |
| 3251 | GCATTTACGA CACCTGCTCC TGTGAGTCCA TTGGGGACTG CGCCGCATTC |
| 3301 | TGCGACACCA TTGCTGCCTA TGCCCACGTG TGTGCCCAGC ATGGCAAGGT |
| 3351 | GGTGACCTGG AGGACGGCCA CATTGTGCCC CCAGAGCTGC GAGGAGAGGA |
| 3401 | ATCTCCGGGA GAACGGGTAT GAGGCTGAGT GGCGCTATAA CAGCTGTGCA |
| 3451 | CCTGCCTGTC AAGTCACGTG TCAGCACCCT GAGCCACTGG CCTGCCCTGT |
| 3501 | GCAGTGTGTG GAGGGCTGCC ATGCCCACTG CCCTCCAGGG AAAATCCTGG |
| 3551 | ATGAGCTTTT GCAGACCTGC GTTGACCCTG AAGACTGTCC AGTGTGTGAG |
| 3601 | GTGGCTGGCC GGCGTTTTGC CTCAGGAAAG AAAGTCACCT TGAATCCCAG |
| 3651 | TGACCCTGAG CACTGCCAGA TTTGCCACTG TGATGTTGTC AACCTCACCT |
| 3701 | GTGAAGCCTG CCAGGAGCCG ATATCTGGCG GTGGAGGTTC CGGTGGCGGG |
| 3751 | GGATCCGGCG GTGGAGGTTC CGGCGGTGGA GGTTCCGGTG GCGGGGGATC |
| 3801 | CGGTGGCGGG GGATCCTTAC CTGAAACTGG AGCCCTGCGG CCCCGGGTCG |
| 3851 | TCGGCGGTGG AGGTTCCGGT GGCGGGGGAT CCGACAAAAC TCACACATGC |
| 3901 | CCACCGTGCC CAGCTCCAGA ACTCCTGGGC GGACCGTCAG TCTTCCTCTT |
| 3951 | CCCCCCAAAA CCCAAGGACA CCCTCATGAT CTCCCGGACC CCTGAGGTCA |
| 4001 | CATGCGTGGT GGTGGACGTG AGCCACGAAG ACCCTGAGGT CAAGTTCAAC |
| 4051 | TGGTACGTGG ACGGCGTGGA GGTGCATAAT GCCAAGACAA AGCCGCGGGA |
| 4101 | GGAGCAGTAC AACAGCACGT ACCGTGTGGT CAGCGTCCTC ACCGTCCTGC |
| 4151 | ACCAGGACTG GCTGAATGGC AAGGAGTACA AGTGCAAGGT CTCCAACAAA |
| 4201 | GCCCTCCCAG CCCCCATCGA GAAAACCATC TCCAAAGCCA AAGGGCAGCC |
| 4251 | CCGAGAACCA CAGGTGTACA CCCTGCCCCC ATCCCGGGAT GAGCTGACCA |
| 4301 | AGAACCAGGT CAGCCTGACC TGCCTGGTCA AAGGCTTCTA TCCCAGCGAC |
| 4351 | ATCGCCGTGG AGTGGGAGAG CAATGGGCAG CCGGAGAACA ACTACAAGAC |
| 4401 | CACGCCTCCC GTGTTGGACT CCGACGGCTC CTTCTTCCTC TACAGCAAGC |
| 4451 | TCACCGTGGA CAAGAGCAGG TGGCAGCAGG GGAACGTCTT CTCATGCTCC |
| 4501 | GTGATGCATG AGGCTCTGCA CAACCACTAC ACGCAGAAGA GCCTCTCCCT |
| 4551 | GTCTCCGGGT AAATGA |

TABLE 16
Protein sequence of VWF051 (VWF D1D2D′D3Fc with sortase A
recognition motif and thrombin cleavable linker in between VWF
fragment and Fc; sortase A site shown in bold) (SEQ ID NO: 101)

| | |
|---|---|
| 1 | MIPARFAGVL LALALILPGT LCAEGTRGRS STARCSLFGS DFVNTFDGSM |
| 51 | YSFAGYCSYL LAGGCQKRSF SIIGDFQNGK RVSLSVYLGE FFDIHLFVNG |
| 101 | TVTQGDQRVS MPYASKGLYL ETEAGYYKLS GEAYGFVARI DGSGNFQVLL |
| 151 | SDRYFNKTCG LCGNFNIFAE DDFMTQEGTL TSDPYDFANS WALSSGEQWC |
| 201 | ERASPPSSSC NISSGEMQKG LWEQCQLLKS TSVFARCHPL VDPEPFVALC |
| 251 | EKTLCECAGG LECACPALLE YARTCAQEGM VLYGWTDHSA CSPVCPAGME |
| 301 | YRQCVSPCAR TCQSLHINEM CQERCVDGCS CPEGQLLDEG LCVESTECPC |
| 351 | VHSGKRYPPG TSLSRDCNTC ICRNSQWICS NEECPGECLV TGQSHFKSFD |
| 401 | NRYFTFSGIC QYLLARDCQD HSFSIVIETV QCADDRDAVC TRSVTVRLPG |
| 451 | LHNSLVKLKH GAGVAMDGQD IQLPLLKGDL RIQHTVTASV RLSYGEDLQM |
| 501 | DWDGRGRLLV KLSPVYAGKT CGLCGNYNGN QGDDFLTPSG LAEPRVEDFG |
| 551 | NAWKLHGDCQ DLQKQHSDPC ALNPRMTRFS EEACAVLTSP TFEACHRAVS |
| 601 | PLPYLRNCRY DVCSCSDGRE CLCGALASYA AACAGRGVRV AWREPGRCEL |
| 651 | NCPKGQVYLQ CGTPCNLTCR SLSYPDEECN EACLEGCFCP PGLYMDERGD |
| 701 | CVPKAQCPCY YDGEIFQPED IFSDHHTMCY CEDGFMHCTM SGVPGSLLPD |
| 751 | AVLSSPLSHR SKRSLSCRPP MVKLVCPADN LRAEGLECTK TCQNYDLECM |
| 801 | SMGCVSGCLC PPGMVRHENR CVALERCPCF HQGKEYAPGE TVKIGCNTCV |
| 851 | CRDRKWNCTD HVCDATCSTI GMAHYLTFDG LKYLFPGECQ YVLVQDYCGS |
| 901 | NPGTFRILVG NKGCSHPSVK CKKRVTILVE GGEIELFDGE VNVKRPMKDE |
| 951 | THFEVVESGR YIILLLGKAL SVVWDRHLSI SVVLKQTYQE KVCGLCGNFD |
| 1001 | GIQNNDLTSS NLQVEEDPVD FGNSWKVSSQ CADTRKVPLD SSPATCHNNI |
| 1051 | MKQTMVDSSC RILTSDVFQD CNKLVDPEPY LDVCIYDTCS CESIGDCAAF |
| 1101 | CDTIAAYAHV CAQHGKVVTW RTATLCPQSC EERNLRENGY EAEWRYNSCA |
| 1151 | PACQVTCQHP EPLACPVQCV EGCHAHCPPG KILDELLQTC VDPEDCPVCE |
| 1201 | VAGRRFASGK KVTLNPSDPE HCQICHCDVV NLTCEACQEP ISGGGGSGGG |
| 1251 | GSGGGGSGGG GSGGGGSGGG GSLPETGALR PRVVGGGGSG GGGSDKTHTC |
| 1301 | PPCPAPELLG GPSVFLFPPK PKDTLMISRT PEVTCVVVDV SHEDPEVKFN |
| 1351 | WYVDGVEVHN AKTKPREEQY NSTYRVVSVL TVLHQDWLNG KEYKCKVSNK |
| 1401 | ALPAPIEKTI SKAKGQPREP QVYTLPPSRD ELTKNQVSLT CLVKGFYPSD |
| 1451 | IAVEWESNGQ PENNYKTTPP VLDSDGSFFL YSKLTVDKSR WQQGNVFSCS |
| 1501 | VMHEALHNHY TQKSLSLSPG K* |

TABLE 17
Nucleotide sequence of FVIII 265 (FVIII single chain
molecule with pentaglycines at N-terminus) (SEQ ID NO: 102)

| | |
|---|---|
| 1 | ATGCAAATAG AGCTCTCCAC CTGCTTCTTT CTGTGCCTTT TGCGATTCTG |
| 51 | CTTTAGTGGA GGAGGAGGAG GAGCCACCAG AAGATACTAC CTGGGTGCAG |
| 101 | TGGAACTGTC ATGGGACTAT ATGCAAAGTG ATCTCGGTGA GCTGCCTGTG |
| 151 | GACGCAAGAT TTCCTCCTAG AGTGCCAAAA TCTTTTCCAT TCAACACCTC |
| 201 | AGTCGTGTAC AAAAAGACTC TGTTTGTAGA ATTCACGGAT CACCTTTTCA |
| 251 | ACATCGCTAA GCCAAGGCCA CCCTGGATGG GTCTGCTAGG TCCTACCATC |
| 301 | CAGGCTGAGG TTTATGATAC AGTGGTCATT ACACTTAAGA ACATGGCTTC |
| 351 | CCATCCTGTC AGTCTTCATG CTGTTGGTGT ATCCTACTGG AAAGCTTCTG |
| 401 | AGGGAGCTGA ATATGATGAT CAGACCAGTC AAAGGGAGAA AGAAGATGAT |
| 451 | AAAGTCTTCC CTGGTGGAAG CCATACATAT GTCTGGCAGG TCCTGAAAGA |
| 501 | GAATGGTCCA ATGGCCTCTG ACCCACTGTG CCTTACCTAC TCATATCTTT |
| 551 | CTCATGTGGA CCTGGTAAAA GACTTGAATT CAGGCCTCAT TGGAGCCCTA |
| 601 | CTAGTATGTA GAGAAGGGAG TCTGGCCAAG GAAAAGACAC AGACCTTGCA |
| 651 | CAAATTTATA CTACTTTTTG CTGTATTTGA TGAAGGGAAA AGTTGGCACT |
| 701 | CAGAAACAAA GAACTCCTTG ATGCAGGATA GGGATGCTGC ATCTGCTCGG |
| 751 | GCCTGGCCTA AAATGCACAC AGTCAATGGT TATGTAAACA GGTCTCTGCC |
| 801 | AGGTCTGATT GGATGCCACA GGAAATCAGT CTATTGGCAT GTGATTGGAA |
| 851 | TGGGCACCAC TCCTGAAGTG CACTCAATAT TCCTCGAAGG TCACACATTT |
| 901 | CTTGTGAGGA ACCATCGCCA GGCGTCCTTG GAAATCTCGC CAATAACTTT |
| 951 | CCTTACTGCT CAAACACTCT TGATGGACCT TGGACAGTTT CTACTGTTTT |
| 1001 | GTCATATCTC TTCCCACCAA CATGATGGCA TGGAAGCTTA TGTCAAAGTA |
| 1051 | GACAGCTGTC CAGAGGAACC CCAACTACGA ATGAAAAATA ATGAAGAAGC |
| 1101 | GGAAGACTAT GATGATGATC TTACTGATTC TGAAATGGAT GTGGTCAGGT |
| 1151 | TTGATGATGA CAACTCTCCT TCCTTTATCC AAATTCGCTC AGTTGCCAAG |
| 1201 | AAGCATCCTA AAACTTGGGT ACATTACATT GCTGCTGAAG AGGAGGACTG |
| 1251 | GGACTATGCT CCCTTAGTCC TCGCCCCCGA TGACAGAAGT TATAAAAGTC |
| 1301 | AATATTTGAA CAATGGCCCT CAGCGGATTG GTAGGAAGTA CAAAAAAGTC |
| 1351 | CGATTTATGG CATACACAGA TGA7ACCTTT AAGACTCGTG AAGCTATTCA |
| 1401 | GCATGAATCA GGAATCTTGG GACCTTTACT TTATGGGGAA GTTGGAGACA |
| 1451 | CACTGTTGAT TATATTTAAG AATCAAGCAA GCAGACCATA TAACATCTAC |
| 1501 | CCTCACGGAA TCACTGATGT CCGTCCTTTG TATTCAAGGA GATTACCAAA |
| 1551 | AGGTGTAAAA CATTTGAAGG ATTTTCCAAT TCTGCCAGGA GAAATATTCA |
| 1601 | AATATAAATG GACAGTGACT GTAGAAGATG GGCCAACTAA ATCAGATCCT |
| 1651 | CGGTGCCTGA CCCGCTATTA CTCTAGTTTC GTTAATATGG AGAGAGATCT |
| 1701 | AGCTTCAGGA CTCATTGGCC CTCTCCTCAT CTGCTACAAA GAATCTGTAG |
| 1751 | ATCAAAGAGG AAACCAGATA ATGTCAGACA AGAGGAATGT CATCCTGTTT |
| 1801 | TCTGTATTTG ATGAGAACCG AAGCTGGTAC CTCACAGAGA ATATACAACG |
| 1851 | CTTTCTCCCC AATCCAGCTG GAGTGCAGCT TGAGGATCCA GAGTTCCAAG |
| 1901 | CCTCCAACAT CATGCACAGC ATCAATGGCT ATGTTTTTGA TAGTTTGCAG |
| 1951 | TTGTCAGTTT GTTTGCATGA GGTGGCATAC TGGTACATTC TAAGCATTGG |
| 2001 | AGCACAGACT GACTTCCTTT CTGTCTTCTT CTCTGGATAT ACCTTCAAAC |
| 2051 | ACAAAATGGT CTATGAAGAC ACACTCACCC TATTCCCATT CTCAGGAGAA |
| 2101 | ACTGTCTTCA TGTCGATGGA AAACCCAGGT CTATGGATTC TGGGGTGCCA |
| 2151 | CAACTCAGAC TTTCGGAACA GAGGCATGAC CGCCTTACTG AAGGTTTCTA |
| 2201 | GTTGTGACAA GAACACTGGT GATTATTACG AGGACAGTTA TGAAGATATT |
| 2251 | TCAGCATACT TGCTGAGTAA AAACAATGCC ATTGAACCAA GAAGCTTCTC |
| 2301 | TCAAAACCCA CCAGTCTTGA AGGCCCATCA GGCCGAAATA ACTCGTACTA |
| 2351 | CTCTTCAGTC AGATCAAGAG GAAATTGACT ATGATGATAC CATATCAGTT |
| 2401 | GAAATGAAGA AGGAAGATTT TGACATTTAT GATGAGGATG AAGATCAGAG |
| 2451 | CCCCCGCAGC TTTCAAAAGA AAACACGACA CTATTTTATT GCTGCAGTGG |
| 2501 | AGAGGCTCTG GGATTATGGG ATGAGTAGCT CCCCACATGT TCTAAGAAAC |
| 2551 | AGGGCTCAGA GTGGCAGTGT CCCTCAGTTC AAGAAAGTTG TTTTCCAGGA |
| 2601 | ATTTACTGAT GGCTCCTTTA CTCAGCCCTT ATACCGTGGA GAACTAAATG |
| 2651 | AACATTTGGG CCTCCTCGGC CCATATATAA GAGCAGAAGT TGAAGATAAT |
| 2701 | ATCATGGTAA CTTTCAGAAA TCAGGCCTCT CGTCCCTATT CCTTCTATTC |
| 2751 | TAGCCTTATT TCTTATGAGG AAGATCAGAG GCAAGGAGCA GAACCTAGAA |
| 2801 | AAAACTTTGT CAAGCCTAAT GAAACCAAAA CTTACTTTTG GAAAGTGCAA |
| 2851 | CATCATATGG CACCCACTAA AGATGAGTTT GACTGCAAAG CCTGGGCTTA |
| 2901 | TTTCTCTGAT GTTGACCTGG AAAAAGATGT GCACTCAGGC CTGATTGGAC |
| 2951 | CCCTTCTGGT CTGCCACACT AACACACTGA ACCCTGCTCA TGGGAGACAA |
| 3001 | GTGACAGTAC AGGAATTTGC TCTGTTTTTC ACCATCTTTG ATGAGACCAA |
| 3051 | AAGCTGGTAC TTCACTGAAA ATATGGAAAG AAACTGCAGG GCTCCCTGCA |
| 3101 | ATATCCAGAT GGAAGATCCC ACTTTTAAAG AGAATTATCG CTTCCATGCA |
| 3151 | ATCAATGGCT ACATAATGGA TACACTACCT GGCTTAGTAA TGGCTCAGGA |
| 3201 | TCAAAGGATT CGATGGTATC TGCTCAGCAT GGGCAGCAAT GAAAACATCC |
| 3251 | ATTCTATTCA TTTCAGTGGA CATGTGTTCA CTGTACGAAA AAAAGAGGAG |
| 3301 | TATAAAATGG CACTGTACAA TCTCTATCCA GGTGTTTTTG AGACAGTGGA |
| 3351 | AATGTTACCA TCCAAAGCTG GAATTTGGCG GGTGGAATGC CTTATTGGCG |
| 3401 | AGCATCTACA TGCTGGGATG AGCACACTTT TTCTGGTGTA CAGCAATAAG |
| 3451 | TGTCAGACTC CCCTGGGAAT GGCTTCTGGA CACATTAGAG ATTTTCAGAT |
| 3501 | TACAGCTTCA GGACAATATG GACAGTGGGC CCCAAAGCTG GCCAGACTTC |
| 3551 | ATTATTCCGG ATCAATCAAT GCCTGGAGCA CCAAGGAGCC CTTTTCTTGG |
| 3601 | ATCAAGGTGG ATCTGTTGGC ACCAATGATT ATTCACGGCA TCAAGACCCA |
| 3651 | GGGTGCCCGT CAGAAGTTCT CCAGCCTCTA CATCTCTCAG TTTATCATCA |
| 3701 | TGTATAGTCT TGATGGGAAG AAGTGGCAGA CTTATCGAGG AAATTCCACT |
| 3751 | GGAACCTTAA TGGTCTTCTT TGGCAATGTG GATTCATCTG GGATAAAACA |
| 3801 | CAATATTTTT AACCCTCCAA TTATTGCTCG ATACATCCGT TTGCACCCAA |
| 3851 | CTCATTATAG CATTCGCAGC ACTCTTCGCA TGGAGTTGAT GGGCTGTGAT |
| 3901 | TTAAATAGTT GCAGCATGCC ATTGGGAATG GAGAGTAAAG CAATATCAGA |
| 3951 | TGCACAGATT ACTGCTTCAT CCTACTTTAC CAATATGTTT GCCACCTGGT |
| 4001 | CTCCTTCAAA AGCTCGACTT CACCTCCAAG GGAGGAGTAA TGCCTGGAGA |
| 4051 | CCTCAGGTGA ATAATCCAAA AGAGTGGCTG CAAGTGGACT TCCAGAAGAC |
| 4101 | AATGAAAGTC ACAGGAGTAA CTACTCAGGG AGTAAAATCT CTGCTTACCA |
| 4151 | GCATGTATGT GAAGGAGTTC CTCATCTCCA GCAGTCAAGA TGGCCATCAG |
| 4201 | TGGACTCTCT TTTTTCAGAA TGGCAAAGTA AAGGTTTTTC AGGGAAATCA |
| 4251 | AGACTCCTTC ACACCTGTGG TGAACTCTCT AGACCCACCG TTACTGACTC |
| 4301 | GCTACCTTCG AATTCACCCC CAGAGTTGGG TGCACCAGAT TGCCCTGAGG |
| 4351 | ATGGAGGTTC TGGGCTGCGA GGCACAGGAC CTCTACTGA |

TABLE 18
Protein sequence of FVIII 265 (FVIII single chain molecule with
pentaglycines at N-terminus; pentaglycine shown in bold)
(SEQ ID NO: 103)

| | |
|---|---|
| 1 | MQIELSTCFF LCLLRFCFSG GGGGATRRYY LGAVELSWDY MQSDLGELPV |
| 51 | DARFPPRVPK SFPFNTSVVY KKTLFVEFTD HLFNIAKPRP PWMGLLGPTI |
| 101 | QAEVYDTVVI TLKNMASHPV SLHAVGVSYW KASEGAEYDD QTSQREKEDD |
| 151 | KVFPGGSHTY VWQVLKENGP MASDPLCLTY SYLSHVDLVK DLNSGLIGAL |
| 201 | LVCREGSLAK EKTQTLHKFI LLFAVFDEGK SWHSETKNSL MQDRDAASAR |
| 251 | AWPKMHTVNG YVNRSLPGLI GCHRKSVYWH VIGMGTTPEV HSIFLEGHTF |
| 301 | LVRNHRQASL EISPITFLTA QTLLMDLGQF LLFCHISSHQ HDGMEAYVKV |
| 351 | DSCPEEPQLR MKNNEEAEDY DDDLTDSEMD VVRFDDDNSP SFIQIRSVAK |
| 401 | KHPKTWVHYI AAEEEDWDYA PLVLAPDDRS YKSQYLNNGP QRIGRKYKKV |
| 451 | RFMAYTDETF KTREAIQHES GILGPLLYGE VGDTLLIIFK NQASRPYNIY |
| 501 | PHGITDVRPL YSRRLPKGVK HLKDFPILPG EIFKYKWTVT VEDGPTKSDP |
| 551 | RCLTRYYSSF VNMERDLASG LIGPLLICYK ESVDQRGNQI MSDKRNVILF |
| 601 | SVFDENRSWY LTENIQRFLP NPAGVQLEDP EFQASNIMHS INGYVFDSLQ |
| 651 | LSVCLHEVAY WYILSIGAQT DFLSVFFSGY TFKHKMVYED TLTLFPFSGE |
| 701 | TVFMSMENPG LWILGCHNSD FRNRGMTALL KVSSCDKNTG DYYEDSYEDI |
| 751 | SAYLLSKNNA IEPRSFSQNP PVLKAHQAEI TRTTLQSDQE EIDYDDTISV |
| 801 | EMKKEDFDIY DEDENQSPRS FQKKTRHYFI AAVERLWDYG MSSSPHVLRN |
| 851 | RAQSGSVPQF KKVVFQEFTD GSFTQPLYRG ELNEHLGLLG PYIRAEVEDN |
| 901 | IMVTFRNQAS RPYSFYSSLI SYEEDQRQGA EPRKNFVKPN ETKTYFWKVQ |
| 951 | HHMAPTKDEF DCKAWAYFSD VDLEKDVHSG LIGPLLVCHT NTLNPAHGRQ |
| 1001 | VTVQEFALFF TIFDETKSWY FTENMERNCR APCNIQMEDP TFKENYRFHA |
| 1051 | INGYIMDTLP GLVMAQDQRI RWYLLSMGSN ENIHSIHFSG HVFTVRKKEE |
| 1101 | YKMALYNLYP GVFETVEMLP SKAGIWRVEC LIGEHLHAGM STLFLVYSNK |
| 1151 | CQTPLGMASG HIRDFQITAS GQYGQWAPKL ARLHYSGSIN AWSTKEPFSW |
| 1201 | IKVDLLAPMI IHGIKTQGAR QKFSSLYISQ FIIMYSLDGK KWQTYRGNST |
| 1251 | GTLMVFFGNV DSSGIKHNIF NPPIIARYIR LHPTHYSIRS TLRMELMGCD |
| 1301 | LNSCSMPLGM ESKAISDAQI TASSYFTNMF ATWSPSKARL HLQGRSNAWR |
| 1351 | PQVNNPKEWL QVDFQKTMKV TGVTTQGVKS LLTSMYVKEF LISSSQDGHQ |
| 1401 | WTLFFQNGKV KVFQGNQDSF TPVVNSLDPP LLTRYLRIHP QSWVHQIALR |
| 1451 | MEVLGCEAQD LY* |

### Example 20: Plasma Stability and PK of FVIII198 in HemA and FVIII/VWF Double Knockout (DKO) Plasma

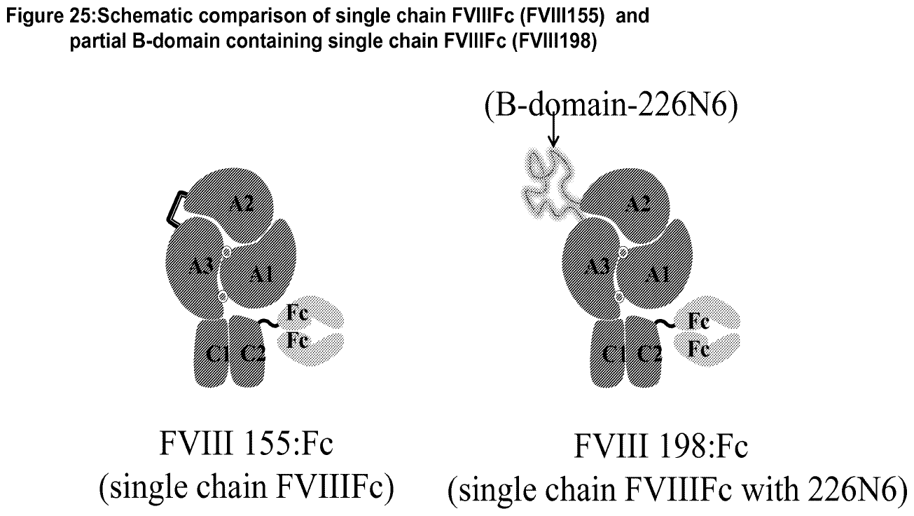

The Plasma stability of FVIII 198 (which is a partial B-domain containing single chain FVIIIFc molecule-226N6; where 226 represents the N-terminus 226 amino acids of FVIII B-domain and N6 represents six N-glycosylation sites in the B-domain) was compared to single chain FVIIIFc (FVIII 155/Fc) in FVIII/VWF double knockout (DKO) plasma. Schematic representation of FVIII155 and FVIII198 can be seen in FIG. 25.

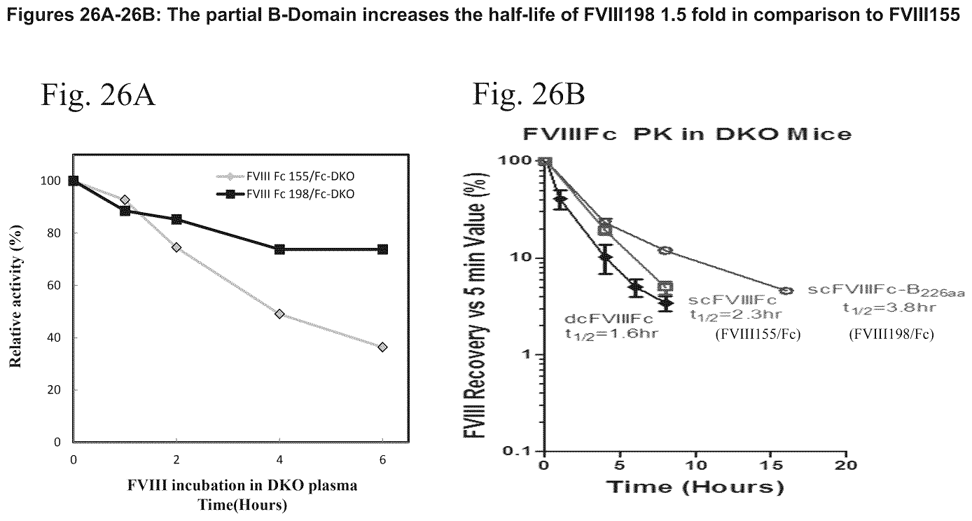

For the stability assay, 5 IU/ml of FVIII 198 or FVIIIFc proteins was incubated with mouse or DKO plasma at 37° C. Aliquots were collected at different time points for activity measurement by FVIII chromogenic assay. Activity at each time point was measured in duplicate and average activity was plotted as a function of time. In the stability assay, the presence of partial B-domain increased the stability of single chain FVIIIFc (FIG. 26A).

The half-life of FVIII 198 (single chain-B226N6) was also compared with FVIII155 (single chain B-domain deleted FVIII) in DKO mice. FVIII 198 has at least about a 1.5 fold longer half-life compared to FVIII155 (FIG. 26B). These experiments suggest that there might be a co-relation between FVIII stability and its in-vivo half-life.

| FVIII198 nucleotide sequence (FVIIIFc with partial B-domain, 226N6) | |
|---|---|
| (SEQ ID NO: 104) | |
| 1 | ATGCAAATAG AGCTCTCCAC CTGCTTCTTT CTGTGCCTTT TGCGATTCTG |
| 51 | CTTTAGTGCC ACCAGAAGAT ACTACCTGGG TGCAGTGGAA CTGTCATGGG |
| 101 | ACTATATGCA AAGTGATCTC GGTGAGCTGC CTGTGGACGC AAGATTTCCT |
| 151 | CCTAGAGTGC CAAAATCTTT TCCATTCAAC ACCTCAGTCG TGTACAAAAA |
| 201 | GACTCTGTTT GTAGAATTCA CGGATCACCT TTTCAACATC GCTAAGCCAA |
| 251 | GGCCACCCTG GATGGGTCTG CTAGGTCCTA CCATCCAGGC TGAGGTTTAT |
| 301 | GATACAGTGG TCATTACACT TAAGAACATG GCTTCCCATC CTGTCAGTCT |
| 351 | TCATGCTGTT GGTGTATCCT ACTGGAAAGC TTCTGAGGGA GCTGAATATG |
| 401 | ATGATCAGAC CAGTCAAAGG GAGAAAGAAG ATGATAAAGT CTTCCCTGGT |
| 451 | GGAAGCCATA CATATGTCTG GCAGGTCCTG AAAGAGAATG GTCCAATGGC |
| 501 | CTCTGACCCA CTGTGCCTTA CCTACTCATA TCTTTCTCAT GTGGACCTGG |
| 551 | TAAAAGACTT GAATTCAGGC CTCATTGGAG CCCTACTAGT ATGTAGAGAA |
| 601 | GGGAGTCTGG CCAAGGAAAA GACACAGACC TTGCACAAAT TTATACTACT |
| 651 | TTTTGCTGTA TTTGATGAAG GGAAAAGTTG GCACTCAGAA ACAAAGAACT |
| 701 | CCTTGATGCA GGATAGGGAT GCTGCATCTG CTCGGGCCTG GCCTAAAATG |
| 751 | CACACAGTCA ATGGTTATGT AAACAGGTCT CTGCCAGGTC TGATTGGATG |
| 801 | CCACAGGAAA TCAGTCTATT GGCATGTGAT TGGAATGGGC ACCACTCCTG |
| 851 | AAGTGCACTC AATATTCCTC GAAGGTCACA CATTTCTTGT GAGGAACCAT |
| 901 | CGCCAGGCGT CCTTGGAAAT CTCGCCAATA ACTTTCCTTA CTGCTCAAAC |
| 951 | ACTCTTGATG GACCTTGGAC AGTTTCTACT GTTTTGTCAT ATCTCTTCCC |
| 1001 | ACCAACATGA TGGCATGGAA GCTTATGTCA AAGTAGACAG CTGTCCAGAG |
| 1051 | GAACCCCAAC TACGAATGAA AAATAATGAA GAAGCGGAAG ACTATGATGA |
| 1101 | TGATCTTACT GATTCTGAAA TGGATGTGGT CAGGTTTGAT GATGACAACT |
| 1151 | CTCCTTCCTT TATCCAAATT CGCTCAGTTG CCAAGAAGCA TCCTAAAACT |
| 1201 | TGGGTACATT ACATTGCTGC TGAAGAGGAG GACTGGGACT ATGCTCCCTT |
| 1251 | AGTCCTCGCC CCCGATGACA GAAGTTATAA AAGTCAATAT TTGAACAATG |
| 1301 | GCCCTCAGCG GATTGGTAGG AAGTACAAAA AAGTCCGATT TATGGCATAC |
| 1351 | ACAGATGAAA CCTTTAAGAC TCGTGAAGCT ATTCAGCATG AATCAGGAAT |
| 1401 | CTTGGGACCT TTACTTTATG GGGAAGTTGG AGACACACTG TTGATTATAT |
| 1451 | TTAAGAATCA AGCAAGCAGA CCATATAACA TCTACCCTCA CGGAATCACT |
| 1501 | GATGTCCGTC CTTTGTATTC AAGGAGATTA CCAAAAGGTG TAAAACATTT |
| 1551 | GAAGGATTTT CCAATTCTGC CAGGAGAAAT ATTCAAATAT AAATGGACAG |
| 1601 | TGACTGTAGA AGATGGGCCA ACTAAATCAG ATCCTCGGTG CCTGACCCGC |
| 1651 | TATTACTCTA GTTTCGTTAA TATGGAGAGA GATCTAGCTT CAGGACTCAT |
| 1701 | TGGCCCTCTC CTCATCTGCT ACAAAGAATC TGTAGATCAA AGAGGAAACC |
| 1751 | AGATAATGTC AGACAAGAGG AATGTCATCC TGTTTTCTGT ATTTGATGAG |
| 1801 | AACCGAAGCT GGTACCTCAC AGAGAATATA CAACGCTTTC TCCCCAATCC |
| 1851 | AGCTGGAGTG CAGCTTGAGG ATCCAGAGTT CCAAGCCTCC AACATCATGC |
| 1901 | ACAGCATCAA TGGCTATGTT TTTGATAGTT TGCAGTTGTC AGTTTGTTTG |
| 1951 | CATGAGGTGG CATACTGGTA CATTCTAAGC ATTGGAGCAC AGACTGACTT |
| 2001 | CCTTTCTGTC TTCTTCTCTG GATATACCTT CAAACACAAA ATGGTCTATG |
| 2051 | AAGACACACT CACCCTATTC CCATTCTCAG GAGAAACTGT CTTCATGTCG |
| 2101 | ATGGAAAACC CAGGTCTATG GATTCTGGGG TGCCACAACT CAGACTTTCG |
| 2151 | GAACAGAGGC ATGACCGCCT TACTGAAGGT TTCTAGTTGT GACAAGAACA |
| 2201 | CTGGTGATTA TTACGAGGAC AGTTATGAAG ATATTTCAGC ATACTTGCTG |
| 2251 | AGTAAAAACA ATGCCATTGA ACCAAGAAGC TTCTCTCAGA ATTCAAGACA |
| 2301 | CCCTAGCACT AGGCAAAAGC AATTTAATGC CACCACAATT CCAGAAAATG |
| 2351 | ACATAGAGAA GACTGACCCT TGGTTTGCAC ACAGAACACC TATGCCTAAA |
| 2401 | ATACAAAATG TCTCCTCTAG TGATTTGTTG ATGCTCTTGC GACAGAGTCC |
| 2451 | TACTCCACAT GGGCTATCCT TATCTGATCT CCAAGAAGCC AAATATGAGA |
| 2501 | CTTTTTCTGA TGATCCATCA CCTGGAGCAA TAGACAGTAA TAACAGCCTG |
| 2551 | TCTGAAATGA CACACTTCAG GCCACAGCTC CATCACAGTG GGGACATGGT |
| 2601 | ATTTACCCCT GAGTCAGGCC TCCAATTAAG ATTAAATGAG AAACTGGGGA |
| 2651 | CAACTGCAGC AACAGAGTTG AAGAAACTTG ATTTCAAAGT TTCTAGTACA |
| 2701 | TCAAATAATC TGATTTCAAC AATTCCATCA GACAATTTGG CAGCAGGTAC |
| 2751 | TGATAATACA AGTTCCTTAG GACCCCCAAG TATGCCAGTT CATTATGATA |
| 2801 | GTCAATTAGA TACCACTCTA TTTGGCAAAA AGTCATCTCC CCTTACTGAG |
| 2851 | TCTGGTGGAC CTCTGAGCTT GAGTGAAGAA AATAATGATT CAAAGTTGTT |
| 2901 | AGAATCAGGT TTAATGAATA GCCAAGAAAG TTCATGGGGA AAAAATGTAT |
| 2951 | CGTCAGAAAT AACTCGTACT ACTCTTCAGT CAGATCAAGA GGAAATTGAC |
| 3001 | TATGATGATA CCATATCAGT TGAAATGAAG AAGGAAGATT TTGACATTTA |
| 3051 | TGATGAGGAT GAAAATCAGA GCCCCCGCAG CTTTCAAAAG AAAACACGAC |
| 3101 | ACTATTTTAT TGCTGCAGTG GAGAGGCTCT GGGATTATGG GATGAGTAGC |
| 3151 | TCCCCACATG TTCTAAGAAA CAGGGCTCAG AGTGGCAGTG TCCCTCAGTT |
| 3201 | CAAGAAAGTT GTTTTCCAGG AATTTACTGA TGGCTCCTTT ACTCAGCCCT |
| 3251 | TATACCGTGG AGAACTAAAT GAACATTTGG GACTCCTGGG GCCATATATA |
| 3301 | AGAGCAGAAG TTGAAGATAA TATCATGGTA ACTTTCAGAA ATCAGGCCTC |
| 3351 | TCGTCCCTAT TCCTTCTATT CTAGCCTTAT TTCTTATGAG GAAGATCAGA |
| 3401 | GGCAAGGAGC AGAACCTAGA AAAAACTTTG TCAAGCCTAA TGAAACCAAA |
| 3451 | ACTTACTTTT GGAAAGTGCA ACATCATATG GCACCCACTA AAGATGAGTT |
| 3501 | TGACTGCAAA GCCTGGGCTT ATTTCTCTGA TGTTGACCTG GAAAAAGATG |
| 3551 | TGCACTCAGG CCTGATTGGA CCCCTTCTGG TCTGCCACAC TAACACACTG |
| 3601 | AACCCTGCTC ATGGGAGACA AGTGACAGTA CAGGAATTTG CTCTGTTTTT |
| 3651 | CACCATCTTT GATGAGACCA AAAGCTGGTA CTTCACTGAA AATATGGAAA |
| 3701 | GAAACTGCAG GGCTCCCTGC AATATCCAGA TGGAAGATCC CACTTTTAAA |
| 3751 | GAGAATTATC GCTTCCATGC AATCAATGGC TACATAATGG ATACACTACC |
| 3801 | TGGCTTAGTA ATGGCTCAGG ATCAAAGGAT TCGATGGTAT CTGCTCAGCA |
| 3851 | TGGGCAGCAA TGAAAACATC CATTCTATTC ATTTCAGTGG ACATGTGTTC |
| 3901 | ACTGTACGAA AAAAAGAGGA GTATAAAATG GCACTGTACA ATCTCTATCC |
| 3951 | AGGTGTTTTT GAGACAGTGG AAATGTTACC ATCCAAAGCT GGAATTTGGC |
| 4001 | GGGTGGAATG CCTTATTGGC GAGCATCTAC ATGCTGGGAT GAGCACACTT |
| 4051 | TTTCTGGTGT ACAGCAATAA GTGTCAGACT CCCCTGGGAA TGGCTTCTGG |
| 4101 | ACACATTAGA GATTTTCAGA TTACAGCTTC AGGACAATAT GGACAGTGGG |
| 4151 | CCCCAAAGCT GGCCAGACTT CATTATTCCG GATCAATCAA TGCCTGGAGC |
| 4201 | ACCAAGGAGC CCTTTTCTTG GATCAAGGTG GATCTGTTGG CACCAATGAT |
| 4251 | TATTCACGGC ATCAAGACCC AGGGTGCCCG TCAGAAGTTC TCCAGCCTCT |
| 4301 | ACATCTCTCA GTTTATCATC ATGTATAGTC TTGATGGGAA GAAGTGGCAG |
| 4351 | ACTTATCGAG GAAATTCCAC TGGAACCTTA ATGGTCTTCT TTGGCAATGT |
| 4401 | GGATTCATCT GGGATAAAAC ACAATATTTT TAACCCTCCA ATTATTGCTC |
| 4451 | GATACATCCG TTTGCACCCA ACTCATTATA GCATTCGCAG CACTCTTCGC |
| 4501 | ATGGAGTTGA TGGGCTGTGA TTTAAATAGT TGCAGCATGC CATTGGGAAT |
| 4551 | GGAGAGTAAA GCAATATCAG ATGCACAGAT TACTGCTTCA TCCTACTTTA |
| 4601 | CCAATATGTT TGCCACCTGG TCTCCTTCAA AAGCTCGACT TCACCTCCAA |
| 4651 | GGGAGGAGTA ATGCCTGGAG ACCTCAGGTG AATAATCCAA AAGAGTGGCT |
| 4701 | GCAAGTGGAC TTCCAGAAGA CAATGAAAGT CACAGGAGTA ACTACTCAGG |
| 4751 | GAGTAAAATC TCTGCTTACC AGCATGTATG TGAAGGAGTT CCTCATCTCC |
| 4801 | AGCAGTCAAG ATGGCCATCA GTGGACTCTC TTTTTTCAGA ATGGCAAAGT |
| 4851 | AAAGGTTTTT CAGGGAAATC AAGACTCCTT CACACCTGTG GTGAACTCTC |
| 4901 | TAGACCCACC GTTACTGACT CGCTACCTTC GAATTCACCC CCAGAGTTGG |
| 4951 | GTGCACCAGA TTGCCCTGAG GATGGAGGTT CTGGGCTGCG AGGCACAGGA |
| 5001 | CCTCTACGAC AAAACTCACA CATGCCCACC GTGCCCAGCT CCAGAACTCC |
| 5051 | TGGGCGGACC GTCAGTCTTC CTCTTCCCCC CAAAACCCAA GGACACCCTC |
| 5101 | ATGATCTCCC GGACCCCTGA GGTCACATGC GTGGTGGTGG ACGTGAGCCA |
| 5151 | CGAAGACCCT GAGGTCAAGT TCAACTGGTA CGTGGACGGC GTGGAGGTGC |
| 5201 | ATAATGCCAA GACAAAGCCG CGGGAGGAGC AGTACAACAG CACGTACCGT |
| 5251 | GTGGTCAGCG TCCTCACCGT CCTGCACCAG GACTGGCTGA ATGGCAAGGA |
| 5301 | GTACAAGTGC AAGGTCTCCA ACAAAGCCCT CCCAGCCCCC ATCGAGAAAA |
| 5351 | CCATCTCCAA AGCCAAAGGG CAGCCCCGAG AACCACAGGT GTACACCCTG |
| 5401 | CCCCCATCCC GGGATGAGCT GACCAAGAAC CAGGTCAGCC TGACCTGCCT |
| 5451 | GGTCAAAGGC TTCTATCCCA GCGACATCGC CGTGGAGTGG GAGAGCAATG |
| 5501 | GGCAGCCGGA GAACAACTAC AAGACCACGC CTCCCGTGTT GGACTCCGAC |
| 5551 | GGCTCCTTCT TCCTCTACAG CAAGCTCACC GTGGACAAGA GCAGGTGGCA |
| 5601 | GCAGGGGAAC GTCTTCTCAT GCTCCGTGAT GCATGAGGCT CTGCACAACC |
| 5651 | ACTACACGCA GAAGAGCCTC TCCCTGTCTC CGGGTAAATG A |
| FVIII 198 protein sequence | |
| (SEQ ID NO: 105) | |
| 1 | MQIELSTCFF LCLLRFCFSA TRRYYLGAVE LSWDYMQSDL GELPVDARFP |
| 51 | PRVPKSFPFN TSVVYKKTLF VEFTDHLFNI AKPRPPWMGL LGPTIQAEVY |
| 101 | DTVVITLKNM ASHPVSLHAV GVSYWKASEG AEYDDQTSQR EKEDDKVFPG |
| 151 | GSHTYVWQVL KENGPMASDP LCLTYSYLSH VDLVKDLNSG LIGALLVCRE |
| 201 | GSLAKEKTQT LHKFILLFAV FDEGKSWHSE TKNSLMQDRD AASARAWPKM |
| 251 | HTVNGYVNRS LPGLIGCHRK SVYWHVIGMG TTPEVHSIFL EGHTFLVRNH |
| 301 | RQASLEISPI TFLTAQTLLM DLGQFLLFCH ISSHQHDGME AYVKVDSCPE |
| 351 | EPQLRMKNNE EAEDYDDDLT DSEMDVVRFD DDNSPSFIQI RSVAKKHPKT |
| 401 | WVHYIAAEEE DWDYAPLVLA PDDRSYKSQY LNNGPQRIGR KYKKVRFMAY |
| 451 | TDETFKTREA IQHESGILGP LLYGEVGDTL LIIFKNQASR PYNIYPHGIT |
| 501 | DVRPLYSRRL PKGVKHLKDF PILPGEIFKY KWTVTVEDGP TKSDPRCLTR |
| 551 | YYSSFVNMER DLASGLIGPL LICYKESVDQ RGNQIMSDKR NVILFSVFDE |
| 601 | NRSWYLTENI QRFLPNPAGV QLEDPEFQAS NIMHSINGYV FDSLQLSVCL |
| 651 | HEVAYWYILS IGAQTDFLSV FFSGYTFKHK MVYEDTLTLF PFSGETVFMS |
| 701 | MENPGLWILG CHNSDFRNRG MTALLKVSSC DKNTGDYYED SYEDISAYLL |
| 751 | SKNNAIEPRS FSQNSRHPST RQKQFNATTI PENDIEKTDP WFAHRTPMPK |
| 801 | IQNVSSSDLL MLLRQSPTPH GLSLSDLQEA KYETFSDDPS PGAIDSNNSL |
| 851 | SEMTHFRPQL HHSGDMVFTP ESGLQLRLNE KLGTTAATEL KKLDFKVSST |
| 901 | SNNLISTIPS DNLAAGTDNT SSLGPPSMPV HYDSQLDTTL FGKKSSPLTE |
| 951 | SGGPLSLSEE NNDSKLLESG LMNSQESSWG KNVSSEITRT TLQSDQEEID |
| 1001 | YDDTISVEMK KEDFDIYDED ENQSPRSFQK KTRHYFIAAV ERLWDYGMSS |
| 1051 | SPHVLRNRAQ SGSVPQFKKV VFQEFTDGSF TQPLYRGELN EHLGLLGPYI |
| 1101 | RAEVEDNIMV TFRNQASRPY SFYSSLISYE EDQRQGAEPR KNFVKPNETK |
| 1151 | TYFWKVQHHM APTKDEFDCK AWAYFSDVDL EKDVHSGLIG PLLVCHTNTL |
| 1201 | NPAHGRQVTV QEFALFFTIF DETKSWYFTE NMERNCRAPC NIQMEDPTFK |
| 1251 | ENYRFHAING YIMDTLPGLV MAQDQRIRWY LLSMGSNENI HSIHFSGHVF |
| 1301 | TVRKKEEYKM ALYNLYPGVF ETVEMLPSKA GIWRVECLIG EHLHAGMSTL |
| 1351 | FLVYSNKCQT PLGMASGHIR DFQITASGQY GQWAPKLARL HYSGSINAWS |
| 1401 | TKEPFSWIKV DLLAPMIIHG IKTQGARQKF SSLYISQFII MYSLDGKKWQ |
| 1451 | TYRGNSTGTL MVFFGNVDSS GIKHNIFNPP IIARYIRLHP THYSIRSTLR |
| 1501 | MELMGCDLNS CSMPLGMESK AISDAQITAS SYFTNMFATW SPSKARLHLQ |
| 1551 | GRSNAWRPQV NNPKEWLQVD FQKTMKVTGV TTQGVKSLLT SMYVKEFLIS |
| 1601 | SSQDGHQWTL FFQNGKVKVF QGNQDSFTPV VNSLDPPLLT RYLRIHPQSW |
| 1651 | VHQIALRMEV LGCEAQDLYD KTHTCPPCPA PELLGGPSVF LFPPKPKDTL |
| 1701 | MISRTPEVTC VVVDVSHEDP EVKFNWYVDG VEVHNAKTKP REEQYNSTYR |
| 1751 | VVSVLTVLHQ DWLNGKEYKC KVSNKALPAP IEKTISKAKG QPREPQVYTL |
| 1801 | PPSRDELTKN QVSLTCLVKG FYPSDIAVEW ESNGQPENNY KTTPPVLDSD |
| 1851 | GSFFLYSKLT VDKSRWQQGN VFSCSVMHEA LHNHYTQKSL SLSPGK* |

### Example 21. Expression of DID2 Protein of VWF

Proper folding of D′D3 domain is essential for its binding to FVIII. VWF propeptide (DID2-amino acids 1-763) is required for efficient disulfide bond formation and folding of D′D3. It acts as an internal chaperone for D′D3 folding. VWF constructs making VWF fragments can either be expressed where VWF propeptide (i.e. DID2 domain) is directly attached to D′D3 domain and removed during the regular intracellular processing of D′D3 (i.e. in cis) or, it can either be expressed from other plasmid i.e. in trans. We designed FVIII-VWF heterodimer in such a way where DID2 can either be expressed in cis or trans.

Cloning VWF 053: VWF 053 clone expresses VWF propeptide (DID2 domain) for in trans expression of DID2. VWF propeptide was PCR amplified from full length using ESC 54 and ESC 124.

ESC54-VWF forward with BsiW1 site
(SEQ ID NO: 111)
(CGCTTCGCGACGTACGGCCGCCACCATGATTCCTGCCAGATTTGCC
GGGGTGCTGCTTGCTC)
ESC 124-D1D2 cloning oligo with Not1 site-reverse
(SEQ ID NO: 112)
(CTAGACTCGAGCGGCCGCTCACCTTTTGCTGCGATGAGACAGGGGAC
TGCTGAGGACAGC)

PCR product was digested with BsiW1 and Not1 and ligated into BsiW1/Not1 digested pCDNA 4.

| Nucleotide sequence of VWF 053 (VWF DID2-propeptide) | |
|---|---|
| (SEQ ID NO: 113) | |
| 1 | ATGATTCCTG CCAGATTTGC CGGGGTGCTG CTTGCTCTGG CCCTCATTTT |
| 51 | GCCAGGGACC CTTTGTGCAG AAGGAACTCG CGGCAGGTCA TCCACGGCCC |
| 101 | GATGCAGCCT TTTCGGAAGT GACTTCGTCA ACACCTTTGA TGGGAGCATG |
| 151 | TACAGCTTTG CGGGATACTG CAGTTACCTC CTGGCAGGGG GCTGCCAGAA |
| 201 | ACGCTCCTTC TCGATTATTG GGGACTTCCA GAATGGCAAG AGAGTGAGCC |
| 251 | TCTCCGTGTA TCTTGGGGAA TTTTTTGACA TCCATTTGTT TGTCAATGGT |
| 301 | ACCGTGACAC AGGGGGACCA AAGAGTCTCC ATGCCCTATG CCTCCAAAGG |
| 351 | GCTGTATCTA GAAACTGAGG CTGGGTACTA CAAGCTGTCC GGTGAGGCCT |
| 401 | ATGGCTTTGT GGCCAGGATC GATGGCAGCG GCAACTTTCA AGTCCTGCTG |
| 451 | TCAGACAGAT ACTTCAACAA GACCTGCGGG CTGTGTGGCA ACTTTAACAT |
| 501 | CTTTGCTGAA GATGACTTTA TGACCCAAGA AGGGACCTTG ACCTCGGACC |
| 551 | CTTATGACTT TGCCAACTCA TGGGCTCTGA GCAGTGGAGA ACAGTGGTGT |
| 601 | GAACGGGCAT CTCCTCCCAG CAGCTCATGC AACATCTCCT CTGGGGAAAT |
| 651 | GCAGAAGGGC CTGTGGGAGC AGTGCCAGCT TCTGAAGAGC ACCTCGGTGT |
| 701 | TTGCCCGCTG CCACCCTCTG GTGGACCCCG AGCCTTTTGT GGCCCTGTGT |
| 751 | GAGAAGACTT TGTGTGAGTG TGCTGGGGGG CTGGAGTGCG CCTGCCCTGC |
| 801 | CCTCCTGGAG TACGCCCGGA CCTGTGCCCA GGAGGGAATG GTGCTGTACG |
| 851 | GCTGGACCGA CCACAGCGCG TGCAGCCCAG TGTGCCCTGC TGGTATGGAG |
| 901 | TATAGGCAGT GTGTGTCCCC TTGCGCCAGG ACCTGCCAGA GCCTGCACAT |
| 951 | CAATGAAATG TGTCAGGAGC GATGCGTGGA TGGCTGCAGC TGCCCTGAGG |
| 1001 | GACAGCTCCT GGATGAAGGC CTCTGCGTGG AGAGCACCGA GTGTCCCTGC |
| 1051 | GTGCATTCCG GAAAGCGCTA CCCTCCCGGC ACCTCCCTCT CTCGAGACTG |
| 1101 | CAACACCTGC ATTTGCCGAA ACAGCCAGTG GATCTGCAGC AATGAAGAAT |
| 1151 | GTCCAGGGGA GTGCCTTGTC ACTGGTCAAT CCCACTTCAA GAGCTTTGAC |
| 1201 | AACAGATACT TCACCTTCAG TGGGATCTGC CAGTACCTGC TGGCCCGGGA |
| 1251 | TTGCCAGGAC CACTCCTTCT CCATTGTCAT TGAGACTGTC CAGTGTGCTG |
| 1301 | ATGACCGCGA CGCTGTGTGC ACCCGCTCCG TCACCGTCCG GCTGCCTGGC |
| 1351 | CTGCACAACA GCCTTGTGAA ACTGAAGCAT GGGGCAGGAG TTGCCATGGA |
| 1401 | TGGCCAGGAC ATCCAGCTCC CCCTCCTGAA AGGTGACCTC CGCATCCAGC |
| 1451 | ATACAGTGAC GGCCTCCGTG CGCCTCAGCT ACGGGGAGGA CCTGCAGATG |
| 1501 | GACTGGGATG GCCGCGGGAG GCTGCTGGTG AAGCTGTCCC CCGTCTATGC |
| 1551 | CGGGAAGACC TGCGGCCTGT GTGGGAATTA CAATGGCAAC CAGGGCGACG |
| 1601 | ACTTCCTTAC CCCCTCTGGG CTGGCGGAGC CCCGGGTGGA GGACTTCGGG |
| 1651 | AACGCCTGGA AGCTGCACGG GGACTGCCAG GACCTGCAGA AGCAGCACAG |
| 1701 | CGATCCCTGC GCCCTCAACC CGCGCATGAC CAGGTTCTCC GAGGAGGCGT |
| 1751 | GCGCGGTCCT GACGTCCCCC ACATTCGAGG CCTGCCATCG TGCCGTCAGC |
| 1801 | CCGCTGCCCT ACCTGCGGAA CTGCCGCTAC GACGTGTGCT CCTGCTCGGA |
| 1851 | CGGCCGCGAG TGCCTGTGCG GCGCCCTGGC CAGCTATGCC GCGGCCTGCG |
| 1901 | CGGGGAGAGG CGTGCGCGTC GCGTGGCGCG AGCCAGGCCG CTGTGAGCTG |
| 1951 | AACTGCCCGA AAGGCCAGGT GTACCTGCAG TGCGGGACCC CCTGCAACCT |
| 2001 | GACCTGCCGC TCTCTCTCTT ACCCGGATGA GGAATGCAAT GAGGCCTGCC |
| 2051 | TGGAGGGCTG CTTCTGCCCC CCAGGGCTCT ACATGGATGA GAGGGGGGAC |
| 2101 | TGCGTGCCCA AGGCCCAGTG CCCCTGTTAC TATGACGGTG AGATCTTCCA |
| 2151 | GCCAGAAGAC ATCTTCTCAG ACCATCACAC CATGTGCTAC TGTGAGGATG |
| 2201 | GCTTCATGCA CTGTACCATG AGTGGAGTCC CCGGAAGCTT GCTGCCTGAC |
| 2251 | GCTGTCCTCA GCAGTCCCCT GTCTCATCGC AGCAAAAGG |
| Protein sequence of VWF 053 (VWF D1D2-Propeptide) | |
| (SEQ ID NO: 114) | |
| 1 | MIPARFAGVL LALALILPGT LCAEGTRGRS STARCSLFGS DFVNTFDGSM |
| 51 | YSFAGYCSYL LAGGCQKRSF SIIGDFQNGK RVSLSVYLGE FFDIHLFVNG |
| 101 | TVTQGDQRVS MPYASKGLYL ETEAGYYKLS GEAYGFVARI DGSGNFQVLL |
| 151 | SDRYFNKTCG LCGNFNIFAE DDFMTQEGTL TSDPYDFANS WALSSGEQWC |
| 201 | ERASPPSSSC NISSGEMQKG LWEQCQLLKS TSVFARCHPL VDPEPFVALC |
| 251 | EKTLCECAGG LECACPALLE YARTCAQEGM VLYGWTDHSA CSPVCPAGME |
| 301 | YRQCVSPCAR TCQSLHINEM CQERCVDGCS CPEGQLLDEG LCVESTECPC |
| 351 | VHSGKRYPPG TSLSRDCNTC ICRNSQWICS NEECPGECLV TGQSHFKSFD |
| 401 | NRYFTFSGIC QYLLARDCQD HSFSIVIETV QCADDRDAVC TRSVTVRLPG |
| 451 | LHNSLVKLKH GAGVAMDGQD IQLPLLKGDL RIQHTVTASV RLSYGEDLQM |
| 501 | DWDGRGRLLV KLSPVYAGKT CGLCGNYNGN QGDDFLTPSG LAEPRVEDFG |
| 551 | NAWKLHGDCQ DLQKQHSDPC ALNPRMTRFS EEACAVLTSP TFEACHRAVS |
| 601 | PLPYLRNCRY DVCSCSDGRE CLCGALASYA AACAGRGVRV AWREPGRCEL |
| 651 | NCPKGQVYLQ CGTPCNLTCR SLSYPDEECN EACLEGCFCP PGLYMDERGD |
| 701 | CVPKAQCPCY YDGEIFQPED IFSDHHTMCY CEDGFMHCTM SGVPGSLLPD |
| 751 | AVLSSPLSHR SKR |

The foregoing description of the specific embodiments will so fully reveal the general nature of the invention that others can, by applying knowledge within the skill of the art, readily modify and/or adapt for various applications such specific embodiments, without undue experimentation, without departing from the general concept of the present invention. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

All patents and publications cited herein are incorporated by reference herein in their entirety.

1-147. (canceled)

148. A chimeric protein comprising (a) a Factor VIII (“FVIII”) protein, (b) a thrombin cleavable linker, and (c) an adjunct moiety,
wherein the FVIII protein comprises a full or partial B domain deletion,
wherein the thrombin cleavable linker links the FVIII protein to the adjunct moiety,
wherein the adjunct moiety comprises a D′ domain of VWF and a D3 domain of VWF, wherein the amino acid sequence of the D′ domain of VWF is at least 95% identical to amino acids 764 to 866 of SEQ ID NO: 2, wherein the amino acid sequence of the D3 domain of VWF is at least 95% identical to amino acids 867 to 1240 of SEQ ID NO: 2, and
wherein the adjunct moiety inhibits or prevents endogenous VWF from binding to the FVIII protein.

149. The chimeric protein of claim 148, wherein the amino acid sequence of the D3 domain of VWF is at least 95% identical to amino acids 867 to 1240 of SEQ ID NO: 2, and wherein alanine is substituted for cysteine at residues corresponding to residues 1099 and 1142 of SEQ ID NO: 2.

150. The chimeric protein of claim 149, wherein the adjunct moiety further comprises a fragment of the A1 domain of VWF.

151. The chimeric protein of claim 149, wherein the adjunct moiety does not comprise the A1 domain, the A2 domain, the A3 domain, the D4 domain, the B1 domain, the B2 domain, the B3 domain, the C1 domain, or the C2 domain of VWF.

152. The chimeric protein of claim 149, wherein the chimeric protein has been expressed by a HEK293 cell and purified.

153. The chimeric protein of claim 149, wherein the D′ domain of VWF is at least 97% identical to amino acids 764 to 866 of SEQ ID NO: 2, and wherein the amino acid sequence of the D3 domain of VWF is at least 97% identical to amino acids 867 to 1240 of SEQ ID NO: 2 with cysteine to alanine substitutions at residues 1099 and 1142 of SEQ ID NO: 2.

154. The chimeric protein of claim 149, wherein the D′ domain of VWF is at least 98% identical to amino acids 764 to 866 of SEQ ID NO: 2, and wherein the amino acid sequence of the D3 domain of VWF is at least 98% identical to amino acids 867 to 1240 of SEQ ID NO: 2 with cysteine to alanine substitutions at residues 1099 and 1142 of SEQ ID NO: 2.

155. The chimeric protein of claim 149, wherein the D′ domain of VWF is at least 99% identical to amino acids 764 to 866 of SEQ ID NO: 2, and wherein the amino acid sequence of the D3 domain of VWF is at least 99% identical to amino acids 867 to 1240 of SEQ ID NO: 2 with cysteine to alanine substitutions at residues corresponding to residues 1099 and 1142 of SEQ ID NO: 2.

156. The chimeric protein of claim 149, wherein the D′ domain of VWF is identical to amino acids 764 to 866 of SEQ ID NO: 2, and wherein the amino acid sequence of the D3 domain of VWF is identical to amino acids 867 to 1240 of SEQ ID NO: 2 with cysteine to alanine substitutions at residues corresponding to residues 1099 and 1142 of SEQ ID NO: 2.

157. The chimeric protein of claim 149, the FVIII protein comprises a partial B domain deletion.

158. The chimeric protein of claim 152, wherein the D′ domain of VWF is at least 97% identical to amino acids 764 to 866 of SEQ ID NO: 2, and wherein the amino acid sequence of the D3 domain of VWF is at least 97% identical to amino acids 867 to 1240 of SEQ ID NO: 2 with cysteine to alanine substitutions at residues 1099 and 1142 of SEQ ID NO: 2.

159. The chimeric protein of claim 152, wherein the D′ domain of VWF is at least 98% identical to amino acids 764 to 866 of SEQ ID NO: 2, and wherein the amino acid sequence of the D3 domain of VWF is at least 98% identical to amino acids 867 to 1240 of SEQ ID NO: 2 with cysteine to alanine substitutions at residues 1099 and 1142 of SEQ ID NO: 2.

160. The chimeric protein of claim 152, wherein the D′ domain of VWF is at least 99% identical to amino acids 764 to 866 of SEQ ID NO: 2, and wherein the amino acid sequence of the D3 domain of VWF is at least 99% identical to amino acids 867 to 1240 of SEQ ID NO: 2 with cysteine to alanine substitutions at residues corresponding to residues 1099 and 1142 of SEQ ID NO: 2.

161. The chimeric protein of claim 152, wherein the D′ domain of VWF is identical to amino acids 764 to 866 of SEQ ID NO: 2, and wherein the amino acid sequence of the D3 domain of VWF is identical to amino acids 867 to 1240 of SEQ ID NO: 2 with cysteine to alanine substitutions at residues corresponding to residues 1099 and 1142 of SEQ ID NO: 2.


SEQUENCE LISTING (114 sequences)

SEQ ID NO: 1 - DNA, 16842 bp, Homo sapiens
  source 1..16842: mol_type = unassigned DNA
atgattcctgccagatttgccggggtgctgcttgctctggccctcattttgccagggaccctttgtgcagaaggaactcgcggcaggtcatccacggccctactaaggacggtctaaacggccccacgacgaacgagaccgggagtaaaacggtccctgggaaacacgtcttccttgagcgccgtccagtaggtgccggggatgcagccttttcggaagtgacttcgtcaacacctttgatgggagcatgtacagctttgcgggatactgcagttacctcctggcagggggctgccagaactacgtcggaaaagccttcactgaagcagttgtggaaactaccctcgtacatgtcgaaacgccctatgacgtcaatggaggaccgtcccccgacggtcttacgctccttctcgattattggggacttccagaatggcaagagagtgagcctctccgtgtatcttggggaattttttgacatccatttgtttgtcaatggttgcgaggaagagctaataacccctgaaggtcttaccgttctctcactcggagaggcacatagaaccccttaaaaaactgtaggtaaacaaacagttaccaaccgtgacacagggggaccaaagagtctccatgccctatgcctccaaagggctgtatctagaaactgaggctgggtactacaagctgtccggtgaggccttggcactgtgtccccctggtttctcagaggtacgggatacggaggtttcccgacatagatctttgactccgacccatgatgttcgacaggccactccggaatggctttgtggccaggatcgatggcagcggcaactttcaagtcctgctgtcagacagatacttcaacaagacctgcgggctgtgtggcaactttaacattaccgaaacaccggtcctagctaccgtcgccgttgaaagttcaggacgacagtctgtctatgaagttgttctggacgcccgacacaccgttgaaattgtactttgctgaagatgactttatgacccaagaagggaccttgacctcggacccttatgactttgccaactcatgggctctgagcagtggagaacagtggtgtgaaacgacttctactgaaatactgggttcttccctggaactggagcctgggaatactgaaacggttgagtacccgagactcgtcacctcttgtcaccacagaacgggcatctcctcccagcagctcatgcaacatctcctctggggaaatgcagaagggcctgtgggagcagtgccagcttctgaagagcacctcggtgtcttgcccgtagaggagggtcgtcgagtacgttgtagaggagacccctttacgtcttcccggacaccctcgtcacggtcgaagacttctcgtggagccacattgcccgctgccaccctctggtggaccccgagccttttgtggccctgtgtgagaagactttgtgtgagtgtgctggggggctggagtgcgcctgccctgcaacgggcgacggtgggagaccacctggggctcggaaaacaccgggacacactcttctgaaacacactcacacgaccccccgacctcacgcggacgggacgcctcctggagtacgcccggacctgtgcccaggagggaatggtgctgtacggctggaccgaccacagcgcgtgcagcccagtgtgccctgctggtatggagggaggacctcatgcgggcctggacacgggtcctcccttaccacgacatgccgacctggctggtgtcgcgcacgtcgggtcacacgggacgaccatacctctataggcagtgtgtgtccccttgcgccaggacctgccagagcctgcacatcaatgaaatgtgtcaggagcgatgcgtggatggctgcagctgccctgaggatatccgtcacacacaggggaacgcggtcctggacggtctcggacgtgtagttactttacacagtcctcgctacgcacctaccgacgtcgacgggactccgacagctcctggatgaaggcctctgcgtggagagcaccgagtgtccctgcgtgcattccggaaagcgctaccctcccggcacctccctctctcgagactgctgtcgaggacctacttccggagacgcacctctcgtggctcacagggacgcacgtaaggcctttcgcgatgggagggccgtggagggagagagctctgaccaacacctgcatttgccgaaacagccagtggatctgcagcaatgaagaatgtccaggggagtgccttgtcactggtcaatcccacttcaagagctttgacgttgtggacgtaaacggctttgtcggtcacctagacgtcgttacttcttacaggtcccctcacggaacagtgaccagttagggtgaagttctcgaaactgaacagatacttcaccttcagtgggatctgccagtacctgctggcccgggattgccaggaccactccttctccattgtcattgagactgtccagtgtgctgttgtctatgaagtggaagtcaccctagacggtcatggacgaccgggccctaacggtcctggtgaggaagaggtaacagtaactctgacaggtcacacgacatgaccgcgacgctgtgtgcacccgctccgtcaccgtccggctgcctggcctgcacaacagccttgtgaaactgaagcatggggcaggagttgccatggatactggcgctgcgacacacgtgggcgaggcagtggcaggccgacggaccggacgtgttgtcggaacactttgacttcgtaccccgtcctcaacggtaccttggccaggacatccagctccccctcctgaaaggtgacctccgcatccagcatacagtgacggcctccgtgcgcctcagctacggggaggacctgcagatgaccggtcctgtaggtcgagggggaggactttccactggaggcgtaggtcgtatgtcactgccggaggcacgcggagtcgatgcccctcctggacgtctacgactgggatggccgcgggaggctgctggtgaagctgtcccccgtctatgccgggaagacctgcggcctgtgtgggaattacaatggcaaccagggcgacgctgaccctaccggcgccctccgacgaccacttcgacagggggcagatacggcccttctggacgccggacacacccttaatgttaccgttggtcccgctgcacttccttaccccctctgggctggcrgagccccgggtggaggacttcgggaacgcctggaagctgcacggggactgccaggacctgcagaagcagcacagtgaaggaatgggggagacccgaccgyctcggggcccacctcctgaagcccttgcggaccttcgacgtgcccctgacggtcctggacgtcttcgtcgtgtccgatccctgcgccctcaacccgcgcatgaccaggttctccgaggaggcgtgcgcggtcctgacgtcccccacattcgaggcctgccatcgtgccgtcagcgctagggacgcgggagttgggcgcgtactggtccaagaggctcctccgcacgcgccaggactgcagggggtgtaagctccggacggtagcacggcagtcgccgctgccctacctgcggaactgccgctacgacgtgtgctcctgctcggacggccgcgagtgcctgtgcggcgccctggccagctatgccgcggcctgcgggcgacgggatggacgccttgacggcgatgctgcacacgaggacgagcctgccggcgctcacggacacgccgcgggaccggtcgatacggcgccggacgccggggagaggcgtgcgcgtcgcgtggcgcgagccaggccgctgtgagctgaactgcccgaaaggccaggtgtacctgcagtgcgggaccccctgcaacctgcccctctccgcacgcgcagcgcaccgcgctcggtccggcgacactcgacttgacgggctttccggtccacatggacgtcacgccctgggggacgttggagacctgccgctctctctcttacccggatgaggaatgcaatgaggcctgcctggagggctgcttctgccccccagggctctacatggatgagaggggggacctggacggcgagagagagaatgggcctactccttacgttactccggacggacctcccgacgaagacggggggtcccgagatgtacctactctcccccctgtgcgtgcccaaggcccagtgcccctgttactatgacggtgagatcttccagccagaagacatcttctcagaccatcacaccatgtgctactgtgaggatgacgcacgggttccgggtcacggggacaatgatactgccactctagaaggtcggtcttctgtagaagagtctggtagtgtggtacacgatgacactcctacgcttcatgcactgtaccatgagtggagtccccggaagcttgctgcctgacgctgtcctcagcagtcccctgtctcatcgcagcaaaaggagcctatcctgcgaagtacgtgacatggtactcacctcaggggccttcgaacgacggactgcgacaggagtcgtcaggggacagagtagcgtcgttttcctcggataggactcggccccccatggtcaagctggtgtgtcccgctgacaacctgcgggctgaagggctcgagtgtaccaaaacgtgccagaactatgacctggagtgcatgagccggggggtaccagttcgaccacacagggcgactgttggacgcccgacttcccgagctcacatggttttgcacggtcttgatactggacctcacgtacagcatgggctgtgtctctggctgcctctgccccccgggcatggtccggcatgagaacagatgtgtggccctggaaaggtgtccctgcttccatcagggcatcgtacccgacacagagaccgacggagacggggggcccgtaccaggccgtactcttgtctacacaccgggacctttccacagggacgaaggtagtcccgtaggagtatgcccctggagaaacagtgaagattggctgcaacacttgtgtctgtcgggaccggaagtggaactgcacagaccatgtgtgtgatgccacgtgtcctcatacggggacctctttgtcacttctaaccgacgttgtgaacacagacagccctggccttcaccttgacgtgtctggtacacacactacggtgcacctccacgatcggcatggcccactacctcaccttcgacgggctcaaatacctgttccccggggagtgccagtacgttctggtgcaggattactgcggcagtgaggtgctagccgtaccgggtgatggagtggaagctgcccgagtttatggacaaggggcccctcacggtcatgcaagaccacgtcctaatgacgccgtcaaaccctgggacctttcggatcctagtggggaataagggatgcagccacccctcagtgaaatgcaagaaacgggtcaccatcctggtggagggaggagagattgggaccctggaaagcctaggatcaccccttattccctacgtcggtggggagtcactttacgttctttgcccagtggtaggaccacctccctcctctctttgagctgtttgacggggaggtgaatgtgaagaggcccatgaaggatgagactcactttgaggtggtggagtctggccggtacatcattctgctgctgggaactcgacaaactgcccctccacttacacttctccgggtacttcctactctgagtgaaactccaccacctcagaccggccatgtagtaagacgacgaccccaaagccctctccgtggtctgggaccgccacctgagcatctccgtggtcctgaagcagacataccaggagaaagtgtgtggcctgtgtgggaattttgatgtttcgggagaggcaccagaccctggcggtggactcgtagaggcaccaggacttcgtctgtatggtcctctttcacacaccggacacacccttaaaactaggcatccagaacaatgacctcaccagcagcaacctccaagtggaggaagaccctgtggactttgggaactcctggaaagtgagctcgcagtgtgctgacaccgtaggtcttgttactggagtggtcgtcgttggaggttcacctccttctgggacacctgaaacccttgaggacctttcactcgagcgtcacacgactgtccagaaaagtgcctctggactcatcccctgccacctgccataacaacatcatgaagcagacgatggtggattcctcctgtagaatccttaccagtgacgtggtcttttcacggagacctgagtaggggacggtggacggtattgttgtagtacttcgtctgctaccacctaaggaggacatcttaggaatggtcactgcacttccaggactgcaacaagctggtggaccccgagccatatctggatgtctgcatttacgacacctgctcctgtgagtccattggggactgcgcctgcttcgaaggtcctgacgttgttcgaccacctggggctcggtatagacctacagacgtaaatgctgtggacgaggacactcaggtaacccctgacgcggacgaagtgcgacaccattgctgcctatgcccacgtgtgtgcccagcatggcaaggtggtgacctggaggacggccacattgtgcccccagagctgcgaggagaggaacgctgtggtaacgacggatacgggtgcacacacgggtcgtaccgttccaccactggacctcctgccggtgtaacacgggggtctcgacgctcctctcctatctccgggagaacgggtatgagtgtgagtggcgctataacagctgtgcacctgcctgtcaagtcacgtgtcagcaccctgagccactggcctgccctgttagaggccctcttgcccatactcacactcaccgcgatattgtcgacacgtggacggacagttcagtgcacagtcgtgggactcggtgaccggacgggacagcagtgtgtggagggctgccatgcccactgccctccagggaaaatcctggatgagcttttgcagacctgcgttgaccctgaagactgtccagtgtgtgagcgtcacacacctcccgacggtacgggtgacgggaggtcccttttaggacctactcgaaaacgtctggacgcaactgggacttctgacaggtcacacactcgtggctggccggcgttttgcctcaggaaagaaagtcaccttgaatcccagtgaccctgagcactgccagatttgccactgtgatgttgtcaacctcacctcaccgaccggccgcaaaacggagtcctttctttcagtggaacttagggtcactgggactcgtgacggtctaaacggtgacactacaacagttggagtggagtgaagcctgccaggagccgggaggcctggtggtgcctcccacagatgccccggtgagccccaccactctgtatgtggaggacatctcggaaccgccgttcacttcggacggtcctcggccctccggaccaccacggagggtgtctacggggccactcggggtggtgagacatacacctcctgtagagccttggcggcaagcacgatttctactgcagcaggctactggacctggtcttcctgctggatggctcctccaggctgtccgaggctgagtttgaagtgctgaaggcctttgtgcgtgctaaagatgacgtcgtccgatgacctggaccagaaggacgacctaccgaggaggtccgacaggctccgactcaaacttcacgacttccggaaacacgtggacatgatggagcggctgcgcatctcccagaagtgggtccgcgtggccgtggtggagtaccacgacggctcccacgcctacatcgggctcaaggacccacctgtactacctcgccgacgcgtagagggtcttcacccaggcgcaccggcaccacctcatggtgctgccgagggtgcggatgtagcccgagttcctggggaagcgaccgtcagagctgcggcgcattgccagccaggtgaagtatgcgggcagccaggtggcctccaccagcgaggtcttgaaatacacactgttccaccttcgctggcagtctcgacgccgcgtaacggtcggtccacttcatacgcccgtcggtccaccggaggtggtcgctccagaactttatgtgtgacaaggtaatcttcagcaagatcgaccgccctgaagcctcccgcatcgccctgctcctgatggccagccaggagccccaacggatgtcccggaactttgtccgctacttagaagtcgttctagctggcgggacttcggagggcgtagcgggacgaggactaccggtcggtcctcggggttgcctacagggccttgaaacaggcgatggtccagggcctgaagaagaagaaggtcattgtgatcccggtgggcattgggccccatgccaacctcaagcagatccgcctcatcgagaagcaggcccctgcaggtcccggacttcttcttcttccagtaacactagggccacccgtaacccggggtacggttggagttcgtctaggcggagtagctcttcgtccggggacagaacaaggccttcgtgctgagcagtgtggatgagctggagcagcaaagggacgagatcgttagctacctctgtgaccttgcccctgaagcccctcctcctcttgttccggaagcacgactcgtcacacctactcgacctcgtcgtttccctgctctagcaatcgatggagacactggaacggggacttcggggaggaggtactctgccccccgacatggcacaagtcactgtgggcccggggctcttgggggtttcgaccctggggcccaagaggaactccatggttctggatgtggcgatgagacggggggctgtaccgtgttcagtgacacccgggccccgagaacccccaaagctgggaccccgggttctccttgaggtaccaagacctacaccgcttcgtcctggaaggatcggacaaaattggtgaagccgacttcaacaggagcaaggagttcatggaggaggtgattcagcggatggatgtgggccaggacaaagcaggaccttcctagcctgttttaaccacttcggctgaagttgtcctcgttcctcaagtacctcctccactaagtcgcctacctacacccggtcctgtgcatccacgtcacggtgctgcagtactcctacatggtgaccgtggagtaccccttcagcgaggcacagtccaaaggggacatcctgcagcgggtgcgagacgtaggtgcagtgccacgacgtcatgaggatgtaccactggcacctcatggggaagtcgctccgtgtcaggtttcccctgtaggacgtcgcccacgctctgatccgctaccagggcggcaacaggaccaacactgggctggccctgcggtacctctctgaccacagcttcttggtcagccagggtgaccgggagcaggcgctaggcgatggtcccgccgttgtcctggttgtgacccgaccgggacgccatggagagactggtgtcgaagaaccagtcggtcccactggccctcgtccgccccaacctggtctacatggtcaccggaaatcctgcctctgatgagatcaagaggctgcctggagacatccaggtggtgcccattggagtgggccctaatggggttggaccagatgtaccagtggcctttaggacggagactactctagttctccgacggacctctgtaggtccaccacgggtaacctcacccgggattacccaacgtgcaggagctggagaggattggctggcccaatgcccctatcctcatccaggactttgagacgctcccccgagaggctcctgacctggtgctgcaggttgcacgtcctcgacctctcctaaccgaccgggttacggggataggagtaggtcctgaaactctgcgagggggctctccgaggactggaccacgacgtgaggtgctgctccggagaggggctgcagatccccaccctctcccctgcacctgactgcagccagcccctggacgtgatccttctcctggatggctcctccctccacgacgaggcctctccccgacgtctaggggtgggagaggggacgtggactgacgtcggtcggggacctgcactaggaagaggacctaccgaggaggagtttcccagcttcttattttgatgaaatgaagagtttcgccaaggctttcatttcaaaagccaatatagggcctcgtctcactcaggtgtcagtgctgctcaaagggtcgaagaataaaactactttacttctcaaagcggttccgaaagtaaagttttcggttatatcccggagcagagtgagtccacagtcacgacgagtatggaagcatcaccaccattgacgtgccatggaacgtggtcccggagaaagcccatttgctgagccttgtggacgtcatgcagcgggagggaggccctcataccttcgtagtggtggtaactgcacggtaccttgcaccagggcctctttcgggtaaacgactcggaacacctgcagtacgtcgccctccctccgggcagccaaatcggggatgccttgggctttgctgtgcgatacttgacttcagaaatgcatggtgccaggccgggagcctcaaaggcggtggtcatcctggtcgtcggtttagcccctacggaacccgaaacgacacgctatgaactgaagtctttacgtaccacggtccggccctcggagtttccgccaccagtaggaccagacggacgtctctgtggattcagtggatgcagcagctgatgccgccaggtccaacagagtgacagtgttccctattggaattggagatcgctacgatgcagtgcctgcagagacacctaagtcacctacgtcgtcgactacggcggtccaggttgtctcactgtcacaagggataaccttaacctctagcgatgctacgtccccagctacggatcttggcaggcccagcaggcgactccaacgtggtgaagctccagcgaatcgaagacctccctaccatggtcaccttgggcaattccttgggtcgatgcctagaaccgtccgggtcgtccgctgaggttgcaccacttcgaggtcgcttagcttctggagggatggtaccagtggaacccgttaaggaacctccacaaactgtgctctggatttgttaggatttgcatggatgaggatgggaatgagaagaggcccggggacgtctggaccttgccagaccagtgccacggaggtgtttgacacgagacctaaacaatcctaaacgtacctactcctacccttactcttctccgggcccctgcagacctggaacggtctggtcacggtgaccgtgacttgccagccagatggccagaccttgctgaagagtcatcgggtcaactgtgaccgggggctgaggccttcgtgccctaacagccagtcccctgtggcactgaacggtcggtctaccggtctggaacgacttctcagtagcccagttgacactggcccccgactccggaagcacgggattgtcggtcaggggacttaaagtggaagagacctgtggctgccgctggacctgcccctgygtgtgcacaggcagctccactcggcacatcgtgacctttgatgggcagaatttcaaaatttcaccttctctggacaccgacggcgacctggacggggacrcacacgtgtccgtcgaggtgagccgtgtagcactggaaactacccgtcttaaagttgctgactggcagctgttcttatgtcctatttcaaaacaaggagcaggacctggaggtgattctccataatggtgcctgcagccctggagcaaggcagggccgactgaccgtcgacaagaatacaggataaagttttgttcctcgtcctggacctccactaagaggtattaccacggacgtcgggacctcgttccgtcccgtgcatgaaatccatcgaggtgaagcacagtgccctctccgtcgagstgcacagtgacatggaggtgacggtgaatgggagactggtctctgttccttacgacgtactttaggtagctccacttcgtgtcacgggagaggcagctcsacgtgtcactgtacctccactgccacttaccctctgaccagagacaaggaatgctgggtgggaacatggaagtcaacgtttatggtgccatcatgcatgaggtcagattcaatcaccttggtcacatcttcacattcactccacaaaacaatgaacccacccttgtaccttcagttgcaaataccacggtagtacgtactccagtctaagttagtggaaccagtgtagaagtgtaagtgaggtgttttgttactgttccaactgcagctcagccccaagacttttgcttcaaagacgtatggtctgtgtgggatctgtgatgagaacggagccaatgacttcatgctgagggatcaaggttgacgtcgagtcggggttctgaaaacgaagtttctgcataccagacacaccctagacactactcttgcctcggttactgaagtacgactccctaggcacagtcaccacagactggaaaacacttgttcaggaatggactgtgcagcggccagggcagacgtgccagcccatcctggaggagcagtgtcttgtccccgtgtcagtggtgtctgaccttttgtgaacaagtccttacctgacacgtcgccggtcccgtctgcacggtcgggtaggacctcctcgtcacagaacaggccgacagctcccactgccaggtcctcctcttaccactgtttgctgaatgccacaaggtcctggctccagccacattctatgccatctgccagcaggacagggctgtcgagggtgacggtccaggaggagaatggtgacaaacgacttacggtgttccaggaccgaggtcggtgtaagatacggtagacggtcgtcctgtcttgccaccaggagcaagtgtgtgaggtgatcgcctcttatgcccacctctgtcggaccaacggggtctgcgttgactggaggacacctgatttctgtgctaacggtggtcctcgttcacacactccactagcggagaatacgggtggagacagcctggttgccccagacgcaactgacctcctgtggactaaagacacgaatgtcatgcccaccatctctggtctacaaccactgtgagcatggctgtccccggcactgtgatggcaacgtgagctcctgtggggaccatccctccgaagtacagtacgggtggtagagaccagatgttggtgacactcgtaccgacaggggccgtgacactaccgttgcactcgaggacacccctggtagggaggcttcgctgtttctgccctccagataaagtcatgttggaaggcagctgtgtccctgaagaggcctgcactcagtgcattggtgaggatggagtccagcaccagttcgacaaagacgggaggtctatttcagtacaaccttccgtcgacacagggacttctccggacgtgagtcacgtaaccactcctacctcaggtcgtggtcaacctggaagcctgggtcccggaccaccagccctgtcagatctgcacatgcctcagcgggcggaaggtcaactgcacaacgcagccctgccccacggccaaaggaccttcggacccagggcctggtggtcgggacagtctagacgtgtacggagtcgcccgccttccagttgacgtgttgcgtcgggacggggtgccggtttgctcccacgtgtggcctgtgtgaagtagcccgcctccgccagaatgcagaccagtgctgccccgagtatgagtgtgtgtgtgacccagtgagctgtgacccgagggtgcacaccggacacacttcatcgggcggaggcggtcttacgtctggtcacgacggggctcatactcacacacacactgggtcactcgacactggtgcccccagtgcctcactgtgaacgtggcctccagcccacactgaccaaccctggcgagtgcagacccaacttcacctgcgcctgcaggaaggaggagtgacgggggtcacggagtgacacttgcaccggaggtcgggtgtgactggttgggaccgctcacgtctgggttgaagtggacgcggacgtccttcctcctcaccaaaagagtgtccccaccctcctgccccccgcaccgtttgcccacccttcggaagacccagtgctgtgatgagtatgagtgtgcctgcaactgtgtcaacgttttctcacaggggtgggaggacggggggcgtggcaaacgggtgggaagccttctgggtcacgacactactcatactcacacggacgttgacacagttgtccacagtgagctgtccccttgggtacttggcctcaaccgccaccaatgactgtggctgtaccacaaccacctgccttcccgacaaggtgtgtgtccaccaggtgtcactcgacaggggaacccatgaaccggagttggcggtggttactgacaccgacatggtgttggtggacggaagggctgttccacacacaggtgggaagcaccatctaccctgtgggccagttctgggaggagggctgcgatgtgtgcacctgcaccgacatggaggatgccgtgatgggcctccgcgtggcccacttcgtggtagatgggacacccggtcaagaccctcctcccgacgctacacacgtggacgtggctgtacctcctacggcactacccggaggcgcaccgggtgtgctcccagaagccctgtgaggacagctgtcggtcgggcttcacttacgttctgcatgaaggcgagtgctgtggaaggtgcctgccatctgcctgtgagcacgagggtcttcgggacactcctgtcgacagccagcccgaagtgaatgcaagacgtacttccgctcacgacaccttccacggacggtagacggacactcgtggtgactggctcaccgcggggggactcccagtcttcctggaagagtgtcggctcccagtgggcctccccggagaacccctgcctcatcaatgagtgtgcaccactgaccgagtggcgcccccctgagggtcagaaggaccttctcacagccgagggtcacccggaggggcctcttggggacggagtagttactcacactccgagtgaaggaggaggtctttatacaacaaaggaacgtctcctgcccccagctggaggtccctgtctgcccctcgggctttcagctgagctgtaagacaggctcacttcctcctccagaaatatgttgtttccttgcagaggacgggggtcgacctccagggacagacggggagcccgaaagtcgactcgacattctgctcagcgtgctgcccaagctgtcgctgtgagcgcatggaggcctgcatgctcaatggcactgtcattgggcccgggaagactgtgatgatcgatgtgtgcgagtcgcacgacgggttcgacagcgacactcgcgtacctccggacgtacgagttaccgtgacagtaacccgggcccttctgacactactagctacacacgacgacctgccgctgcatggtgcaggtgggggtcatctctggattcaagctggagtgcaggaagaccacctgcaacccctgccccctgggttacaaggaagtgctggacggcgacgtaccacgtccacccccagtagagacctaagttcgacctcacgtccttctggtggacgttggggacgggggacccaatgttccttcaaaataacacaggtgaatgttgtgggagatgtttgcctacggcttgcaccattcagctaagaggaggacagatcatgacactgaagcgtgatgagacgctttttattgtgtccacttacaacaccctctacaaacggatgccgaacgtggtaagtcgattctcctcctgtctagtactgtgacttcgcactactctgcgaccaggatggctgtgatactcacttctgcaaggtcaatgagagaggagagtacttctgggagaagagggtcacaggctgcccaccctttgatgaacacaagggtcctaccgacactatgagtgaagacgttccagttactctctcctctcatgaagaccctcttctcccagtgtccgacgggtgggaaactacttgtgttctgtcttgctgagggaggtaaaattatgaaaattccaggcacctgctgtgacacatgtgaggagcctgagtgcaacgacatcactgccaggctgcagtatgacagaacgactccctccattttaatacttttaaggtccgtggacgacactgtgtacactcctcggactcacgttgctgtagtgacggtccgacgtcatactcaaggtgggaagctgtaagtctgaagtagaggtggatatccactactgccagggcaaatgtgccagcaaagccatgtactccattgacatcaacgatgtagttccacccttcgacattcagacttcatctccacctataggtgatgacggtcccgtttacacggtcgtttcggtacatgaggtaactgtagttgctacagcaggaccagtgctcctgctgctctccgacacggacggagcccatgcaggtggccctgcactgcaccaatggctctgttgtgtaccatgaggttctcaatcgtcctggtcacgaggacgacgagaggctgtgcctgcctcgggtacgtccaccgggacgtgacgtggttaccgagacaacacatggtactccaagagttagccatggagtgcaaatgctcccccaggaagtgcagcaagtga

SEQ ID NO: 2 - protein, 2813 aa, Homo sapiens
  REGION 1..22: note = misc_feature - VWF Signal Peptide
  REGION 23..763: note = misc_feature - VWF D1D2 region
  REGION 764..866: note = misc_feature - VWF D'Domain
  REGION 867..1240: note = misc_feature - VWF D3 Domain
  REGION 1241..1479: note = misc_feature - VWF A1 Domain
  SITE 2016: note = misc_feature - Xaa can be any naturally occurring amino acid
MIPARFAGVLLALALILPGTLCAEGTRGRSSTARCSLFGSDFVNTFDGSMYSFAGYCSYLLAGGCQKRSFSIIGDFQNGKRVSLSVYLGEFFDIHLFVNGTVTQGDQRVSMPYASKGLYLETEAGYYKLSGEAYGFVARIDGSGNFQVLLSDRYFNKTCGLCGNFNIFAEDDFMTQEGTLTSDPYDFANSWALSSGEQWCERASPPSSSCNISSGEMQKGLWEQCQLLKSTSVFARCHPLVDPEPFVALCEKTLCECAGGLECACPALLEYARTCAQEGMVLYGWTDHSACSPVCPAGMEYRQCVSPCARTCQSLHINEMCQERCVDGCSCPEGQLLDEGLCVESTECPCVHSGKRYPPGTSLSRDCNTCICRNSQWICSNEECPGECLVTGQSHFKSFDNRYFTFSGICQYLLARDCQDHSFSIVIETVQCADDRDAVCTRSVTVRLPGLHNSLVKLKHGAGVAMDGQDIQLPLLKGDLRIQHTVTASVRLSYGEDLQMDWDGRGRLLVKLSPVYAGKTCGLCGNYNGNQGDDFLTPSGLAEPRVEDFGNAWKLHGDCQDLQKQHSDPCALNPRMTRFSEEACAVLTSPTFEACHRAVSPLPYLRNCRYDVCSCSDGRECLCGALASYAAACAGRGVRVAWREPGRCELNCPKGQVYLQCGTPCNLTCRSLSYPDEECNEACLEGCFCPPGLYMDERGDCVPKAQCPCYYDGEIFQPEDIFSDHHTMCYCEDGFMHCTMSGVPGSLLPDAVLSSPLSHRSKRSLSCRPPMVKLVCPADNLRAEGLECTKTCQNYDLECMSMGCVSGCLCPPGMVRHENRCVALERCPCFHQGKEYAPGETVKIGCNTCVCRDRKWNCTDHVCDATCSTIGMAHYLTFDGLKYLFPGECQYVLVQDYCGSNPGTFRILVGNKGCSHPSVKCKKRVTILVEGGEIELFDGEVNVKRPMKDETHFEVVESGRYIILLLGKALSVVWDRHLSISVVLKQTYQEKVCGLCGNFDGIQNNDLTSSNLQVEEDPVDFGNSWKVSSQCADTRKVPLDSSPATCHNNIMKQTMVDSSCRILTSDVFQDCNKLVDPEPYLDVCIYDTCSCESIGDCACFCDTIAAYAHVCAQHGKVVTWRTATLCPQSCEERNLRENGYECEWRYNSCAPACQVTCQHPEPLACPVQCVEGCHAHCPPGKILDELLQTCVDPEDCPVCEVAGRRFASGKKVTLNPSDPEHCQICHCDVVNLTCEACQEPGGLVVPPTDAPVSPTTLYVEDISEPPLHDFYCSRLLDLVFLLDGSSRLSEAEFEVLKAFVVDMMERLRISQKWVRVAVVEYHDGSHAYIGLKDRKRPSELRRIASQVKYAGSQVASTSEVLKYTLFQIFSKIDRPEASRIALLLMASQEPQRMSRNFVRYVQGLKKKKVIVIPVGIGPHANLKQIRLIEKQAPENKAFVLSSVDELEQQRDEIVSYLCDLAPEAPPPTLPPDMAQVTVGPGLLGVSTLGPKRNSMVLDVAFVLEGSDKIGEADFNRSKEFMEEVIQRMDVGQDSIHVTVLQYSYMVTVEYPFSEAQSKGDILQRVREIRYQGGNRTNTGLALRYLSDHSFLVSQGDREQAPNLVYMVTGNPASDEIKRLPGDIQVVPIGVGPNANVQELERIGWPNAPILIQDFETLPREAPDLVLQRCCSGEGLQIPTLSPAPDCSQPLDVILLLDGSSSFPASYFDEMKSFAKAFISKANIGPRLTQVSVLQYGSITTIDVPWNVVPEKAHLLSLVDVMQREGGPSQIGDALGFAVRYLTSEMHGARPGASKAVVILVTDVSVDSVDAAADAARSNRVTVFPIGIGDRYDAAQLRILAGPAGDSNVVKLQRIEDLPTMVTLGNSFLHKLCSGFVRICMDEDGNEKRPGDVWTLPDQCHTVTCQPDGQTLLKSHRVNCDRGLRPSCPNSQSPVKVEETCGCRWTCPCVCTGSSTRHIVTFDGQNFKLTGSCSYVLFQNKEQDLEVILHNGACSPGARQGCMKSIEVKHSALSVEXHSDMEVTVNGRLVSVPYVGGNMEVNVYGAIMHEVRFNHLGHIFTFTPQNNEFQLQLSPKTFASKTYGLCGICDENGANDFMLRDGTVTTDWKTLVQEWTVQRPGQTCQPILEEQCLVPDSSHCQVLLLPLFAECHKVLAPATFYAICQQDSCHQEQVCEVIASYAHLCRTNGVCVDWRTPDFCAMSCPPSLVYNHCEHGCPRHCDGNVSSCGDHPSEGCFCPPDKVMLEGSCVPEEACTQCIGEDGVQHQFLEAWVPDHQPCQICTCLSGRKVNCTTQPCPTAKAPTCGLCEVARLRQNADQCCPEYECVCDPVSCDLPPVPHCERGLQPTLTNPGECRPNFTCACRKEECKRVSPPSCPPHRLPTLRKTQCCDEYECACNCVNSTVSCPLGYLASTATNDCGCTTTTCLPDKVCVHRSTIYPVGQFWEEGCDVCTCTDMEDAVMGLRVAQCSQKPCEDSCRSGFTYVLHEGECCGRCLPSACEVVTGSPRGDSQSSWKSVGSQWASPENPCLINECVRVKEEVFIQQRNVSCPQLEVPVCPSGFQLSCKTSACCPSCRCERMEACMLNGTVIGPGKTVMIDVCTTCRCMVQVGVISGFKLECRKTTCNPCPLGYKEENNTGECCGRCLPTACTIQLRGGQIMTLKRDETLQDGCDTHFCKVNERGEYFWEKRVTGCPPFDEHKCLAEGGKIMKIPGTCCDTCEEPECNDITARLQYVKVGSCKSEVEVDIHYCQGKCASKAMYSIDINDVQDQCSCCSPTRTEPMQVALHCTNGSVVYHEVLNAMECKCSPRKCSK

SEQ ID NO: 3 - protein, 11 aa, synthetic construct
  REGION 1..11: note = polypeptide
PKNSSMISNTP

SEQ ID NO: 4 - protein, 7 aa, synthetic construct
  REGION 1..7: note = polypeptide
HQSLGTQ

SEQ ID NO: 5 - protein, 8 aa, synthetic construct
  REGION 1..8: note = polypeptide
HQNLSDGK

SEQ ID NO: 6 - protein, 8 aa, synthetic construct
  REGION 1..8: note = polypeptide
HQNISDGK

SEQ ID NO: 7 - protein, 8 aa, synthetic construct
  REGION 1..8: note = polypeptide
VISSHLGQ

SEQ ID NO: 8 - protein, 20 aa, synthetic construct
  REGION 1..20: note = PAS sequence
ASPAAPAPASPAAPAPSAPA

SEQ ID NO: 9 - protein, 20 aa, synthetic construct
  REGION 1..20: note = PAS sequence
AAPASPAPAAPSAPAPAAPS

SEQ ID NO: 10 - protein, 20 aa, synthetic construct
  REGION 1..20: note = PAS sequence
APSSPSPSAPSSPSPASPSS

SEQ ID NO: 11 - protein, 19 aa, synthetic construct
  REGION 1..19: note = PAS sequence
APSSPSPSAPSSPSPASPS

SEQ ID NO: 12 - protein, 20 aa, synthetic construct
  REGION 1..20: note = PAS sequence
SSPSAPSPSSPASPSPSSPA

SEQ ID NO: 13 - protein, 24 aa, synthetic construct
  REGION 1..24: note = PAS sequence
AASPAAPSAPPAAASPAAPSAPPA

SEQ ID NO: 14 - protein, 20 aa, synthetic construct
  REGION 1..20: note = PAS sequence
ASAAAPAAASAAASAPSAAA

SEQ ID NO: 15 - protein, 19 aa, synthetic construct
  REGION 1..19: note = FVIII signal peptide
MQIELSTCFFLCLLRFCFS

SEQ ID NO: 16 - protein, 2332 aa, Homo sapiens
ATRRYYLGAVELSWDYMQSDLGELPVDARFPPRVPKSFPFNTSVVYKKTLFVEFTDHLFNIAKPRPPWMGLLGPTIQAEVYDTVVITLKNMASHPVSLHAVGVSYWKASEGAEYDDQTSQREKEDDKVFPGGSHTYVWQVLKENGPMASDPLCLTYSYLSHVDLVKDLNSGLIGALLVCREGSLAKEKTQTLHKFILLFAVFDEGKSWHSETKNSLMQDRDAASARAWPKMHTVNGYVNRSLPGLIGCHRKSVYWHVIGMGTTPEVHSIFLEGHTFLVRNHRQASLEISPITFLTAQTLLMDLGQFLLFCHISSHQHDGMEAYVKVDSCPEEPQLRMKNNEEAEDYDDDLTDSEMDVVRFDDDNSPSFIQIRSVAKKHPKTWVHYIAAEEEDWDYAPLVLAPDDRSYKSQYLNNGPQRIGRKYKKVRFMAYTDETFKTREAIQHESGILGPLLYGEVGDTLLIIFKNQASRPYNIYPHGITDVRPLYSRRLPKGVKHLKDFPILPGEIFKYKWTVTVEDGPTKSDPRCLTRYYSSFVNMERDLASGLIGPLLICYKESVDQRGNQIMSDKRNVILFSVFDENRSWYLTENIQRFLPNPAGVQLEDPEFQASNIMHSINGYVFDSLQLSVCLHEVAYWYILSIGAQTDFLSVFFSGYTFKHKMVYEDTLTLFPFSGETVFMSMENPGLWILGCHNSDFRNRGMTALLKVSSCDKNTGDYYEDSYEDISAYLLSKNNAIEPRSFSQNSRHPSTRQKQFNATTIPENDIEKTDPWFAHRTPMPKIQNVSSSDLLMLLRQSPTPHGLSLSDLQEAKYETFSDDPSPGAIDSNNSLSEMTHFRPQLHHSGDMVFTPESGLQLRLNEKLGTTAATELKKLDFKVSSTSNNLISTIPSDNLAAGTDNTSSLGPPSMPVHYDSQLDTTLFGKKSSPLTESGGPLSLSEENNDSKLLESGLMNSQESSWGKNVSSTESGRLFKGKRAHGPALLTKDNALFKVSISLLKTNKTSNNSATNRKTHIDGPSLLIENSPSVWQNILESDTEFKKVTPLIHDRMLMDKNATALRLNHMSNKTTSSKNMEMVQQKKEGPIPPDAQNPDMSFFKMLFLPESARWIQRTHGKNSLNSGQGPSPKQLVSLGPEKSVEGQNFLSEKNKVVVGKGEFTKDVGLKEMVFPSSRNLFLTNLDNLHENNTHNQEKKIQEEIEKKETLIQENVVLPQIHTVTGTKNFMKNLFLLSTRQNVEGSYDGAYAPVLQDFRSLNDSTNRTKKHTAHFSKKGEEENLEGLGNQTKQIVEKYACTTRISPNTSQQNFVTQRSKRALKQFRLPLEETELEKRIIVDDTSTQWSKNMKHLTPSTLTQIDYNEKEKGAITQSPLSDCLTRSHSIPQANRSPLPIAKVSSFPSIRPIYLTRVLFQDNSSHLPAASYRKKDSGVQESSHFLQGAKKNNLSLAILTLEMTGDQREVGSLGTSATNSVTYKKVENTVLPKPDLPKTSGKVELLPKVHIYQKDLFPTETSNGSPGHLDLVEGSLLQGTEGAIKWNEANRPGKVPFLRVATESSAKTPSKLLDPLAWDNHYGTQIPKEEWKSQEKSPEKTAFKKKDTILSLNACESNHAIAAINEGQNKPEIEVTWAKQGRTERLCSQNPPVLKRHQREITRTTLQSDQEEIDYDDTISVEMKKEDFDIYDEDENQSPRSFQKKTRHYFIAAVERLWDYGMSSSPHVLRNRAQSGSVPQFKKVVFQEFTDGSFTQPLYRGELNEHLGLLGPYIRAEVEDNIMVTFRNQASRPYSFYSSLISYEEDQRQGAEPRKNFVKPNETKTYFWKVQHHMAPTKDEFDCKAWAYFSDVDLEKDVHSGLIGPLLVCHTNTLNPAHGRQVTVQEFALFFTIFDETKSWYFTENMERNCRAPCNIQMEDPTFKENYRFHAINGYIMDTLPGLVMAQDQRIRWYLLSMGSNENIHSIHFSGHVFTVRKKEEYKMALYNLYPGVFETVEMLPSKAGIWRVECLIGEHLHAGMSTLFLVYSNKCQTPLGMASGHIRDFQITASGQYGQWAPKLARLHYSGSINAWSTKEPFSWIKVDLLAPMIIHGIKTQGARQKFSSLYISQFIIMYSLDGKKWQTYRGNSTGTLMVFFGNVDSSGIKHNIFNPPIIARYIRLHPTHYSIRSTLRMELMGCDLNSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNSLDPPLLTRYLRIHPQSWVHQIALRMEVLGCEAQDLY

SEQ ID NO: 17 - DNA, 7053 bp, Homo sapiens
  source 1..7053: mol_type = unassigned DNA
atgcaaatagagctctccacctgcttctttctgtgccttttgcgattctgctttagtgccaccagaagatactacctgggtgcagtggaactgtcatgggactatatgcaaagtgatctcggtgagctgcctgtggacgcaagatttcctcctagagtgccaaaatcttttccattcaacacctcagtcgtgtacaaaaagactctgtttgtagaattcacggatcaccttttcaacatcgctaagccaaggccaccctggatgggtctgctaggtcctaccatccaggctgaggtttatgatacagtggtcattacacttaagaacatggcttcccatcctgtcagtcttcatgctgttggtgtatcctactggaaagcttctgagggagctgaatatgatgatcagaccagtcaaagggagaaagaagatgataaagtcttccctggtggaagccatacatatgtctggcaggtcctgaaagagaatggtccaatggcctctgacccactgtgccttacctactcatatctttctcatgtggacctggtaaaagacttgaattcaggcctcattggagccctactagtatgtagagaagggagtctggccaaggaaaagacacagaccttgcacaaatttatactactttttgctgtatttgatgaagggaaaagttggcactcagaaacaaagaactccttgatgcaggatagggatgctgcatctgctcgggcctggcctaaaatgcacacagtcaatggttatgtaaacaggtctctgccaggtctgattggatgccacaggaaatcagtctattggcatgtgattggaatgggcaccactcctgaagtgcactcaatattcctcgaaggtcacacatttcttgtgaggaaccatcgccaggcgtccttggaaatctcgccaataactttccttactgctcaaacactcttgatggaccttggacagtttctactgttttgtcatatctcttcccaccaacatgatggcatggaagcttatgtcaaagtagacagctgtccagaggaaccccaactacgaatgaaaaataatgaagaagcggaagactatgatgatgatcttactgattctgaaatggatgtggtcaggtttgatgatgacaactctccttcctttatccaaattcgctcagttgccaagaagcatcctaaaacttgggtacattacattgctgctgaagaggaggactgggactatgctcccttagtcctcgcccccgatgacagaagttataaaagtcaatatttgaacaatggccctcagcggattggtaggaagtacaaaaaagtccgatttatggcatacacagatgaaacctttaagactcgtgaagctattcagcatgaatcaggaatcttgggacctttactttatggggaagttggagacacactgttgattatatttaagaatcaagcaagcagaccatataacatctaccctcacggaatcactgatgtccgtcctttgtattcaaggagattaccaaaaggtgtaaaacatttgaaggattttccaattctgccaggagaaatattcaaatataaatggacagtgactgtagaagatgggccaactaaatcagatcctcggtgcctgacccgctattactctagtttcgttaatatggagagagatctagcttcaggactcattggccctctcctcatctgctacaaagaatctgtagatcaaagaggaaaccagataatgtcagacaagaggaatgtcatcctgttttctgtatttgatgagaaccgaagctggtacctcacagagaatatacaacgctttctccccaatccagctggagtgcagcttgaggatccagagttccaagcctccaacatcatgcacagcatcaatggctatgtttttgatagtttgcagttgtcagtttgtttgcatgaggtggcatactggtacattctaagcattggagcacagactgacttcctttctgtcttcttctctggatataccttcaaacacaaaatggtctatgaagacacactcaccctattcccattctcaggagaaactgtcttcatgtcgatggaaaacccaggtctatggattctggggtgccacaactcagactttcggaacagaggcatgaccgccttactgaaggtttctagttgtgacaagaacactggtgattattacgaggacagttatgaagatatttcagcatacttgctgagtaaaaacaatgccattgaaccaagaagcttctcccagaattcaagacaccctagcactaggcaaaagcaatttaatgccaccacaattccagaaaatgacatagagaagactgacccttggtttgcacacagaacacctatgcctaaaatacaaaatgtctcctctagtgatttgttgatgctcttgcgacagagtcctactccacatgggctatccttatctgatctccaagaagccaaatatgagactttttctgatgatccatcacctggagcaatagacagtaataacagcctgtctgaaatgacacacttcaggccacagctccatcacagtggggacatggtatttacccctgagtcaggcctccaattaagattaaatgagaaactggggacaactgcagcaacagagttgaagaaacttgatttcaaagtttctagtacatcaaataatctgatttcaacaattccatcagacaatttggcagcaggtactgataatacaagttccttaggacccccaagtatgccagttcattatgatagtcaattagataccactctatttggcaaaaagtcatctccccttactgagtctggtggacctctgagcttgagtgaagaaaataatgattcaaagttgttagaatcaggtttaatgaatagccaagaaagttcatggggaaaaaatgtatcgtcaacagagagtggtaggttatttaaagggaaaagagctcatggacctgctttgttgactaaagataatgccttattcaaagttagcatctctttgttaaagacaaacaaaacttccaataattcagcaactaatagaaagactcacattgatggcccatcattattaattgagaatagtccatcagtctggcaaaatatattagaaagtgacactgagtttaaaaaagtgacacctttgattcatgacagaatgcttatggacaaaaatgctacagctttgaggctaaatcatatgtcaaataaaactacttcatcaaaaaacatggaaatggtccaacagaaaaaagagggccccattccaccagatgcacaaaatccagatatgtcgttctttaagatgctattcttgccagaatcagcaaggtggatacaaaggactcatggaaagaactctctgaactctgggcaaggccccagtccaaagcaattagtatccttaggaccagaaaaatctgtggaaggtcagaatttcttgtctgagaaaaacaaagtggtagtaggaaagggtgaatttacaaaggacgtaggactcaaagagatggtttttccaagcagcagaaacctatttcttactaacttggataatttacatgaaaataatacacacaatcaagaaaaaaaaattcaggaagaaatagaaaagaaggaaacattaatccaagagaatgtagttttgcctcagatacatacagtgactggcactaagaatttcatgaagaaccttttcttactgagcactaggcaaaatgtagaaggttcatatgacggggcatatgctccagtacttcaagattttaggtcattaaatgattcaacaaatagaacaaagaaacacacagctcatttctcaaaaaaaggggaggaagaaaacttggaaggcttgggaaatcaaaccaagcaaattgtagagaaatatgcatgcaccacaaggatatctcctaatacaagccagcagaattttgtcacgcaacgtagtaagagagctttgaaacaattcagactcccactagaagaaacagaacttgaaaaaaggataattgtggatgacacctcaacccagtggtccaaaaacatgaaacatttgaccccgagcaccctcacacagatagactacaatgagaaggagaaaggggccattactcagtctcccttatcagattgccttacgaggagtcatagcatccctcaagcaaatagatctccattacccattgcaaaggtatcatcatttccatctattagacctatatatctgaccagggtcctattccaagacaactcttctcatcttccagcagcatcttatagaaagaaagattctggggtccaagaaagcagtcatttcttacaaggagccaaaaaaaataacctttctttagccattctaaccttggagatgactggtgatcaaagagaggttggctccctggggacaagtgccacaaattcagtcacatacaagaaagttgagaacactgttctcccgaaaccagacttgcccaaaacatctggcaaagttgaattgcttccaaaagttcacatttatcagaaggacctattccctacggaaactagcaatgggtctcctggccatctggatctcgtggaagggagccttcttcagggaacagagggagcgattaagtggaatgaagcaaacagacctggaaaagttccctttctgagagtagcaacagaaagctctgcaaagactccctccaagctattggatcctcttgcttgggataaccactatggtactcagataccaaaagaagagtggaaatcccaagagaagtcaccagaaaaaacagcttttaagaaaaaggataccattttgtccctgaacgcttgtgaaagcaatcatgcaatagcagcaataaatgagggacaaaataagcccgaaatagaagtcacctgggcaaagcaaggtaggactgaaaggctgtgctctcaaaacccaccagtcttgaaacgccatcaacgggaaataactcgtactactcttcagtcagatcaagaggaaattgactatgatgataccatatcagttgaaatgaagaaggaagattttgacatttatgatgaggatgaaaatcagagcccccgcagctttcaaaagaaaacacgacactattttattgctgcagtggagaggctctgggattatgggatgagtagctccccacatgttctaagaaacagggctcagagtggcagtgtccctcagttcaagaaagttgttttccaggaatttactgatggctcctttactcagcccttataccgtggagaactaaatgaacatttgggactcctggggccatatataagagcagaagttgaagataatatcatggtaactttcagaaatcaggcctctcgtccctattccttctattctagccttatttcttatgaggaagatcagaggcaaggagcagaacctagaaaaaactttgtcaagcctaatgaaaccaaaacttacttttggaaagtgcaacatcatatggcacccactaaagatgagtttgactgcaaagcctgggcttatttctctgatgttgacctggaaaaagatgtgcactcaggcctgattggaccccttctggtctgccacactaacacactgaaccctgctcatgggagacaagtgacagtacaggaatttgctctgtttttcaccatctttgatgagaccaaaagctggtacttcactgaaaatatggaaagaaactgcagggctccctgcaatatccagatggaagatcccacttttaaagagaattatcgcttccatgcaatcaatggctacataatggatacactacctggcttagtaatggctcaggatcaaaggattcgatggtatctgctcagcatgggcagcaatgaaaacatccattctattcatttcagtggacatgtgttcactgtacgaaaaaaagaggagtataaaatggcactgtacaatctctatccaggtgtttttgagacagtggaaatgttaccatccaaagctggaatttggcgggtggaatgccttattggcgagcatctacatgctgggatgagcacactttttctggtgtacagcaataagtgtcagactcccctgggaatggcttctggacacattagagattttcagattacagcttcaggacaatatggacagtgggccccaaagctggccagacttcattattccggatcaatcaatgcctggagcaccaaggagcccttttcttggatcaaggtggatctgttggcaccaatgattattcacggcatcaagacccagggtgcccgtcagaagttctccagcctctacatctctcagtttatcatcatgtatagtcttgatgggaagaagtggcagacttatcgaggaaattccactggaaccttaatggtcttctttggcaatgtggattcatctgggataaaacacaatatttttaaccctccaattattgctcgatacatccgtttgcacccaactcattatagcattcgcagcactcttcgcatggagttgatgggctgtgatttaaatagttgcagcatgccattgggaatggagagtaaagcaatatcagatgcacagattactgcttcatcctactttaccaatatgtttgccacctggtctccttcaaaagctcgacttcacctccaagggaggagtaatgcctggagacctcaggtgaataatccaaaagagtggctgcaagtggacttccagaagacaatgaaagtcacaggagtaactactcagggagtaaaatctctgcttaccagcatgtatgtgaaggagttcctcatctccagcagtcaagatggccatcagtggactctcttttttcagaatggcaaagtaaaggtttttcagggaaatcaagactccttcacacctgtggtgaactctctagacccaccgttactgactcgctaccttcgaattcacccccagagttgggtgcaccagattgccctgaggatggaggttctgggctgcgaggcacaggacctctac

SEQ ID NO: 18 - protein, 1438 aa, synthetic construct
  REGION 1..1438: note = BDD FVIII
ATRRYYLGAVELSWDYMQSDLGELPVDARFPPRVPKSFPFNTSVVYKKTLFVEFTDHLFNIAKPRPPWMGLLGPTIQAEVYDTVVITLKNMASHPVSLHAVGVSYWKASEGAEYDDQTSQREKEDDKVFPGGSHTYVWQVLKENGPMASDPLCLTYSYLSHVDLVKDLNSGLIGALLVCREGSLAKEKTQTLHKFILLFAVFDEGKSWHSETKNSLMQDRDAASARAWPKMHTVNGYVNRSLPGLIGCHRKSVYWHVIGMGTTPEVHSIFLEGHTFLVRNHRQASLEISPITFLTAQTLLMDLGQFLLFCHISSHQHDGMEAYVKVDSCPEEPQLRMKNNEEAEDYDDDLTDSEMDVVRFDDDNSPSFIQIRSVAKKHPKTWVHYIAAEEEDWDYAPLVLAPDDRSYKSQYLNNGPQRIGRKYKKVRFMAYTDETFKTREAIQHESGILGPLLYGEVGDTLLIIFKNQASRPYNIYPHGITDVRPLYSRRLPKGVKHLKDFPILPGEIFKYKWTVTVEDGPTKSDPRCLTRYYSSFVNMERDLASGLIGPLLICYKESVDQRGNQIMSDKRNVILFSVFDENRSWYLTENIQRFLPNPAGVQLEDPEFQASNIMHSINGYVFDSLQLSVCLHEVAYWYILSIGAQTDFLSVFFSGYTFKHKMVYEDTLTLFPFSGETVFMSMENPGLWILGCHNSDFRNRGMTALLKVSSCDKNTGDYYEDSYEDISAYLLSKNNAIEPRSFSQNPPVLKRHQREITRTTLQSDQEEIDYDDTISVEMKKEDFDIYDEDENQSPRSFQKKTRHYFIAAVERLWDYGMSSSPHVLRNRAQSGSVPQFKKVVFQEFTDGSFTQPLYRGELNEHLGLLGPYIRAEVEDNIMVTFRNQASRPYSFYSSLISYEEDQRQGAEPRKNFVKPNETKTYFWKVQHHMAPTKDEFDCKAWAYFSDVDLEKDVHSGLIGPLLVCHTNTLNPAHGRQVTVQEFALFFTIFDETKSWYFTENMERNCRAPCNIQMEDPTFKENYRFHAINGYIMDTLPGLVMAQDQRIRWYLLSMGSNENIHSIHFSGHVFTVRKKEEYKMALYNLYPGVFETVEMLPSKAGIWRVECLIGEHLHAGMSTLFLVYSNKCQTPLGMASGHIRDFQITASGQYGQWAPKLARLHYSGSINAWSTKEPFSWIKVDLLAPMIIHGIKTQGARQKFSSLYISQFIIMYSLDGKKWQTYRGNSTGTLMVFFGNVDSSGIKHNIFNPPIIARYIRLHPTHYSIRSTLRMELMGCDLNSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNSLDPPLLTRYLRIHPQSWVHQIALRMEVLGCEAQDLY

SEQ ID NO: 19 - DNA, 4371 bp, synthetic construct
  misc_feature 1..4371: note = BDD FVIII
  source 1..4371: mol_type = other DNA
atgcaaatagagctctccacctgcttctttctgtgccttttgcgattctgctttagtgccaccagaagatactacctgggtgcagtggaactgtcatgggactatatgcaaagtgatctcggtgagctgcctgtggacgcaagatttcctcctagagtgccaaaatcttttccattcaacacctcagtcgtgtacaaaaagactctgtttgtagaattcacggatcaccttttcaacatcgctaagccaaggccaccctggatgggtctgctaggtcctaccatccaggctgaggtttatgatacagtggtcattacacttaagaacatggcttcccatcctgtcagtcttcatgctgttggtgtatcctactggaaagcttctgagggagctgaatatgatgatcagaccagtcaaagggagaaagaagatgataaagtcttccctggtggaagccatacatatgtctggcaggtcctgaaagagaatggtccaatggcctctgacccactgtgccttacctactcatatctttctcatgtggacctggtaaaagacttgaattcaggcctcattggagccctactagtatgtagagaagggagtctggccaaggaaaagacacagaccttgcacaaatttatactactttttgctgtatttgatgaagggaaaagttggcactcagaaacaaagaactccttgatgcaggatagggatgctgcatctgctcgggcctggcctaaaatgcacacagtcaatggttatgtaaacaggtctctgccaggtctgattggatgccacaggaaatcagtctattggcatgtgattggaatgggcaccactcctgaagtgcactcaatattcctcgaaggtcacacatttcttgtgaggaaccatcgccaggcgtccttggaaatctcgccaataactttccttactgctcaaacactcttgatggaccttggacagtttctactgttttgtcatatctcttcccaccaacatgatggcatggaagcttatgtcaaagtagacagctgtccagaggaaccccaactacgaatgaaaaataatgaagaagcggaagactatgatgatgatcttactgattctgaaatggatgtggtcaggtttgatgatgacaactctccttcctttatccaaattcgctcagttgccaagaagcatcctaaaacttgggtacattacattgctgctgaagaggaggactgggactatgctcccttagtcctcgcccccgatgacagaagttataaaagtcaatatttgaacaatggccctcagcggattggtaggaagtacaaaaaagtccgatttatggcatacacagatgaaacctttaagactcgtgaagctattcagcatgaatcaggaatcttgggacctttactttatggggaagttggagacacactgttgattatatttaagaatcaagcaagcagaccatataacatctaccctcacggaatcactgatgtccgtcctttgtattcaaggagattaccaaaaggtgtaaaacatttgaaggattttccaattctgccaggagaaatattcaaatataaatggacagtgactgtagaagatgggccaactaaatcagatcctcggtgcctgacccgctattactctagtttcgttaatatggagagagatctagcttcaggactcattggccctctcctcatctgctacaaagaatctgtagatcaaagaggaaaccagataatgtcagacaagaggaatgtcatcctgttttctgtatttgatgagaaccgaagctggtacctcacagagaatatacaacgctttctccccaatccagctggagtgcagcttgaggatccagagttccaagcctccaacatcatgcacagcatcaatggctatgtttttgatagtttgcagttgtcagtttgtttgcatgaggtggcatactggtacattctaagcattggagcacagactgacttcctttctgtcttcttctctggatataccttcaaacacaaaatggtctatgaagacacactcaccctattcccattctcaggagaaactgtcttcatgtcgatggaaaacccaggtctatggattctggggtgccacaactcagactttcggaacagaggcatgaccgccttactgaaggtttctagttgtgacaagaacactggtgattattacgaggacagttatgaagatatttcagcatacttgctgagtaaaaacaatgccattgaaccaagaagcttctctcaaaacccaccagtcttgaaacgccatcaacgggaaataactcgtactactcttcagtcagatcaagaggaaattgactatgatgataccatatcagttgaaatgaagaaggaagattttgacatttatgatgaggatgaaaatcagagcccccgcagctttcaaaagaaaacacgacactattttattgctgcagtggagaggctctgggattatgggatgagtagctccccacatgttctaagaaacagggctcagagtggcagtgtccctcagttcaagaaagttgttttccaggaatttactgatggctcctttactcagcccttataccgtggagaactaaatgaacatttgggactcctggggccatatataagagcagaagttgaagataatatcatggtaactttcagaaatcaggcctctcgtccctattccttctattctagccttatttcttatgaggaagatcagaggcaaggagcagaacctagaaaaaactttgtcaagcctaatgaaaccaaaacttacttttggaaagtgcaacatcatatggcacccactaaagatgagtttgactgcaaagcctgggcttatttctctgatgttgacctggaaaaagatgtgcactcaggcctgattggaccccttctggtctgccacactaacacactgaaccctgctcatgggagacaagtgacagtacaggaatttgctctgtttttcaccatctttgatgagaccaaaagctggtacttcactgaaaatatggaaagaaactgcagggctccctgcaatatccagatggaagatcccacttttaaagagaattatcgcttccatgcaatcaatggctacataatggatacactacctggcttagtaatggctcaggatcaaaggattcgatggtatctgctcagcatgggcagcaatgaaaacatccattctattcatttcagtggacatgtgttcactgtacgaaaaaaagaggagtataaaatggcactgtacaatctctatccaggtgtttttgagacagtggaaatgttaccatccaaagctggaatttggcgggtggaatgccttattggcgagcatctacatgctgggatgagcacactttttctggtgtacagcaataagtgtcagactcccctgggaatggcttctggacacattagagattttcagattacagcttcaggacaatatggacagtgggccccaaagctggccagacttcattattccggatcaatcaatgcctggagcaccaaggagcccttttcttggatcaaggtggatctgttggcaccaatgattattcacggcatcaagacccagggtgcccgtcagaagttctccagcctctacatctctcagtttatcatcatgtatagtcttgatgggaagaagtggcagacttatcgaggaaattccactggaaccttaatggtcttctttggcaatgtggattcatctgggataaaacacaatatttttaaccctccaattattgctcgatacatccgtttgcacccaactcattatagcattcgcagcactcttcgcatggagttgatgggctgtgatttaaatagttgcagcatgccattgggaatggagagtaaagcaatatcagatgcacagattactgcttcatcctactttaccaatatgtttgccacctggtctccttcaaaagctcgacttcacctccaagggaggagtaatgcctggagacctcaggtgaataatccaaaagagtggctgcaagtggacttccagaagacaatgaaagtcacaggagtaactactcagggagtaaaatctctgcttaccagcatgtatgtgaaggagttcctcatctccagcagtcaagatggccatcagtggactctcttttttcagaatggcaaagtaaaggtttttcagggaaatcaagactccttcacacctgtggtgaactctctagacccaccgttactgactcgctaccttcgaattcacccccagagttgggtgcaccagattgccctgaggatggaggttctgggctgcgaggcacaggacctctac

SEQ ID NO: 20 - protein, 500 aa, synthetic construct
  REGION 1..500: note = linker
GGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGS

SEQ ID NO: 21 - protein, 800 aa, synthetic construct
  REGION 1..800: note = linker
  REPEAT 1..300: note = Gly-Gly-Ser repeats 1 to 100 times
  REPEAT 301..800: note = Gly-Gly-Gly-Gly-Ser repeats 1 to 100 times
GGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGS

SEQ ID NO: 22 - protein, 7 aa, synthetic construct
  REGION 1..7: note = linker
SGGSGGS

SEQ ID NO: 23 - protein, 15 aa, synthetic construct
  REGION 1..15: note = linker
GGSGGSGGSGGSGGG

SEQ ID NO: 24 - protein, 16 aa, synthetic construct
  REGION 1..16: note = linker
GGSGGSGGGGSGGGGS

SEQ ID NO: 25 - protein, 18 aa, synthetic construct
  REGION 1..18: note = linker
GGSGGSGGSGGSGGSGGS

SEQ ID NO: 26 - protein, 15 aa, synthetic construct
  REGION 1..15: note = linker
  REPEAT 1..15: note = Gly-Gly-Gly-Gly-Ser repeats 1 to 3 times
GGGGSGGGGSGGGGS

SEQ ID NO: 27 - protein, 100 aa, synthetic construct
  REGION 1..100: note = linker
  REPEAT 1..100: note = Gly-Gly-Gly-Gly-Ser repeats 1 to 20 times
GGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGS

SEQ ID NO: 28 - protein, 15 aa, synthetic construct
  REGION 1..15: note = linker
  REPEAT 1..15: note = Gly-Gly-Gly-Gly-Ser is repeated 1 to 3 times
GGGGSGGGGSGGGGS

SEQ ID NO: 29 - protein, 7 aa, synthetic construct
  REGION 1..7: note = cleavable linker
  REGION 4..5: note = MISC_FEATURE - FXIa cleavage site
KLTRAET

SEQ ID NO: 30 - protein, 7 aa, synthetic construct
  REGION 1..7: note = cleavable linker
  REGION 4..5: note = MISC_FEATURE - FXIa cleavage site
DFTRVVG

SEQ ID NO: 31 - protein, 8 aa, synthetic construct
  REGION 1..8: note = cleavable linker
  REGION 4..5: note = MISC_FEATURE - FXIIa cleavage site
TMTRIVGG

SEQ ID NO: 32 - protein, 8 aa, synthetic construct
  REGION 1..8: note = cleavable linker
  REGION 4..5: note = MISC_FEATURE - Kallikrein cleavage site
SPFRSTGG

SEQ ID NO: 33 - protein, 8 aa, synthetic construct
  REGION 1..8: note = cleavable linker
  REGION 4..5: note = misc_feature - FVIIa cleavage site
LQVRIVGG

SEQ ID NO: 34 - protein, 8 aa, synthetic construct
  REGION 1..8: note = cleavable linker
  REGION 4..5: note = misc_feature - FIXa cleavage site
PLGRIVGG

SEQ ID NO: 35 - protein, 8 aa, synthetic construct
  REGION 1..8: note = cleavable linker
  REGION 4..5: note = misc_feature - FXa cleavage site
IEGRTVGG

SEQ ID NO: 36 - protein, 8 aa, synthetic construct
  REGION 1..8: note = cleavable linker
  REGION 4..5: note = misc_feature - FIIa (thrombin) cleavage site
LTPRSLLV

SEQ ID NO: 37 - protein, 8 aa, synthetic construct
  REGION 1..8: note = cleavable linker
  REGION 4..5: note = misc_feature - Elastase-2 cleavage site
LGPVSGVP

SEQ ID NO: 38 - protein, 8 aa, synthetic construct
  REGION 1..8: note = cleavable linker
  REGION 4..5: note = misc_feature - Granzyme-B cleavage site
VAGDSLEE

SEQ ID NO: 39 - protein, 8 aa, synthetic construct
  REGION 1..8: note = cleavable linker
  REGION 4..5: note = misc_feature - MMP-12 cleavage site
GPAGLGGA

SEQ ID NO: 40 - protein, 8 aa, synthetic construct
  REGION 1..8: note = cleavable linker
  REGION 4..5: note = misc_feature - MMP-13 cleavage site
GPAGLRGA

SEQ ID NO: 41 - protein, 8 aa, synthetic construct
  REGION 1..8: note = cleavable linker
  REGION 4..5: note = misc_feature - MMP-17 cleavage site
APLGLRLR

SEQ ID NO: 42 - protein, 8 aa, synthetic construct
  REGION 1..8: note = cleavable linker
  REGION 4..5: note = misc_feature - MMP-20 cleavage site
PALPLVAQ

SEQ ID NO: 43 - protein, 7 aa, synthetic construct
  REGION 1..7: note = cleavable linker
  REGION 6..7: note = misc_feature - TEV cleavage site
ENLYFQG

SEQ ID NO: 44 - protein, 8 aa, synthetic construct
  REGION 1..8: note = cleavable linker
  REGION 4..5: note = misc_feature - Enterokinase cleavage site
DDDKIVGG

SEQ ID NO: 45 - protein, 8 aa, synthetic construct
  REGION 1..8: note = cleavable linker
  REGION 6..7: note = misc_feature - Protease C (PRESCISSION) cleavage site
LEVLFQGP

SEQ ID NO: 46 - protein, 8 aa, synthetic construct
  REGION 1..8: note = cleavable linker
  REGION 4..5: note = misc_feature - Sortase A
LPKTGSES

SEQ ID NO: 47 - protein, 9 aa, synthetic construct
  REGION 1..9: note = cleavable linker
TQSFNDFTR

SEQ ID NO: 48 - protein, 10 aa, synthetic construct
  REGION 1..10: note = cleavage site
SVSQTSKLTR

SEQ ID NO: 49 - protein, 10 aa, synthetic construct
  REGION 1..10: note = cleavage site
DFLAEGGGVR

SEQ ID NO: 50 - protein, 7 aa, synthetic construct
  REGION 1..7: note = cleavage site
TTKIKPR

SEQ ID NO: 51 - protein, 10 aa, synthetic construct
  REGION 1..10: note = cleavge site
ALRPRVVGGA

SEQ ID NO: 52 - protein, 4 aa, synthetic construct
  REGION 1..4: note = cleavage site
RRRR

SEQ ID NO: 53 - protein, 6 aa, synthetic construct
  REGION 1..6: note = cleavage site
RKRRKR

SEQ ID NO: 54 - protein, 5 aa, synthetic construct
  REGION 1..5: note = cleavage site
RRRRS

SEQ ID NO: 55 - protein, 5 aa, synthetic construct
  REGION 1..5: note = cleavage site
LVPRG

SEQ ID NO: 56 - protein, 25 aa, synthetic construct
  REGION 1..25: note = cleavage and linker site
TLDPRSFLLRNPNDKYEPFWEDEEK

SEQ ID NO: 57 - DNA, 99 bp, synthetic construct
  misc_feature 1..99: note = ESC48-Fwd-VWF-D'D3 with VIII signal and BsiW1 site for pSYN VWF-001
  source 1..99: mol_type = other DNA
tcgcgacgtacggccgccaccatgcaaatagagctctccacctgcttctttctgtgccttttgcgattctgctttagcctatcctgtcggccccccatg

SEQ ID NO: 58 - DNA, 68 bp, synthetic construct
  misc_feature 1..68: note = ESC50- Rev- VWF- partial D'D3 (1-276 amino acid) with 6 His and Not1 site for pSYN VWF- 001
  source 1..68: mol_type = other DNA
tgacctcgagcggccgctcagtggtgatggtgatgatgcagaggcacttttctggtgtcagcacactg

SEQ ID NO: 59 - DNA, 99 bp, synthetic construct; identical to SEQ ID NO: 57
  misc_feature 1..99: note = ESC48- Fwd - VWF-D'D3 with VIII signal and BsiW1 site for pSYN VWF- 002
  source 1..99: mol_type = other DNA

SEQ ID NO: 60 - DNA, 71 bp, synthetic construct
  misc_feature 1..71: note = ESC51- Rev- VWF D'D3 (1-477 amino acid) with 6His and Not 1 site for pSYN VWF- 002
  source 1..71: mol_type = other DNA
tgacctcgagcggccgctcagtggtgatggtgatgatgcctgctgcagtagaaatcgtgcaacggcggttc

SEQ ID NO: 61 - DNA, 99 bp, synthetic construct; identical to SEQ ID NO: 57
  misc_feature 1..99: note = ESC48- Fwd - VWF-D'D3 with VIII signal and BsiW1 site for pSYN VWF- 003
  source 1..99: mol_type = other DNA

SEQ ID NO: 62 - DNA, 65 bp, synthetic construct
  misc_feature 1..65: note = ESC52- Rev-VWF-D'D3 Partial A1 (1-511 amino acids) with 6His and Not1 site for pSYN VWF- 003
  source 1..65: mol_type = other DNA
tgacctcgagcggccgctcagtggtgatggtgatgatggcccacagtgacttgtgccatgtgggg

SEQ ID NO: 63 - DNA, 99 bp, synthetic construct; identical to SEQ ID NO: 57
  misc_feature 1..99: note = ESC48- Fwd - VWF-D'D3 with VIII signal and BsiW1 site for pSYN VWF- 004
  source 1..99: mol_type = other DNA

SEQ ID NO: 64 - DNA, 65 bp, synthetic construct; identical to SEQ ID NO: 62
  misc_feature 1..65: note = ESC53-Rev- VWF-D'D3A1 (1-716 amino acids) with 6His and Not1 site for pSYN VWF- 004
  source 1..65: mol_type = other DNA

SEQ ID NO: 65 - DNA, 1313 bp, synthetic construct
  misc_feature 1..1313: note = cloned vector
  source 1..1313: mol_type = other DNA
ggatcctagtggggaataagggatgcagccacccctcagtgaaatgcaagaaacgggtcaccatcctggtggagggaggagagattgagctgtttgacggggaggtgaatgtgaagaggcccatgaaggatgagactcactttgaggtggtggagtctggccggtacatcattctgctgctgggcaaagccctctccgtggtctgggaccgccacctgagcatctccgtggtcctgaagcagacataccaggagaaagtgtgtggcctgtgtgggaattttgatggcatccagaacaatgacctcaccagcagcaacctccaagtggaggaagaccctgtggactttgggaactcctggaaagtgagctcgcagtgtgctgacaccagaaaagtgcctctggactcatcccctgccacctgccataacaacatcatgaagcagacgatggtggattcctcctgtagaatccttaccagtgacgtcttccaggactgcaacaagctggtggaccccgagccatatctggatgtctgcatttacgacacctgctcctgtgagtccattggggactgcgcctgcttctgcgacaccattgctgcctatgcccacgtgtgtgcccagcatggcaaggtggtgacctggaggacggccacattgtgcccccagagctgcgaggagaggaatctccgggagaacgggtatgagtgtgagtggcgctataacagctgtgcacctgcctgtcaagtcacgtgtcagcaccctgagccactggcctgccctgtgcagtgtgtggagggctgccatgcccactgccctccagggaaaatcctggatgagcttttgcagacctgcgttgaccctgaagactgtccagtgtgtgaggtggctggccggcgttttgcctcaggaaagaaagtcaccttgaatcccagtgaccctgagcactgccagatttgccactgtgatgttgtcaacctcacctgtgaagcctgccaggagccgggaggcctggtggtgcctcccacagatgccccggtgagccccaccactctgtatgtggatgagacgctccaggatggctgtgatactcacttctgcaaggtcaatgagagaggagagtacttctgggagaagagggtcacaggctgcccaccctttgatgaacacaagtgtcttgctgagggaggtaaaattatgaaaattccaggcacctgctgtgacacatgtgaggagcctgagtgcaacgacatcactgccaggctgcagtatgtcaaggtgggaagctgtaagtctgaagtagaggtggatatc

SEQ ID NO: 66 - DNA, 32 bp, synthetic construct
  misc_feature 1..32: note = ESC 89-fwd with Nhe1site
  source 1..32: mol_type = other DNA
ctcactatagggagacccaagctggctagccg

SEQ ID NO: 67 - DNA, 43 bp, synthetic construct
  misc_feature 1..43: note = ESC 91-rev with Sal1
  source 1..43: mol_type = other DNA
ctggatcccgggagtcgactcgtcagtggtgatggtgatgatg

SEQ ID NO: 68 - DNA, 92 bp, synthetic construct
  misc_feature 1..92: note = LW 22-FWD-VWF-D'D3 with FVIII signal sequence and BsiW1 site
  source 1..92: mol_type = other DNA
gcgccggccgtacgatgcaaatagagctctccacctgcttctttctgtgccttttgcgattctgctttagcctatcctgtcggccccccatg

SEQ ID NO: 69 - DNA, 47 bp, synthetic construct
  misc_feature 1..47: note = LW 23-Rev- Fc with stop codon and Not1 site
  source 1..47: mol_type = other DNA
tcatcaatgtatcttatcatgtctgaattcgcggccgctcatttacc

SEQ ID NO: 70 - DNA, 41 bp, synthetic construct
  misc_feature 1..41: note = LW24- Fwd- VWF D1D2D'D3 cloning oligo with BsiW1 site
  source 1..41: mol_type = other DNA
gcgccggccgtacgatgattcctgccagatttgccggggtg

SEQ ID NO: 71 - DNA, 41 bp, synthetic construct
  misc_feature 1..41: note = LW27-Rev-VWF D'D3 oligo with EcoRV
  source 1..41: mol_type = other DNA
ccaccgccagatatcggctcctggcaggcttcacaggtgag

SEQ ID NO: 72 - protein, 1240 aa, synthetic construct
  REGION 1..1240: note = VWF-D1D2D'D3
MIPARFAGVLLALALILPGTLCAEGTRGRSSTARCSLFGSDFVNTFDGSMYSFAGYCSYLLAGGCQKRSFSIIGDFQNGKRVSLSVYLGEFFDIHLFVNGTVTQGDQRVSMPYASKGLYLETEAGYYKLSGEAYGFVARIDGSGNFQVLLSDRYFNKTCGLCGNFNIFAEDDFMTQEGTLTSDPYDFANSWALSSGEQWCERASPPSSSCNISSGEMQKGLWEQCQLLKSTSVFARCHPLVDPEPFVALCEKTLCECAGGLECACPALLEYARTCAQEGMVLYGWTDHSACSPVCPAGMEYRQCVSPCARTCQSLHINEMCQERCVDGCSCPEGQLLDEGLCVESTECPCVHSGKRYPPGTSLSRDCNTCICRNSQWICSNEECPGECLVTGQSHFKSFDNRYFTFSGICQYLLARDCQDHSFSIVIETVQCADDRDAVCTRSVTVRLPGLHNSLVKLKHGAGVAMDGQDIQLPLLKGDLRIQHTVTASVRLSYGEDLQMDWDGRGRLLVKLSPVYAGKTCGLCGNYNGNQGDDFLTPSGLAEPRVEDFGNAWKLHGDCQDLQKQHSDPCALNPRMTRFSEEACAVLTSPTFEACHRAVSPLPYLRNCRYDVCSCSDGRECLCGALASYAAACAGRGVRVAWREPGRCELNCPKGQVYLQCGTPCNLTCRSLSYPDEECNEACLEGCFCPPGLYMDERGDCVPKAQCPCYYDGEIFQPEDIFSDHHTMCYCEDGFMHCTMSGVPGSLLPDAVLSSPLSHRSKRSLSCRPPMVKLVCPADNLRAEGLECTKTCQNYDLECMSMGCVSGCLCPPGMVRHENRCVALERCPCFHQGKEYAPGETVKIGCNTCVCRDRKWNCTDHVCDATCSTIGMAHYLTFDGLKYLFPGECQYVLVQDYCGSNPGTFRILVGNKGCSHPSVKCKKRVTILVEGGEIELFDGEVNVKRPMKDETHFEVVESGRYIILLLGKALSVVWDRHLSISVVLKQTYQEKVCGLCGNFDGIQNNDLTSSNLQVEEDPVDFGNSWKVSSQCADTRKVPLDSSPATCHNNIMKQTMVDSSCRILTSDVFQDCNKLVDPEPYLDVCIYDTCSCESIGDCACFCDTIAAYAHVCAQHGKVVTWRTATLCPQSCEERNLRENGYECEWRYNSCAPACQVTCQHPEPLACPVQCVEGCHAHCPPGKILDELLQTCVDPEDCPVCEVAGRRFASGKKVTLNPSDPEHCQICHCDVVNLTCEACQEP

SEQ ID NO: 73 - protein, 477 aa, synthetic construct
  REGION 1..477: note = VWF-D'D3
SLSCRPPMVKLVCPADNLRAEGLECTKTCQNYDLECMSMGCVSGCLCPPGMVRHENRCVALERCPCFHQGKEYAPGETVKIGCNTCVCRDRKWNCTDHVCDATCSTIGMAHYLTFDGLKYLFPGECQYVLVQDYCGSNPGTFRILVGNKGCSHPSVKCKKRVTILVEGGEIELFDGEVNVKRPMKDETHFEVVESGRYIILLLGKALSVVWDRHLSISVVLKQTYQEKVCGLCGNFDGIQNNDLTSSNLQVEEDPVDFGNSWKVSSQCADTRKVPLDSSPATCHNNIMKQTMVDSSCRILTSDVFQDCNKLVDPEPYLDVCIYDTCSCESIGDCACFCDTIAAYAHVCAQHGKVVTWRTATLCPQSCEERNLRENGYECEWRYNSCAPACQVTCQHPEPLACPVQCVEGCHAHCPPGKILDELLQTCVDPEDCPVCEVAGRRFASGKKVTLNPSDPEHCQICHCDVVNLTCEACQEP

SEQ ID NO: 74 - protein, 30 aa, synthetic construct
  REGION 1..30: note = VWF-D'D3 domain (1-477aa; C336A/C379A mutation)
  REPEAT 1..30: note = Gly-Gly-Gly-Gly-Ser repeats 6 times
GGGGSGGGGSGGGGSGGGGSGGGGSGGGGS

SEQ ID NO: 75 - protein, 5 aa, synthetic construct
  REGION 1..5: note = linker
RRRRS

SEQ ID NO: 76 - protein, 6 aa, synthetic construct
  REGION 1..6: note = linker
RKRRKR

SEQ ID NO: 77 - DNA, 24 bp, synthetic construct
  misc_feature 1..24: note = ESC17-Fwd- VWF cloning oligo with Cla1
  source 1..24: mol_type = other DNA
gtccggcatgagaatcgatgtgtg

SEQ ID NO: 78 - DNA, 31 bp, synthetic construct
  misc_feature 1..31: note = ESC41- Rev-VWF with EcoRV
  source 1..31: mol_type = other DNA
cctccaccgccagatatcagaggcacttttc

SEQ ID NO: 79 - DNA, 105 bp, synthetic construct
  misc_feature 1..105: note = ESC78- Fwd with EcoRV site
  source 1..105: mol_type = other DNA
aaagtgcctctgatatctggcggtggaggttccggtggcgggggatccggtggcgggggatccggtggcgggggatccggtggcgggggatccctggtcccccgg

SEQ ID NO: 80 - DNA, 107 bp, synthetic construct
  misc_feature 1..107: note = ESC79- Rev with RsRII site
  source 1..107: mol_type = other DNA
gaagaggaagactgacggtccgcccaggagttctggagctgggcacggtgggcatgtgtgagttttgtcgcctccgctgccccgggggaccagggatcccccgccac

SEQ ID NO: 81 - protein, 48 aa, synthetic construct
  REGION 1..48: note = linker
ISGGGGSGGGGSGGGGSGGGGSGGGGSGGGGSLVPRGSGGGGSGGGGS

SEQ ID NO: 82 - DNA, 1781 bp, synthetic construct
  misc_feature 1..1781: note = pSYN-FVIII-049, which is FVIII-Fc construct with a cleavable linker in between two Fc domains; Genscript-Sequence number 103069
  source 1..1781: mol_type = other DNA
ccgtcgacaagagcaggtggcagcaggggaacgtcttctcatgctccgtgatgcatgaggctctgcacaaccactacacgcagaagagcctctccctgtctccgggtaaacggcgccgccggagcggtggcggcggatcaggtgggggtggatcaggcggtggaggttccggtggcgggggatccggcggtggaggttccggtgggggtggatcaaggaagaggaggaagagaagcctatcctgtcggccccccatggtcaagctggtgtgtcccgctgacaacctgcgggctgaagggctcgagtgtaccaaaacgtgccagaactatgacctggagtgcatgagcatgggctgtgtctctggctgcctctgccccccgggcatggtccggcatgagaatcgatgtgtggccctggaaaggtgtccctgcttccatcagggcaaggagtatgcccctggagaaacagtgaagattggctgcaacacttgtgtctgtcgggaccggaagtggaactgcacagaccatgtgtgtgatgccacgtgctccacgatcggcatggcccactacctcaccttcgacgggctcaaatacctgttccccggggagtgccagtacgttctggtgcaggattactgcggcagtaaccctgggacctttcggatcctagtggggaataagggatgcagccacccctcagtgaaatgcaagaaacgggtcaccatcctggtggagggaggagagattgagctgtttgacggggaggtgaatgtgaagaggcccatgaaggatgagactcactttgaggtggtggagtctggccggtacatcattctgctgctgggcaaagccctctccgtggtctgggaccgccacctgagcatctccgtggtcctgaagcagacataccaggagaaagtgtgtggcctgtgtgggaattttgatggcatccagaacaatgacctcaccagcagcaacctccaagtggaggaagaccctgtggactttgggaactcctggaaagtgagctcgcagtgtgctgacaccagaaaagtgcctctggactcatcccctgccacctgccataacaacatcatgaagcagacgatggtggattcctcctgtagaatccttaccagtgacgtcttccaggactgcaacaagctggtggaccccgagccatatctggatgtctgcatttacgacacctgctcctgtgagtccattggggactgcgccgcattctgcgacaccattgctgcctatgcccacgtgtgtgcccagcatggcaaggtggtgacctggaggacggccacattgtgcccccagagctgcgaggagaggaatctccgggagaacgggtatgaggctgagtggcgctataacagctgtgcacctgcctgtcaagtcacgtgtcagcaccctgagccactggcctgccctgtgcagtgtgtggagggctgccatgcccactgccctccagggaaaatcctggatgagcttttgcagacctgcgttgaccctgaagactgtccagtgtgtgaggtggctggccggcgttttgcctcaggaaagaaagtcaccttgaatcccagtgaccctgagcactgccagatttgccactgtgatgttgtcaacctcacctgtgaagcctgccaggagccgatcgatggcggtggaggttccggtggcgggggatccctggtcccccggggcagcggaggcgacaaaactcacacatgcccaccgtgcccagctccagaactcctgggcggaccgtca

SEQ ID NO: 83 - DNA, 220 bp, synthetic construct
  misc_feature 1..220: note = A fragment of the Genscript construct was sub cloned into the EcoRV/RsRII digested pSYN-FVIII-0159; Genscript-Sequence no-132601
  source 1..220: mol_type = other DNA
aaagtgcctctgatatctggcggtggaggttccggtggcgggggatccggcggtggaggttccggcggtggaggttccggtggcgggggatccggtggcgggggatccctggtcccccggggcagcggcggtggaggttccggtggcgggggatccgacaaaactcacacatgcccaccgtgcccagctccagaactcctgggcggaccgtcagtcttcc

SEQ ID NO: 84 - protein, 73 aa, synthetic construct
  REGION 1..73: note = pSYN-VIII-178 has a 73 amino acids linker in between the VWF fragment and the Fc region; synthesis of DNA fragment coding for 73 amino acids linker
ISGGGGSGGGGSGGGGSGGGGSGGGGSGGGGSGGGGSGGGGSGGGGSGGGGSGGGGSLVPRGSGGGGSGGGGS

SEQ ID NO: 85 - DNA, 299 bp, synthetic construct
  misc_feature 1..299: note = Genscript-Sequence #-144849
  source 1..299: mol_type = other DNA
gcctgccaggagccgatatctggcggtggaggttccggtggcgggggatccggcggtggaggttccggcggtggaggttccggtggcgggggatccggcggtggaggttccggtggcgggggatccggcggtggaggttccggcggtggaggttccggtggcgggggatccggtggcgggggatccctggtcccccggggcagcggcggtggaggttccggtggcgggggatccgacaaaactcacacatgcccccgtgcccagctccagaactcctgggcggaccgtcagtcttcctc

SEQ ID NO: 86 - protein, 98 aa, synthetic construct
  REGION 1..98: note = linker
ISGGGGSGGGGSGGGGSGGGGSGGGGSGGGGSGGGGSGGGGSGGGGSGGGGSGGGGSGGGGSGGGGSGGGGSGGGGSGGGGSLVPRGSGGGGSGGGGS

SEQ ID NO: 87 - DNA, 380 bp, synthetic construct
  misc_feature 1..380: note = Genscript-Sequence #-144849
  source 1..380: mol_type = other DNA
gcctgccaggagccgatatctggcggtggaggttccggtggcgggggatccggcggtggaggttccggcggtggaggttccggtggcgggggatccggcggtggaggttccggtggcgggggatccggcggtggaggttccggcggtggaggttccggtggcgggggatccggcggtggaggttccggtggcgggggatccggcggtggaggttccggcggtggaggttccggtggcgggggatccggtggcgggggatccctggtcccccggggcagcggcggtggaggttccggtggcgggggatccgacaaaactcacacatgcccaccgtgcccagctccagaactcctgggcggaccgtcagtcttcctcttccc

SEQ ID NO: 88 - protein, 2449 aa, synthetic construct [sequence omitted]

SEQ ID NO: 89 - protein, 48 aa, synthetic construct; identical to SEQ ID NO: 81
  REGION 1..48: note = a 48-amino acid linker

SEQ ID NO: 90 - protein, 1665 aa, synthetic construct
  REGION 1..1665: note = pSYN-FVIII-155 mature protein
ATRRYYLGAVELSWDYMQSDLGELPVDARFPPRVPKSFPFNTSVVYKKTLFVEFTDHLFNIAKPRPPWMGLLGPTIQAEVYDTVVITLKNMASHPVSLHAVGVSYWKASEGAEYDDQTSQREKEDDKVFPGGSHTYVWQVLKENGPMASDPLCLTYSYLSHVDLVKDLNSGLIGALLVCREGSLAKEKTQTLHKFILLFAVFDEGKSWHSETKNSLMQDRDAASARAWPKMHTVNGYVNRSLPGLIGCHRKSVYWHVIGMGTTPEVHSIFLEGHTFLVRNHRQASLEISPITFLTAQTLLMDLGQFLLFCHISSHQHDGMEAYVKVDSCPEEPQLRMKNNEEAEDYDDDLTDSEMDVVRFDDDNSPSFIQIRSVAKKHPKTWVHYIAAEEEDWDYAPLVLAPDDRSYKSQYLNNGPQRIGRKYKKVRFMAYTDETFKTREAIQHESGILGPLLYGEVGDTLLIIFKNQASRPYNIYPHGITDVRPLYSRRLPKGVKHLKDFPILPGEIFKYKWTVTVEDGPTKSDPRCLTRYYSSFVNMERDLASGLIGPLLICYKESVDQRGNQIMSDKRNVILFSVFDENRSWYLTENIQRFLPNPAGVQLEDPEFQASNIMHSINGYVFDSLQLSVCLHEVAYWYILSIGAQTDFLSVFFSGYTFKHKMVYEDTLTLFPFSGETVFMSMENPGLWILGCHNSDFRNRGMTALLKVSSCDKNTGDYYEDSYEDISAYLLSKNNAIEPRSFSQNPPVLKAHQAEITRTTLQSDQEEIDYDDTISVEMKKEDFDIYDEDENQSPRSFQKKTRHYFIAAVERLWDYGMSSSPHVLRNRAQSGSVPQFKKVVFQEFTDGSFTQPLYRGELNEHLGLLGPYIRAEVEDNIMVTFRNQASRPYSFYSSLISYEEDQRQGAEPRKNFVKPNETKTYFWKVQHHMAPTKDEFDCKAWAYFSDVDLEKDVHSGLIGPLLVCHTNTLNPAHGRQVTVQEFALFFTIFDETKSWYFTENMERNCRAPCNIQMEDPTFKENYRFHAINGYIMDTLPGLVMAQDQRIRWYLLSMGSNENIHSIHFSGHVFTVRKKEEYKMALYNLYPGVFETVEMLPSKAGIWRVECLIGEHLHAGMSTLFLVYSNKCQTPLGMASGHIRDFQITASGQYGQWAPKLARLHYSGSINAWSTKEPFSWIKVDLLAPMIIHGIKTQGARQKFSSLYISQFIIMYSLDGKKWQTYRGNSTGTLMVFFGNVDSSGIKHNIFNPPIIARYIRLHPTHYSIRSTLRMELMGCDLNSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNSLDPPLLTRYLRIHPQSWVHQIALRMEVLGCEAQDLYDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 91 - DNA, 5052 bp, synthetic construct
  misc_feature 1..5052: note = pSYN-FVIII-155
  source 1..5052: mol_type = other DNA
atgcaaatagagctctccacctgcttctttctgtgccttttgcgattctgctttagtgccaccagaagatactacctgggtgcagtggaactgtcatgggactatatgcaaagtgatctcggtgagctgcctgtggacgcaagatttcctcctagagtgccaaaatcttttccattcaacacctcagtcgtgtacaaaaagactctgtttgtagaattcacggatcaccttttcaacatcgctaagccaaggccaccctggatgggtctgctaggtcctaccatccaggctgaggtttatgatacagtggtcattacacttaagaacatggcttcccatcctgtcagtcttcatgctgttggtgtatcctactggaaagcttctgagggagctgaatatgatgatcagaccagtcaaagggagaaagaagatgataaagtcttccctggtggaagccatacatatgtctggcaggtcctgaaagagaatggtccaatggcctctgacccactgtgccttacctactcatatctttctcatgtggacctggtaaaagacttgaattcaggcctcattggagccctactagtatgtagagaagggagtctggccaaggaaaagacacagaccttgcacaaatttatactactttttgctgtatttgatgaagggaaaagttggcactcagaaacaaagaactccttgatgcaggatagggatgctgcatctgctcgggcctggcctaaaatgcacacagtcaatggttatgtaaacaggtctctgccaggtctgattggatgccacaggaaatcagtctattggcatgtgattggaatgggcaccactcctgaagtgcactcaatattcctcgaaggtcacacatttcttgtgaggaaccatcgccaggcgtccttggaaatctcgccaataactttccttactgctcaaacactcttgatggaccttggacagtttctactgttttgtcatatctcttcccaccaacatgatggcatggaagcttatgtcaaagtagacagctgtccagaggaaccccaactacgaatgaaaaataatgaagaagcggaagactatgatgatgatcttactgattctgaaatggatgtggtcaggtttgatgatgacaactctccttcctttatccaaattcgctcagttgccaagaagcatcctaaaacttgggtacattacattgctgctgaagaggaggactgggactatgctcccttagtcctcgcccccgatgacagaagttataaaagtcaatatttgaacaatggccctcagcggattggtaggaagtacaaaaaagtccgatttatggcatacacagatgaaacctttaagactcgtgaagctattcagcatgaatcaggaatcttgggacctttactttatggggaagttggagacacactgttgattatatttaagaatcaagcaagcagaccatataacatctaccctcacggaatcactgatgtccgtcctttgtattcaaggagattaccaaaaggtgtaaaacatttgaaggattttccaattctgccaggagaaatattcaaatataaatggacagtgactgtagaagatgggccaactaaatcagatcctcggtgcctgacccgctattactctagtttcgttaatatggagagagatctagcttcaggactcattggccctctcctcatctgctacaaagaatctgtagatcaaagaggaaaccagataatgtcagacaagaggaatgtcatcctgttttctgtatttgatgagaaccgaagctggtacctcacagagaatatacaacgctttctccccaatccagctggagtgcagcttgaggatccagagttccaagcctccaacatcatgcacagcatcaatggctatgtttttgatagtttgcagttgtcagtttgtttgcatgaggtggcatactggtacattctaagcattggagcacagactgacttcctttctgtcttcttctctggatataccttcaaacacaaaatggtctatgaagacacactcaccctattcccattctcaggagaaactgtcttcatgtcgatggaaaacccaggtctatggattctggggtgccacaactcagactttcggaacagaggcatgaccgccttactgaaggtttctagttgtgacaagaacactggtgattattacgaggacagttatgaagatatttcagcatacttgctgagtaaaaacaatgccattgaaccaagaagcttctctcaaaacccaccagtcttgaaagcccatcaggcggaaataactcgtactactcttcagtcagatcaagaggaaattgactatgatgataccatatcagttgaaatgaagaaggaagattttgacatttatgatgaggatgaaaatcagagcccccgcagctttcaaaagaaaacacgacactattttattgctgcagtggagaggctctgggattatgggatgagtagctccccacatgttctaagaaacagggctcagagtggcagtgtccctcagttcaagaaagttgttttccaggaatttactgatggctcctttactcagcccttataccgtggagaactaaatgaacatttgggactcctggggccatatataagagcagaagttgaagataatatcatggtaactttcagaaatcaggcctctcgtccctattccttctattctagccttatttcttatgaggaagatcagaggcaaggagcagaacctagaaaaaactttgtcaagcctaatgaaaccaaaacttacttttggaaagtgcaacatcatatggcacccactaaagatgagtttgactgcaaagcctgggcttatttctctgatgttgacctggaaaaagatgtgcactcaggcctgattggaccccttctggtctgccacactaacacactgaaccctgctcatgggagacaagtgacagtacaggaatttgctctgtttttcaccatctttgatgagaccaaaagctggtacttcactgaaaatatggaaagaaactgcagggctccctgcaatatccagatggaagatcccacttttaaagagaattatcgcttccatgcaatcaatggctacataatggatacactacctggcttagtaatggctcaggatcaaaggattcgatggtatctgctcagcatgggcagcaatgaaaacatccattctattcatttcagtggacatgtgttcactgtacgaaaaaaagaggagtataaaatggcactgtacaatctctatccaggtgtttttgagacagtggaaatgttaccatccaaagctggaatttggcgggtggaatgccttattggcgagcatctacatgctgggatgagcacactttttctggtgtacagcaataagtgtcagactcccctgggaatggcttctggacacattagagattttcagattacagcttcaggacaatatggacagtgggccccaaagctggccagacttcattattccggatcaatcaatgcctggagcaccaaggagcccttttcttggatcaaggtggatctgttggcaccaatgattattcacggcatcaagacccagggtgcccgtcagaagttctccagcctctacatctctcagtttatcatcatgtatagtcttgatgggaagaagtggcagacttatcgaggaaattccactggaaccttaatggtcttctttggcaatgtggattcatctgggataaaacacaatatttttaaccctccaattattgctcgatacatccgtttgcacccaactcattatagcattcgcagcactcttcgcatggagttgatgggctgtgatttaaatagttgcagcatgccattgggaatggagagtaaagcaatatcagatgcacagattactgcttcatcctactttaccaatatgtttgccacctggtctccttcaaaagctcgacttcacctccaagggaggagtaatgcctggagacctcaggtgaataatccaaaagagtggctgcaagtggacttccagaagacaatgaaagtcacaggagtaactactcagggagtaaaatctctgcttaccagcatgtatgtgaaggagttcctcatctccagcagtcaagatggccatcagtggactctcttttttcagaatggcaaagtaaaggtttttcagggaaatcaagactccttcacacctgtggtgaactctctagacccaccgttactgactcgctaccttcgaattcacccccagagttgggtgcaccagattgccctgaggatggaggttctgggctgcgaggcacaggacctctacgacaaaactcacacatgcccaccgtgcccagctccagaactcctgggcggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgttggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 92 - protein, 19 aa, synthetic construct
  REGION 1..19: note = GFVIII-159
IDGGGGSGGGGSLVPRGSG

SEQ ID NO: 93 - protein, 34 aa, synthetic construct
  REGION 1..34: note = GFVIII-160
ISGGGGSGGGGSGGGGSGGGGSGGGGSLVPRGSG

SEQ ID NO: 94 - protein, 48 aa, synthetic construct; identical to SEQ ID NO: 81
  REGION 1..48: note = FVIII-064

SEQ ID NO: 95 - protein, 48 aa, synthetic construct; identical to SEQ ID NO: 81
  REGION 1..48: note = VWF031

SEQ ID NO: 96 - protein, 73 aa, synthetic construct; identical to SEQ ID NO: 84
  REGION 1..73: note = VWF035

SEQ ID NO: 97 - protein, 98 aa, synthetic construct; identical to SEQ ID NO: 86
  REGION 1..98: note = VWF036

SEQ ID NO: 98 - protein, 54 aa, synthetic construct
  REGION 1..54: note = pSYN-VWF-051 linker
ISGGGGSGGGGSGGGGSGGGGSGGGGSGGGGSLPETGALRPRVVGGGGSGGGGS

SEQ ID NO: 99 - DNA, 232 bp, synthetic construct
  misc_feature 1..232: note = Genewiz-Sequence no-10-210746313
  source 1..232: mol_type = other DNA
aggagccgatatctggcggtggaggttccggtggcgggggatccggcggtggaggttccggcggtggaggttccggtggcgggggatccggtggcgggggatccttacctgaaactggagccctgcggccccgggtcgtcggcggtggaggttccggtggcgggggatccgacaaaactcacacatgcccaccgtgcccagctccagaactcctgggcggaccgtcagtctt

SEQ ID NO: 100 - DNA, 4566 bp, synthetic construct
  misc_feature 1..4566: note = pSYN-VWF051
  source 1..4566: mol_type = other DNA
atgattcctgccagatttgccggggtgctgcttgctctggccctcattttgccagggaccctttgtgcagaaggaactcgcggcaggtcatccacggcccgatgcagccttttcggaagtgacttcgtcaacacctttgatgggagcatgtacagctttgcgggatactgcagttacctcctggcagggggctgccagaaacgctccttctcgattattggggacttccagaatggcaagagagtgagcctctccgtgtatcttggggaattttttgacatccatttgtttgtcaatggtaccgtgacacagggggaccaaagagtctccatgccctatgcctccaaagggctgtatctagaaactgaggctgggtactacaagctgtccggtgaggcctatggctttgtggccaggatcgatggcagcggcaactttcaagtcctgctgtcagacagatacttcaacaagacctgcgggctgtgtggcaactttaacatctttgctgaagatgactttatgacccaagaagggaccttgacctcggacccttatgactttgccaactcatgggctctgagcagtggagaacagtggtgtgaacgggcatctcctcccagcagctcatgcaacatctcctctggggaaatgcagaagggcctgtgggagcagtgccagcttctgaagagcacctcggtgtttgcccgctgccaccctctggtggaccccgagccttttgtggccctgtgtgagaagactttgtgtgagtgtgctggggggctggagtgcgcctgccctgccctcctggagtacgcccggacctgtgcccaggagggaatggtgctgtacggctggaccgaccacagcgcgtgcagcccagtgtgccctgctggtatggagtataggcagtgtgtgtccccttgcgccaggacctgccagagcctgcacatcaatgaaatgtgtcaggagcgatgcgtggatggctgcagctgccctgagggacagctcctggatgaaggcctctgcgtggagagcaccgagtgtccctgcgtgcattccggaaagcgctaccctcccggcacctccctctctcgagactgcaacacctgcatttgccgaaacagccagtggatctgcagcaatgaagaatgtccaggggagtgccttgtcactggtcaatcccacttcaagagctttgacaacagatacttcaccttcagtgggatctgccagtacctgctggcccgggattgccaggaccactccttctccattgtcattgagactgtccagtgtgctgatgaccgcgacgctgtgtgcacccgctccgtcaccgtccggctgcctggcctgcacaacagccttgtgaaactgaagcatggggcaggagttgccatggatggccaggacatccagctccccctcctgaaaggtgacctccgcatccagcatacagtgacggcctccgtgcgcctcagctacggggaggacctgcagatggactgggatggccgcgggaggctgctggtgaagctgtcccccgtctatgccgggaagacctgcggcctgtgtgggaattacaatggcaaccagggcgacgacttccttaccccctctgggctggcggagccccgggtggaggacttcgggaacgcctggaagctgcacggggactgccaggacctgcagaagcagcacagcgatccctgcgccctcaacccgcgcatgaccaggttctccgaggaggcgtgcgcggtcctgacgtcccccacattcgaggcctgccatcgtgccgtcagcccgctgccctacctgcggaactgccgctacgacgtgtgctcctgctcggacggccgcgagtgcctgtgcggcgccctggccagctatgccgcggcctgcgcggggagaggcgtgcgcgtcgcgtggcgcgagccaggccgctgtgagctgaactgcccgaaaggccaggtgtacctgcagtgcgggaccccctgcaacctgacctgccgctctctctcttacccggatgaggaatgcaatgaggcctgcctggagggctgcttctgccccccagggctctacatggatgagaggggggactgcgtgcccaaggcccagtgcccctgttactatgacggtgagatcttccagccagaagacatcttctcagaccatcacaccatgtgctactgtgaggatggcttcatgcactgtaccatgagtggagtccccggaagcttgctgcctgacgctgtcctcagcagtcccctgtctcatcgcagcaaaaggagcctatcctgtcggccccccatggtcaagctggtgtgtcccgctgacaacctgcgggctgaagggctcgagtgtaccaaaacgtgccagaactatgacctggagtgcatgagcatgggctgtgtctctggctgcctctgccccccgggcatggtccggcatgagaacagatgtgtggccctggaaaggtgtccctgcttccatcagggcaaggagtatgcccctggagaaacagtgaagattggctgcaacacttgtgtctgtcgggaccggaagtggaactgcacagaccatgtgtgtgatgccacgtgctccacgatcggcatggcccactacctcaccttcgacgggctcaaatacctgttccccggggagtgccagtacgttctggtgcaggattactgcggcagtaaccctgggacctttcggatcctagtggggaataagggatgcagccacccctcagtgaaatgcaagaaacgggtcaccatcctggtggagggaggagagattgagctgtttgacggggaggtgaatgtgaagaggcccatgaaggatgagactcactttgaggtggtggagtctggccggtacatcattctgctgctgggcaaagccctctccgtggtctgggaccgccacctgagcatctccgtggtcctgaagcagacataccaggagaaagtgtgtggcctgtgtgggaattttgatggcatccagaacaatgacctcaccagcagcaacctccaagtggaggaagaccctgtggactttgggaactcctggaaagtgagctcgcagtgtgctgacaccagaaaagtgcctctggactcatcccctgccacctgccataacaacatcatgaagcagacgatggtggattcctcctgtagaatccttaccagtgacgtcttccaggactgcaacaagctggtggaccccgagccatatctggatgtctgcatttacgacacctgctcctgtgagtccattggggactgcgccgcattctgcgacaccattgctgcctatgcccacgtgtgtgcccagcatggcaaggtggtgacctggaggacggccacattgtgcccccagagctgcgaggagaggaatctccgggagaacgggtatgaggctgagtggcgctataacagctgtgcacctgcctgtcaagtcacgtgtcagcaccctgagccactggcctgccctgtgcagtgtgtggagggctgccatgcccactgccctccagggaaaatcctggatgagcttttgcagacctgcgttgaccctgaagactgtccagtgtgtgaggtggctggccggcgttttgcctcaggaaagaaagtcaccttgaatcccagtgaccctgagcactgccagatttgccactgtgatgttgtcaacctcacctgtgaagcctgccaggagccgatatctggcggtggaggttccggtggcgggggatccggcggtggaggttccggcggtggaggttccggtggcgggggatccggtggcgggggatccttacctgaaactggagccctgcggccccgggtcgtcggcggtggaggttccggtggcgggggatccgacaaaactcacacatgcccaccgtgcccagctccagaactcctgggcggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgttggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 101 - protein, 1521 aa, synthetic construct
  REGION 1..1521: note = VWF051
MIPARFAGVLLALALILPGTLCAEGTRGRSSTARCSLFGSDFVNTFDGSMYSFAGYCSYLLAGGCQKRSFSIIGDFQNGKRVSLSVYLGEFFDIHLFVNGTVTQGDQRVSMPYASKGLYLETEAGYYKLSGEAYGFVARIDGSGNFQVLLSDRYFNKTCGLCGNFNIFAEDDFMTQEGTLTSDPYDFANSWALSSGEQWCERASPPSSSCNISSGEMQKGLWEQCQLLKSTSVFARCHPLVDPEPFVALCEKTLCECAGGLECACPALLEYARTCAQEGMVLYGWTDHSACSPVCPAGMEYRQCVSPCARTCQSLHINEMCQERCVDGCSCPEGQLLDEGLCVESTECPCVHSGKRYPPGTSLSRDCNTCICRNSQWICSNEECPGECLVTGQSHFKSFDNRYFTFSGICQYLLARDCQDHSFSIVIETVQCADDRDAVCTRSVTVRLPGLHNSLVKLKHGAGVAMDGQDIQLPLLKGDLRIQHTVTASVRLSYGEDLQMDWDGRGRLLVKLSPVYAGKTCGLCGNYNGNQGDDFLTPSGLAEPRVEDFGNAWKLHGDCQDLQKQHSDPCALNPRMTRFSEEACAVLTSPTFEACHRAVSPLPYLRNCRYDVCSCSDGRECLCGALASYAAACAGRGVRVAWREPGRCELNCPKGQVYLQCGTPCNLTCRSLSYPDEECNEACLEGCFCPPGLYMDERGDCVPKAQCPCYYDGEIFQPEDIFSDHHTMCYCEDGFMHCTMSGVPGSLLPDAVLSSPLSHRSKRSLSCRPPMVKLVCPADNLRAEGLECTKTCQNYDLECMSMGCVSGCLCPPGMVRHENRCVALERCPCFHQGKEYAPGETVKIGCNTCVCRDRKWNCTDHVCDATCSTIGMAHYLTFDGLKYLFPGECQYVLVQDYCGSNPGTFRILVGNKGCSHPSVKCKKRVTILVEGGEIELFDGEVNVKRPMKDETHFEVVESGRYIILLLGKALSVVWDRHLSISVVLKQTYQEKVCGLCGNFDGIQNNDLTSSNLQVEEDPVDFGNSWKVSSQCADTRKVPLDSSPATCHNNIMKQTMVDSSCRILTSDVFQDCNKLVDPEPYLDVCIYDTCSCESIGDCAAFCDTIAAYAHVCAQHGKVVTWRTATLCPQSCEERNLRENGYEAEWRYNSCAPACQVTCQHPEPLACPVQCVEGCHAHCPPGKILDELLQTCVDPEDCPVCEVAGRRFASGKKVTLNPSDPEHCQICHCDVVNLTCEACQEPISGGGGSGGGGSGGGGSGGGGSGGGGSGGGGSLPETGALRPRVVGGGGSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 102 - DNA, 4389 bp, synthetic construct
  misc_feature 1..4389: note = FVIII 265
  source 1..4389: mol_type = other DNA
atgcaaatagagctctccacctgcttctttctgtgccttttgcgattctgctttagtggaggaggaggaggagccaccagaagatactacctgggtgcagtggaactgtcatgggactatatgcaaagtgatctcggtgagctgcctgtggacgcaagatttcctcctagagtgccaaaatcttttccattcaacacctcagtcgtgtacaaaaagactctgtttgtagaattcacggatcaccttttcaacatcgctaagccaaggccaccctggatgggtctgctaggtcctaccatccaggctgaggtttatgatacagtggtcattacacttaagaacatggcttcccatcctgtcagtcttcatgctgttggtgtatcctactggaaagcttctgagggagctgaatatgatgatcagaccagtcaaagggagaaagaagatgataaagtcttccctggtggaagccatacatatgtctggcaggtcctgaaagagaatggtccaatggcctctgacccactgtgccttacctactcatatctttctcatgtggacctggtaaaagacttgaattcaggcctcattggagccctactagtatgtagagaagggagtctggccaaggaaaagacacagaccttgcacaaatttatactactttttgctgtatttgatgaagggaaaagttggcactcagaaacaaagaactccttgatgcaggatagggatgctgcatctgctcgggcctggcctaaaatgcacacagtcaatggttatgtaaacaggtctctgccaggtctgattggatgccacaggaaatcagtctattggcatgtgattggaatgggcaccactcctgaagtgcactcaatattcctcgaaggtcacacatttcttgtgaggaaccatcgccaggcgtccttggaaatctcgccaataactttccttactgctcaaacactcttgatggaccttggacagtttctactgttttgtcatatctcttcccaccaacatgatggcatggaagcttatgtcaaagtagacagctgtccagaggaaccccaactacgaatgaaaaataatgaagaagcggaagactatgatgatgatcttactgattctgaaatggatgtggtcaggtttgatgatgacaactctccttcctttatccaaattcgctcagttgccaagaagcatcctaaaacttgggtacattacattgctgctgaagaggaggactgggactatgctcccttagtcctcgcccccgatgacagaagttataaaagtcaatatttgaacaatggccctcagcggattggtaggaagtacaaaaaagtccgatttatggcatacacagatgaaacctttaagactcgtgaagctattcagcatgaatcaggaatcttgggacctttactttatggggaagttggagacacactgttgattatatttaagaatcaagcaagcagaccatataacatctaccctcacggaatcactgatgtccgtcctttgtattcaaggagattaccaaaaggtgtaaaacatttgaaggattttccaattctgccaggagaaatattcaaatataaatggacagtgactgtagaagatgggccaactaaatcagatcctcggtgcctgacccgctattactctagtttcgttaatatggagagagatctagcttcaggactcattggccctctcctcatctgctacaaagaatctgtagatcaaagaggaaaccagataatgtcagacaagaggaatgtcatcctgttttctgtatttgatgagaaccgaagctggtacctcacagagaatatacaacgctttctccccaatccagctggagtgcagcttgaggatccagagttccaagcctccaacatcatgcacagcatcaatggctatgtttttgatagtttgcagttgtcagtttgtttgcatgaggtggcatactggtacattctaagcattggagcacagactgacttcctttctgtcttcttctctggatataccttcaaacacaaaatggtctatgaagacacactcaccctattcccattctcaggagaaactgtcttcatgtcgatggaaaacccaggtctatggattctggggtgccacaactcagactttcggaacagaggcatgaccgccttactgaaggtttctagttgtgacaagaacactggtgattattacgaggacagttatgaagatatttcagcatacttgctgagtaaaaacaatgccattgaaccaagaagcttctctcaaaacccaccagtcttgaaggcccatcaggccgaaataactcgtactactcttcagtcagatcaagaggaaattgactatgatgataccatatcagttgaaatgaagaaggaagattttgacatttatgatgaggatgaaaatcagagcccccgcagctttcaaaagaaaacacgacactattttattgctgcagtggagaggctctgggattatgggatgagtagctccccacatgttctaagaaacagggctcagagtggcagtgtccctcagttcaagaaagttgttttccaggaatttactgatggctcctttactcagcccttataccgtggagaactaaatgaacatttgggcctcctcggcccatatataagagcagaagttgaagataatatcatggtaactttcagaaatcaggcctctcgtccctattccttctattctagccttatttcttatgaggaagatcagaggcaaggagcagaacctagaaaaaactttgtcaagcctaatgaaaccaaaacttacttttggaaagtgcaacatcatatggcacccactaaagatgagtttgactgcaaagcctgggcttatttctctgatgttgacctggaaaaagatgtgcactcaggcctgattggaccccttctggtctgccacactaacacactgaaccctgctcatgggagacaagtgacagtacaggaatttgctctgtttttcaccatctttgatgagaccaaaagctggtacttcactgaaaatatggaaagaaactgcagggctccctgcaatatccagatggaagatcccacttttaaagagaattatcgcttccatgcaatcaatggctacataatggatacactacctggcttagtaatggctcaggatcaaaggattcgatggtatctgctcagcatgggcagcaatgaaaacatccattctattcatttcagtggacatgtgttcactgtacgaaaaaaagaggagtataaaatggcactgtacaatctctatccaggtgtttttgagacagtggaaatgttaccatccaaagctggaatttggcgggtggaatgccttattggcgagcatctacatgctgggatgagcacactttttctggtgtacagcaataagtgtcagactcccctgggaatggcttctggacacattagagattttcagattacagcttcaggacaatatggacagtgggccccaaagctggccagacttcattattccggatcaatcaatgcctggagcaccaaggagcccttttcttggatcaaggtggatctgttggcaccaatgattattcacggcatcaagacccagggtgcccgtcagaagttctccagcctctacatctctcagtttatcatcatgtatagtcttgatgggaagaagtggcagacttatcgaggaaattccactggaaccttaatggtcttctttggcaatgtggattcatctgggataaaacacaatatttttaaccctccaattattgctcgatacatccgtttgcacccaactcattatagcattcgcagcactcttcgcatggagttgatgggctgtgatttaaatagttgcagcatgccattgggaatggagagtaaagcaatatcagatgcacagattactgcttcatcctactttaccaatatgtttgccacctggtctccttcaaaagctcgacttcacctccaagggaggagtaatgcctggagacctcaggtgaataatccaaaagagtggctgcaagtggacttccagaagacaatgaaagtcacaggagtaactactcagggagtaaaatctctgcttaccagcatgtatgtgaaggagttcctcatctccagcagtcaagatggccatcagtggactctcttttttcagaatggcaaagtaaaggtttttcagggaaatcaagactccttcacacctgtggtgaactctctagacccaccgttactgactcgctaccttcgaattcacccccagagttgggtgcaccagattgccctgaggatggaggttctgggctgcgaggcacaggacctctactga

SEQ ID NO: 103 - protein, 1462 aa, synthetic construct
  REGION 1..1462: note = FVIII 265
MQIELSTCFFLCLLRFCFSGGGGGATRRYYLGAVELSWDYMQSDLGELPVDARFPPRVPKSFPFNTSVVYKKTLFVEFTDHLFNIAKPRPPWMGLLGPTIQAEVYDTVVITLKNMASHPVSLHAVGVSYWKASEGAEYDDQTSQREKEDDKVFPGGSHTYVWQVLKENGPMASDPLCLTYSYLSHVDLVKDLNSGLIGALLVCREGSLAKEKTQTLHKFILLFAVFDEGKSWHSETKNSLMQDRDAASARAWPKMHTVNGYVNRSLPGLIGCHRKSVYWHVIGMGTTPEVHSIFLEGHTFLVRNHRQASLEISPITFLTAQTLLMDLGQFLLFCHISSHQHDGMEAYVKVDSCPEEPQLRMKNNEEAEDYDDDLTDSEMDVVRFDDDNSPSFIQIRSVAKKHPKTWVHYIAAEEEDWDYAPLVLAPDDRSYKSQYLNNGPQRIGRKYKKVRFMAYTDETFKTREAIQHESGILGPLLYGEVGDTLLIIFKNQASRPYNIYPHGITDVRPLYSRRLPKGVKHLKDFPILPGEIFKYKWTVTVEDGPTKSDPRCLTRYYSSFVNMERDLASGLIGPLLICYKESVDQRGNQIMSDKRNVILFSVFDENRSWYLTENIQRFLPNPAGVQLEDPEFQASNIMHSINGYVFDSLQLSVCLHEVAYWYILSIGAQTDFLSVFFSGYTFKHKMVYEDTLTLFPFSGETVFMSMENPGLWILGCHNSDFRNRGMTALLKVSSCDKNTGDYYEDSYEDISAYLLSKNNAIEPRSFSQNPPVLKAHQAEITRTTLQSDQEEIDYDDTISVEMKKEDFDIYDEDENQSPRSFQKKTRHYFIAAVERLWDYGMSSSPHVLRNRAQSGSVPQFKKVVFQEFTDGSFTQPLYRGELNEHLGLLGPYIRAEVEDNIMVTFRNQASRPYSFYSSLISYEEDQRQGAEPRKNFVKPNETKTYFWKVQHHMAPTKDEFDCKAWAYFSDVDLEKDVHSGLIGPLLVCHTNTLNPAHGRQVTVQEFALFFTIFDETKSWYFTENMERNCRAPCNIQMEDPTFKENYRFHAINGYIMDTLPGLVMAQDQRIRWYLLSMGSNENIHSIHFSGHVFTVRKKEEYKMALYNLYPGVFETVEMLPSKAGIWRVECLIGEHLHAGMSTLFLVYSNKCQTPLGMASGHIRDFQITASGQYGQWAPKLARLHYSGSINAWSTKEPFSWIKVDLLAPMIIHGIKTQGARQKFSSLYISQFIIMYSLDGKKWQTYRGNSTGTLMVFFGNVDSSGIKHNIFNPPIIARYIRLHPTHYSIRSTLRMELMGCDLNSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNSLDPPLLTRYLRIHPQSWVHQIALRMEVLGCEAQDLY

SEQ ID NO: 104 - DNA, 5691 bp, synthetic construct
  misc_feature 1..5691: note = FVIII198
  source 1..5691: mol_type = other DNA
atgcaaatagagctctccacctgcttctttctgtgccttttgcgattctgctttagtgccaccagaagatactacctgggtgcagtggaactgtcatgggactatatgcaaagtgatctcggtgagctgcctgtggacgcaagatttcctcctagagtgccaaaatcttttccattcaacacctcagtcgtgtacaaaaagactctgtttgtagaattcacggatcaccttttcaacatcgctaagccaaggccaccctggatgggtctgctaggtcctaccatccaggctgaggtttatgatacagtggtcattacacttaagaacatggcttcccatcctgtcagtcttcatgctgttggtgtatcctactggaaagcttctgagggagctgaatatgatgatcagaccagtcaaagggagaaagaagatgataaagtcttccctggtggaagccatacatatgtctggcaggtcctgaaagagaatggtccaatggcctctgacccactgtgccttacctactcatatctttctcatgtggacctggtaaaagacttgaattcaggcctcattggagccctactagtatgtagagaagggagtctggccaaggaaaagacacagaccttgcacaaatttatactactttttgctgtatttgatgaagggaaaagttggcactcagaaacaaagaactccttgatgcaggatagggatgctgcatctgctcgggcctggcctaaaatgcacacagtcaatggttatgtaaacaggtctctgccaggtctgattggatgccacaggaaatcagtctattggcatgtgattggaatgggcaccactcctgaagtgcactcaatattcctcgaaggtcacacatttcttgtgaggaaccatcgccaggcgtccttggaaatctcgccaataactttccttactgctcaaacactcttgatggaccttggacagtttctactgttttgtcatatctcttcccaccaacatgatggcatggaagcttatgtcaaagtagacagctgtccagaggaaccccaactacgaatgaaaaataatgaagaagcggaagactatgatgatgatcttactgattctgaaatggatgtggtcaggtttgatgatgacaactctccttcctttatccaaattcgctcagttgccaagaagcatcctaaaacttgggtacattacattgctgctgaagaggaggactgggactatgctcccttagtcctcgcccccgatgacagaagttataaaagtcaatatttgaacaatggccctcagcggattggtaggaagtacaaaaaagtccgatttatggcatacacagatgaaacctttaagactcgtgaagctattcagcatgaatcaggaatcttgggacctttactttatggggaagttggagacacactgttgattatatttaagaatcaagcaagcagaccatataacatctaccctcacggaatcactgatgtccgtcctttgtattcaaggagattaccaaaaggtgtaaaacatttgaaggattttccaattctgccaggagaaatattcaaatataaatggacagtgactgtagaagatgggccaactaaatcagatcctcggtgcctgacccgctattactctagtttcgttaatatggagagagatctagcttcaggactcattggccctctcctcatctgctacaaagaatctgtagatcaaagaggaaaccagataatgtcagacaagaggaatgtcatcctgttttctgtatttgatgagaaccgaagctggtacctcacagagaatatacaacgctttctccccaatccagctggagtgcagcttgaggatccagagttccaagcctccaacatcatgcacagcatcaatggctatgtttttgatagtttgcagttgtcagtttgtttgcatgaggtggcatactggtacattctaagcattggagcacagactgacttcctttctgtcttcttctctggatataccttcaaacacaaaatggtctatgaagacacactcaccctattcccattctcaggagaaactgtcttcatgtcgatggaaaacccaggtctatggattctggggtgccacaactcagactttcggaacagaggcatgaccgccttactgaaggtttctagttgtgacaagaacactggtgattattacgaggacagttatgaagatatttcagcatacttgctgagtaaaaacaatgccattgaaccaagaagcttctctcagaattcaagacaccctagcactaggcaaaagcaatttaatgccaccacaattccagaaaatgacatagagaagactgacccttggtttgcacacagaacacctatgcctaaaatacaaaatgtctcctctagtgatttgttgatgctcttgcgacagagtcctactccacatgggctatccttatctgatctccaagaagccaaatatgagactttttctgatgatccatcacctggagcaatagacagtaataacagcctgtctgaaatgacacacttcaggccacagctccatcacagtggggacatggtatttacccctgagtcaggcctccaattaagattaaatgagaaactggggacaactgcagcaacagagttgaagaaacttgatttcaaagtttctagtacatcaaataatctgatttcaacaattccatcagacaatttggcagcaggtactgataatacaagttccttaggacccccaagtatgccagttcattatgatagtcaattagataccactctatttggcaaaaagtcatctccccttactgagtctggtggacctctgagcttgagtgaagaaaataatgattcaaagttgttagaatcaggtttaatgaatagccaagaaagttcatggggaaaaaatgtatcgtcagaaataactcgtactactcttcagtcagatcaagaggaaattgactatgatgataccatatcagttgaaatgaagaaggaagattttgacatttatgatgaggatgaaaatcagagcccccgcagctttcaaaagaaaacacgacactattttattgctgcagtggagaggctctgggattatgggatgagtagctccccacatgttctaagaaacagggctcagagtggcagtgtccctcagttcaagaaagttgttttccaggaatttactgatggctcctttactcagcccttataccgtggagaactaaatgaacatttgggactcctggggccatatataagagcagaagttgaagataatatcatggtaactttcagaaatcaggcctctcgtccctattccttctattctagccttatttcttatgaggaagatcagaggcaaggagcagaacctagaaaaaactttgtcaagcctaatgaaaccaaaacttacttttggaaagtgcaacatcatatggcacccactaaagatgagtttgactgcaaagcctgggcttatttctctgatgttgacctggaaaaagatgtgcactcaggcctgattggaccccttctggtctgccacactaacacactgaaccctgctcatgggagacaagtgacagtacaggaatttgctctgtttttcaccatctttgatgagaccaaaagctggtacttcactgaaaatatggaaagaaactgcagggctccctgcaatatccagatggaagatcccacttttaaagagaattatcgcttccatgcaatcaatggctacataatggatacactacctggcttagtaatggctcaggatcaaaggattcgatggtatctgctcagcatgggcagcaatgaaaacatccattctattcatttcagtggacatgtgttcactgtacgaaaaaaagaggagtataaaatggcactgtacaatctctatccaggtgtttttgagacagtggaaatgttaccatccaaagctggaatttggcgggtggaatgccttattggcgagcatctacatgctgggatgagcacactttttctggtgtacagcaataagtgtcagactcccctgggaatggcttctggacacattagagattttcagattacagcttcaggacaatatggacagtgggccccaaagctggccagacttcattattccggatcaatcaatgcctggagcaccaaggagcccttttcttggatcaaggtggatctgttggcaccaatgattattcacggcatcaagacccagggtgcccgtcagaagttctccagcctctacatctctcagtttatcatcatgtatagtcttgatgggaagaagtggcagacttatcgaggaaattccactggaaccttaatggtcttctttggcaatgtggattcatctgggataaaacacaatatttttaaccctccaattattgctcgatacatccgtttgcacccaactcattatagcattcgcagcactcttcgcatggagttgatgggctgtgatttaaatagttgcagcatgccattgggaatggagagtaaagcaatatcagatgcacagattactgcttcatcctactttaccaatatgtttgccacctggtctccttcaaaagctcgacttcacctccaagggaggagtaatgcctggagacctcaggtgaataatccaaaagagtggctgcaagtggacttccagaagacaatgaaagtcacaggagtaactactcagggagtaaaatctctgcttaccagcatgtatgtgaaggagttcctcatctccagcagtcaagatggccatcagtggactctcttttttcagaatggcaaagtaaaggtttttcagggaaatcaagactccttcacacctgtggtgaactctctagacccaccgttactgactcgctaccttcgaattcacccccagagttgggtgcaccagattgccctgaggatggaggttctgggctgcgaggcacaggacctctacgacaaaactcacacatgcccaccgtgcccagctccagaactcctgggcggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgttggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 105 - protein, 1896 aa, synthetic construct
  REGION 1..1896: note = FVIII 198
MQIELSTCFFLCLLRFCFSATRRYYLGAVELSWDYMQSDLGELPVDARFPPRVPKSFPFNTSVVYKKTLFVEFTDHLFNIAKPRPPWMGLLGPTIQAEVYDTVVITLKNMASHPVSLHAVGVSYWKASEGAEYDDQTSQREKEDDKVFPGGSHTYVWQVLKENGPMASDPLCLTYSYLSHVDLVKDLNSGLIGALLVCREGSLAKEKTQTLHKFILLFAVFDEGKSWHSETKNSLMQDRDAASARAWPKMHTVNGYVNRSLPGLIGCHRKSVYWHVIGMGTTPEVHSIFLEGHTFLVRNHRQASLEISPITFLTAQTLLMDLGQFLLFCHISSHQHDGMEAYVKVDSCPEEPQLRMKNNEEAEDYDDDLTDSEMDVVRFDDDNSPSFIQIRSVAKKHPKTWVHYIAAEEEDWDYAPLVLAPDDRSYKSQYLNNGPQRIGRKYKKVRFMAYTDETFKTREAIQHESGILGPLLYGEVGDTLLIIFKNQASRPYNIYPHGITDVRPLYSRRLPKGVKHLKDFPILPGEIFKYKWTVTVEDGPTKSDPRCLTRYYSSFVNMERDLASGLIGPLLICYKESVDQRGNQIMSDKRNVILFSVFDENRSWYLTENIQRFLPNPAGVQLEDPEFQASNIMHSINGYVFDSLQLSVCLHEVAYWYILSIGAQTDFLSVFFSGYTFKHKMVYEDTLTLFPFSGETVFMSMENPGLWILGCHNSDFRNRGMTALLKVSSCDKNTGDYYEDSYEDISAYLLSKNNAIEPRSFSQNSRHPSTRQKQFNATTIPENDIEKTDPWFAHRTPMPKIQNVSSSDLLMLLRQSPTPHGLSLSDLQEAKYETFSDDPSPGAIDSNNSLSEMTHFRPQLHHSGDMVFTPESGLQLRLNEKLGTTAATELKKLDFKVSSTSNNLISTIPSDNLAAGTDNTSSLGPPSMPVHYDSQLDTTLFGKKSSPLTESGGPLSLSEENNDSKLLESGLMNSQESSWGKNVSSEITRTTLQSDQEEIDYDDTISVEMKKEDFDIYDEDENQSPRSFQKKTRHYFIAAVERLWDYGMSSSPHVLRNRAQSGSVPQFKKVVFQEFTDGSFTQPLYRGELNEHLGLLGPYIRAEVEDNIMVTFRNQASRPYSFYSSLISYEEDQRQGAEPRKNFVKPNETKTYFWKVQHHMAPTKDEFDCKAWAYFSDVDLEKDVHSGLIGPLLVCHTNTLNPAHGRQVTVQEFALFFTIFDETKSWYFTENMERNCRAPCNIQMEDPTFKENYRFHAINGYIMDTLPGLVMAQDQRIRWYLLSMGSNENIHSIHFSGHVFTVRKKEEYKMALYNLYPGVFETVEMLPSKAGIWRVECLIGEHLHAGMSTLFLVYSNKCQTPLGMASGHIRDFQITASGQYGQWAPKLARLHYSGSINAWSTKEPFSWIKVDLLAPMIIHGIKTQGARQKFSSLYISQFIIMYSLDGKKWQTYRGNSTGTLMVFFGNVDSSGIKHNIFNPPIIARYIRLHPTHYSIRSTLRMELMGCDLNSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNSLDPPLLTRYLRIHPQSWVHQIALRMEVLGCEAQDLYDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 106 - protein, 5 aa, synthetic construct
  REGION 1..5: note = sortase recognition motif
  VAR_SEQ 3: note = Xaa can be any naturally occurring amino acid
LPXTG

SEQ ID NO: 107 - protein, 10 aa, synthetic construct
  REGION 1..10: note = cysteine containing peptide
GGGSGCGGGS

SEQ ID NO: 108 - DNA, 4548 bp, synthetic construct
  misc_feature 1..4548: note = VWF 031
  source 1..4548: mol_type = other DNA
atgattcctgccagatttgccggggtgctgcttgctctggccctcattttgccagggaccctttgtgcagaaggaactcgcggcaggtcatccacggcccgatgcagccttttcggaagtgacttcgtcaacacctttgatgggagcatgtacagctttgcgggatactgcagttacctcctggcagggggctgccagaaacgctccttctcgattattggggacttccagaatggcaagagagtgagcctctccgtgtatcttggggaattttttgacatccatttgtttgtcaatggtaccgtgacacagggggaccaaagagtctccatgccctatgcctccaaagggctgtatctagaaactgaggctgggtactacaagctgtccggtgaggcctatggctttgtggccaggatcgatggcagcggcaactttcaagtcctgctgtcagacagatacttcaacaagacctgcgggctgtgtggcaactttaacatctttgctgaagatgactttatgacccaagaagggaccttgacctcggacccttatgactttgccaactcatgggctctgagcagtggagaacagtggtgtgaacgggcatctcctcccagcagctcatgcaacatctcctctggggaaatgcagaagggcctgtgggagcagtgccagcttctgaagagcacctcggtgtttgcccgctgccaccctctggtggaccccgagccttttgtggccctgtgtgagaagactttgtgtgagtgtgctggggggctggagtgcgcctgccctgccctcctggagtacgcccggacctgtgcccaggagggaatggtgctgtacggctggaccgaccacagcgcgtgcagcccagtgtgccctgctggtatggagtataggcagtgtgtgtccccttgcgccaggacctgccagagcctgcacatcaatgaaatgtgtcaggagcgatgcgtggatggctgcagctgccctgagggacagctcctggatgaaggcctctgcgtggagagcaccgagtgtccctgcgtgcattccggaaagcgctaccctcccggcacctccctctctcgagactgcaacacctgcatttgccgaaacagccagtggatctgcagcaatgaagaatgtccaggggagtgccttgtcactggtcaatcccacttcaagagctttgacaacagatacttcaccttcagtgggatctgccagtacctgctggcccgggattgccaggaccactccttctccattgtcattgagactgtccagtgtgctgatgaccgcgacgctgtgtgcacccgctccgtcaccgtccggctgcctggcctgcacaacagccttgtgaaactgaagcatggggcaggagttgccatggatggccaggacatccagctccccctcctgaaaggtgacctccgcatccagcatacagtgacggcctccgtgcgcctcagctacggggaggacctgcagatggactgggatggccgcgggaggctgctggtgaagctgtcccccgtctatgccgggaagacctgcggcctgtgtgggaattacaatggcaaccagggcgacgacttccttaccccctctgggctggcggagccccgggtggaggacttcgggaacgcctggaagctgcacggggactgccaggacctgcagaagcagcacagcgatccctgcgccctcaacccgcgcatgaccaggttctccgaggaggcgtgcgcggtcctgacgtcccccacattcgaggcctgccatcgtgccgtcagcccgctgccctacctgcggaactgccgctacgacgtgtgctcctgctcggacggccgcgagtgcctgtgcggcgccctggccagctatgccgcggcctgcgcggggagaggcgtgcgcgtcgcgtggcgcgagccaggccgctgtgagctgaactgcccgaaaggccaggtgtacctgcagtgcgggaccccctgcaacctgacctgccgctctctctcttacccggatgaggaatgcaatgaggcctgcctggagggctgcttctgccccccagggctctacatggatgagaggggggactgcgtgcccaaggcccagtgcccctgttactatgacggtgagatcttccagccagaagacatcttctcagaccatcacaccatgtgctactgtgaggatggcttcatgcactgtaccatgagtggagtccccggaagcttgctgcctgacgctgtcctcagcagtcccctgtctcatcgcagcaaaaggagcctatcctgtcggccccccatggtcaagctggtgtgtcccgctgacaacctgcgggctgaagggctcgagtgtaccaaaacgtgccagaactatgacctggagtgcatgagcatgggctgtgtctctggctgcctctgccccccgggcatggtccggcatgagaacagatgtgtggccctggaaaggtgtccctgcttccatcagggcaaggagtatgcccctggagaaacagtgaagattggctgcaacacttgtgtctgtcgggaccggaagtggaactgcacagaccatgtgtgtgatgccacgtgctccacgatcggcatggcccactacctcaccttcgacgggctcaaatacctgttccccggggagtgccagtacgttctggtgcaggattactgcggcagtaaccctgggacctttcggatcctagtggggaataagggatgcagccacccctcagtgaaatgcaagaaacgggtcaccatcctggtggagggaggagagattgagctgtttgacggggaggtgaatgtgaagaggcccatgaaggatgagactcactttgaggtggtggagtctggccggtacatcattctgctgctgggcaaagccctctccgtggtctgggaccgccacctgagcatctccgtggtcctgaagcagacataccaggagaaagtgtgtggcctgtgtgggaattttgatggcatccagaacaatgacctcaccagcagcaacctccaagtggaggaagaccctgtggactttgggaactcctggaaagtgagctcgcagtgtgctgacaccagaaaagtgcctctggactcatcccctgccacctgccataacaacatcatgaagcagacgatggtggattcctcctgtagaatccttaccagtgacgtcttccaggactgcaacaagctggtggaccccgagccatatctggatgtctgcatttacgacacctgctcctgtgagtccattggggactgcgccgcattctgcgacaccattgctgcctatgcccacgtgtgtgcccagcatggcaaggtggtgacctggaggacggccacattgtgcccccagagctgcgaggagaggaatctccgggagaacgggtatgaggctgagtggcgctataacagctgtgcacctgcctgtcaagtcacgtgtcagcaccctgagccactggcctgccctgtgcagtgtgtggagggctgccatgcccactgccctccagggaaaatcctggatgagcttttgcagacctgcgttgaccctgaagactgtccagtgtgtgaggtggctggccggcgttttgcctcaggaaagaaagtcaccttgaatcccagtgaccctgagcactgccagatttgccactgtgatgttgtcaacctcacctgtgaagcctgccaggagccgatatctggcggtggaggttccggtggcgggggatccggcggtggaggttccggcggtggaggttccggtggcgggggatccggtggcgggggatccctggtcccccggggcagcggcggtggaggttccggtggcgggggatccgacaaaactcacacatgcccaccgtgcccagctccagaactcctgggcggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgttggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 109 - protein, 1515 aa, synthetic construct
  REGION 1..1515: note = VWF 031
MIPARFAGVLLALALILPGTLCAEGTRGRSSTARCSLFGSDFVNTFDGSMYSFAGYCSYLLAGGCQKRSFSIIGDFQNGKRVSLSVYLGEFFDIHLFVNGTVTQGDQRVSMPYASKGLYLETEAGYYKLSGEAYGFVARIDGSGNFQVLLSDRYFNKTCGLCGNFNIFAEDDFMTQEGTLTSDPYDFANSWALSSGEQWCERASPPSSSCNISSGEMQKGLWEQCQLLKSTSVFARCHPLVDPEPFVALCEKTLCECAGGLECACPALLEYARTCAQEGMVLYGWTDHSACSPVCPAGMEYRQCVSPCARTCQSLHINEMCQERCVDGCSCPEGQLLDEGLCVESTECPCVHSGKRYPPGTSLSRDCNTCICRNSQWICSNEECPGECLVTGQSHFKSFDNRYFTFSGICQYLLARDCQDHSFSIVIETVQCADDRDAVCTRSVTVRLPGLHNSLVKLKHGAGVAMDGQDIQLPLLKGDLRIQHTVTASVRLSYGEDLQMDWDGRGRLLVKLSPVYAGKTCGLCGNYNGNQGDDFLTPSGLAEPRVEDFGNAWKLHGDCQDLQKQHSDPCALNPRMTRFSEEACAVLTSPTFEACHRAVSPLPYLRNCRYDVCSCSDGRECLCGALASYAAACAGRGVRVAWREPGRCELNCPKGQVYLQCGTPCNLTCRSLSYPDEECNEACLEGCFCPPGLYMDERGDCVPKAQCPCYYDGEIFQPEDIFSDHHTMCYCEDGFMHCTMSGVPGSLLPDAVLSSPLSHRSKRSLSCRPPMVKLVCPADNLRAEGLECTKTCQNYDLECMSMGCVSGCLCPPGMVRHENRCVALERCPCFHQGKEYAPGETVKIGCNTCVCRDRKWNCTDHVCDATCSTIGMAHYLTFDGLKYLFPGECQYVLVQDYCGSNPGTFRILVGNKGCSHPSVKCKKRVTILVEGGEIELFDGEVNVKRPMKDETHFEVVESGRYIILLLGKALSVVWDRHLSISVVLKQTYQEKVCGLCGNFDGIQNNDLTSSNLQVEEDPVDFGNSWKVSSQCADTRKVPLDSSPATCHNNIMKQTMVDSSCRILTSDVFQDCNKLVDPEPYLDVCIYDTCSCESIGDCAAFCDTIAAYAHVCAQHGKVVTWRTATLCPQSCEERNLRENGYEAEWRYNSCAPACQVTCQHPEPLACPVQCVEGCHAHCPPGKILDELLQTCVDPEDCPVCEVAGRRFASGKKVTLNPSDPEHCQICHCDVVNLTCEACQEPISGGGGSGGGGSGGGGSGGGGSGGGGSGGGGSLVPRGSGGGGSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 110 - protein, 40 aa, synthetic construct
  REGION 1..40: note = linker
  REPEAT 1..5: note = Gly-Gly-Gly-Gly-Ser repeat
GGGGSGGGGSGGGGSGGGGSGGGGSGGGGSGGGGSGGGGS

SEQ ID NO: 111 - DNA, 62 bp, synthetic construct
  misc_feature 1..62: note = ESC54-VWF forward with BsiW1 site
  source 1..62: mol_type = other DNA
cgcttcgcgacgtacggccgccaccatgattcctgccagatttgccggggtgctgcttgctc

SEQ ID NO: 112 - DNA, 60 bp, synthetic construct
  misc_feature 1..60: note = ESC 124 - D1D2 cloning oligo with Not1 site-reverse
  source 1..60: mol_type = other DNA
ctagactcgagcggccgctcaccttttgctgcgatgagacaggggactgctgaggacagc

SEQ ID NO: 113 - DNA, 2289 bp, synthetic construct
  misc_feature 1..2289: note = VWF 053 (VWF D1D2-propeptide)
  source 1..2289: mol_type = other DNA
atgattcctgccagatttgccggggtgctgcttgctctggccctcattttgccagggaccctttgtgcagaaggaactcgcggcaggtcatccacggcccgatgcagccttttcggaagtgacttcgtcaacacctttgatgggagcatgtacagctttgcgggatactgcagttacctcctggcagggggctgccagaaacgctccttctcgattattggggacttccagaatggcaagagagtgagcctctccgtgtatcttggggaattttttgacatccatttgtttgtcaatggtaccgtgacacagggggaccaaagagtctccatgccctatgcctccaaagggctgtatctagaaactgaggctgggtactacaagctgtccggtgaggcctatggctttgtggccaggatcgatggcagcggcaactttcaagtcctgctgtcagacagatacttcaacaagacctgcgggctgtgtggcaactttaacatctttgctgaagatgactttatgacccaagaagggaccttgacctcggacccttatgactttgccaactcatgggctctgagcagtggagaacagtggtgtgaacgggcatctcctcccagcagctcatgcaacatctcctctggggaaatgcagaagggcctgtgggagcagtgccagcttctgaagagcacctcggtgtttgcccgctgccaccctctggtggaccccgagccttttgtggccctgtgtgagaagactttgtgtgagtgtgctggggggctggagtgcgcctgccctgccctcctggagtacgcccggacctgtgcccaggagggaatggtgctgtacggctggaccgaccacagcgcgtgcagcccagtgtgccctgctggtatggagtataggcagtgtgtgtccccttgcgccaggacctgccagagcctgcacatcaatgaaatgtgtcaggagcgatgcgtggatggctgcagctgccctgagggacagctcctggatgaaggcctctgcgtggagagcaccgagtgtccctgcgtgcattccggaaagcgctaccctcccggcacctccctctctcgagactgcaacacctgcatttgccgaaacagccagtggatctgcagcaatgaagaatgtccaggggagtgccttgtcactggtcaatcccacttcaagagctttgacaacagatacttcaccttcagtgggatctgccagtacctgctggcccgggattgccaggaccactccttctccattgtcattgagactgtccagtgtgctgatgaccgcgacgctgtgtgcacccgctccgtcaccgtccggctgcctggcctgcacaacagccttgtgaaactgaagcatggggcaggagttgccatggatggccaggacatccagctccccctcctgaaaggtgacctccgcatccagcatacagtgacggcctccgtgcgcctcagctacggggaggacctgcagatggactgggatggccgcgggaggctgctggtgaagctgtcccccgtctatgccgggaagacctgcggcctgtgtgggaattacaatggcaaccagggcgacgacttccttaccccctctgggctggcggagccccgggtggaggacttcgggaacgcctggaagctgcacggggactgccaggacctgcagaagcagcacagcgatccctgcgccctcaacccgcgcatgaccaggttctccgaggaggcgtgcgcggtcctgacgtcccccacattcgaggcctgccatcgtgccgtcagcccgctgccctacctgcggaactgccgctacgacgtgtgctcctgctcggacggccgcgagtgcctgtgcggcgccctggccagctatgccgcggcctgcgcggggagaggcgtgcgcgtcgcgtggcgcgagccaggccgctgtgagctgaactgcccgaaaggccaggtgtacctgcagtgcgggaccccctgcaacctgacctgccgctctctctcttacccggatgaggaatgcaatgaggcctgcctggagggctgcttctgccccccagggctctacatggatgagaggggggactgcgtgcccaaggcccagtgcccctgttactatgacggtgagatcttccagccagaagacatcttctcagaccatcacaccatgtgctactgtgaggatggcttcatgcactgtaccatgagtggagtccccggaagcttgctgcctgacgctgtcctcagcagtcccctgtctcatcgcagcaaaagg

SEQ ID NO: 114 - protein, 763 aa, synthetic construct
  REGION 1..763: note = VWF 053 (VWF D1D2-Propeptide)
MIPARFAGVLLALALILPGTLCAEGTRGRSSTARCSLFGSDFVNTFDGSMYSFAGYCSYLLAGGCQKRSFSIIGDFQNGKRVSLSVYLGEFFDIHLFVNGTVTQGDQRVSMPYASKGLYLETEAGYYKLSGEAYGFVARIDGSGNFQVLLSDRYFNKTCGLCGNFNIFAEDDFMTQEGTLTSDPYDFANSWALSSGEQWCERASPPSSSCNISSGEMQKGLWEQCQLLKSTSVFARCHPLVDPEPFVALCEKTLCECAGGLECACPALLEYARTCAQEGMVLYGWTDHSACSPVCPAGMEYRQCVSPCARTCQSLHINEMCQERCVDGCSCPEGQLLDEGLCVESTECPCVHSGKRYPPGTSLSRDCNTCICRNSQWICSNEECPGECLVTGQSHFKSFDNRYFTFSGICQYLLARDCQDHSFSIVIETVQCADDRDAVCTRSVTVRLPGLHNSLVKLKHGAGVAMDGQDIQLPLLKGDLRIQHTVTASVRLSYGEDLQMDWDGRGRLLVKLSPVYAGKTCGLCGNYNGNQGDDFLTPSGLAEPRVEDFGNAWKLHGDCQDLQKQHSDPCALNPRMTRFSEEACAVLTSPTFEACHRAVSPLPYLRNCRYDVCSCSDGRECLCGALASYAAACAGRGVRVAWREPGRCELNCPKGQVYLQCGTPCNLTCRSLSYPDEECNEACLEGCFCPPGLYMDERGDCVPKAQCPCYYDGEIFQPEDIFSDHHTMCYCEDGFMHCTMSGVPGSLLPDAVLSSPLSHRSKR